(19) United States
(12) Patent Application Publication
(10) Pub. No.: US 2025/0250286 A1
(43) Pub. Date: Aug. 7, 2025

(54) FUSED PYRIMIDINE DERIVATIVES AS KRAS ONCOPROTEIN INHIBITORS

(71) Applicant: BETA PHARMA, INC., Wilmington, DE (US)

(72) Inventors: Don Zhang, Princeton, NJ (US); Jirong Peng, Mequon, WI (US); Michael John Costanzo, Bonney Lake, WA (US); Michael Alan Green, Easton, PA (US); Michael Nicholas Greco, Lansdale, PA (US); Stephen Bolgunas, Swedesboro, NJ (US)

(21) Appl. No.: 18/705,262

(22) PCT Filed: Nov. 29, 2022
(86) PCT No.: PCT/US2022/051157

§ 371 Date: Apr. 26, 2024

Related U.S. Application Data
(60) Provisional application No. 63/284,067, filed on Nov. 30, 2021.
(60) Provisional application No. 63/350,987, filed on Jun. 10, 2022.

(51) Int. Cl. C07D 519/00; A61K 31/519; A61K 31/5377; A61K 45/06

(52) U.S. Cl. CPC C07D 519/00; A61K 31/519; A61K 31/5377; A61K 45/06

(57) ABSTRACT

The present invention is directed to certain pyridopyrimidine and pyrimido[4,5-d]pyrimidine inhibitors of Kirsten rat sarcoma virus (KRAS) oncoproteins, and more particularly to compounds of Formula I as well as compositions comprising Formula I and methods of using the compound of Formula I for the treatment or prevention of a disease, disorder, or medical condition mediated through KRAS, especially the KRAS G12D oncoprotein.

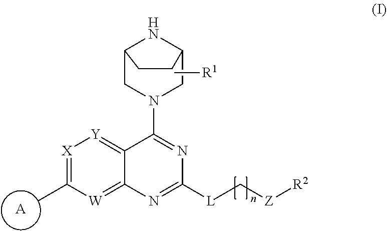

### CROSS-REFERENCE TO RELATED APPLICATIONS

This application claims the benefit of U.S. Provisional Application Ser. No. 63/284,067 filed 30 Nov. 2021 and U.S. Provisional Application Ser. No. 63/350,987 filed 10 Jun. 2022, both of which are incorporated by reference herein in their entireties.

### BACKGROUND OF THE INVENTION

### Field of the Invention

The present invention is directed to inhibitors of Kirsten rat sarcoma virus (KRAS) oncoproteins, and more particularly to certain pyridopyrimidine and pyrimido[4,5-d]pyrimidine compounds, compositions and methods for the treatment or prevention of a disease, disorder, or medical condition mediated through KRAS, especially the KRAS G12D oncoprotein. The diseases include various cancers.

### Brief Description of the Related Art

Ras is a superfamily of small guanosine triphosphate (GTP) binding proteins consisting of various isoforms. Ras genes can mutate to oncogenes that are associated with numerous cancers such as lung, pancreas, and colon. Ras is one of the most frequently mutated oncogenes. KRAS, (Kirsten rat sarcoma virus) an isoform of Ras, is one of the most frequently mutated Ras genes, comprising approximately 86% of all known mutations. KRAS functions as an on/off switch in cell signaling. KRAS proteins are GTPases that operate between inactive (GDP-bound) and active (GTP-bound) states to control a variety of functions, including cell proliferation. However, mutated KRAS proteins lead to uncontrolled cell proliferation and cancer. The KRAS-4B proteoform is the major isoform in cancers of the colon (30-40%), lung (15-20%) and pancreas (90%) (Liu, P. et al., Acta Pharmaceutica Sinica B 2019, 9 (5), 871-879). Consequently, inhibitors of mutated KRAS proteins binding to GTP represent potential therapeutic agents for the treatment of various cancers.

Past attempts to design KRAS oncoprotein inhibitors have been mostly unsuccessful, due in large part to the high affinity of the KRAS oncoproteins for GTP. However, more recent approaches that target KRAS G12C have shown promise. This mutation exists in roughly 50% of lung cancers and approximately 10-20% of all KRAS G12 mutations. The cysteine residue of the mutation is positioned within the active site such that the sulfhydryl functionality can form a covalent bond with a suitably functionalized bound ligand (Liu, Acta Pharmaceutica Sinica B 2019). This approach has identified irreversible, covalent inhibitors of KRAS G12C that are undergoing clinical study. The KRAS G12D mutation is present in approximately 4% of all non-small cell lung cancers, 13% of all colorectal cancers, 25% of pancreatic ductal adenocarcinomas, and 1.7% of small cell lung cancers (Cerami, E. and Sawyers, C. L. Cancer Discovery 2017, 7 (8), 818-831). Given the prominent role that the KRAS G12D plays as a driver of many malignancies, a need for new KRAS G12D inhibitors with improved selectivity, safety, and efficacy exists.

### SUMMARY OF THE INVENTION

In one aspect, the present invention is directed to a compound of Formula I:

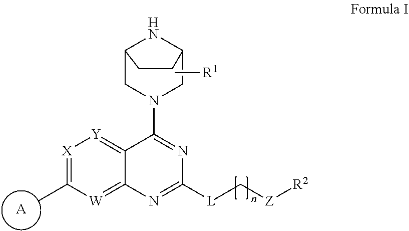

or a pharmaceutically acceptable salt, solvate, or prodrug thereof, wherein
A is chosen from aryl or heteroaryl optionally substituted with one or more of hydrogen, halogen, hydroxy, —C₁₋₆ alkyl, —C₂₋₆ alkenyl, —C₂₋₆ alkynyl, —C₀₋₃ alkyl(C₃₋₆ cycloalkyl), —C₁₋₆ alkyl(halo), —C₁₋₆ alkyl(OH), —O(C₁₋₄ alkyl), —C₁₋₃ alkyl(C₁₋₄ alkoxy), —CN, —CO₂R³,
   —CO₂N(R³)₂, —NO₂, —N(R³)₂, —P(O)(R⁴)₂, —SR³, —S(O)R³, —SO₂R³ or a 5-6 membered heterocyclic ring;
W is chosen from CR⁵ or N with the proviso that if W is N, then Y is CR⁵;
X is chosen from CR⁵ or N with the proviso that if X is N, then Y is CR⁵ and W is N;
Y is chosen from CR⁵ or N with the proviso that if Y is N, then X and W are both CR⁵;
L is a bond, O, S, or NR³;
n is 0-2;
Z is C(R³)₂ or a cyclic compound chosen from C₃₋₇ cycloalkyl, a saturated or partially unsaturated 4- to 7-membered nitrogen-containing ring, a saturated or partially unsaturated 7- to 10-membered nitrogen-containing bridged bicyclic ring;
R¹ is chosen from hydrogen, hydroxy, halogen, —C₁₋₃ alkyl, —C₁₋₃ alkyl(OH), —C₁₋₃ alkyl(halo), —C₁₋₃ alkyl(C₁₋₃ alkoxy), —C₁₋₃ alkyl(CN) or —C₁₋₃ alkyl(P(O)R³₂);
R² is chosen from hydrogen, halogen, hydroxy, —C₁₋₄ alkyl, —C₂₋₄ alkenyl, —C₂₋₄ alkynyl, —C₀₋₃ alkyl(C₃₋₆ cycloalkyl), —C₁₋₄ alkyl(halo), —C₁₋₄ alkyl(OH), —O(C₁₋₄ alkyl), —C₁₋₃ alkyl(C₁₋₃ alkoxy), —CN, —CO₂R³, —CO₂N(R³)₂, —NO₂, —N(R³)₂, —PO(R⁴)₂, —SR³, —S(O)R³, —SO₂R³, or —(C₀₋₃ alkyl)R⁶;
Each R³ is the same or different and is chosen from hydrogen, C₁₋₄ alkyl, aryl or heteroaryl;
Each R⁴ is the same or different and is chosen from hydrogen, hydroxy, C₁₋₄ alkyl, aryl, heteroaryl, C₁₋₄ alkoxy, aryloxy or heteroaryloxy;
Each R⁵ may be the same or different and chosen from hydrogen, halogen, C₁₋₄ alkyl, C₁₋₄ perdeuteroalkyl, —(C₀₋₂ alkyl)alkenyl, —(C₀₋₂ alkyl)alkynyl, —(C₀₋₂ alkyl)cycloalkyl, —C₁₋₄ haloalkyl, —O(C₁₋₄ alkyl), —S(C₁₋₄ alkyl), —(C₀₋₂ alkyl)cyano, —O(C₁₋₄ haloalkyl) or —S(C₁₋₄ haloalkyl);
R⁶ is chosen from N(R³)₂ or a 4- to 7-membered saturated or unsaturated heterocyclic ring containing one or more heteroatoms selected from the group N, O and S.

In another aspect, the present invention is directed to a pharmaceutical composition comprising a compound of Formula I, or a salt, solvate, or prodrug thereof, together with a pharmaceutically acceptable carrier.

In another aspect, the present invention is directed to a method of treating a disease, disorder, or medical condition in a patient, comprising the step of providing to a patient in need thereof a therapeutic agent, wherein the therapeutic agent comprises the compound of Formula I or a salt, solvate, or prodrug thereof.

These and other aspects will become apparent upon reading the following detailed description.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

All formulae disclosed herein include all salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The terms C₁₋₆ alkyl, C₁-C₆ alkyl and C1-C6 alkyl as used herein all indicate an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁₋₈ alkyl, C₁₋₄ alkyl, and C₁₋₂ alkyl. When C₀₋ₙ alkyl is used herein in conjunction with another group, for example, —C₀₋₄ alkyl(phenyl), the indicated group, in this case phenyl, is either directly bound by a single covalent bond (C₀ alkyl), or attached by an alkyl chain having the specified number of carbon atoms, in this case 1, 2, 3, or 4 carbon atoms. Alkyls can also be attached via other groups such as heteroatoms as in —OC₀₋₄ alkyl(C₃₋₇ cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes will be readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula I, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression, or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers (e.g., asymmetric carbon atoms), stereogenic axes, rotamers with restricted rotation (e.g., atropisomers) and the like, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, prodrugs, free base compound and salts) of the compounds of the invention may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

The term “solvate” refers to a chemical complex formed by the interaction of a solvent and a solute, such as the chemical compounds of the present invention.

The term “prodrug” refers to a biologically inactive compound which can be metabolized inside or outside the body to produce a drug.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Salts” include derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts. In an embodiment, the compounds of the present invention are synthesized or isolated as trifluoroacetic acid (TFA) salts.

In one embodiment, the salt forms of the compounds of the present invention described above may include pharmaceutically acceptable salts. Examples of pharmaceutically acceptable salts include, but are not limited to, non-toxic mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

In the preferred embodiments, the compounds of Formula I are represented by the structures 1-aa through 1-gd and 2-aa through 2-gc shown below, including pharmaceutically acceptable salts, solvates, or prodrugs thereof:

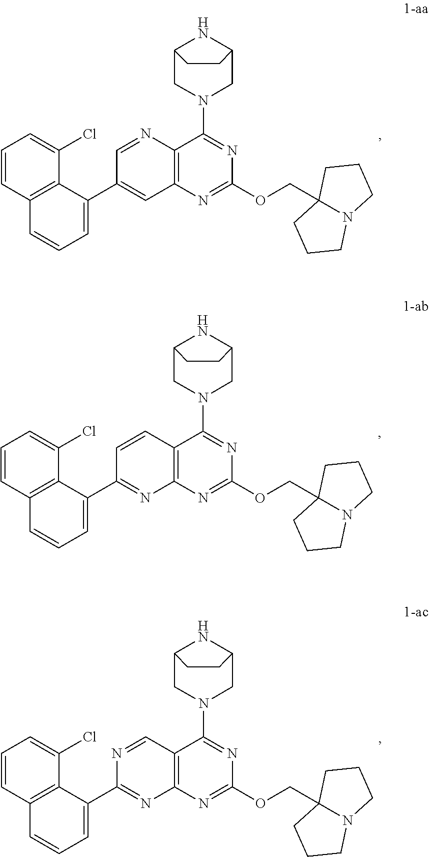 [SMILES: Clc1cccc2cccc(-c3cnc4c(N5CC6CCC(C5)N6)nc(OCC56CCCN5CCC6)nc4c3)c12 ; Clc1cccc2cccc(-c3ccc4c(N5CC6CCC(C5)N6)nc(OCC56CCCN5CCC6)nc4n3)c12 ; Clc1cccc2cccc(-c3ncc4c(N5CC6CCC(C5)N6)nc(OCC56CCCN5CCC6)nc4n3)c12]

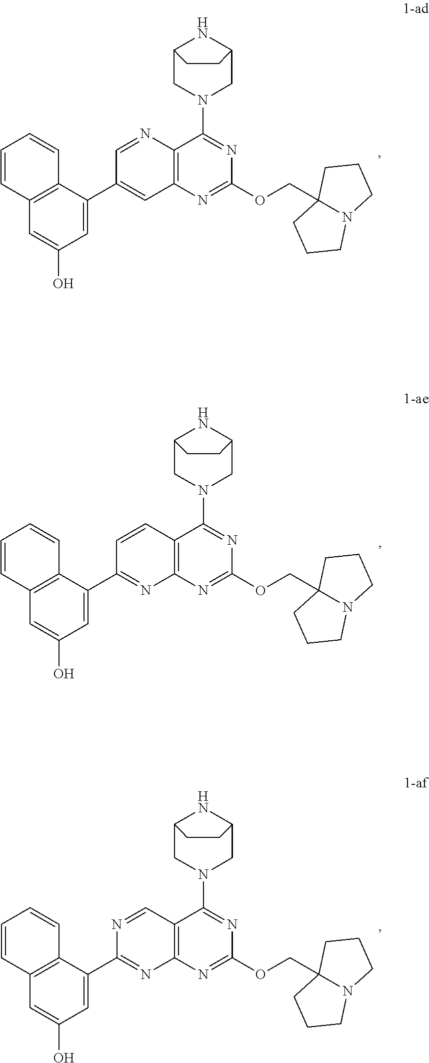 [SMILES: Oc1cc(-c2cnc3c(N4CC5CCC(C4)N5)nc(OCC45CCCN4CCC5)nc3c2)c2ccccc2c1 ; Oc1cc(-c2ccc3c(N4CC5CCC(C4)N5)nc(OCC45CCCN4CCC5)nc3n2)c2ccccc2c1 ; Oc1cc(-c2ncc3c(N4CC5CCC(C4)N5)nc(OCC45CCCN4CCC5)nc3n2)c2ccccc2c1]

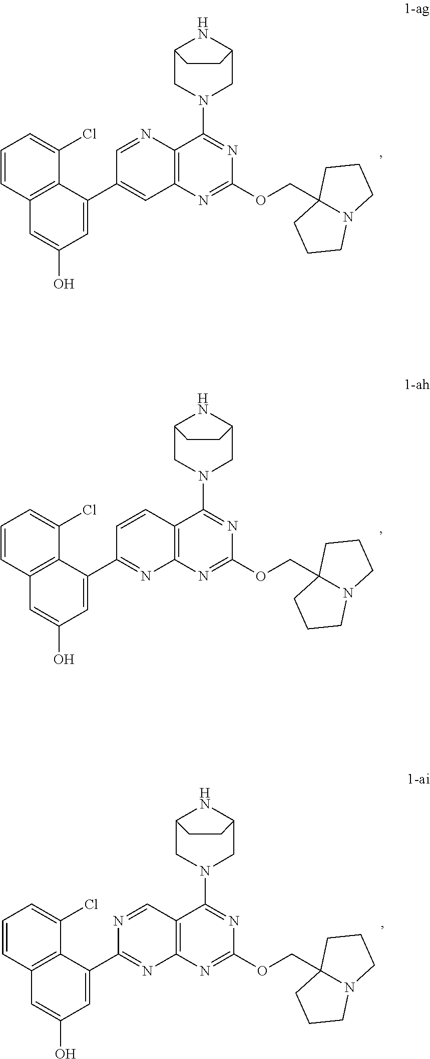 [SMILES: Oc1cc(-c2cnc3c(N4CC5CCC(C4)N5)nc(OCC45CCCN4CCC5)nc3c2)c2c(Cl)cccc2c1 ; Oc1cc(-c2ccc3c(N4CC5CCC(C4)N5)nc(OCC45CCCN4CCC5)nc3n2)c2c(Cl)cccc2c1 ; Oc1cc(-c2ncc3c(N4CC5CCC(C4)N5)nc(OCC45CCCN4CCC5)nc3n2)c2c(Cl)cccc2c1]

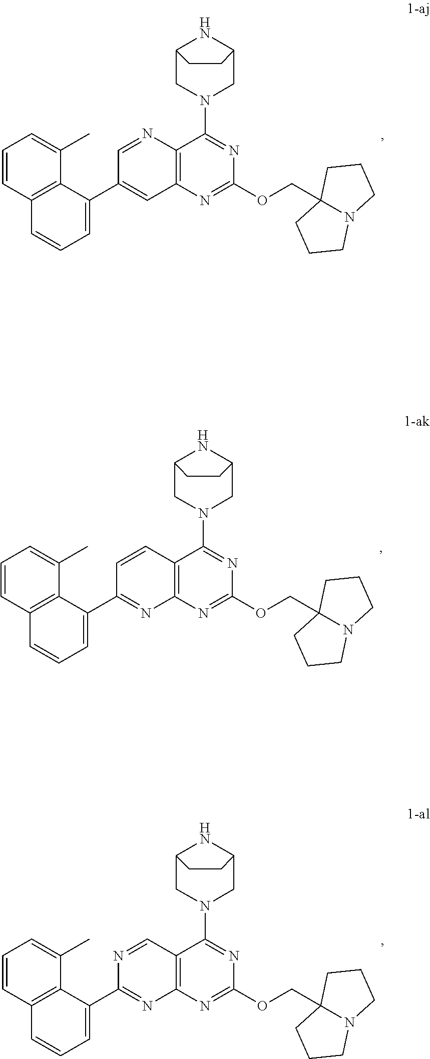 [SMILES: Cc1cccc2cccc(-c3cnc4c(N5CC6CCC(C5)N6)nc(OCC56CCCN5CCC6)nc4c3)c12 ; Cc1cccc2cccc(-c3ccc4c(N5CC6CCC(C5)N6)nc(OCC56CCCN5CCC6)nc4n3)c12 ; Cc1cccc2cccc(-c3ncc4c(N5CC6CCC(C5)N6)nc(OCC56CCCN5CCC6)nc4n3)c12]

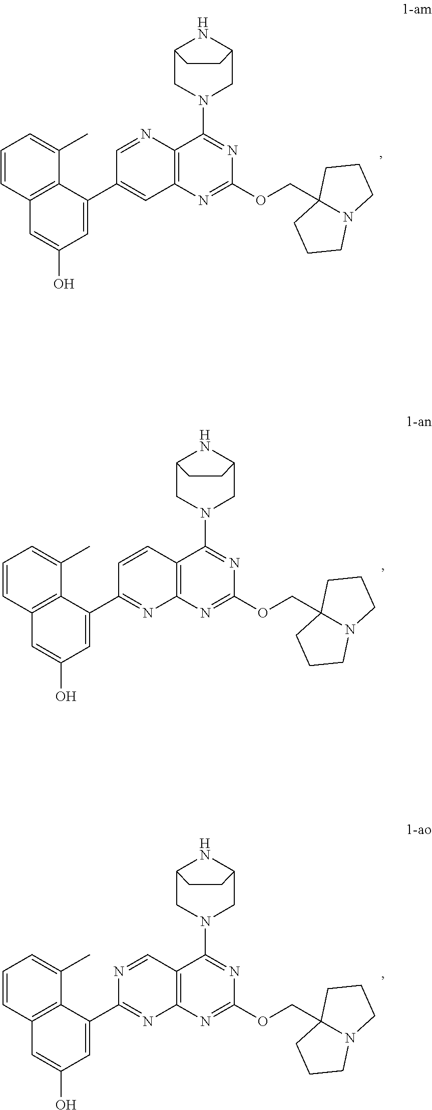 [SMILES: Cc1cccc2cc(O)cc(-c3cnc4c(N5CC6CCC(C5)N6)nc(OCC56CCCN5CCC6)nc4c3)c12 ; Cc1cccc2cc(O)cc(-c3ccc4c(N5CC6CCC(C5)N6)nc(OCC56CCCN5CCC6)nc4n3)c12 ; Cc1cccc2cc(O)cc(-c3ncc4c(N5CC6CCC(C5)N6)nc(OCC56CCCN5CCC6)nc4n3)c12]

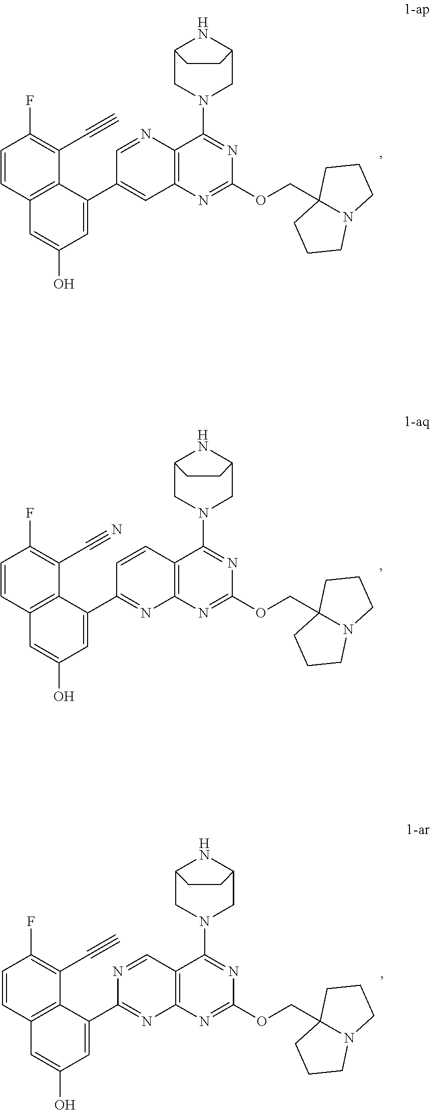 [SMILES: C#Cc1c(F)ccc2cc(O)cc(-c3cnc4c(N5CC6CCC(C5)N6)nc(OCC56CCCN5CCC6)nc4c3)c12 ; N#Cc1c(F)ccc2cc(O)cc(-c3ccc4c(N5CC6CCC(C5)N6)nc(OCC56CCCN5CCC6)nc4n3)c12 ; C#Cc1c(F)ccc2cc(O)cc(-c3ncc4c(N5CC6CCC(C5)N6)nc(OCC56CCCN5CCC6)nc4n3)c12]

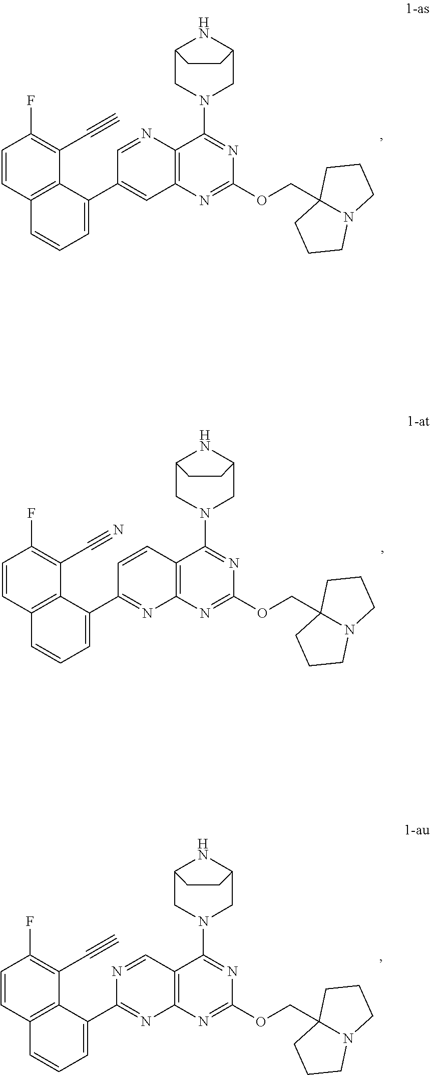 [SMILES: C#Cc1c(F)ccc2cccc(-c3cnc4c(N5CC6CCC(C5)N6)nc(OCC56CCCN5CCC6)nc4c3)c12 ; N#Cc1c(F)ccc2cccc(-c3ccc4c(N5CC6CCC(C5)N6)nc(OCC56CCCN5CCC6)nc4n3)c12 ; C#Cc1c(F)ccc2cccc(-c3ncc4c(N5CC6CCC(C5)N6)nc(OCC56CCCN5CCC6)nc4n3)c12]

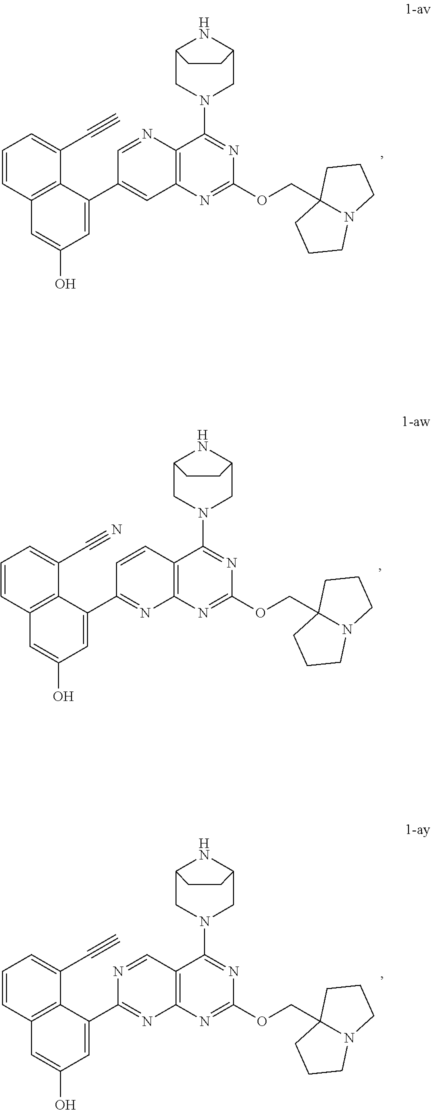 [SMILES: C#Cc1cccc2cc(O)cc(-c3cnc4c(N5CC6CCC(C5)N6)nc(OCC56CCCN5CCC6)nc4c3)c12 ; N#Cc1cccc2cc(O)cc(-c3ccc4c(N5CC6CCC(C5)N6)nc(OCC56CCCN5CCC6)nc4n3)c12 ; C#Cc1cccc2cc(O)cc(-c3ncc4c(N5CC6CCC(C5)N6)nc(OCC56CCCN5CCC6)nc4n3)c12]

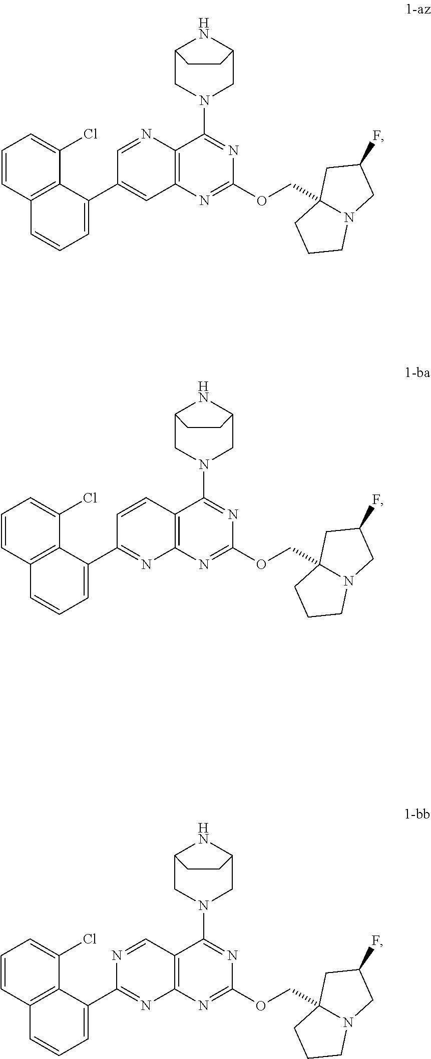 [SMILES: F[C@H]1CN2CCC[C@@]2(COc2nc(N3CC4CCC(C3)N4)c3ncc(-c4cccc5cccc(Cl)c45)cc3n2)C1 ; F[C@H]1CN2CCC[C@@]2(COc2nc(N3CC4CCC(C3)N4)c3ccc(-c4cccc5cccc(Cl)c45)nc3n2)C1 ; F[C@H]1CN2CCC[C@@]2(COc2nc(N3CC4CCC(C3)N4)c3cnc(-c4cccc5cccc(Cl)c45)nc3n2)C1]

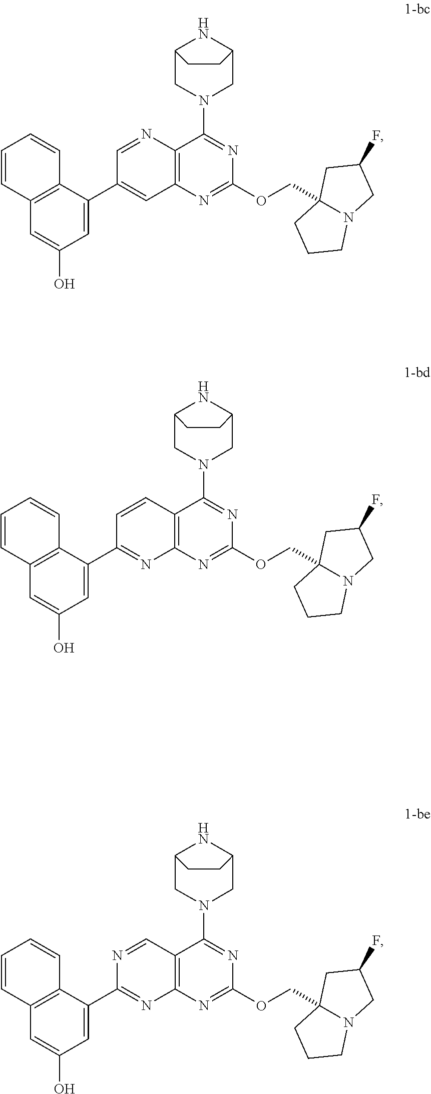 [SMILES: Oc1cc(-c2cnc3c(N4CC5CCC(C4)N5)nc(OC[C@@]45CCCN4C[C@H](F)C5)nc3c2)c2ccccc2c1 ; Oc1cc(-c2ccc3c(N4CC5CCC(C4)N5)nc(OC[C@@]45CCCN4C[C@H](F)C5)nc3n2)c2ccccc2c1 ; Oc1cc(-c2ncc3c(N4CC5CCC(C4)N5)nc(OC[C@@]45CCCN4C[C@H](F)C5)nc3n2)c2ccccc2c1]

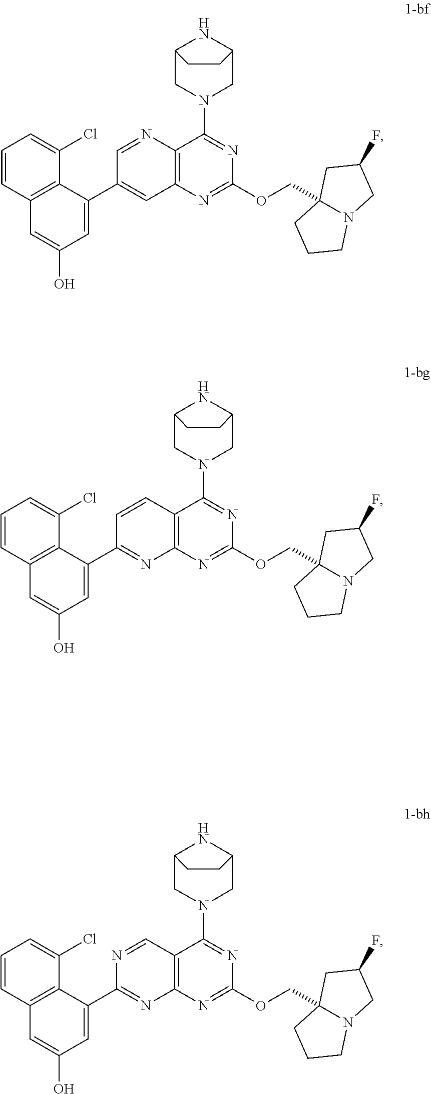 [SMILES: Oc1cc(-c2cnc3c(N4CC5CCC(C4)N5)nc(OC[C@@]45CCCN4C[C@H](F)C5)nc3c2)c2c(Cl)cccc2c1 ; Oc1cc(-c2ccc3c(N4CC5CCC(C4)N5)nc(OC[C@@]45CCCN4C[C@H](F)C5)nc3n2)c2c(Cl)cccc2c1 ; Oc1cc(-c2ncc3c(N4CC5CCC(C4)N5)nc(OC[C@@]45CCCN4C[C@H](F)C5)nc3n2)c2c(Cl)cccc2c1]

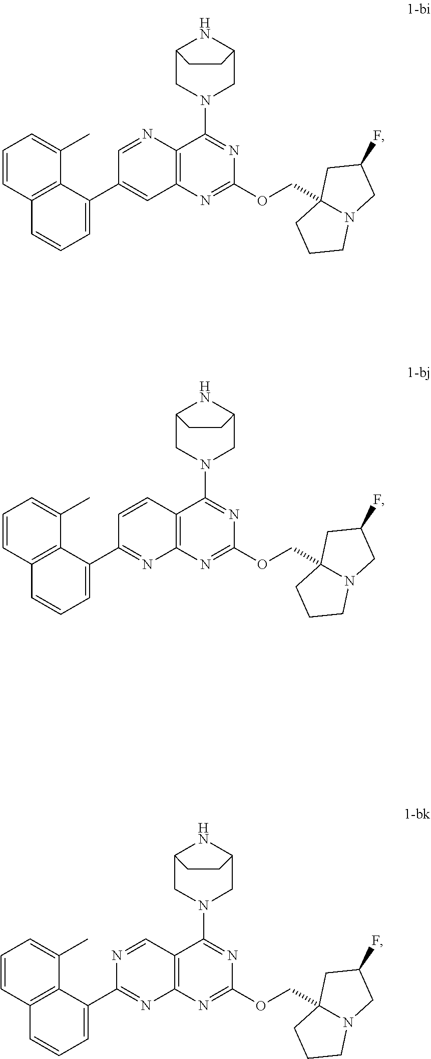 [SMILES: Cc1cccc2cccc(-c3cnc4c(N5CC6CCC(C5)N6)nc(OC[C@@]56CCCN5C[C@H](F)C6)nc4c3)c12 ; Cc1cccc2cccc(-c3ccc4c(N5CC6CCC(C5)N6)nc(OC[C@@]56CCCN5C[C@H](F)C6)nc4n3)c12 ; Cc1cccc2cccc(-c3ncc4c(N5CC6CCC(C5)N6)nc(OC[C@@]56CCCN5C[C@H](F)C6)nc4n3)c12]

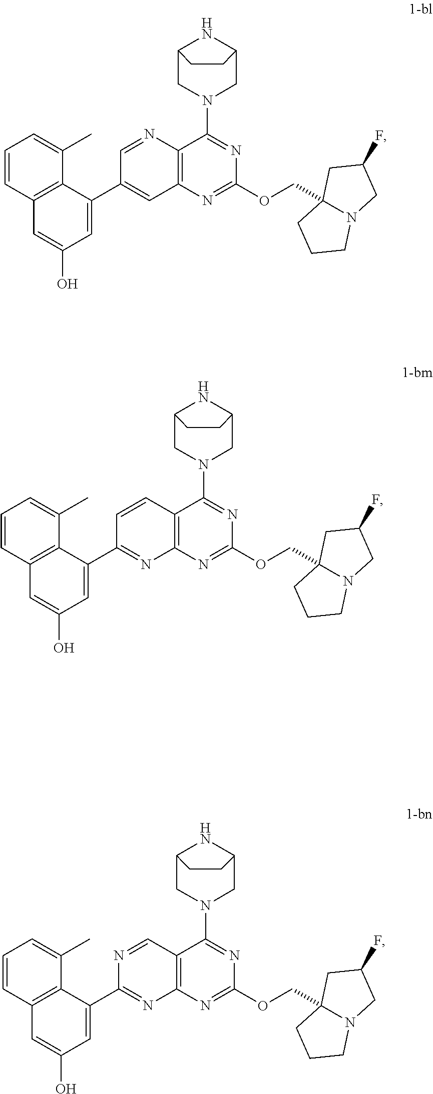 [SMILES: Cc1cccc2cc(O)cc(-c3cnc4c(N5CC6CCC(C5)N6)nc(OC[C@@]56CCCN5C[C@H](F)C6)nc4c3)c12 ; Cc1cccc2cc(O)cc(-c3ccc4c(N5CC6CCC(C5)N6)nc(OC[C@@]56CCCN5C[C@H](F)C6)nc4n3)c12 ; Cc1cccc2cc(O)cc(-c3ncc4c(N5CC6CCC(C5)N6)nc(OC[C@@]56CCCN5C[C@H](F)C6)nc4n3)c12]

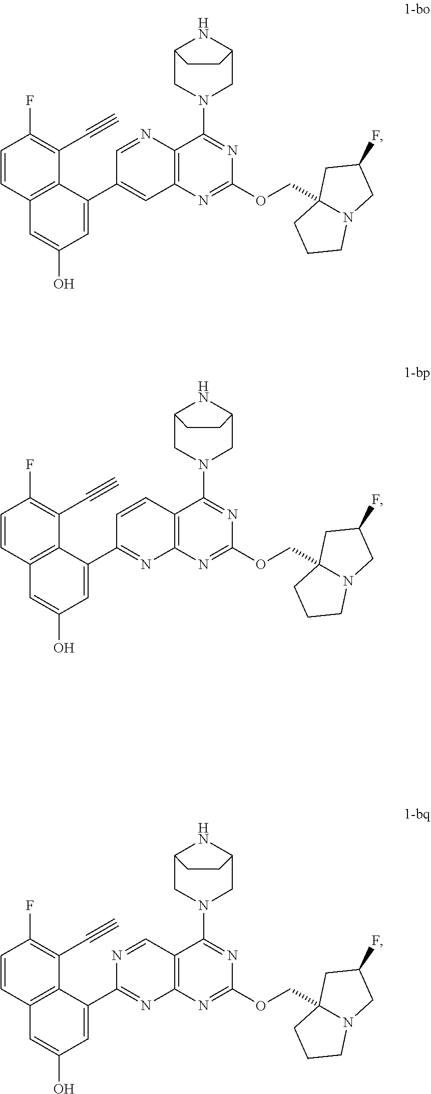 [SMILES: C#Cc1c(F)ccc2cc(O)cc(-c3cnc4c(N5CC6CCC(C5)N6)nc(OC[C@@]56CCCN5C[C@H](F)C6)nc4c3)c12 ; C#Cc1c(F)ccc2cc(O)cc(-c3ccc4c(N5CC6CCC(C5)N6)nc(OC[C@@]56CCCN5C[C@H](F)C6)nc4n3)c12 ; C#Cc1c(F)ccc2cc(O)cc(-c3ncc4c(N5CC6CCC(C5)N6)nc(OC[C@@]56CCCN5C[C@H](F)C6)nc4n3)c12]

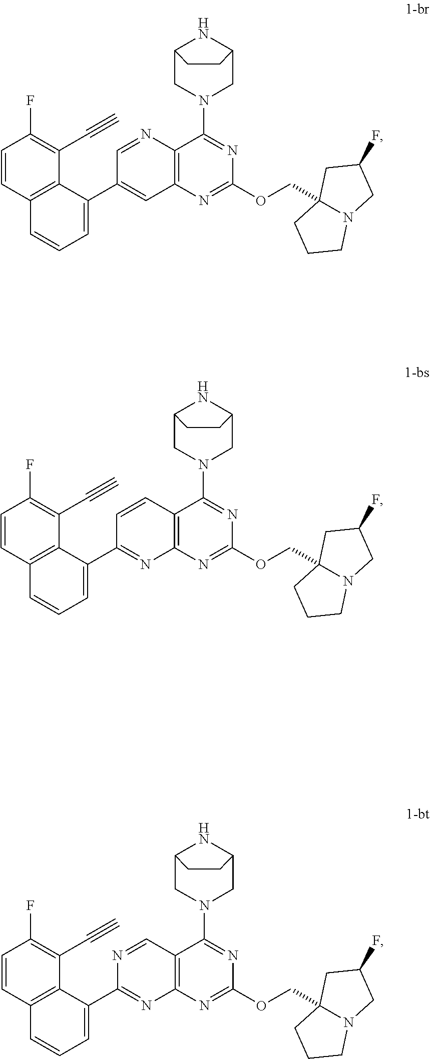 [SMILES: C#Cc1c(F)ccc2cccc(-c3cnc4c(N5CC6CCC(C5)N6)nc(OC[C@@]56CCCN5C[C@H](F)C6)nc4c3)c12 ; C#Cc1c(F)ccc2cccc(-c3ccc4c(N5CC6CCC(C5)N6)nc(OC[C@@]56CCCN5C[C@H](F)C6)nc4n3)c12 ; C#Cc1c(F)ccc2cccc(-c3ncc4c(N5CC6CCC(C5)N6)nc(OC[C@@]56CCCN5C[C@H](F)C6)nc4n3)c12]

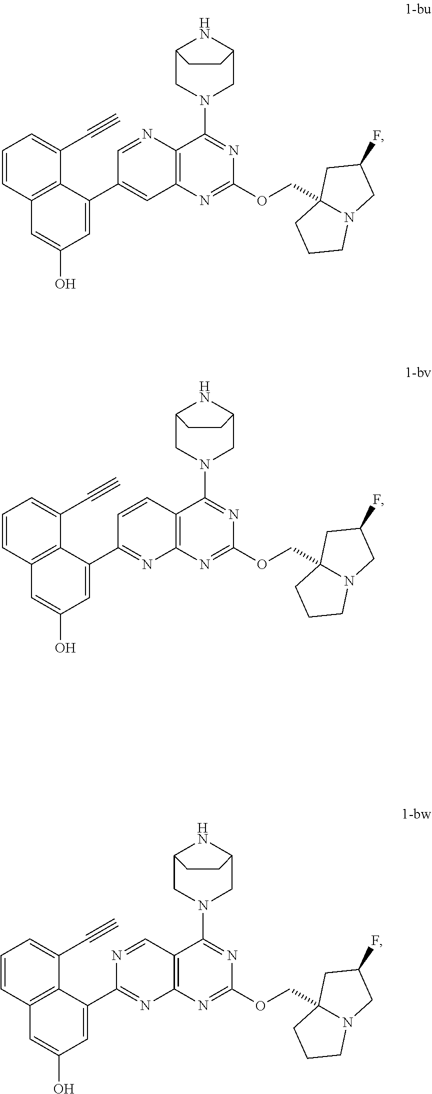 [SMILES: C#Cc1cccc2cc(O)cc(-c3cnc4c(N5CC6CCC(C5)N6)nc(OC[C@@]56CCCN5C[C@H](F)C6)nc4c3)c12 ; C#Cc1cccc2cc(O)cc(-c3ccc4c(N5CC6CCC(C5)N6)nc(OC[C@@]56CCCN5C[C@H](F)C6)nc4n3)c12 ; C#Cc1cccc2cc(O)cc(-c3ncc4c(N5CC6CCC(C5)N6)nc(OC[C@@]56CCCN5C[C@H](F)C6)nc4n3)c12]

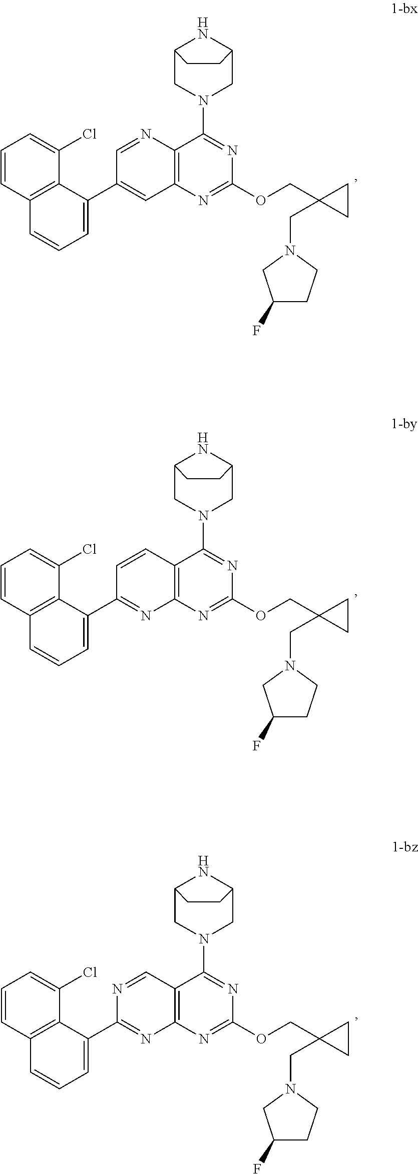 [SMILES: F[C@@H]1CCN(CC2(COc3nc(N4CC5CCC(C4)N5)c4ncc(-c5cccc6cccc(Cl)c56)cc4n3)CC2)C1 ; F[C@@H]1CCN(CC2(COc3nc(N4CC5CCC(C4)N5)c4ccc(-c5cccc6cccc(Cl)c56)nc4n3)CC2)C1 ; F[C@@H]1CCN(CC2(COc3nc(N4CC5CCC(C4)N5)c4cnc(-c5cccc6cccc(Cl)c56)nc4n3)CC2)C1]

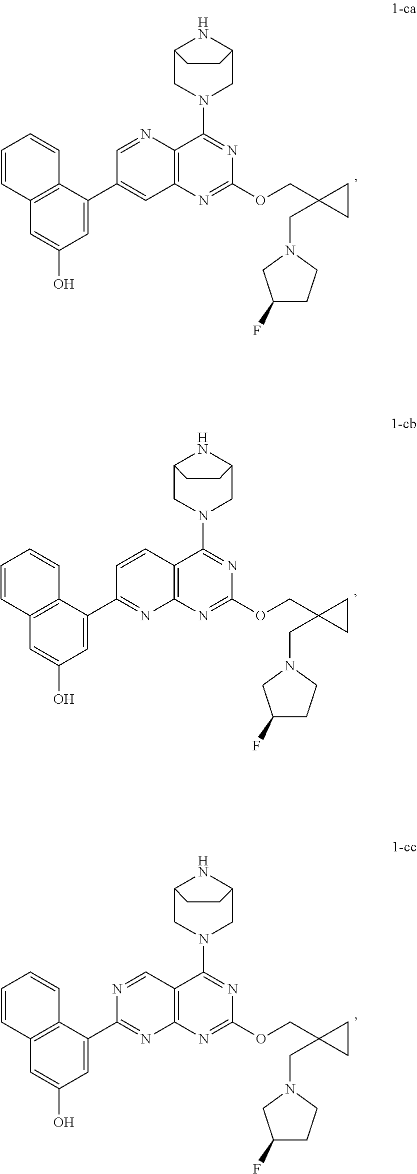 [SMILES: Oc1cc(-c2cnc3c(N4CC5CCC(C4)N5)nc(OCC4(CN5CC[C@@H](F)C5)CC4)nc3c2)c2ccccc2c1 ; Oc1cc(-c2ccc3c(N4CC5CCC(C4)N5)nc(OCC4(CN5CC[C@@H](F)C5)CC4)nc3n2)c2ccccc2c1 ; Oc1cc(-c2ncc3c(N4CC5CCC(C4)N5)nc(OCC4(CN5CC[C@@H](F)C5)CC4)nc3n2)c2ccccc2c1]

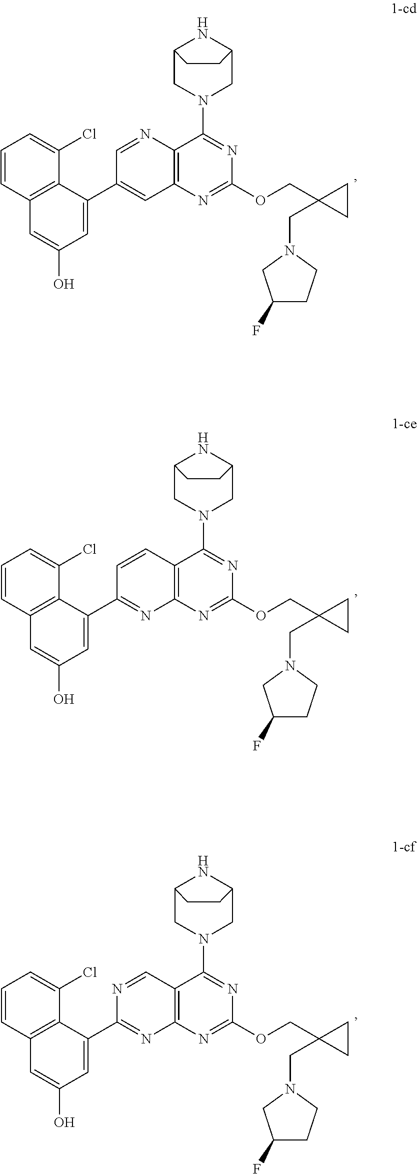 [SMILES: Oc1cc(-c2cnc3c(N4CC5CCC(C4)N5)nc(OCC4(CN5CC[C@@H](F)C5)CC4)nc3c2)c2c(Cl)cccc2c1 ; Oc1cc(-c2ccc3c(N4CC5CCC(C4)N5)nc(OCC4(CN5CC[C@@H](F)C5)CC4)nc3n2)c2c(Cl)cccc2c1 ; Oc1cc(-c2ncc3c(N4CC5CCC(C4)N5)nc(OCC4(CN5CC[C@@H](F)C5)CC4)nc3n2)c2c(Cl)cccc2c1]

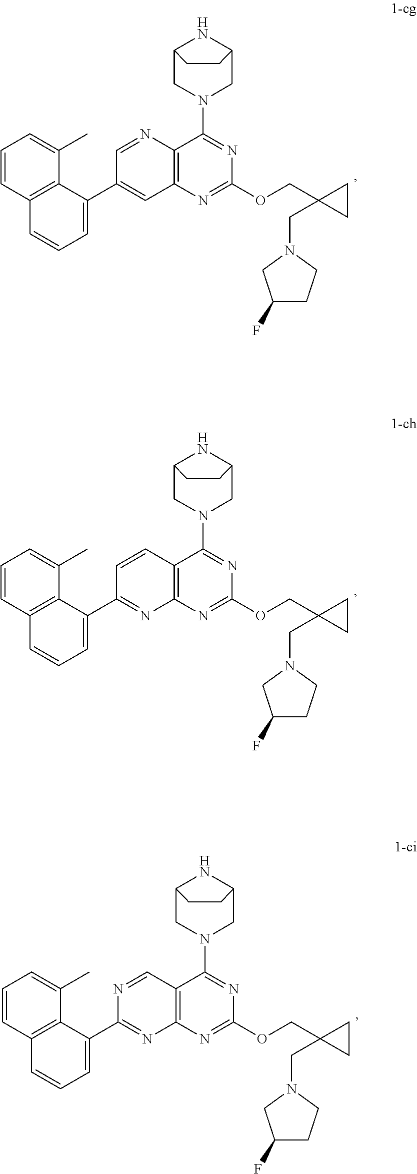 [SMILES: Cc1cccc2cccc(-c3cnc4c(N5CC6CCC(C5)N6)nc(OCC5(CN6CC[C@@H](F)C6)CC5)nc4c3)c12 ; Cc1cccc2cccc(-c3ccc4c(N5CC6CCC(C5)N6)nc(OCC5(CN6CC[C@@H](F)C6)CC5)nc4n3)c12 ; Cc1cccc2cccc(-c3ncc4c(N5CC6CCC(C5)N6)nc(OCC5(CN6CC[C@@H](F)C6)CC5)nc4n3)c12]

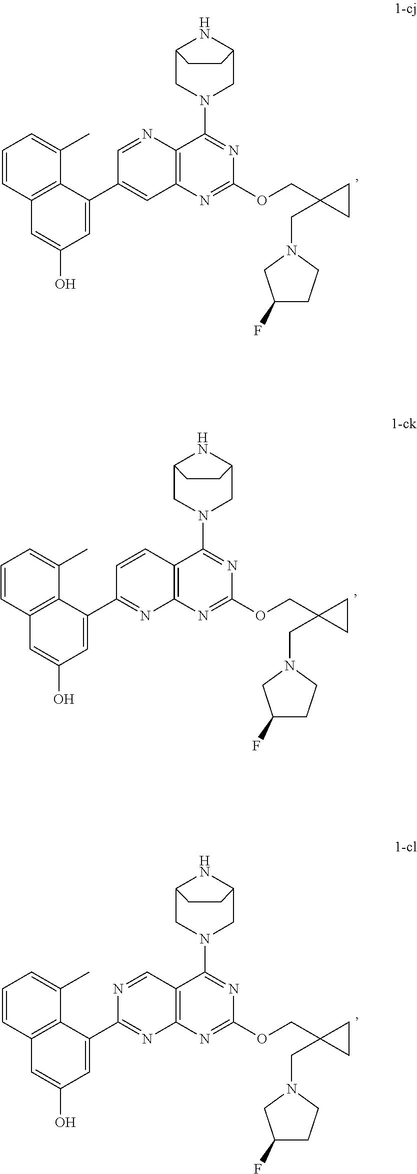 [SMILES: Cc1cccc2cc(O)cc(-c3cnc4c(N5CC6CCC(C5)N6)nc(OCC5(CN6CC[C@@H](F)C6)CC5)nc4c3)c12 ; Cc1cccc2cc(O)cc(-c3ccc4c(N5CC6CCC(C5)N6)nc(OCC5(CN6CC[C@@H](F)C6)CC5)nc4n3)c12 ; Cc1cccc2cc(O)cc(-c3ncc4c(N5CC6CCC(C5)N6)nc(OCC5(CN6CC[C@@H](F)C6)CC5)nc4n3)c12]

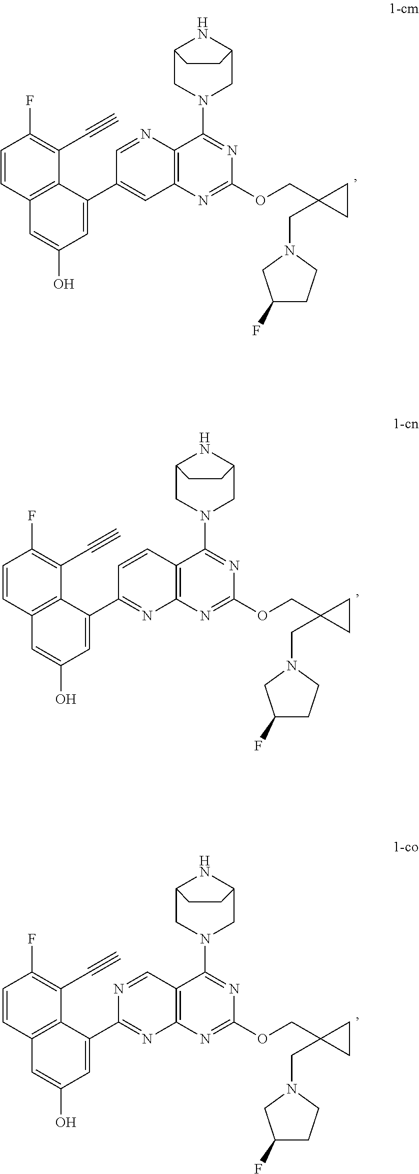 [SMILES: C#Cc1c(F)ccc2cc(O)cc(-c3cnc4c(N5CC6CCC(C5)N6)nc(OCC5(CN6CC[C@@H](F)C6)CC5)nc4c3)c12 ; C#Cc1c(F)ccc2cc(O)cc(-c3ccc4c(N5CC6CCC(C5)N6)nc(OCC5(CN6CC[C@@H](F)C6)CC5)nc4n3)c12 ; C#Cc1c(F)ccc2cc(O)cc(-c3ncc4c(N5CC6CCC(C5)N6)nc(OCC5(CN6CC[C@@H](F)C6)CC5)nc4n3)c12]

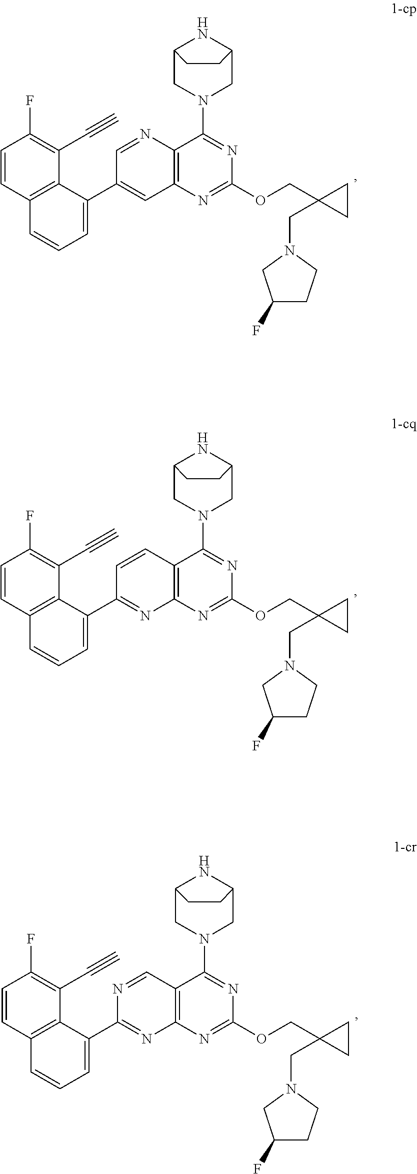 [SMILES: C#Cc1c(F)ccc2cccc(-c3cnc4c(N5CC6CCC(C5)N6)nc(OCC5(CN6CC[C@@H](F)C6)CC5)nc4c3)c12 ; C#Cc1c(F)ccc2cccc(-c3ccc4c(N5CC6CCC(C5)N6)nc(OCC5(CN6CC[C@@H](F)C6)CC5)nc4n3)c12 ; C#Cc1c(F)ccc2cccc(-c3ncc4c(N5CC6CCC(C5)N6)nc(OCC5(CN6CC[C@@H](F)C6)CC5)nc4n3)c12]

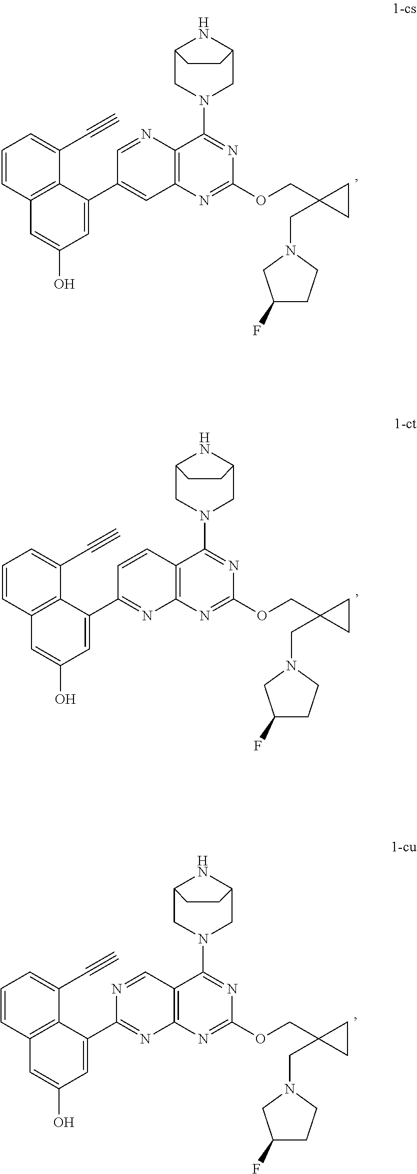 [SMILES: C#Cc1cccc2cc(O)cc(-c3cnc4c(N5CC6CCC(C5)N6)nc(OCC5(CN6CC[C@@H](F)C6)CC5)nc4c3)c12 ; C#Cc1cccc2cc(O)cc(-c3ccc4c(N5CC6CCC(C5)N6)nc(OCC5(CN6CC[C@@H](F)C6)CC5)nc4n3)c12 ; C#Cc1cccc2cc(O)cc(-c3ncc4c(N5CC6CCC(C5)N6)nc(OCC5(CN6CC[C@@H](F)C6)CC5)nc4n3)c12]

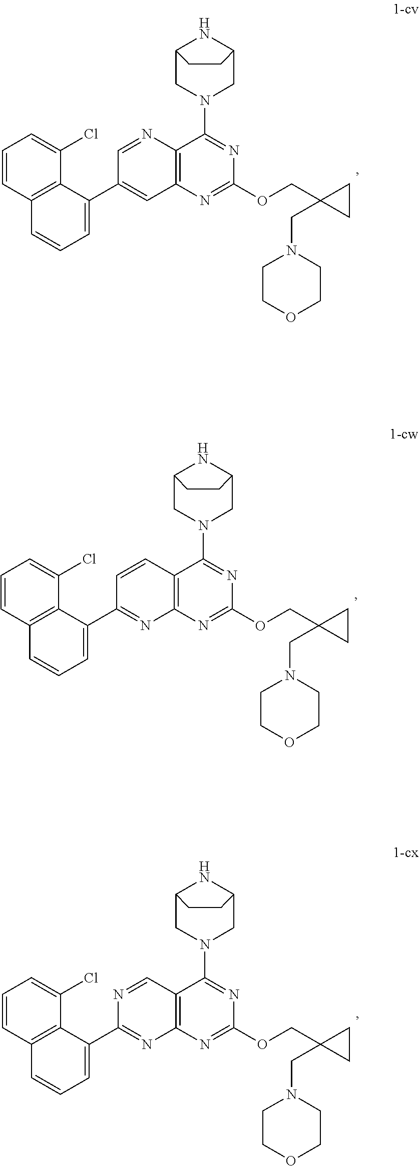 [SMILES: Clc1cccc2cccc(-c3cnc4c(N5CC6CCC(C5)N6)nc(OCC5(CN6CCOCC6)CC5)nc4c3)c12 ; Clc1cccc2cccc(-c3ccc4c(N5CC6CCC(C5)N6)nc(OCC5(CN6CCOCC6)CC5)nc4n3)c12 ; Clc1cccc2cccc(-c3ncc4c(N5CC6CCC(C5)N6)nc(OCC5(CN6CCOCC6)CC5)nc4n3)c12]

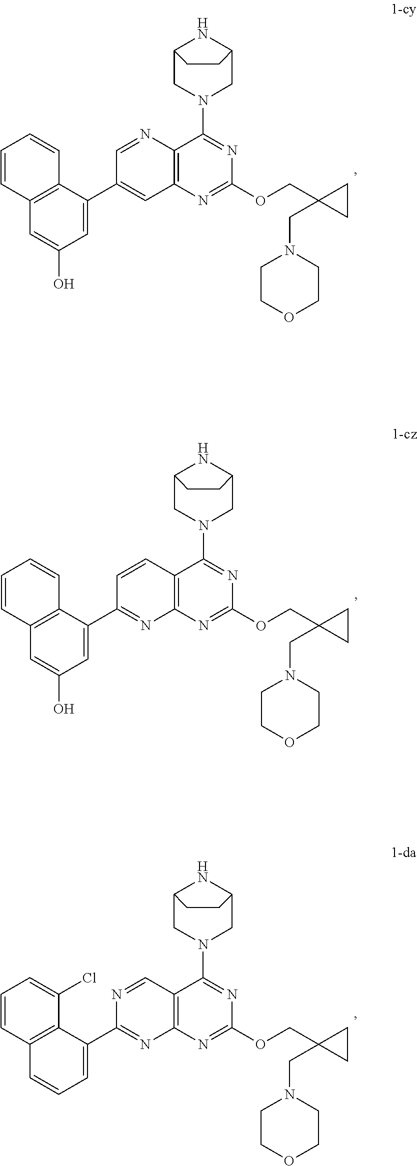 [SMILES: Oc1cc(-c2cnc3c(N4CC5CCC(C4)N5)nc(OCC4(CN5CCOCC5)CC4)nc3c2)c2ccccc2c1 ; Oc1cc(-c2ccc3c(N4CC5CCC(C4)N5)nc(OCC4(CN5CCOCC5)CC4)nc3n2)c2ccccc2c1 ; Clc1cccc2cccc(-c3ncc4c(N5CC6CCC(C5)N6)nc(OCC5(CN6CCOCC6)CC5)nc4n3)c12]

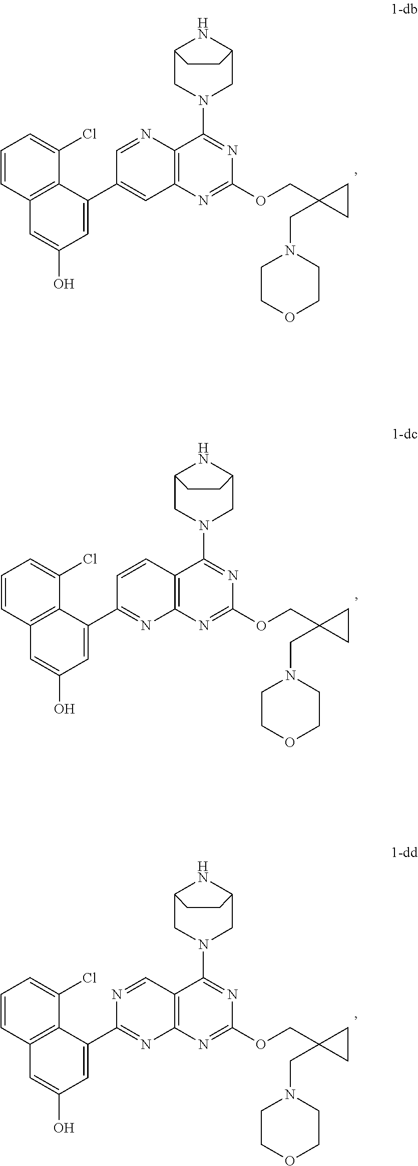 [SMILES: Oc1cc(-c2cnc3c(N4CC5CCC(C4)N5)nc(OCC4(CN5CCOCC5)CC4)nc3c2)c2c(Cl)cccc2c1 ; Oc1cc(-c2ccc3c(N4CC5CCC(C4)N5)nc(OCC4(CN5CCOCC5)CC4)nc3n2)c2c(Cl)cccc2c1 ; Oc1cc(-c2ncc3c(N4CC5CCC(C4)N5)nc(OCC4(CN5CCOCC5)CC4)nc3n2)c2c(Cl)cccc2c1]

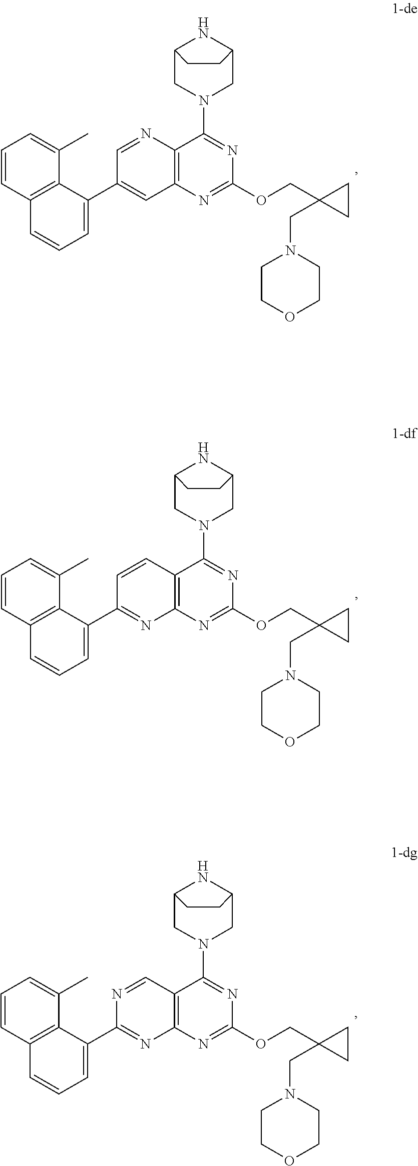 [SMILES: Cc1cccc2cccc(-c3cnc4c(N5CC6CCC(C5)N6)nc(OCC5(CN6CCOCC6)CC5)nc4c3)c12 ; Cc1cccc2cccc(-c3ccc4c(N5CC6CCC(C5)N6)nc(OCC5(CN6CCOCC6)CC5)nc4n3)c12 ; Cc1cccc2cccc(-c3ncc4c(N5CC6CCC(C5)N6)nc(OCC5(CN6CCOCC6)CC5)nc4n3)c12]

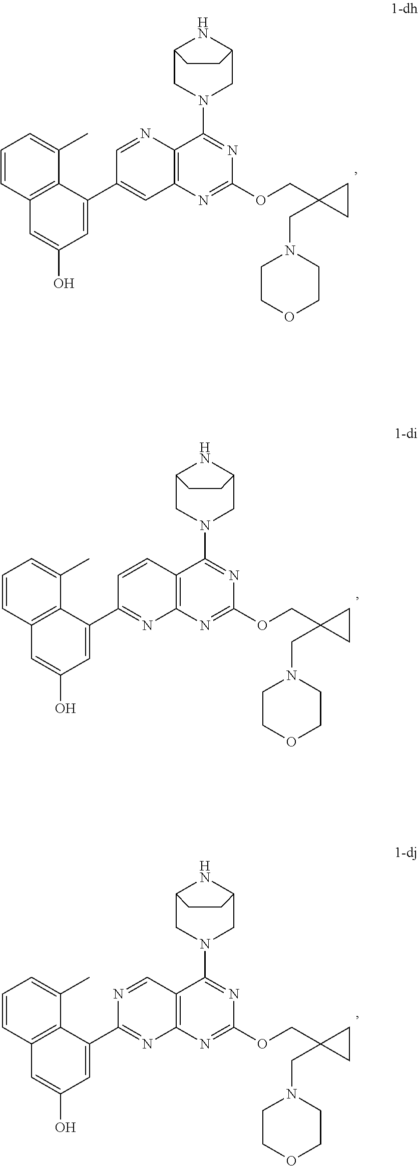 [SMILES: Cc1cccc2cc(O)cc(-c3cnc4c(N5CC6CCC(C5)N6)nc(OCC5(CN6CCOCC6)CC5)nc4c3)c12 ; Cc1cccc2cc(O)cc(-c3ccc4c(N5CC6CCC(C5)N6)nc(OCC5(CN6CCOCC6)CC5)nc4n3)c12 ; Cc1cccc2cc(O)cc(-c3ncc4c(N5CC6CCC(C5)N6)nc(OCC5(CN6CCOCC6)CC5)nc4n3)c12]

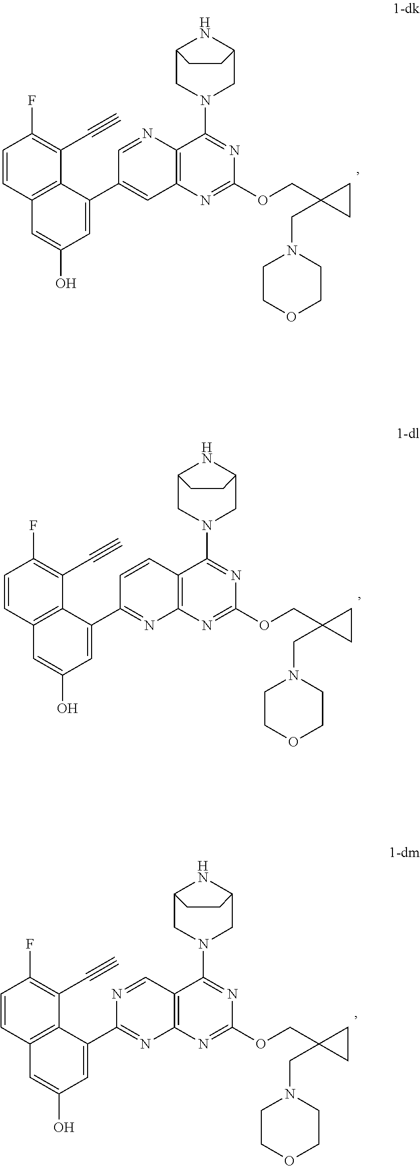 [SMILES: C#Cc1c(F)ccc2cc(O)cc(-c3cnc4c(N5CC6CCC(C5)N6)nc(OCC5(CN6CCOCC6)CC5)nc4c3)c12 ; C#Cc1c(F)ccc2cc(O)cc(-c3ccc4c(N5CC6CCC(C5)N6)nc(OCC5(CN6CCOCC6)CC5)nc4n3)c12 ; C#Cc1c(F)ccc2cc(O)cc(-c3ncc4c(N5CC6CCC(C5)N6)nc(OCC5(CN6CCOCC6)CC5)nc4n3)c12]

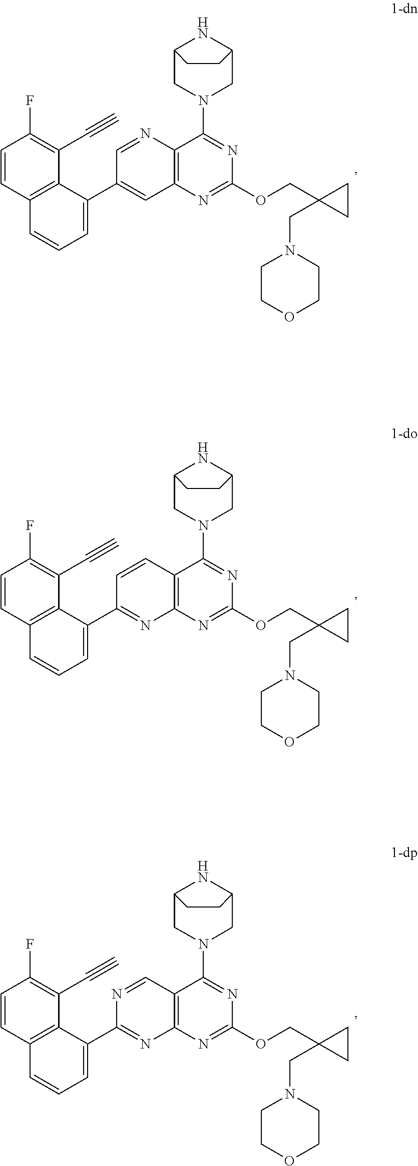 [SMILES: C#Cc1c(F)ccc2cccc(-c3cnc4c(N5CC6CCC(C5)N6)nc(OCC5(CN6CCOCC6)CC5)nc4c3)c12 ; C#Cc1c(F)ccc2cccc(-c3ccc4c(N5CC6CCC(C5)N6)nc(OCC5(CN6CCOCC6)CC5)nc4n3)c12 ; C#Cc1c(F)ccc2cccc(-c3ncc4c(N5CC6CCC(C5)N6)nc(OCC5(CN6CCOCC6)CC5)nc4n3)c12]

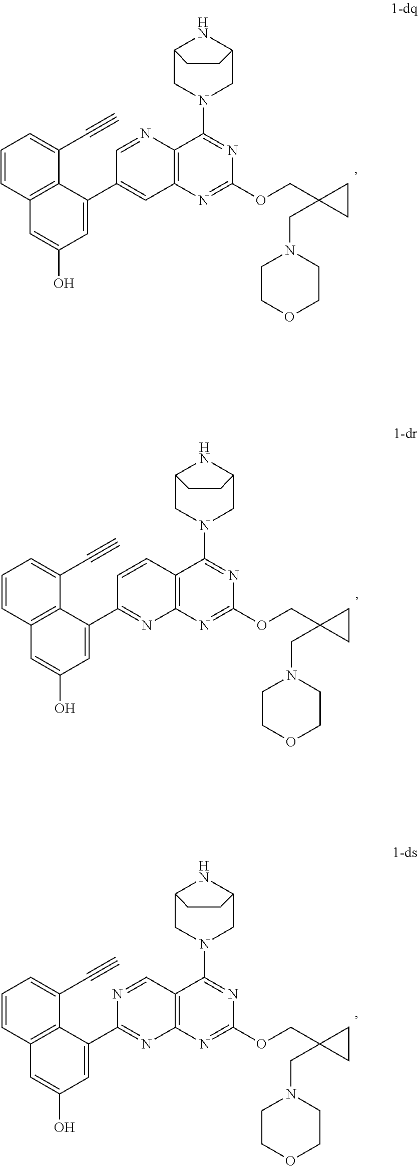 [SMILES: C#Cc1cccc2cc(O)cc(-c3cnc4c(N5CC6CCC(C5)N6)nc(OCC5(CN6CCOCC6)CC5)nc4c3)c12 ; C#Cc1cccc2cc(O)cc(-c3ccc4c(N5CC6CCC(C5)N6)nc(OCC5(CN6CCOCC6)CC5)nc4n3)c12 ; C#Cc1cccc2cc(O)cc(-c3ncc4c(N5CC6CCC(C5)N6)nc(OCC5(CN6CCOCC6)CC5)nc4n3)c12]

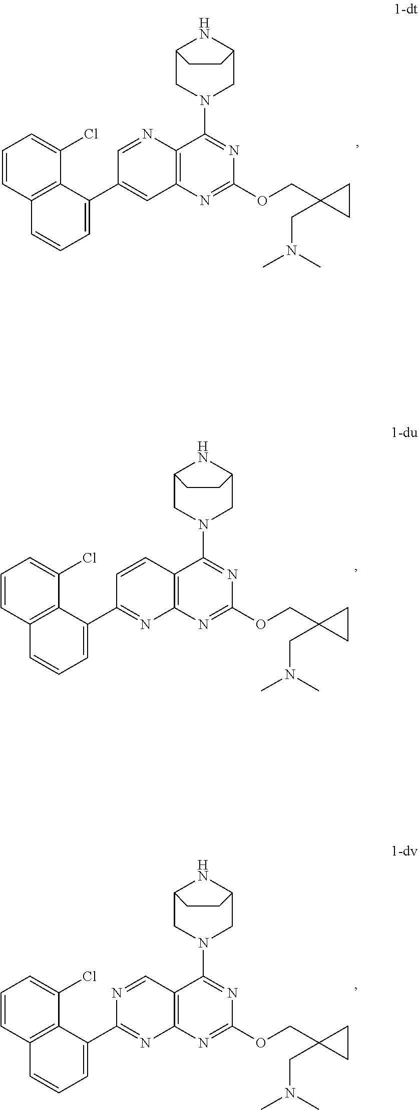 [SMILES: CN(C)CC1(COc2nc(N3CC4CCC(C3)N4)c3ncc(-c4cccc5cccc(Cl)c45)cc3n2)CC1 ; CN(C)CC1(COc2nc(N3CC4CCC(C3)N4)c3ccc(-c4cccc5cccc(Cl)c45)nc3n2)CC1 ; CN(C)CC1(COc2nc(N3CC4CCC(C3)N4)c3cnc(-c4cccc5cccc(Cl)c45)nc3n2)CC1]

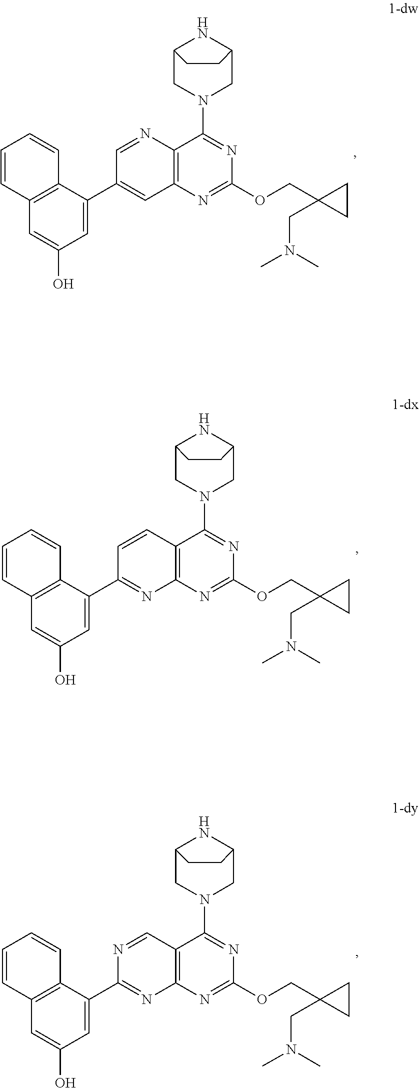 [SMILES: CN(C)CC1(COc2nc(N3CC4CCC(C3)N4)c3ncc(-c4cc(O)cc5ccccc45)cc3n2)CC1 ; CN(C)CC1(COc2nc(N3CC4CCC(C3)N4)c3ccc(-c4cc(O)cc5ccccc45)nc3n2)CC1 ; CN(C)CC1(COc2nc(N3CC4CCC(C3)N4)c3cnc(-c4cc(O)cc5ccccc45)nc3n2)CC1]

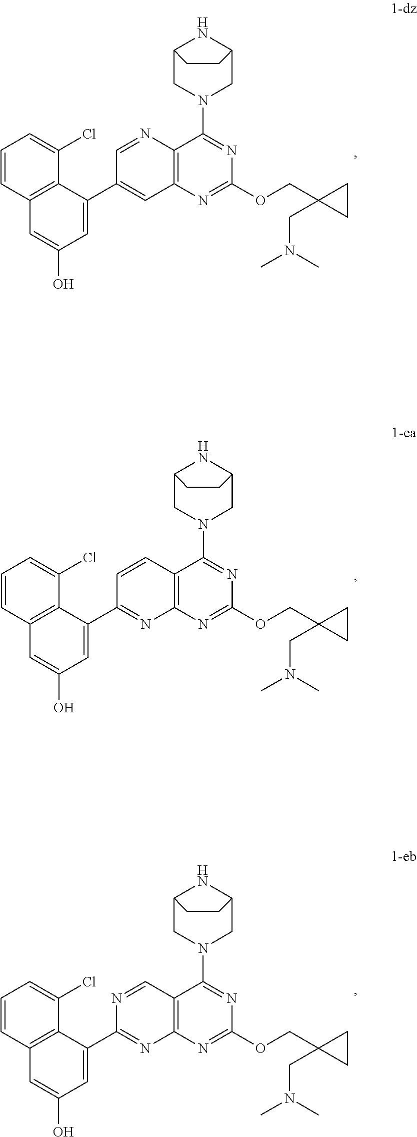 [SMILES: CN(C)CC1(COc2nc(N3CC4CCC(C3)N4)c3ncc(-c4cc(O)cc5cccc(Cl)c45)cc3n2)CC1 ; CN(C)CC1(COc2nc(N3CC4CCC(C3)N4)c3ccc(-c4cc(O)cc5cccc(Cl)c45)nc3n2)CC1 ; CN(C)CC1(COc2nc(N3CC4CCC(C3)N4)c3cnc(-c4cc(O)cc5cccc(Cl)c45)nc3n2)CC1]

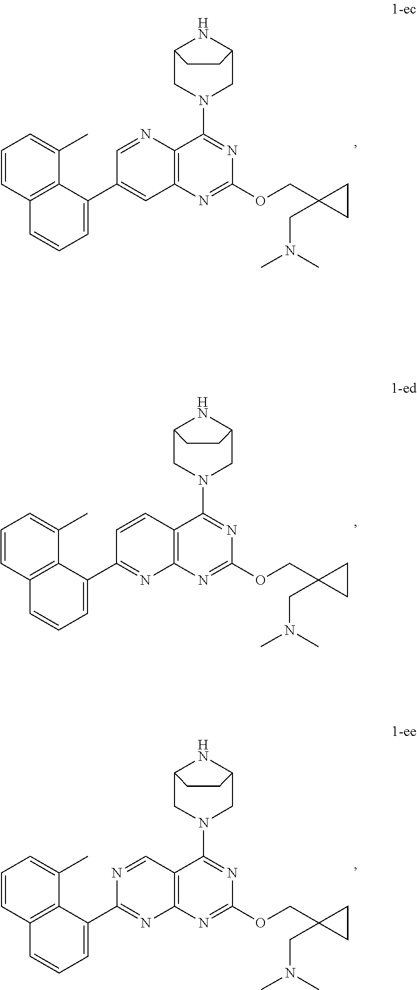 [SMILES: Cc1cccc2cccc(-c3cnc4c(N5CC6CCC(C5)N6)nc(OCC5(CN(C)C)CC5)nc4c3)c12 ; Cc1cccc2cccc(-c3ccc4c(N5CC6CCC(C5)N6)nc(OCC5(CN(C)C)CC5)nc4n3)c12 ; Cc1cccc2cccc(-c3ncc4c(N5CC6CCC(C5)N6)nc(OCC5(CN(C)C)CC5)nc4n3)c12]

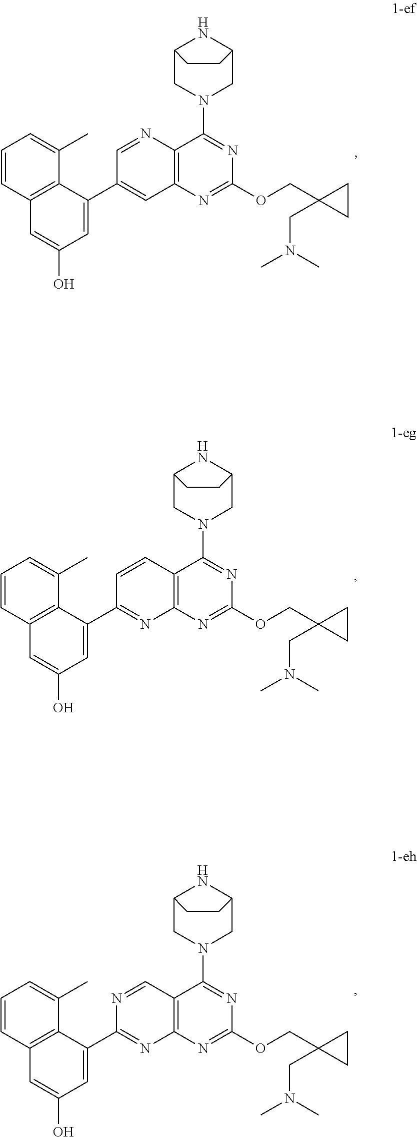 [SMILES: Cc1cccc2cc(O)cc(-c3cnc4c(N5CC6CCC(C5)N6)nc(OCC5(CN(C)C)CC5)nc4c3)c12 ; Cc1cccc2cc(O)cc(-c3ccc4c(N5CC6CCC(C5)N6)nc(OCC5(CN(C)C)CC5)nc4n3)c12 ; Cc1cccc2cc(O)cc(-c3ncc4c(N5CC6CCC(C5)N6)nc(OCC5(CN(C)C)CC5)nc4n3)c12]

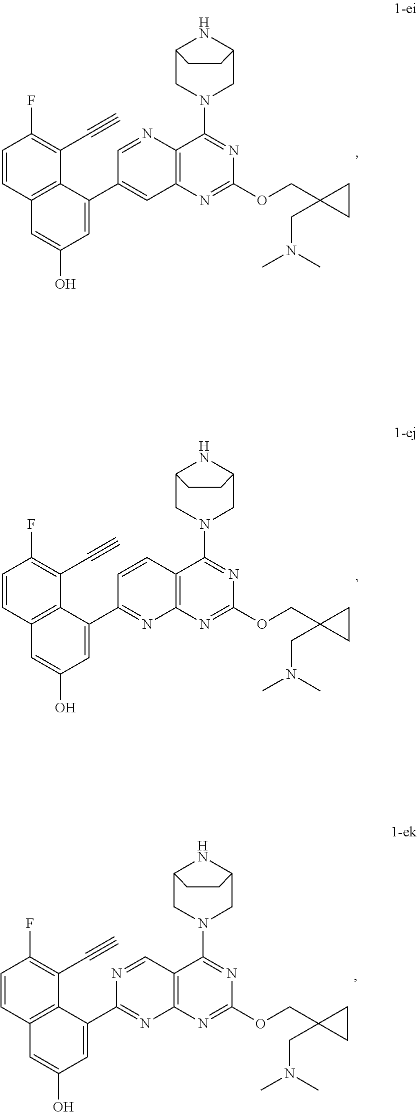 [SMILES: C#Cc1c(F)ccc2cc(O)cc(-c3cnc4c(N5CC6CCC(C5)N6)nc(OCC5(CN(C)C)CC5)nc4c3)c12 ; C#Cc1c(F)ccc2cc(O)cc(-c3ccc4c(N5CC6CCC(C5)N6)nc(OCC5(CN(C)C)CC5)nc4n3)c12 ; C#Cc1c(F)ccc2cc(O)cc(-c3ncc4c(N5CC6CCC(C5)N6)nc(OCC5(CN(C)C)CC5)nc4n3)c12]

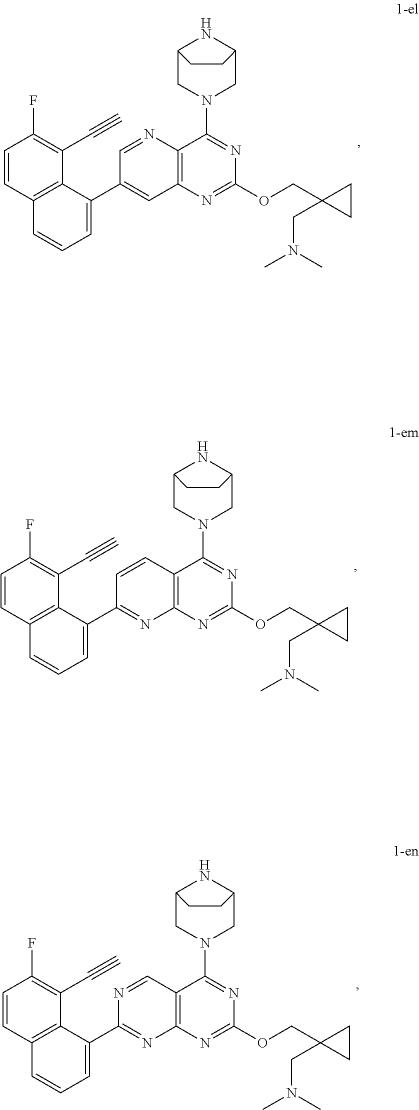 [SMILES: C#Cc1c(F)ccc2cccc(-c3cnc4c(N5CC6CCC(C5)N6)nc(OCC5(CN(C)C)CC5)nc4c3)c12 ; C#Cc1c(F)ccc2cccc(-c3ccc4c(N5CC6CCC(C5)N6)nc(OCC5(CN(C)C)CC5)nc4n3)c12 ; C#Cc1c(F)ccc2cccc(-c3ncc4c(N5CC6CCC(C5)N6)nc(OCC5(CN(C)C)CC5)nc4n3)c12]

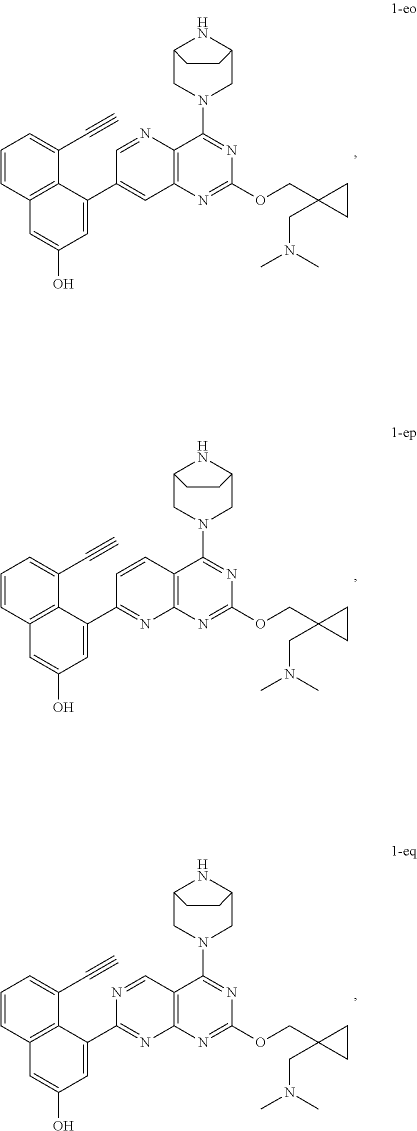 [SMILES: C#Cc1cccc2cc(O)cc(-c3cnc4c(N5CC6CCC(C5)N6)nc(OCC5(CN(C)C)CC5)nc4c3)c12 ; C#Cc1cccc2cc(O)cc(-c3ccc4c(N5CC6CCC(C5)N6)nc(OCC5(CN(C)C)CC5)nc4n3)c12 ; C#Cc1cccc2cc(O)cc(-c3ncc4c(N5CC6CCC(C5)N6)nc(OCC5(CN(C)C)CC5)nc4n3)c12]

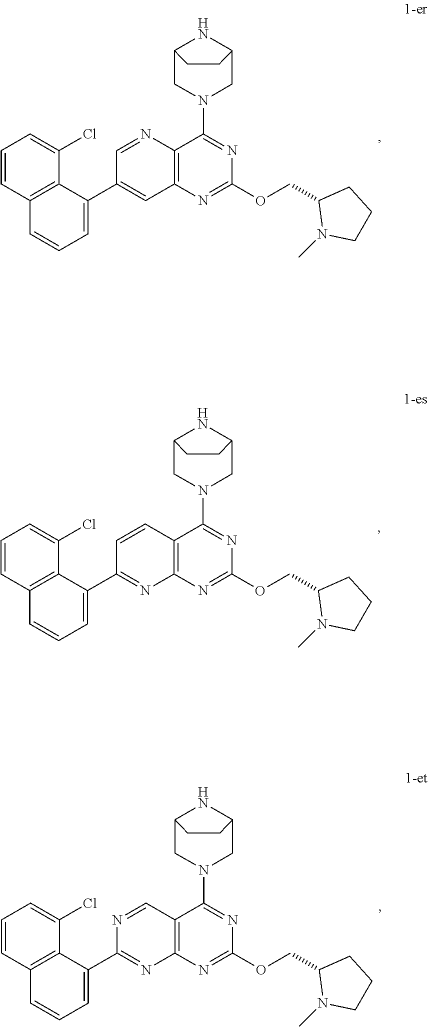

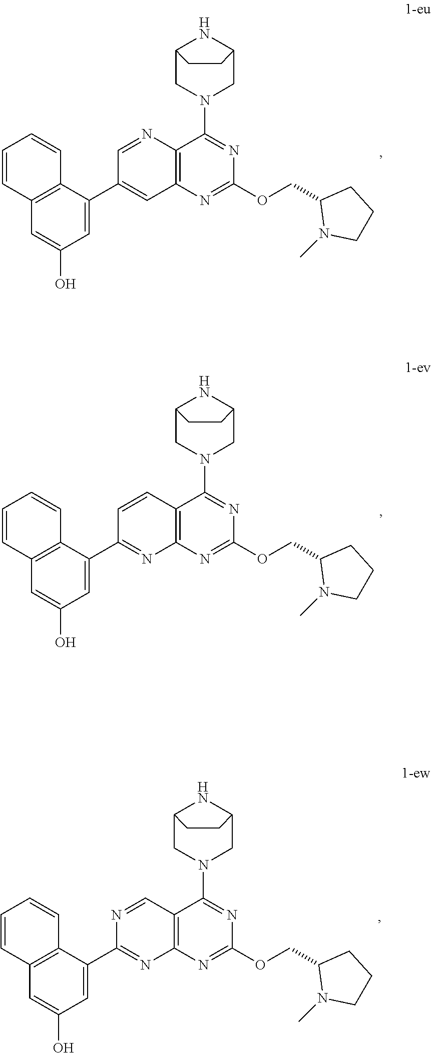

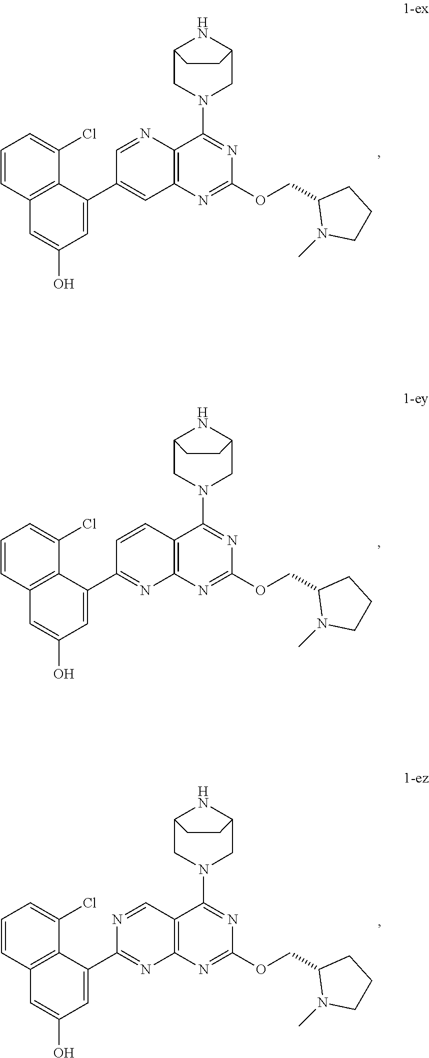

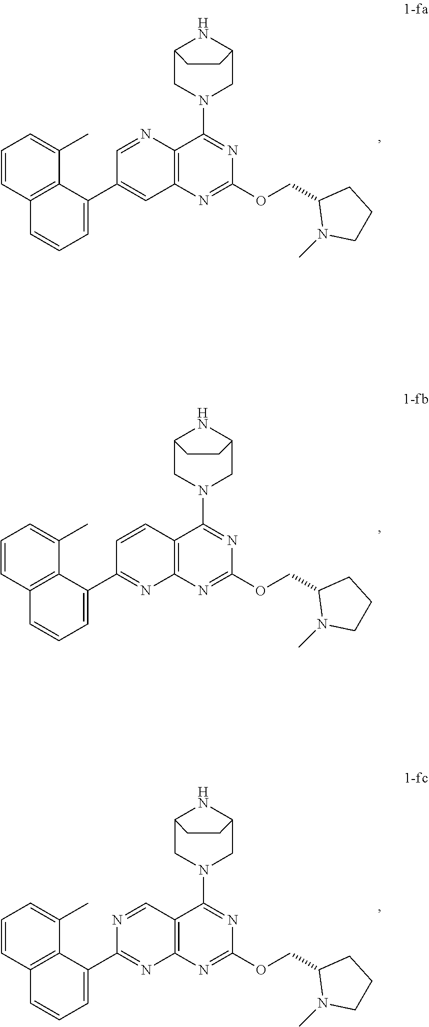

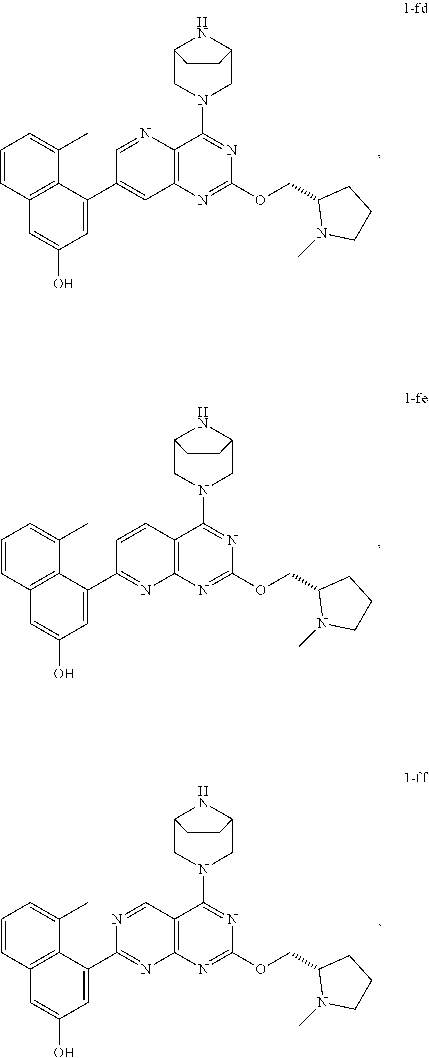

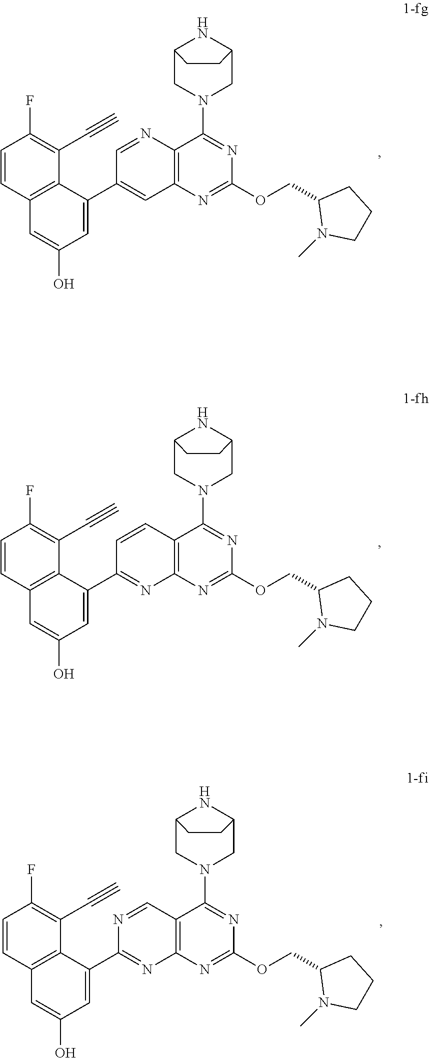

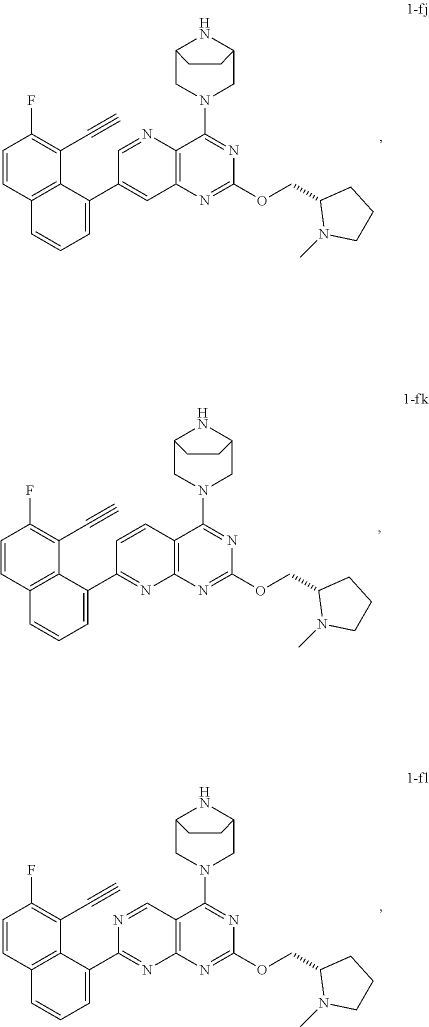

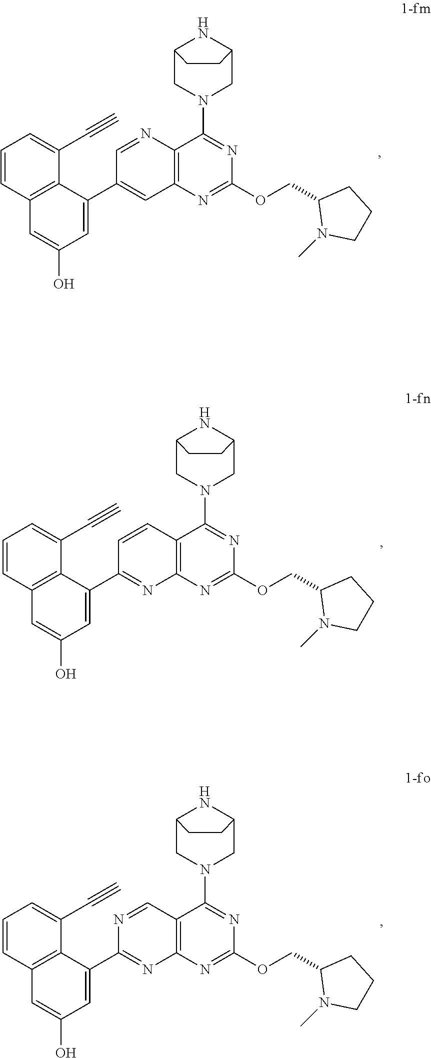

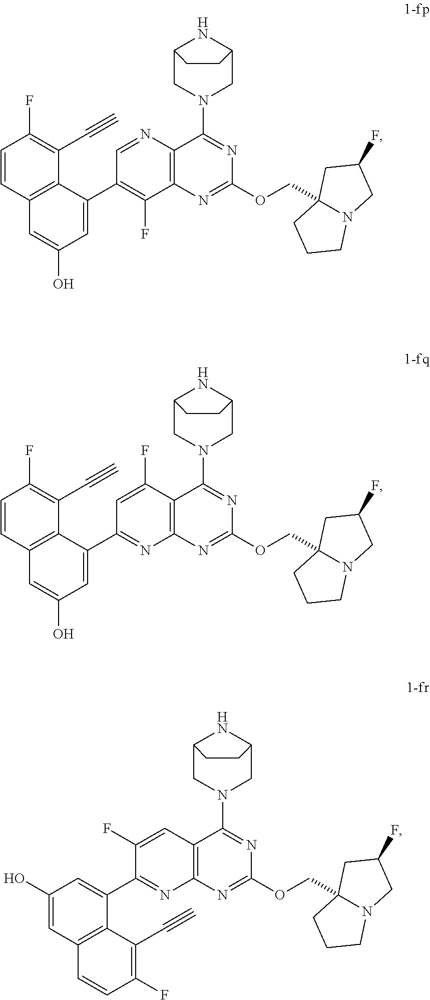 [SMILES: C#Cc1c(F)ccc2cc(O)cc(-c3cnc4c(N5CC6CCC(C5)N6)nc(OC[C@@]56CCCN5C[C@H](F)C6)nc4c3F)c12 ; C#Cc1c(F)ccc2cc(O)cc(-c3cc(F)c4c(N5CC6CCC(C5)N6)nc(OC[C@@]56CCCN5C[C@H](F)C6)nc4n3)c12 ; C#Cc1c(F)ccc2cc(O)cc(-c3nc4nc(OC[C@@]56CCCN5C[C@H](F)C6)nc(N5CC6CCC(C5)N6)c4cc3F)c12]

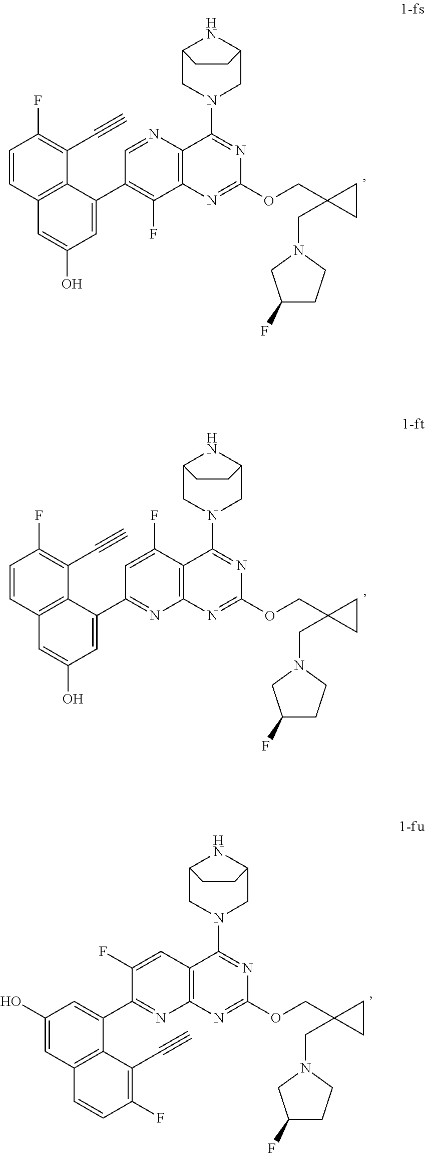 [SMILES: C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cc(F)c4c(N5CC6CCC(C5)N6)nc(OCC5(CN6CC[C@@H](F)C6)CC5)nc4n3)c12 ; C#Cc1c(F)ccc2cc(O)cc(-c3nc4nc(OCC5(CN6CC[C@@H](F)C6)CC5)nc(N5CC6CCC(C5)N6)c4cc3F)c12]

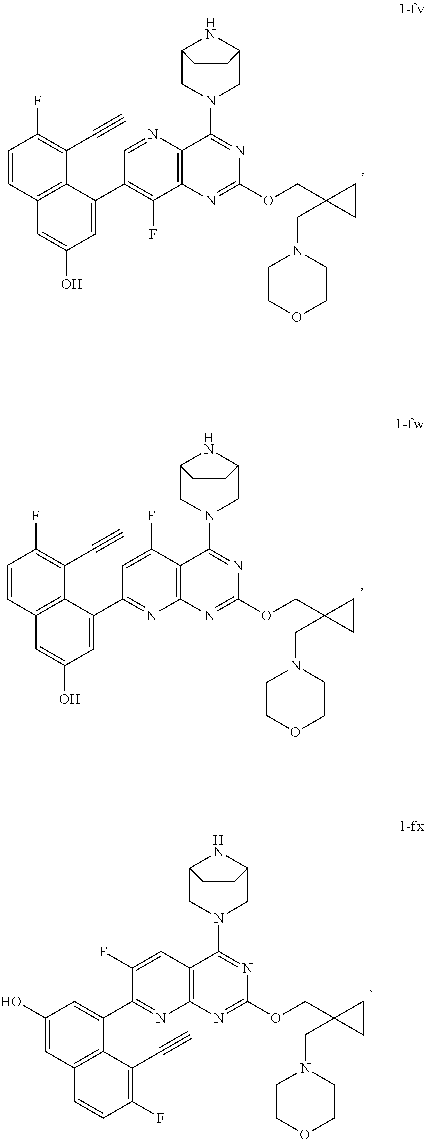 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cc(F)c4c(N5CC6CCC(C5)N6)nc(OCC5(CN6CCOCC6)CC5)nc4n3)c12 ; C#Cc1c(F)ccc2cc(O)cc(-c3nc4nc(OCC5(CN6CCOCC6)CC5)nc(N5CC6CCC(C5)N6)c4cc3F)c12]

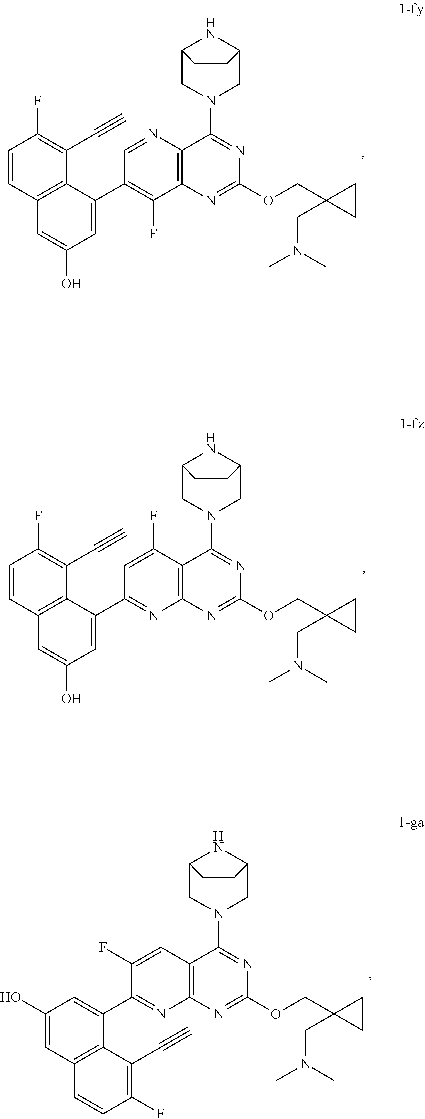 [SMILES: C#Cc1c(F)ccc2cc(O)cc(-c3cnc4c(N5CC6CCC(C5)N6)nc(OCC5(CN(C)C)CC5)nc4c3F)c12 ; C#Cc1c(F)ccc2cc(O)cc(-c3cc(F)c4c(N5CC6CCC(C5)N6)nc(OCC5(CN(C)C)CC5)nc4n3)c12 ; C#Cc1c(F)ccc2cc(O)cc(-c3nc4nc(OCC5(CN(C)C)CC5)nc(N5CC6CCC(C5)N6)c4cc3F)c12]

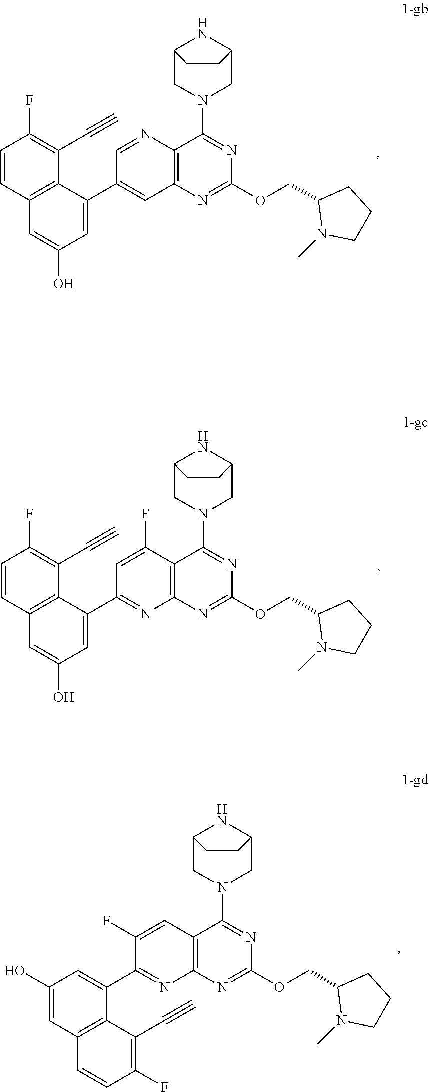

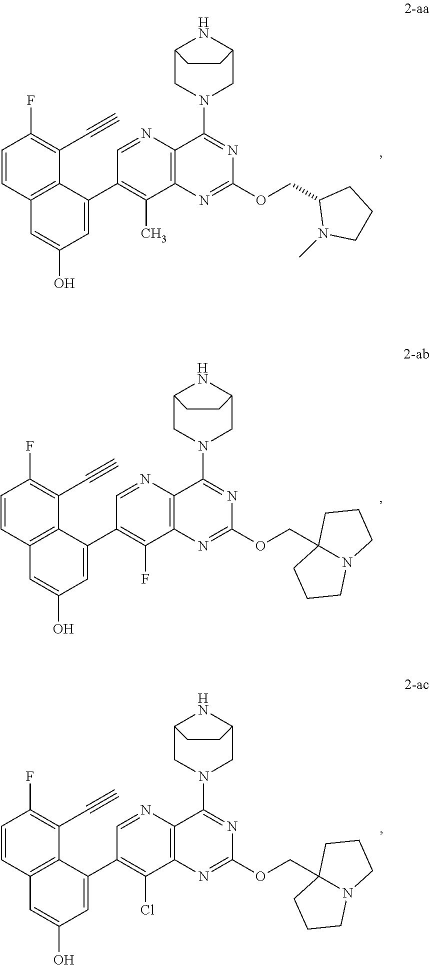 [SMILES: C#Cc1c(F)ccc2cc(O)cc(-c3cnc4c(N5CC6CCC(C5)N6)nc(OC[C@@H]5CCCN5C)nc4c3C)c12 ; C#Cc1c(F)ccc2cc(O)cc(-c3cnc4c(N5CC6CCC(C5)N6)nc(OCC56CCCN5CCC6)nc4c3F)c12 ; C#Cc1c(F)ccc2cc(O)cc(-c3cnc4c(N5CC6CCC(C5)N6)nc(OCC56CCCN5CCC6)nc4c3Cl)c12]

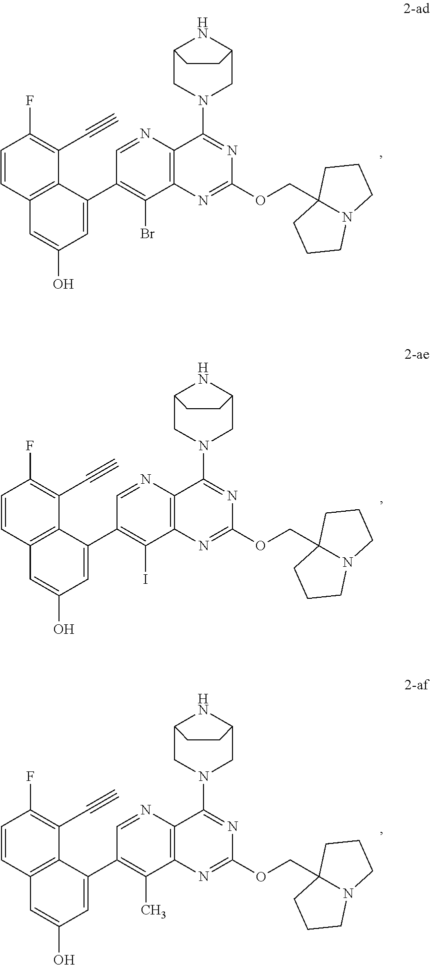 [SMILES: C#Cc1c(F)ccc2cc(O)cc(-c3cnc4c(N5CC6CCC(C5)N6)nc(OCC56CCCN5CCC6)nc4c3Br)c12 ; C#Cc1c(F)ccc2cc(O)cc(-c3cnc4c(N5CC6CCC(C5)N6)nc(OCC56CCCN5CCC6)nc4c3I)c12 ; C#Cc1c(F)ccc2cc(O)cc(-c3cnc4c(N5CC6CCC(C5)N6)nc(OCC56CCCN5CCC6)nc4c3C)c12]

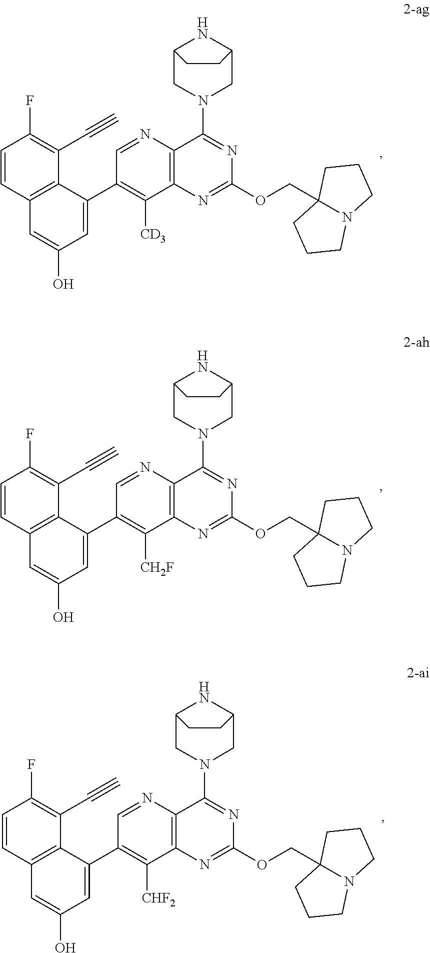

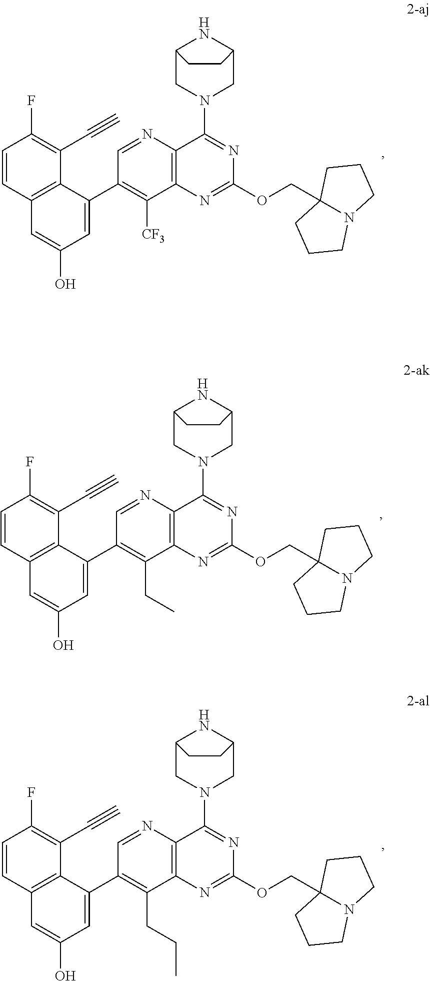 [SMILES: C#Cc1c(F)ccc2cc(O)cc(-c3cnc4c(N5CC6CCC(C5)N6)nc(OCC56CCCN5CCC6)nc4c3C(F)(F)F)c12 ; C#Cc1c(F)ccc2cc(O)cc(-c3cnc4c(N5CC6CCC(C5)N6)nc(OCC56CCCN5CCC6)nc4c3CC)c12 ; C#Cc1c(F)ccc2cc(O)cc(-c3cnc4c(N5CC6CCC(C5)N6)nc(OCC56CCCN5CCC6)nc4c3CCC)c12]

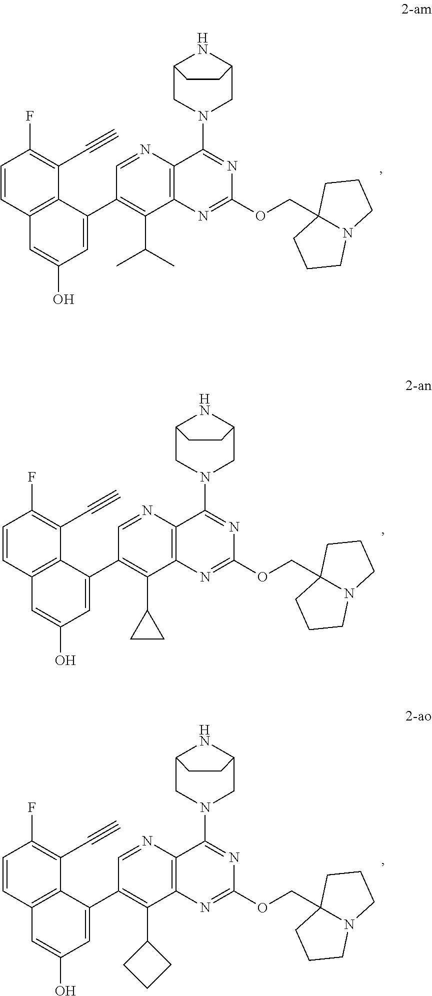 [SMILES: C#Cc1c(F)ccc2cc(O)cc(-c3cnc4c(N5CC6CCC(C5)N6)nc(OCC56CCCN5CCC6)nc4c3C(C)C)c12 ; C#Cc1c(F)ccc2cc(O)cc(-c3cnc4c(N5CC6CCC(C5)N6)nc(OCC56CCCN5CCC6)nc4c3C3CC3)c12 ; C#Cc1c(F)ccc2cc(O)cc(-c3cnc4c(N5CC6CCC(C5)N6)nc(OCC56CCCN5CCC6)nc4c3C3CCC3)c12]

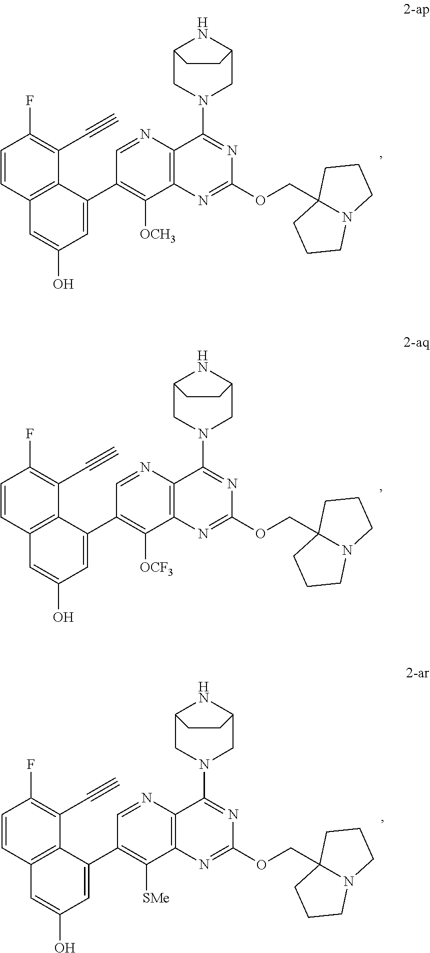 [SMILES: C#Cc1c(F)ccc2cc(O)cc(-c3cnc4c(N5CC6CCC(C5)N6)nc(OCC56CCCN5CCC6)nc4c3OC)c12 ; C#Cc1c(F)ccc2cc(O)cc(-c3cnc4c(N5CC6CCC(C5)N6)nc(OCC56CCCN5CCC6)nc4c3OC(F)(F)F)c12 ; C#Cc1c(F)ccc2cc(O)cc(-c3cnc4c(N5CC6CCC(C5)N6)nc(OCC56CCCN5CCC6)nc4c3SC)c12]

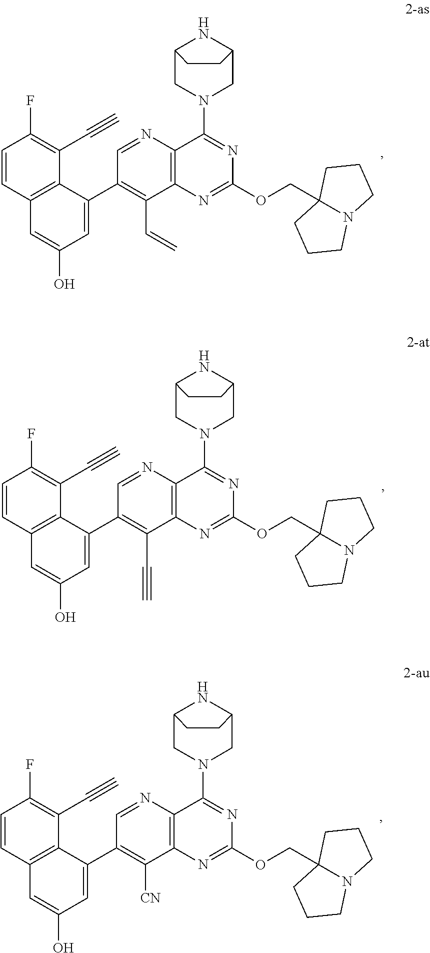 [SMILES: C#Cc1c(F)ccc2cc(O)cc(-c3cnc4c(N5CC6CCC(C5)N6)nc(OCC56CCCN5CCC6)nc4c3C=C)c12 ; C#Cc1c(-c2cc(O)cc3ccc(F)c(C#C)c23)cnc2c(N3CC4CCC(C3)N4)nc(OCC34CCCN3CCC4)nc12 ; C#Cc1c(F)ccc2cc(O)cc(-c3cnc4c(N5CC6CCC(C5)N6)nc(OCC56CCCN5CCC6)nc4c3C#N)c12]

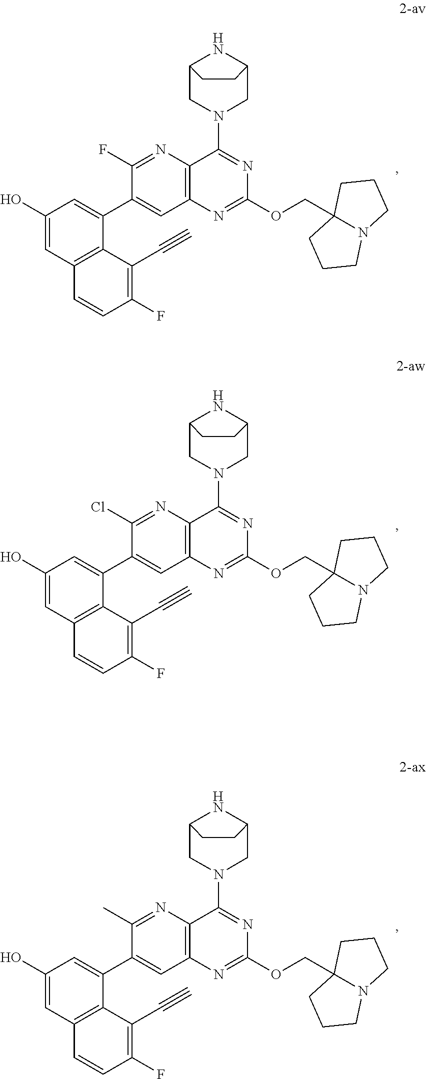 [SMILES: C#Cc1c(F)ccc2cc(O)cc(-c3cc4nc(OCC56CCCN5CCC6)nc(N5CC6CCC(C5)N6)c4nc3F)c12 ; C#Cc1c(F)ccc2cc(O)cc(-c3cc4nc(OCC56CCCN5CCC6)nc(N5CC6CCC(C5)N6)c4nc3Cl)c12 ; C#Cc1c(F)ccc2cc(O)cc(-c3cc4nc(OCC56CCCN5CCC6)nc(N5CC6CCC(C5)N6)c4nc3C)c12]

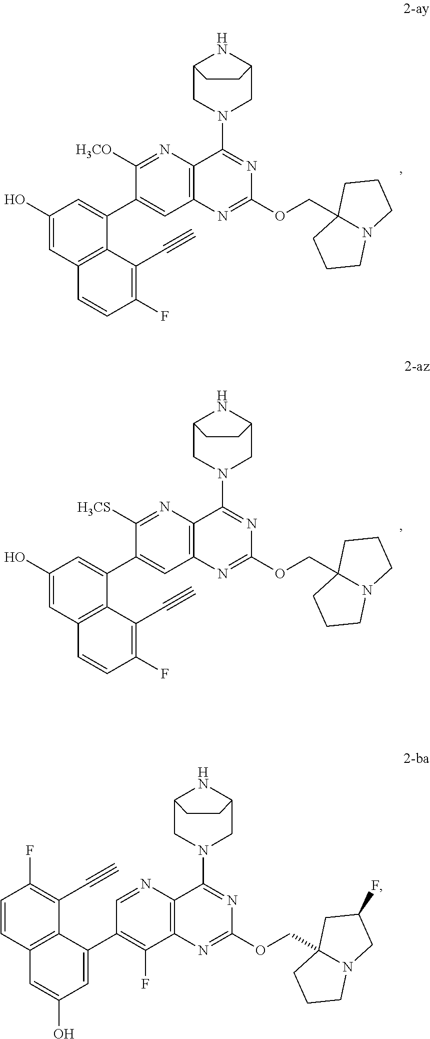

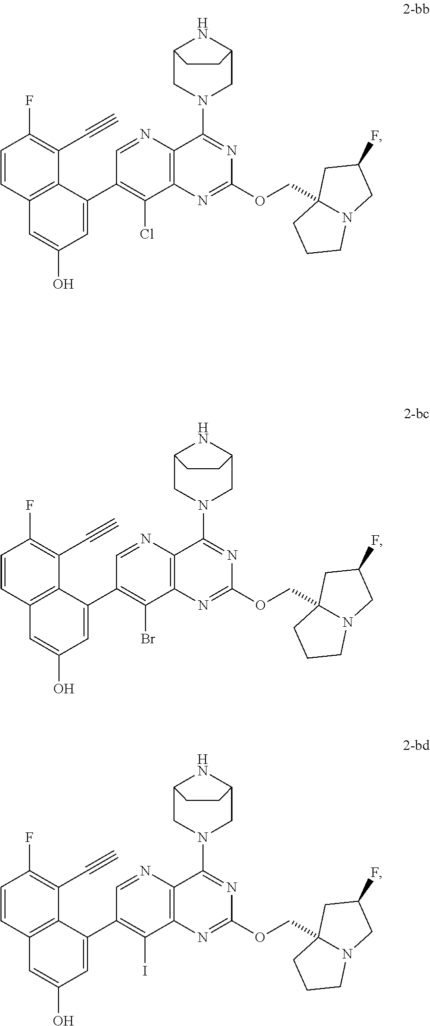 [SMILES: C#Cc1c(F)ccc2cc(O)cc(-c3cnc4c(N5CC6CCC(C5)N6)nc(OC[C@@]56CCCN5C[C@H](F)C6)nc4c3Cl)c12 ; C#Cc1c(F)ccc2cc(O)cc(-c3cnc4c(N5CC6CCC(C5)N6)nc(OC[C@@]56CCCN5C[C@H](F)C6)nc4c3Br)c12 ; C#Cc1c(F)ccc2cc(O)cc(-c3cnc4c(N5CC6CCC(C5)N6)nc(OC[C@@]56CCCN5C[C@H](F)C6)nc4c3I)c12]

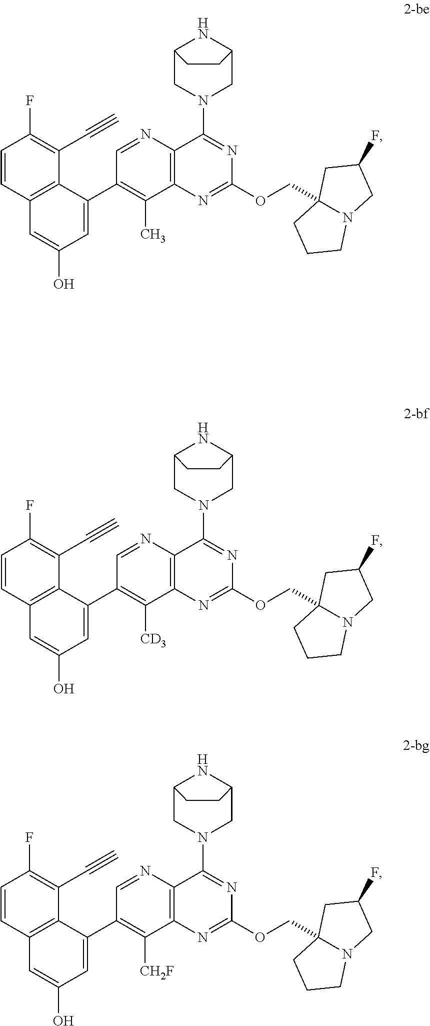

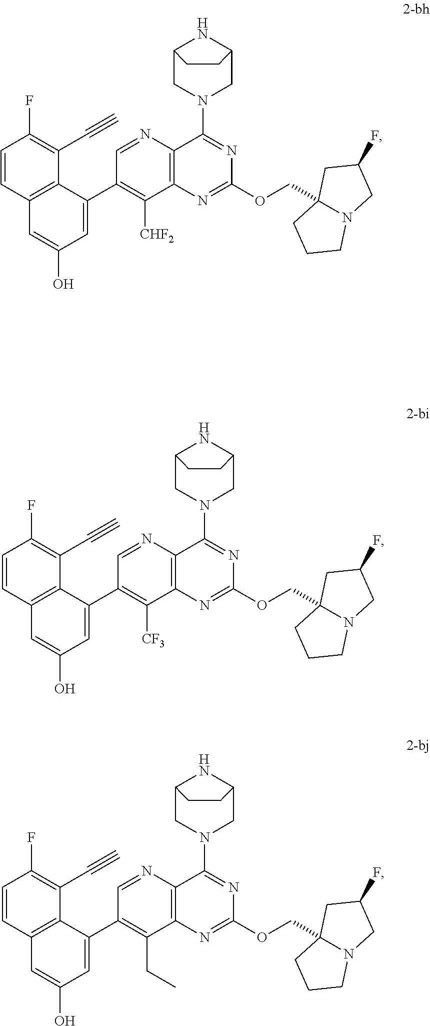 [SMILES: C#Cc1c(F)ccc2cc(O)cc(-c3cnc4c(N5CC6CCC(C5)N6)nc(OC[C@@]56CCCN5C[C@H](F)C6)nc4c3C(F)F)c12 ; C#Cc1c(F)ccc2cc(O)cc(-c3cnc4c(N5CC6CCC(C5)N6)nc(OC[C@@]56CCCN5C[C@H](F)C6)nc4c3C(F)(F)F)c12 ; C#Cc1c(F)ccc2cc(O)cc(-c3cnc4c(N5CC6CCC(C5)N6)nc(OC[C@@]56CCCN5C[C@H](F)C6)nc4c3CC)c12]

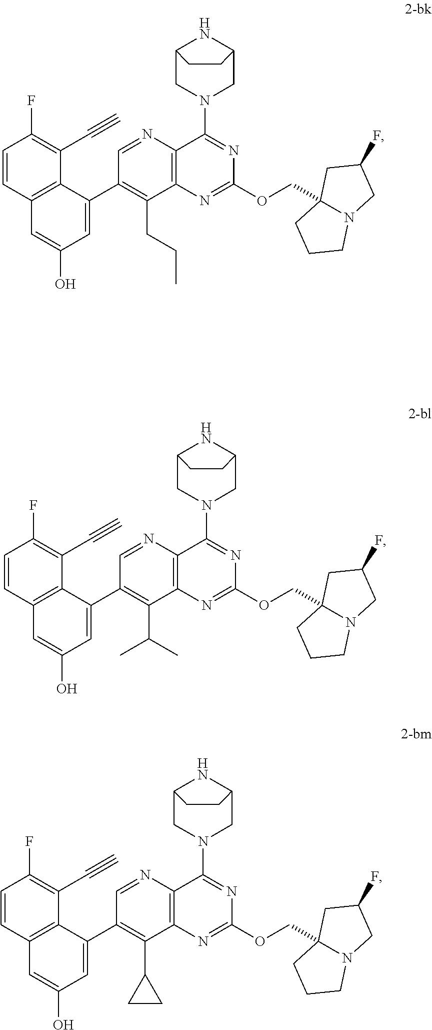 [SMILES: C#Cc1c(F)ccc2cc(O)cc(-c3cnc4c(N5CC6CCC(C5)N6)nc(OC[C@@]56CCCN5C[C@H](F)C6)nc4c3CCC)c12 ; C#Cc1c(F)ccc2cc(O)cc(-c3cnc4c(N5CC6CCC(C5)N6)nc(OC[C@@]56CCCN5C[C@H](F)C6)nc4c3C(C)C)c12 ; C#Cc1c(F)ccc2cc(O)cc(-c3cnc4c(N5CC6CCC(C5)N6)nc(OC[C@@]56CCCN5C[C@H](F)C6)nc4c3C3CC3)c12]

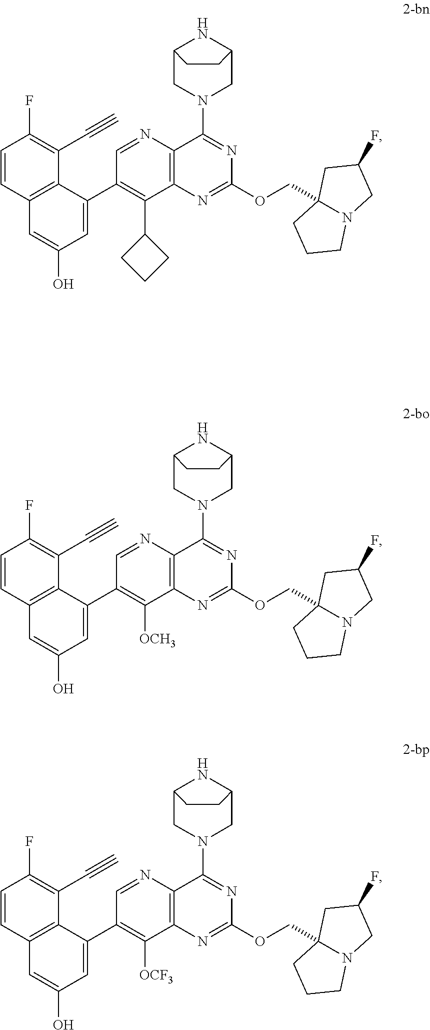 [SMILES: C#Cc1c(F)ccc2cc(O)cc(-c3cnc4c(N5CC6CCC(C5)N6)nc(OC[C@@]56CCCN5C[C@H](F)C6)nc4c3C3CCC3)c12 ; C#Cc1c(F)ccc2cc(O)cc(-c3cnc4c(N5CC6CCC(C5)N6)nc(OC[C@@]56CCCN5C[C@H](F)C6)nc4c3OC)c12 ; C#Cc1c(F)ccc2cc(O)cc(-c3cnc4c(N5CC6CCC(C5)N6)nc(OC[C@@]56CCCN5C[C@H](F)C6)nc4c3OC(F)(F)F)c12]

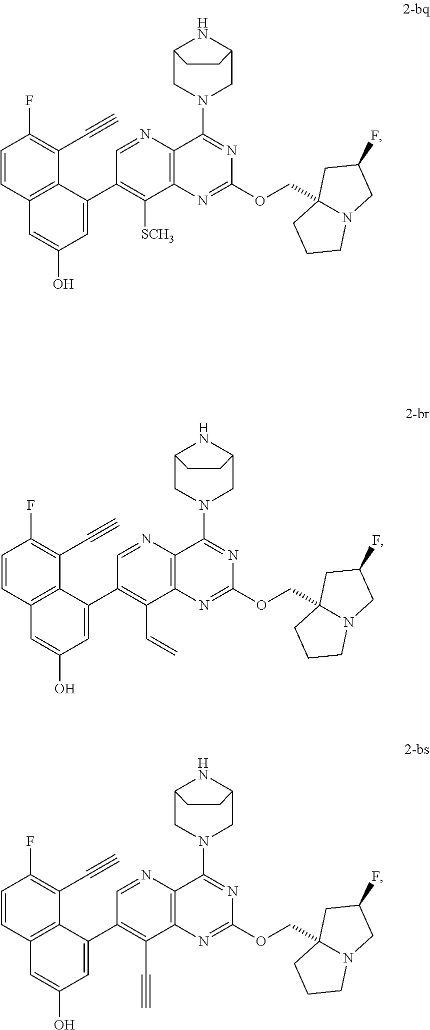 [SMILES: C#Cc1c(F)ccc2cc(O)cc(-c3cnc4c(N5CC6CCC(C5)N6)nc(OC[C@@]56CCCN5C[C@H](F)C6)nc4c3SC)c12 ; C#Cc1c(F)ccc2cc(O)cc(-c3cnc4c(N5CC6CCC(C5)N6)nc(OC[C@@]56CCCN5C[C@H](F)C6)nc4c3C=C)c12 ; C#Cc1c(-c2cc(O)cc3ccc(F)c(C#C)c23)cnc2c(N3CC4CCC(C3)N4)nc(OC[C@@]34CCCN3C[C@H](F)C4)nc12]

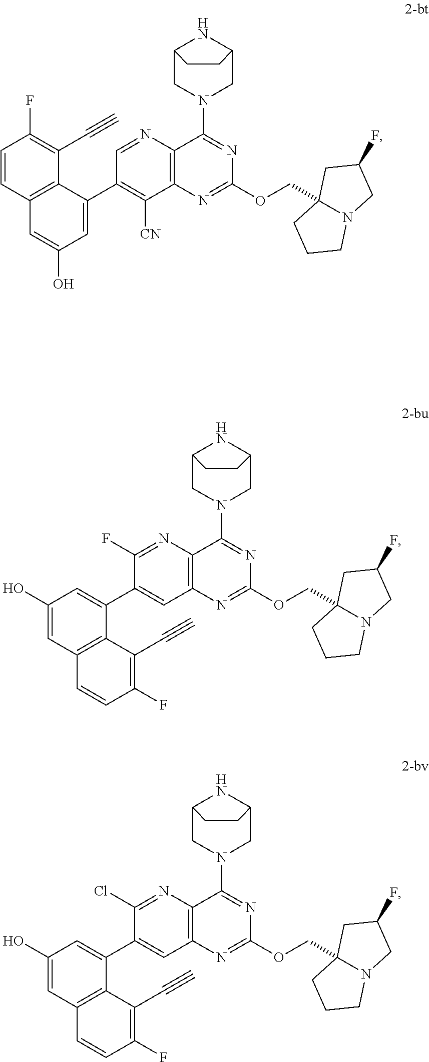 [SMILES: C#Cc1c(F)ccc2cc(O)cc(-c3cnc4c(N5CC6CCC(C5)N6)nc(OC[C@@]56CCCN5C[C@H](F)C6)nc4c3C#N)c12 ; C#Cc1c(F)ccc2cc(O)cc(-c3cc4nc(OC[C@@]56CCCN5C[C@H](F)C6)nc(N5CC6CCC(C5)N6)c4nc3F)c12 ; C#Cc1c(F)ccc2cc(O)cc(-c3cc4nc(OC[C@@]56CCCN5C[C@H](F)C6)nc(N5CC6CCC(C5)N6)c4nc3Cl)c12]

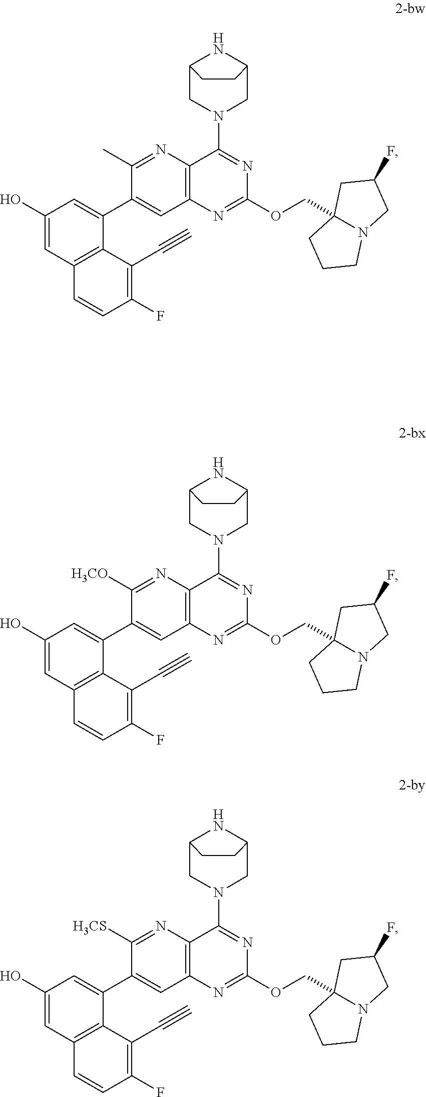

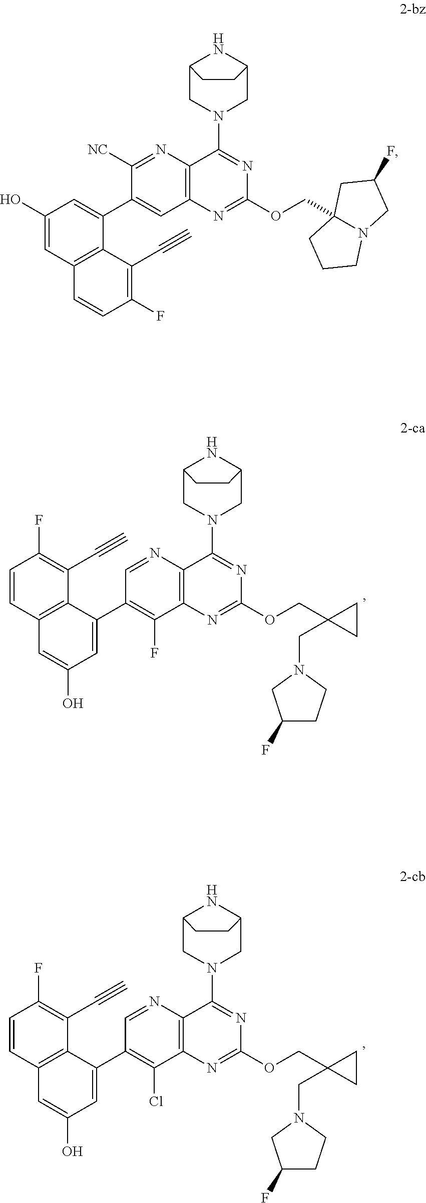 [SMILES: C#Cc1c(F)ccc2cc(O)cc(-c3cc4nc(OC[C@@]56CCCN5C[C@H](F)C6)nc(N5CC6CCC(C5)N6)c4nc3C#N)c12 ; C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cnc4c(N5CC6CCC(C5)N6)nc(OCC5(CN6CC[C@@H](F)C6)CC5)nc4c3Cl)c12]

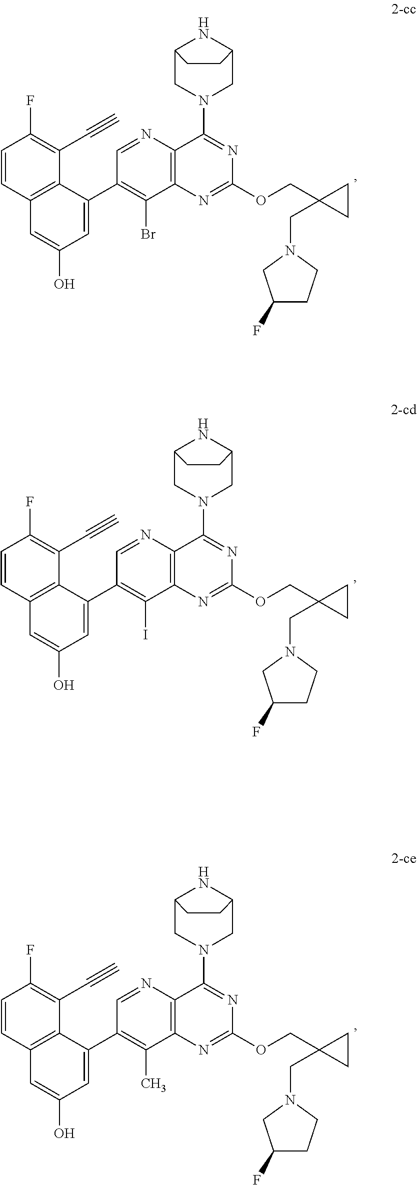 [SMILES: C#Cc1c(F)ccc2cc(O)cc(-c3cnc4c(N5CC6CCC(C5)N6)nc(OCC5(CN6CC[C@@H](F)C6)CC5)nc4c3Br)c12 ; C#Cc1c(F)ccc2cc(O)cc(-c3cnc4c(N5CC6CCC(C5)N6)nc(OCC5(CN6CC[C@@H](F)C6)CC5)nc4c3I)c12 ; C#Cc1c(F)ccc2cc(O)cc(-c3cnc4c(N5CC6CCC(C5)N6)nc(OCC5(CN6CC[C@@H](F)C6)CC5)nc4c3C)c12]

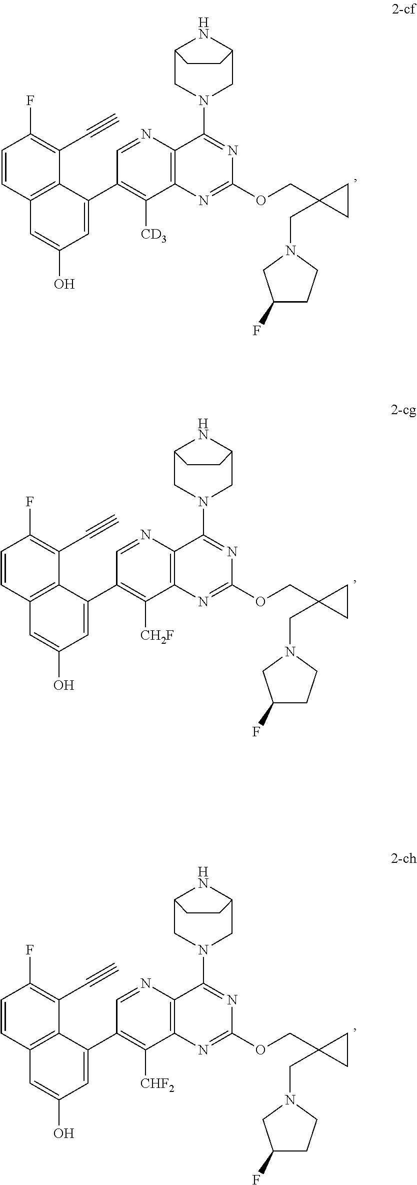

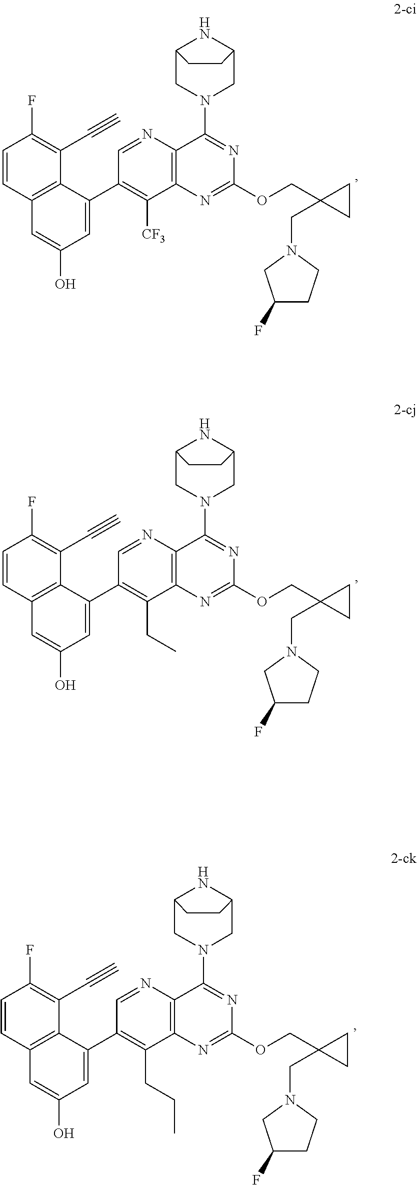 [SMILES: C#Cc1c(F)ccc2cc(O)cc(-c3cnc4c(N5CC6CCC(C5)N6)nc(OCC5(CN6CC[C@@H](F)C6)CC5)nc4c3C(F)(F)F)c12 ; C#Cc1c(F)ccc2cc(O)cc(-c3cnc4c(N5CC6CCC(C5)N6)nc(OCC5(CN6CC[C@@H](F)C6)CC5)nc4c3CC)c12 ; C#Cc1c(F)ccc2cc(O)cc(-c3cnc4c(N5CC6CCC(C5)N6)nc(OCC5(CN6CC[C@@H](F)C6)CC5)nc4c3CCC)c12]

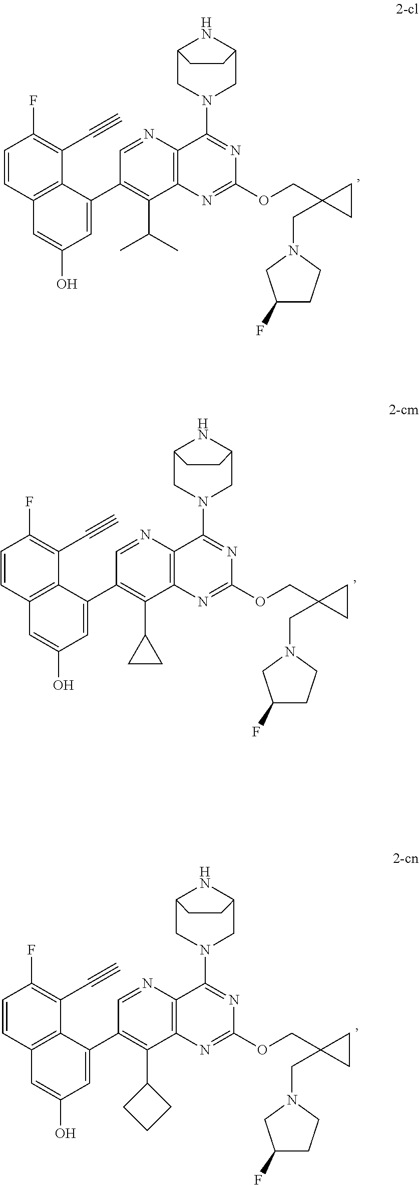 [SMILES: C#Cc1c(F)ccc2cc(O)cc(-c3cnc4c(N5CC6CCC(C5)N6)nc(OCC5(CN6CC[C@@H](F)C6)CC5)nc4c3C(C)C)c12 ; C#Cc1c(F)ccc2cc(O)cc(-c3cnc4c(N5CC6CCC(C5)N6)nc(OCC5(CN6CC[C@@H](F)C6)CC5)nc4c3C3CC3)c12 ; C#Cc1c(F)ccc2cc(O)cc(-c3cnc4c(N5CC6CCC(C5)N6)nc(OCC5(CN6CC[C@@H](F)C6)CC5)nc4c3C3CCC3)c12]

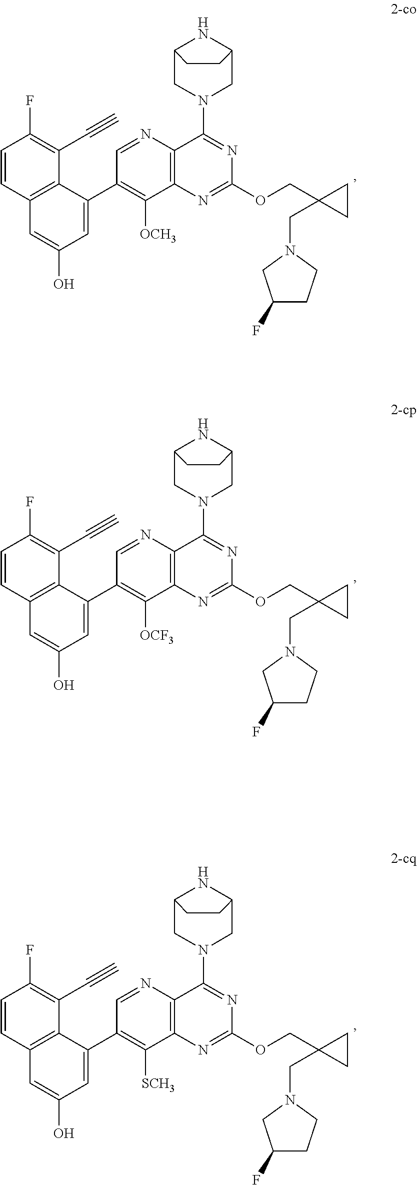 [SMILES: C#Cc1c(F)ccc2cc(O)cc(-c3cnc4c(N5CC6CCC(C5)N6)nc(OCC5(CN6CC[C@@H](F)C6)CC5)nc4c3OC)c12 ; C#Cc1c(F)ccc2cc(O)cc(-c3cnc4c(N5CC6CCC(C5)N6)nc(OCC5(CN6CC[C@@H](F)C6)CC5)nc4c3OC(F)(F)F)c12 ; C#Cc1c(F)ccc2cc(O)cc(-c3cnc4c(N5CC6CCC(C5)N6)nc(OCC5(CN6CC[C@@H](F)C6)CC5)nc4c3SC)c12]

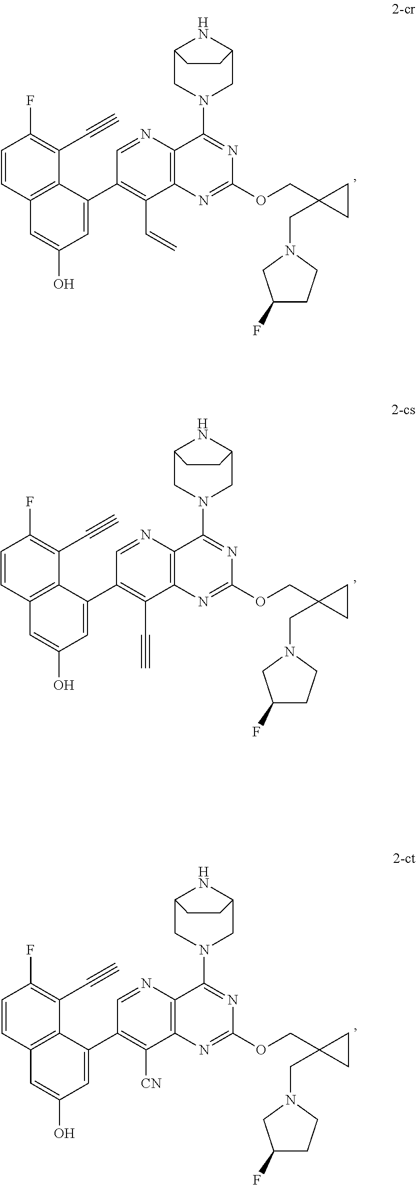 [SMILES: C#Cc1c(F)ccc2cc(O)cc(-c3cnc4c(N5CC6CCC(C5)N6)nc(OCC5(CN6CC[C@@H](F)C6)CC5)nc4c3C=C)c12 ; C#Cc1c(-c2cc(O)cc3ccc(F)c(C#C)c23)cnc2c(N3CC4CCC(C3)N4)nc(OCC3(CN4CC[C@@H](F)C4)CC3)nc12 ; C#Cc1c(F)ccc2cc(O)cc(-c3cnc4c(N5CC6CCC(C5)N6)nc(OCC5(CN6CC[C@@H](F)C6)CC5)nc4c3C#N)c12]

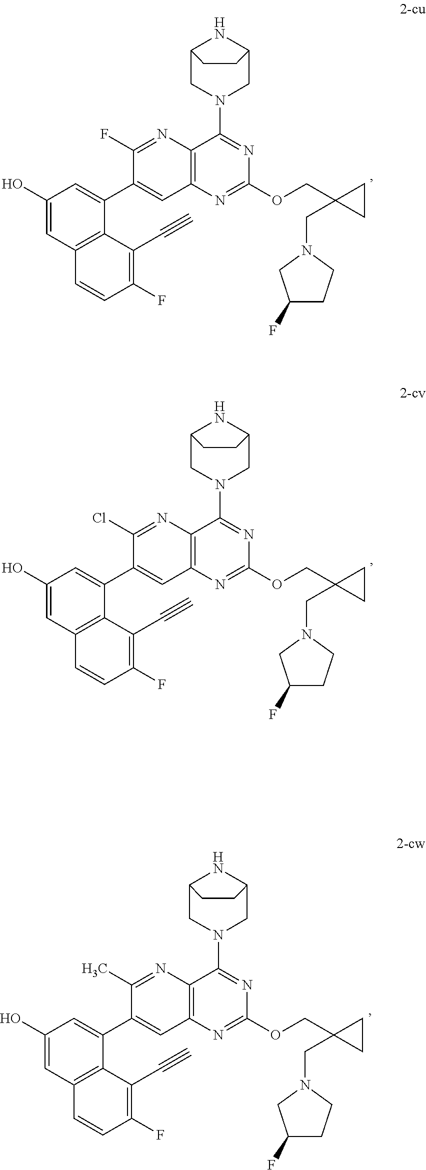 [SMILES: C#Cc1c(F)ccc2cc(O)cc(-c3cc4nc(OCC5(CN6CC[C@@H](F)C6)CC5)nc(N5CC6CCC(C5)N6)c4nc3F)c12 ; C#Cc1c(F)ccc2cc(O)cc(-c3cc4nc(OCC5(CN6CC[C@@H](F)C6)CC5)nc(N5CC6CCC(C5)N6)c4nc3Cl)c12 ; C#Cc1c(F)ccc2cc(O)cc(-c3cc4nc(OCC5(CN6CC[C@@H](F)C6)CC5)nc(N5CC6CCC(C5)N6)c4nc3C)c12]

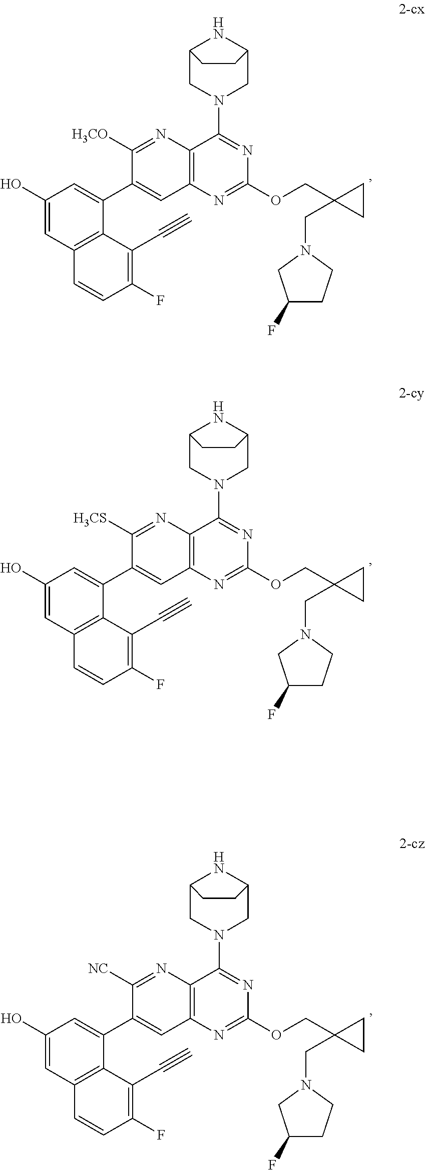

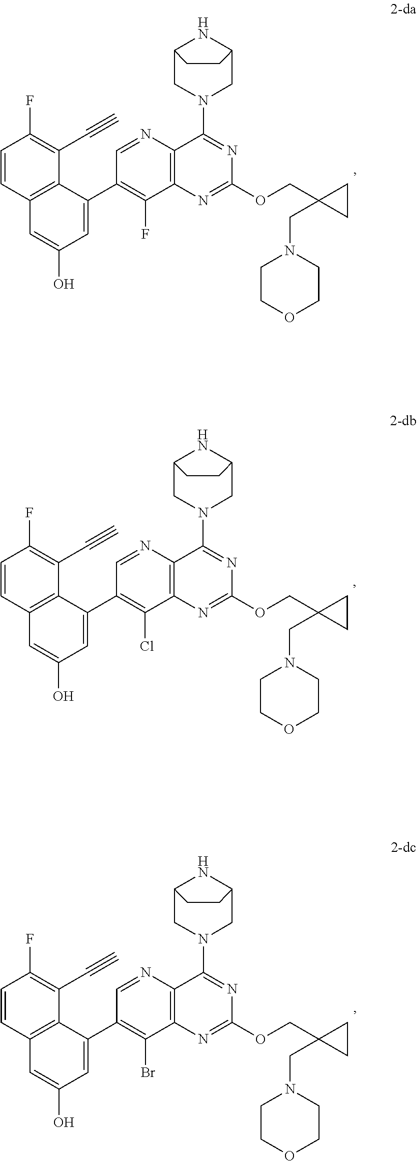 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cnc4c(N5CC6CCC(C5)N6)nc(OCC5(CN6CCOCC6)CC5)nc4c3Cl)c12 ; C#Cc1c(F)ccc2cc(O)cc(-c3cnc4c(N5CC6CCC(C5)N6)nc(OCC5(CN6CCOCC6)CC5)nc4c3Br)c12]

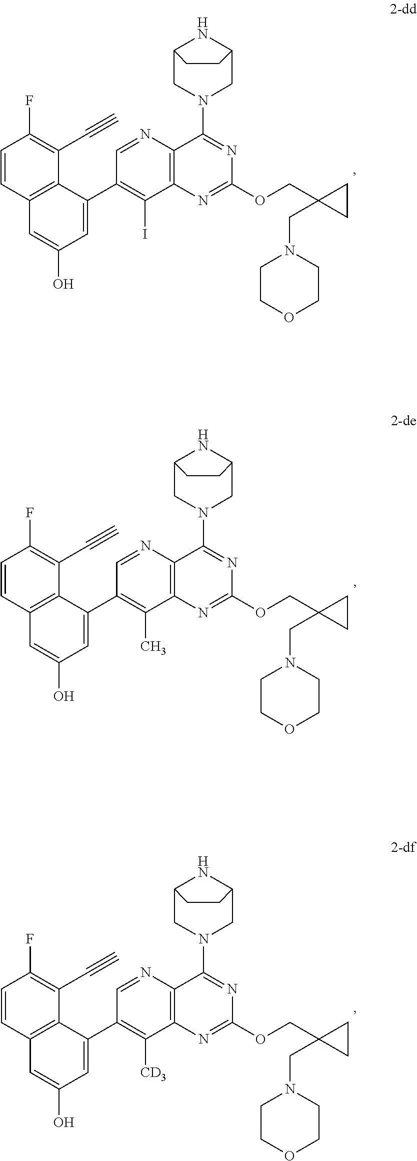 [SMILES: C#Cc1c(F)ccc2cc(O)cc(-c3cnc4c(N5CC6CCC(C5)N6)nc(OCC5(CN6CCOCC6)CC5)nc4c3I)c12 ; C#Cc1c(F)ccc2cc(O)cc(-c3cnc4c(N5CC6CCC(C5)N6)nc(OCC5(CN6CCOCC6)CC5)nc4c3C)c12 ; [2H]C([2H])([2H])c1c(-c2cc(O)cc3ccc(F)c(C#C)c23)cnc2c(N3CC4CCC(C3)N4)nc(OCC3(CN4CCOCC4)CC3)nc12]

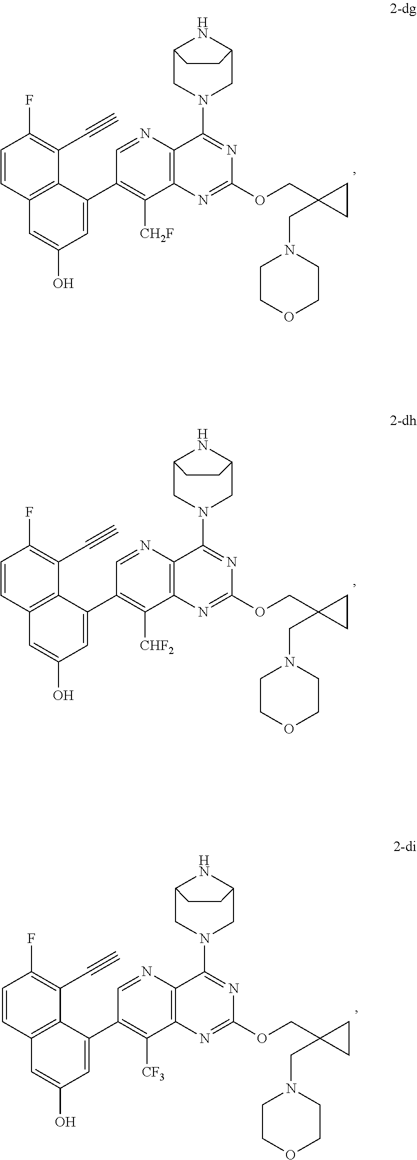

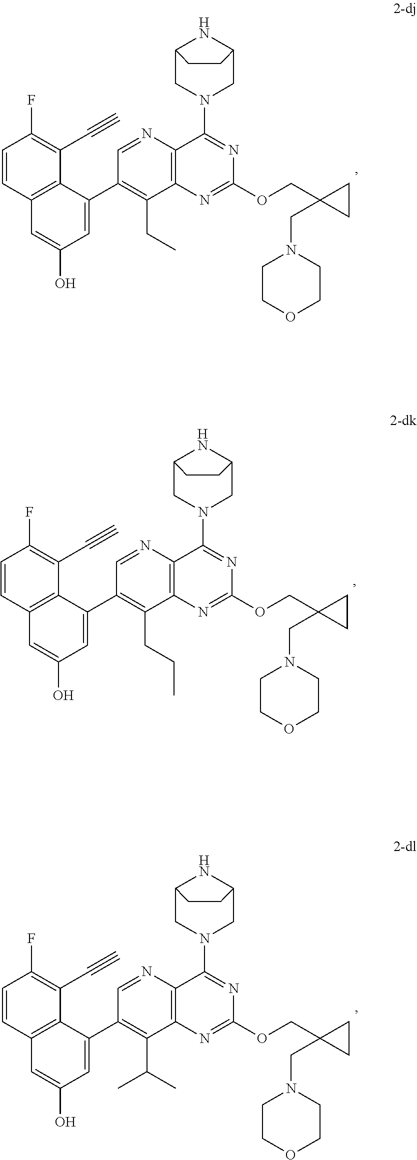 [SMILES: C#Cc1c(F)ccc2cc(O)cc(-c3cnc4c(N5CC6CCC(C5)N6)nc(OCC5(CN6CCOCC6)CC5)nc4c3CC)c12 ; C#Cc1c(F)ccc2cc(O)cc(-c3cnc4c(N5CC6CCC(C5)N6)nc(OCC5(CN6CCOCC6)CC5)nc4c3CCC)c12 ; C#Cc1c(F)ccc2cc(O)cc(-c3cnc4c(N5CC6CCC(C5)N6)nc(OCC5(CN6CCOCC6)CC5)nc4c3C(C)C)c12]

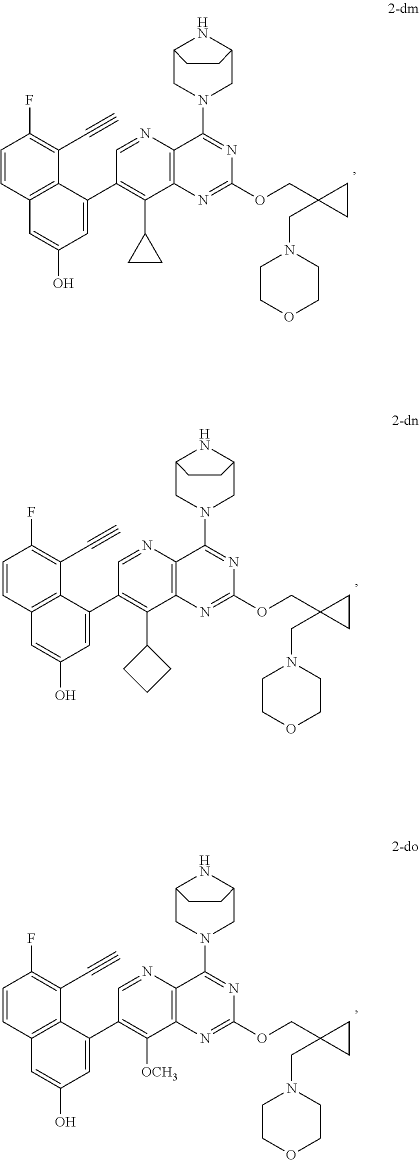 [SMILES: C#Cc1c(F)ccc2cc(O)cc(-c3cnc4c(N5CC6CCC(C5)N6)nc(OCC5(CN6CCOCC6)CC5)nc4c3C3CC3)c12 ; C#Cc1c(F)ccc2cc(O)cc(-c3cnc4c(N5CC6CCC(C5)N6)nc(OCC5(CN6CCOCC6)CC5)nc4c3C3CCC3)c12 ; C#Cc1c(F)ccc2cc(O)cc(-c3cnc4c(N5CC6CCC(C5)N6)nc(OCC5(CN6CCOCC6)CC5)nc4c3OC)c12]

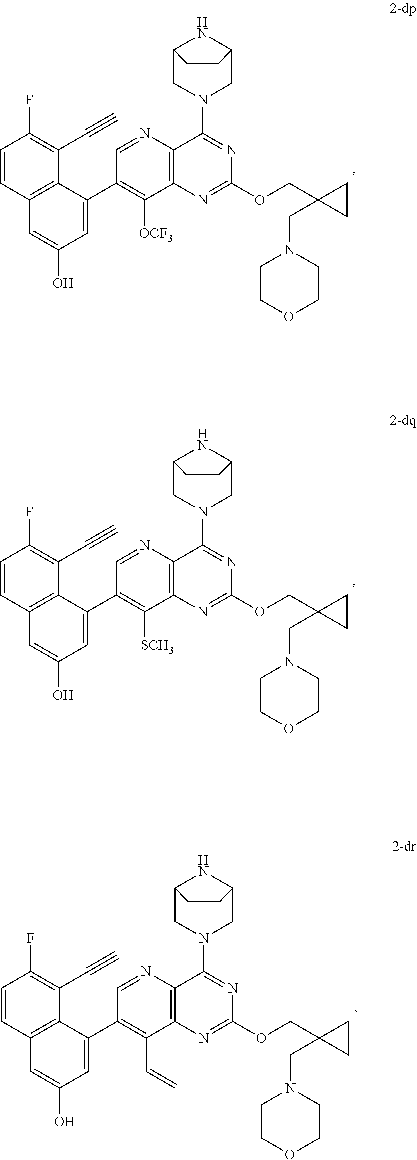 [SMILES: C#Cc1c(F)ccc2cc(O)cc(-c3cnc4c(N5CC6CCC(C5)N6)nc(OCC5(CN6CCOCC6)CC5)nc4c3OC(F)(F)F)c12 ; C#Cc1c(F)ccc2cc(O)cc(-c3cnc4c(N5CC6CCC(C5)N6)nc(OCC5(CN6CCOCC6)CC5)nc4c3SC)c12 ; C#Cc1c(F)ccc2cc(O)cc(-c3cnc4c(N5CC6CCC(C5)N6)nc(OCC5(CN6CCOCC6)CC5)nc4c3C=C)c12]

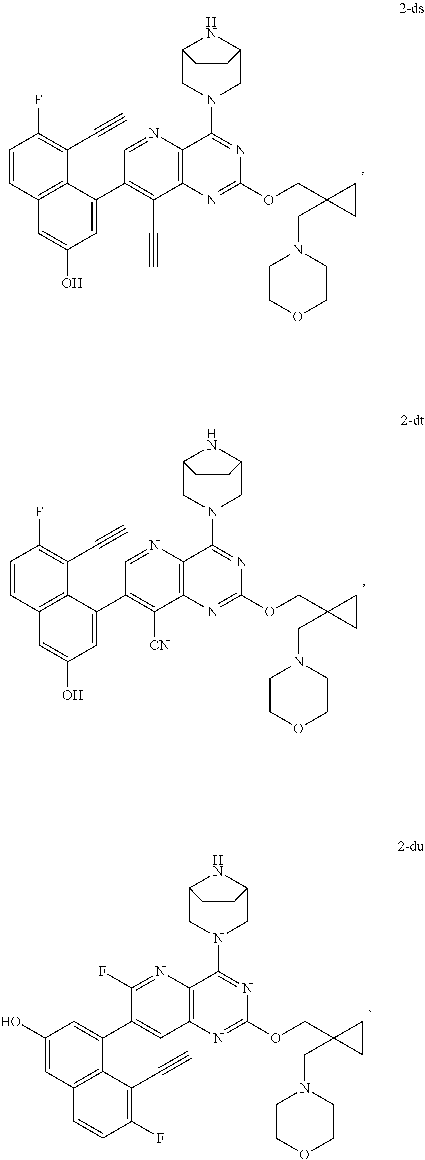 [SMILES: C#Cc1c(-c2cc(O)cc3ccc(F)c(C#C)c23)cnc2c(N3CC4CCC(C3)N4)nc(OCC3(CN4CCOCC4)CC3)nc12 ; C#Cc1c(F)ccc2cc(O)cc(-c3cnc4c(N5CC6CCC(C5)N6)nc(OCC5(CN6CCOCC6)CC5)nc4c3C#N)c12 ; C#Cc1c(F)ccc2cc(O)cc(-c3cc4nc(OCC5(CN6CCOCC6)CC5)nc(N5CC6CCC(C5)N6)c4nc3F)c12]

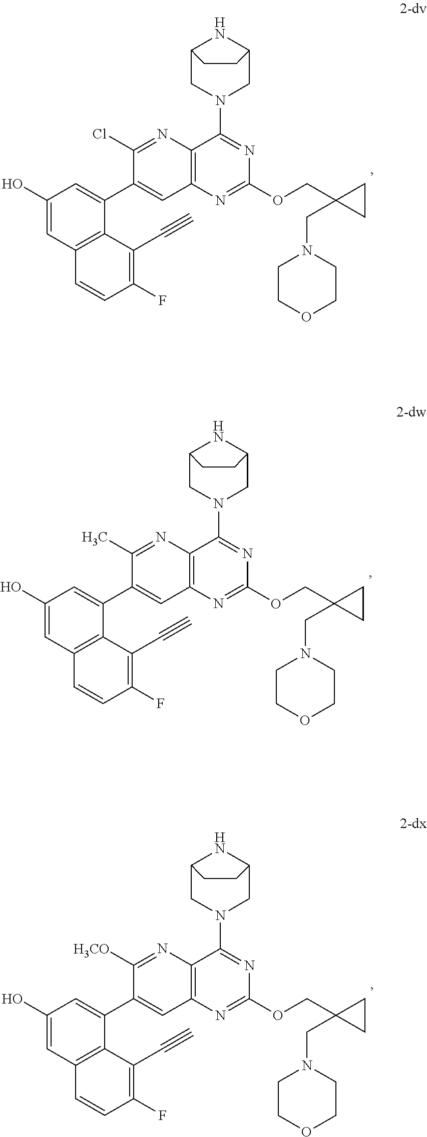 [SMILES: C#Cc1c(F)ccc2cc(O)cc(-c3cc4nc(OCC5(CN6CCOCC6)CC5)nc(N5CC6CCC(C5)N6)c4nc3Cl)c12 ; C#Cc1c(F)ccc2cc(O)cc(-c3cc4nc(OCC5(CN6CCOCC6)CC5)nc(N5CC6CCC(C5)N6)c4nc3C)c12 ; C#Cc1c(F)ccc2cc(O)cc(-c3cc4nc(OCC5(CN6CCOCC6)CC5)nc(N5CC6CCC(C5)N6)c4nc3OC)c12]

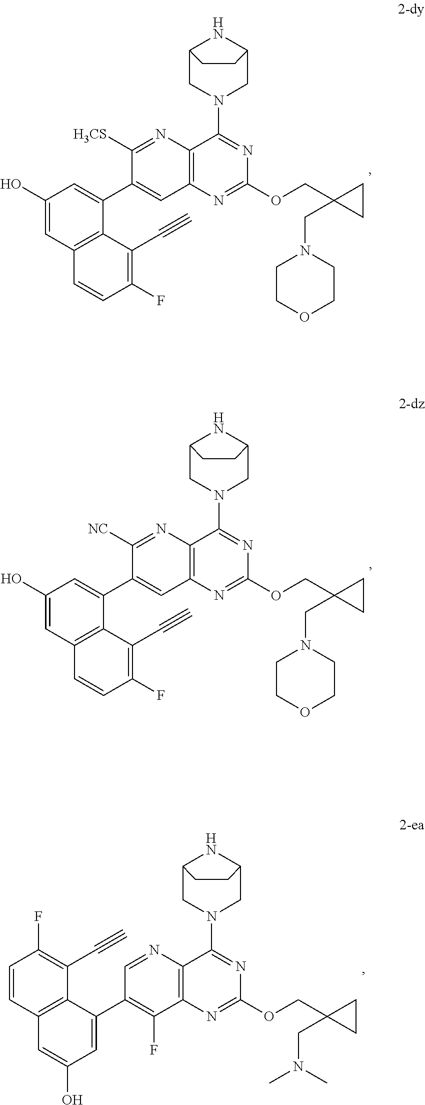

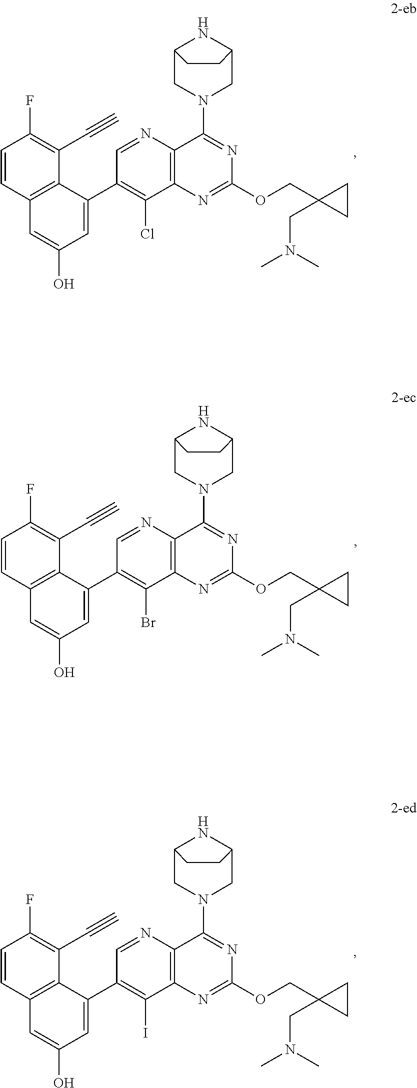 [SMILES: C#Cc1c(F)ccc2cc(O)cc(-c3cnc4c(N5CC6CCC(C5)N6)nc(OCC5(CN(C)C)CC5)nc4c3Cl)c12 ; C#Cc1c(F)ccc2cc(O)cc(-c3cnc4c(N5CC6CCC(C5)N6)nc(OCC5(CN(C)C)CC5)nc4c3Br)c12 ; C#Cc1c(F)ccc2cc(O)cc(-c3cnc4c(N5CC6CCC(C5)N6)nc(OCC5(CN(C)C)CC5)nc4c3I)c12]

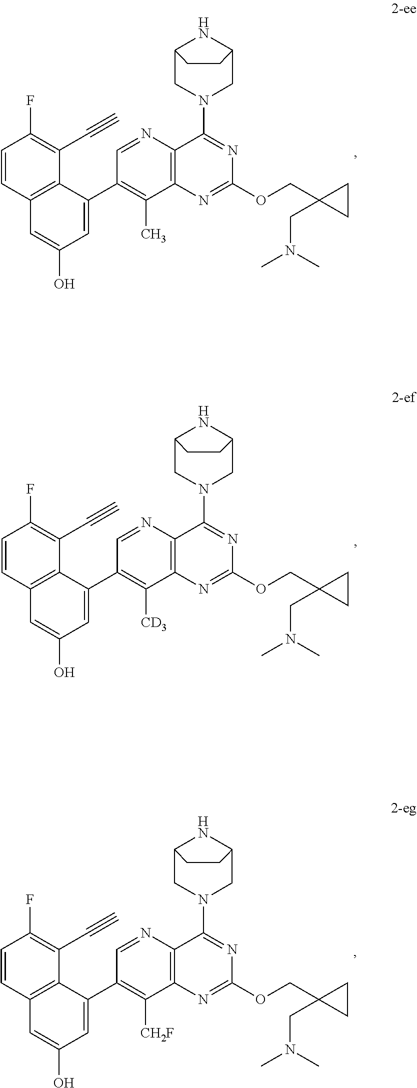

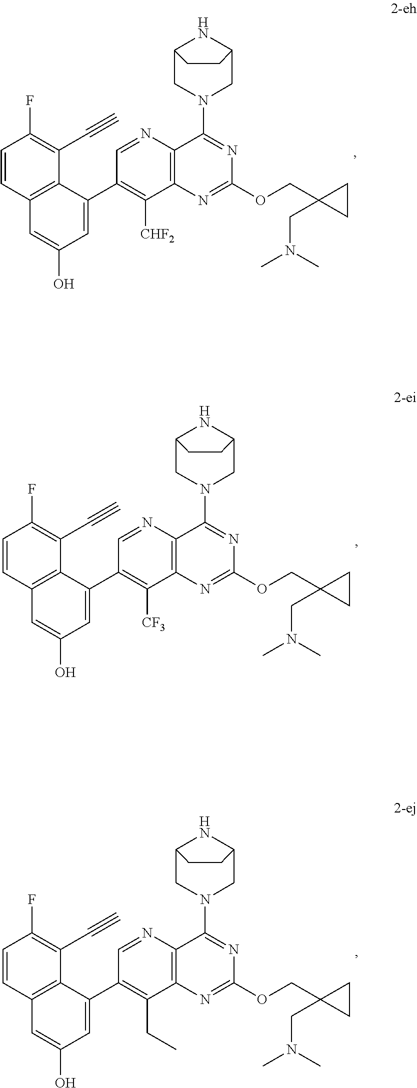 [SMILES: C#Cc1c(F)ccc2cc(O)cc(-c3cnc4c(N5CC6CCC(C5)N6)nc(OCC5(CN(C)C)CC5)nc4c3C(F)F)c12 ; C#Cc1c(F)ccc2cc(O)cc(-c3cnc4c(N5CC6CCC(C5)N6)nc(OCC5(CN(C)C)CC5)nc4c3C(F)(F)F)c12 ; C#Cc1c(F)ccc2cc(O)cc(-c3cnc4c(N5CC6CCC(C5)N6)nc(OCC5(CN(C)C)CC5)nc4c3CC)c12]

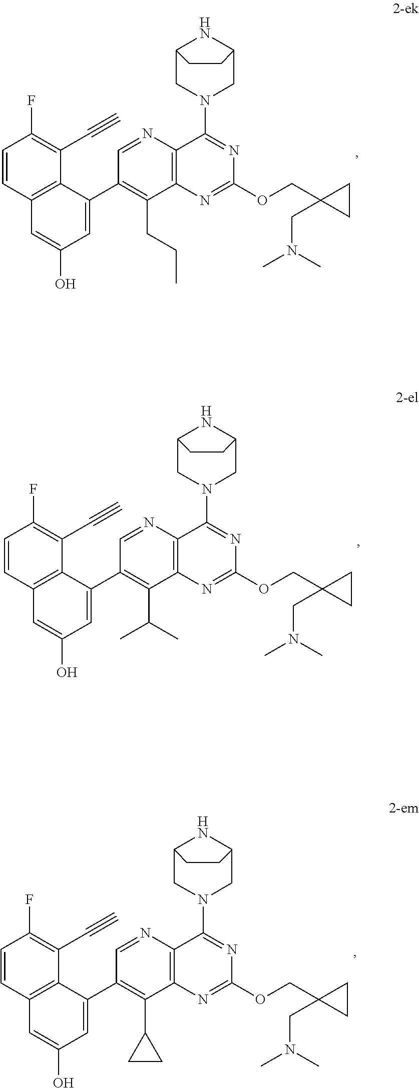 [SMILES: C#Cc1c(F)ccc2cc(O)cc(-c3cnc4c(N5CC6CCC(C5)N6)nc(OCC5(CN(C)C)CC5)nc4c3CCC)c12 ; C#Cc1c(F)ccc2cc(O)cc(-c3cnc4c(N5CC6CCC(C5)N6)nc(OCC5(CN(C)C)CC5)nc4c3C(C)C)c12 ; C#Cc1c(F)ccc2cc(O)cc(-c3cnc4c(N5CC6CCC(C5)N6)nc(OCC5(CN(C)C)CC5)nc4c3C3CC3)c12]

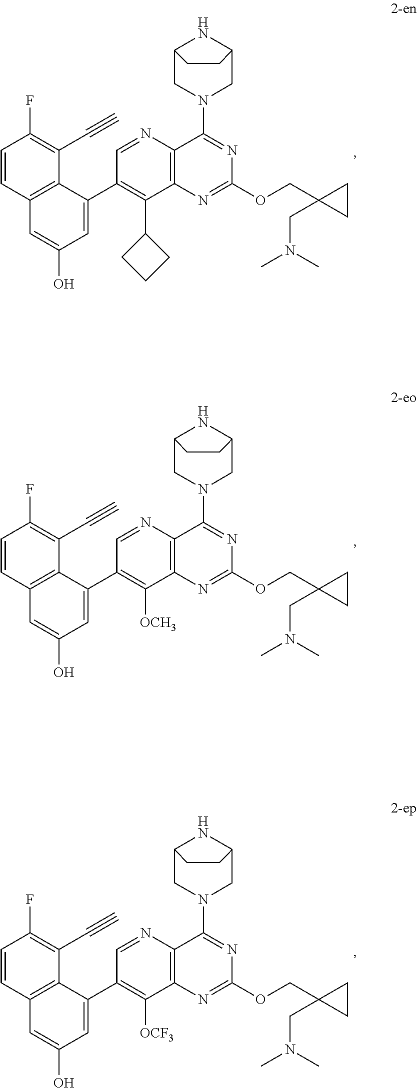 [SMILES: C#Cc1c(F)ccc2cc(O)cc(-c3cnc4c(N5CC6CCC(C5)N6)nc(OCC5(CN(C)C)CC5)nc4c3C3CCC3)c12 ; C#Cc1c(F)ccc2cc(O)cc(-c3cnc4c(N5CC6CCC(C5)N6)nc(OCC5(CN(C)C)CC5)nc4c3OC)c12 ; C#Cc1c(F)ccc2cc(O)cc(-c3cnc4c(N5CC6CCC(C5)N6)nc(OCC5(CN(C)C)CC5)nc4c3OC(F)(F)F)c12]

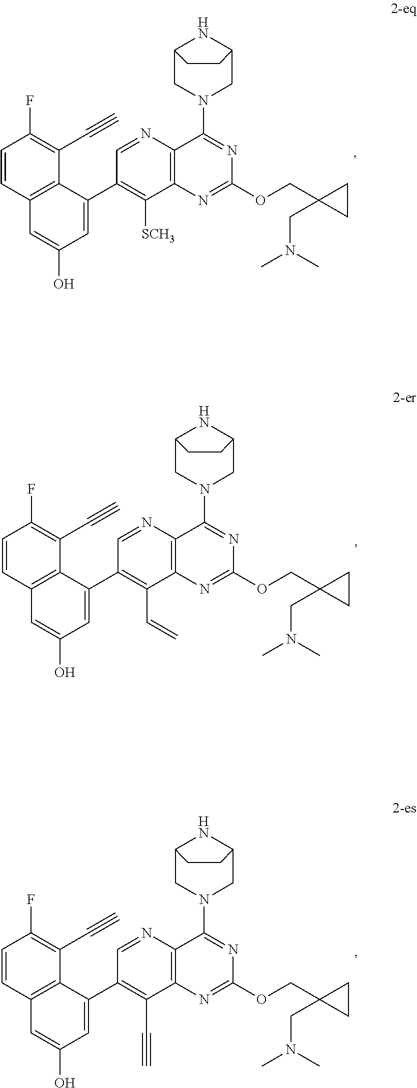 [SMILES: C#Cc1c(F)ccc2cc(O)cc(-c3cnc4c(N5CC6CCC(C5)N6)nc(OCC5(CN(C)C)CC5)nc4c3SC)c12 ; C#Cc1c(F)ccc2cc(O)cc(-c3cnc4c(N5CC6CCC(C5)N6)nc(OCC5(CN(C)C)CC5)nc4c3C=C)c12 ; C#Cc1c(-c2cc(O)cc3ccc(F)c(C#C)c23)cnc2c(N3CC4CCC(C3)N4)nc(OCC3(CN(C)C)CC3)nc12]

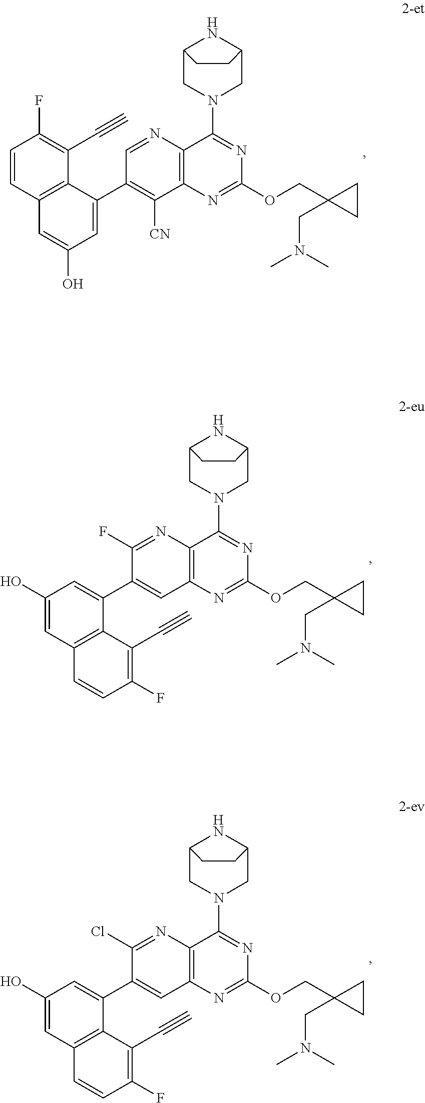 [SMILES: C#Cc1c(F)ccc2cc(O)cc(-c3cnc4c(N5CC6CCC(C5)N6)nc(OCC5(CN(C)C)CC5)nc4c3C#N)c12 ; C#Cc1c(F)ccc2cc(O)cc(-c3cc4nc(OCC5(CN(C)C)CC5)nc(N5CC6CCC(C5)N6)c4nc3F)c12 ; C#Cc1c(F)ccc2cc(O)cc(-c3cc4nc(OCC5(CN(C)C)CC5)nc(N5CC6CCC(C5)N6)c4nc3Cl)c12]

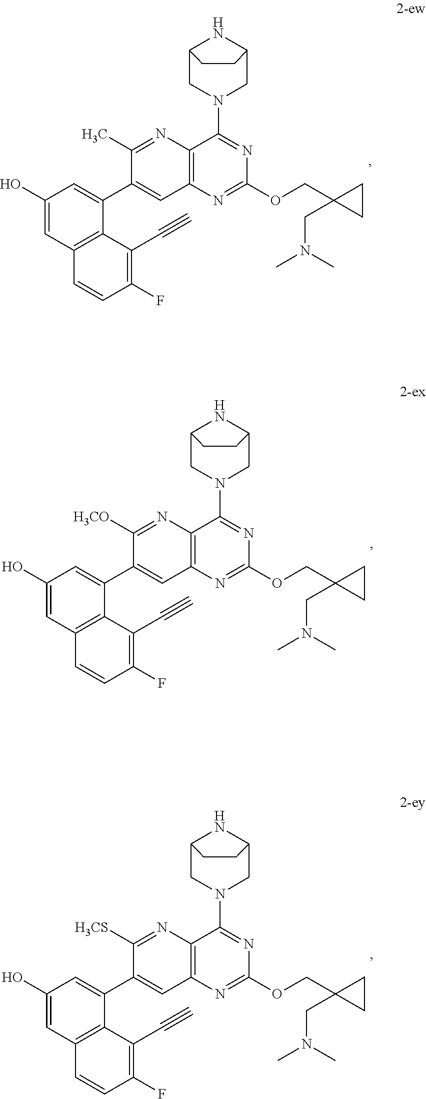

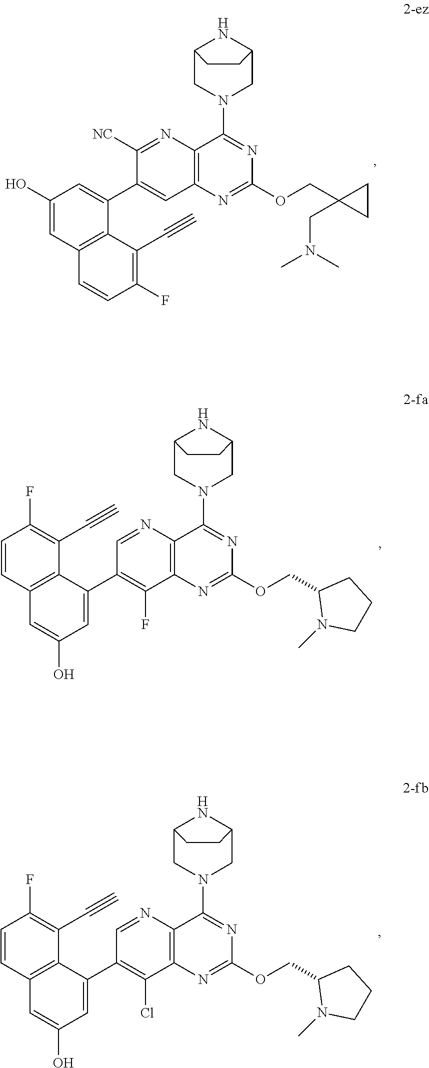

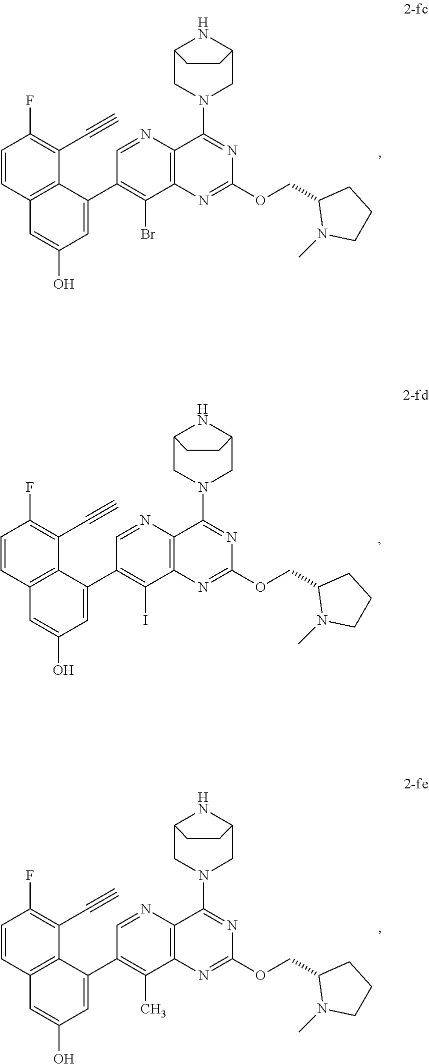

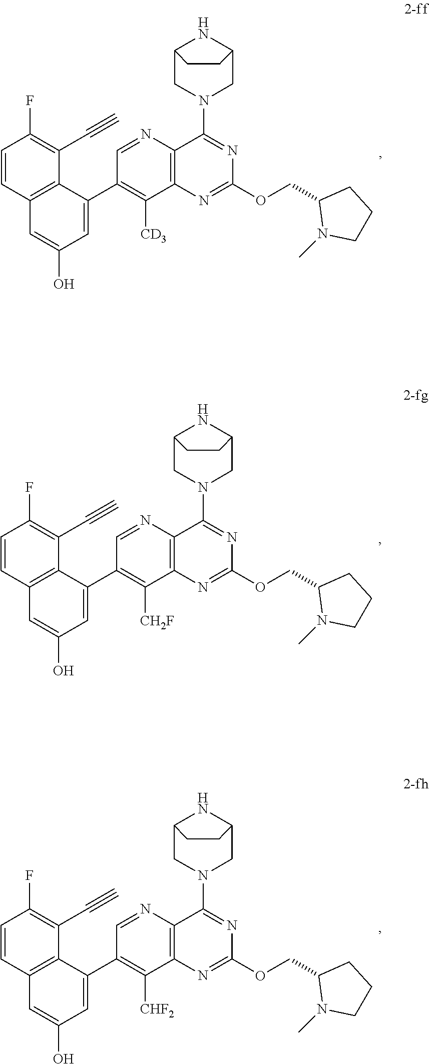

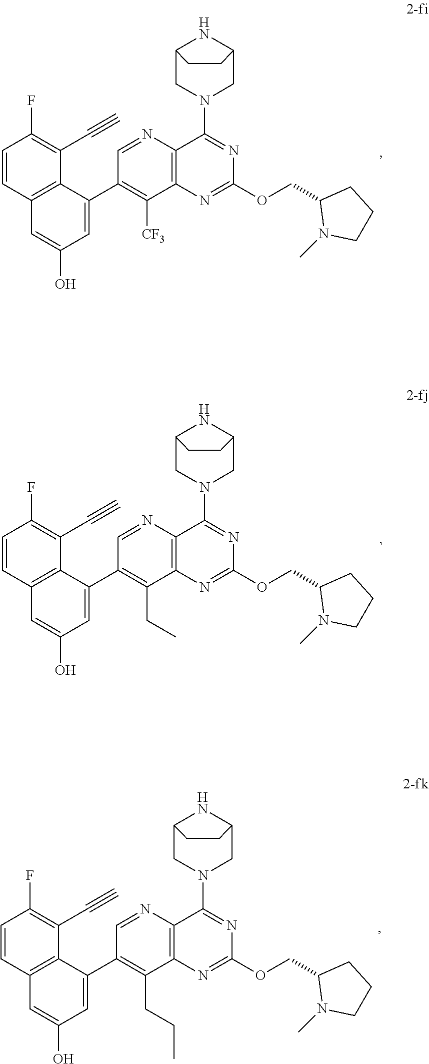

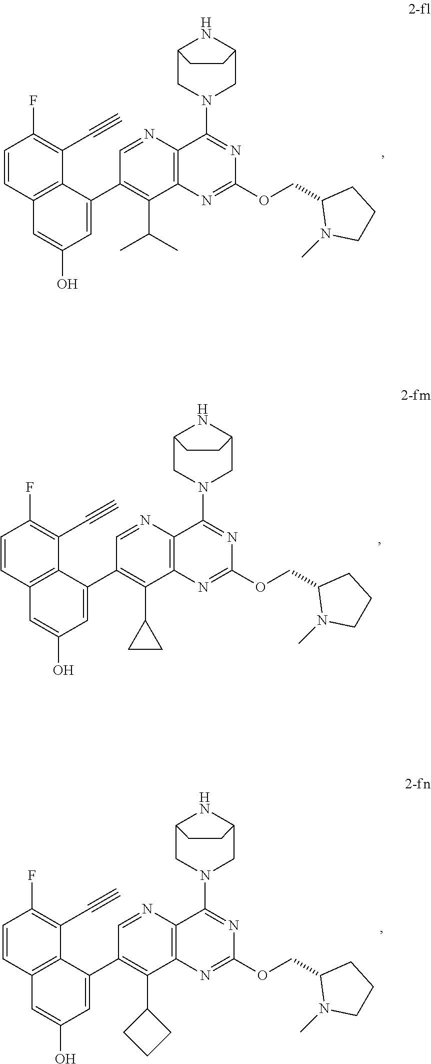

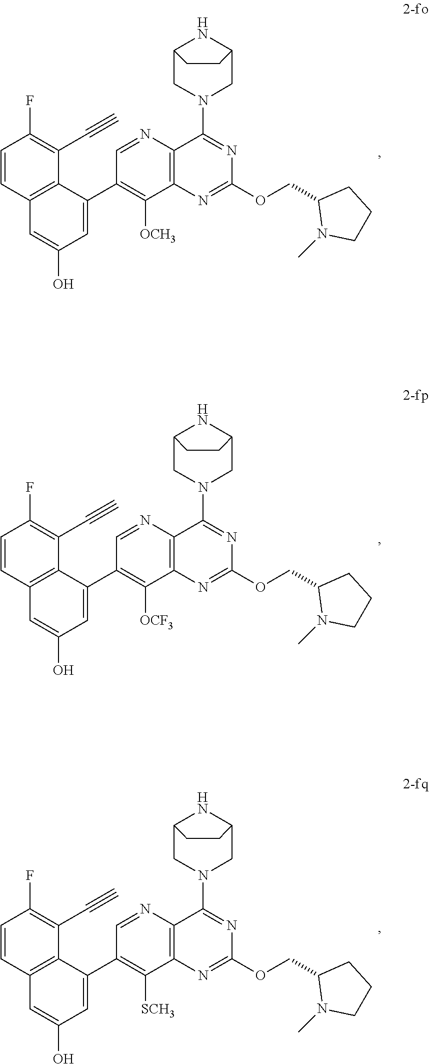

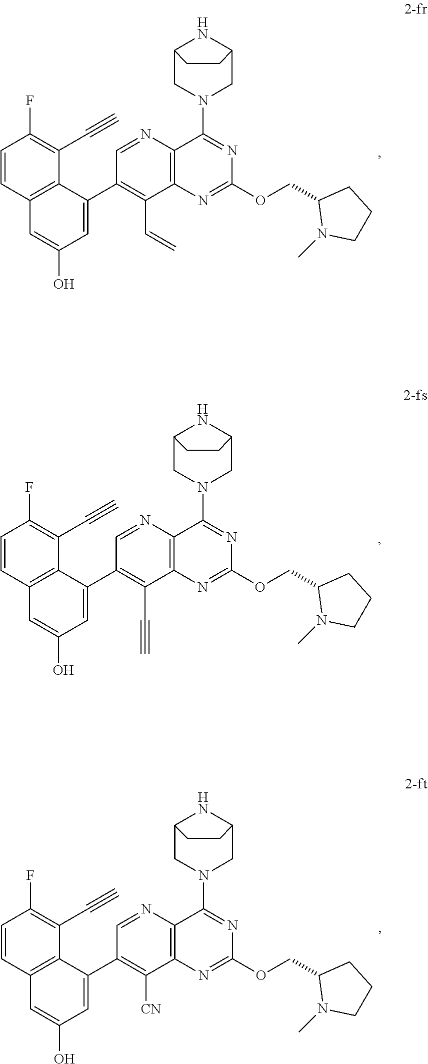

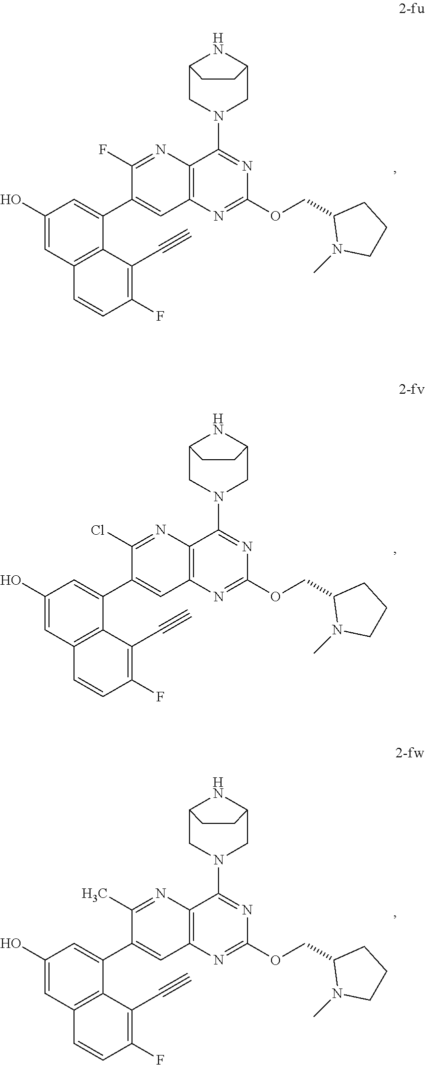

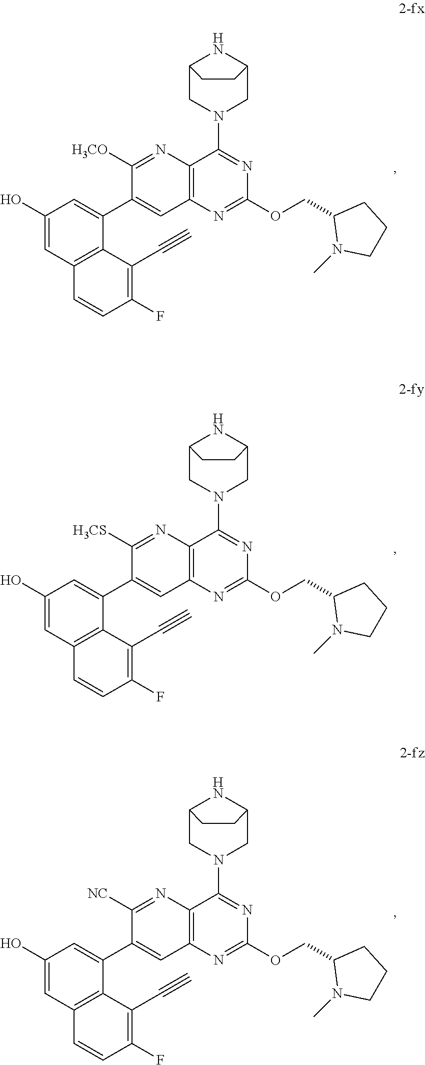

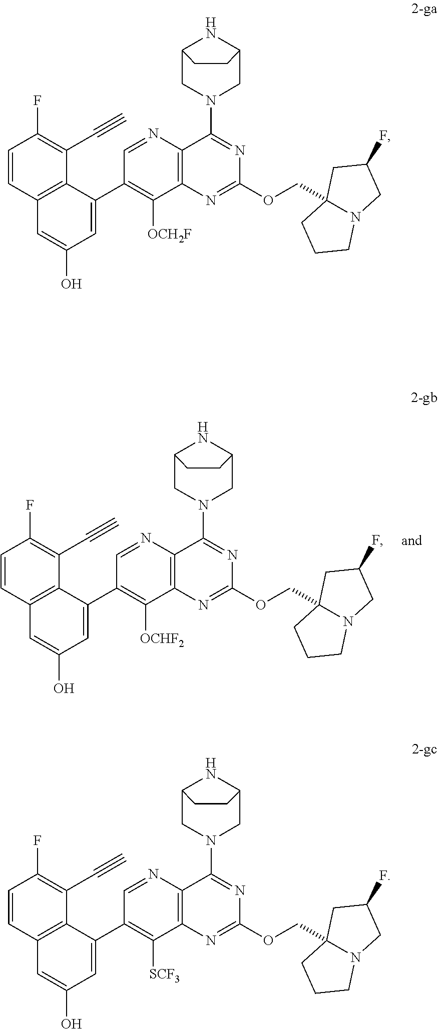

Particularly preferred compounds shown below are 1-ap, 1-bo, 1-ca, 1-dk, 1-ei, 1-gb, 2-ab, 2-af, 2-ba, 2-be, 2-ca, 2-ce, 2-da, 2-de, 2-ea, 2-ee, and 2-fa:

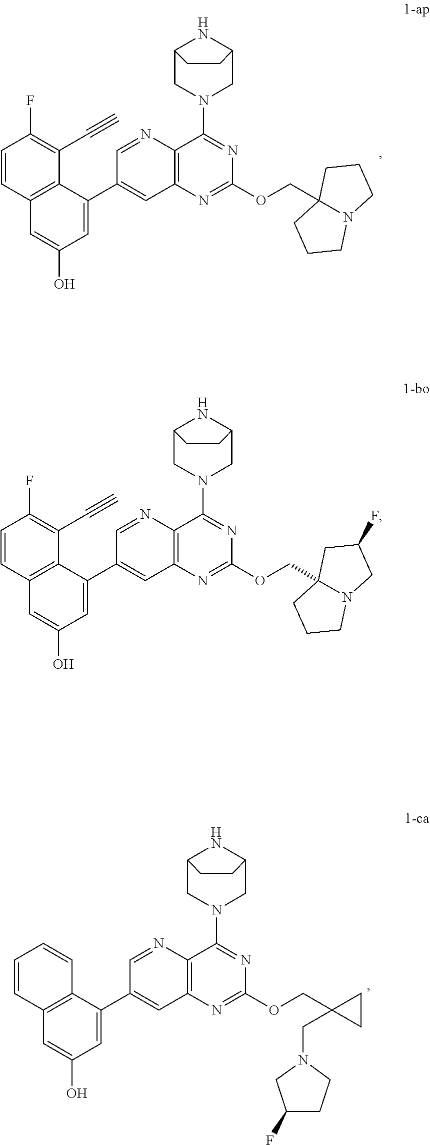 [SMILES: C#Cc1c(F)ccc2cc(O)cc(-c3cnc4c(N5CC6CCC(C5)N6)nc(OCC56CCCN5CCC6)nc4c3)c12 ; C#Cc1c(F)ccc2cc(O)cc(-c3cnc4c(N5CC6CCC(C5)N6)nc(OC[C@@]56CCCN5C[C@H](F)C6)nc4c3)c12 ; Oc1cc(-c2cnc3c(N4CC5CCC(C4)N5)nc(OCC4(CN5CC[C@@H](F)C5)CC4)nc3c2)c2ccccc2c1]

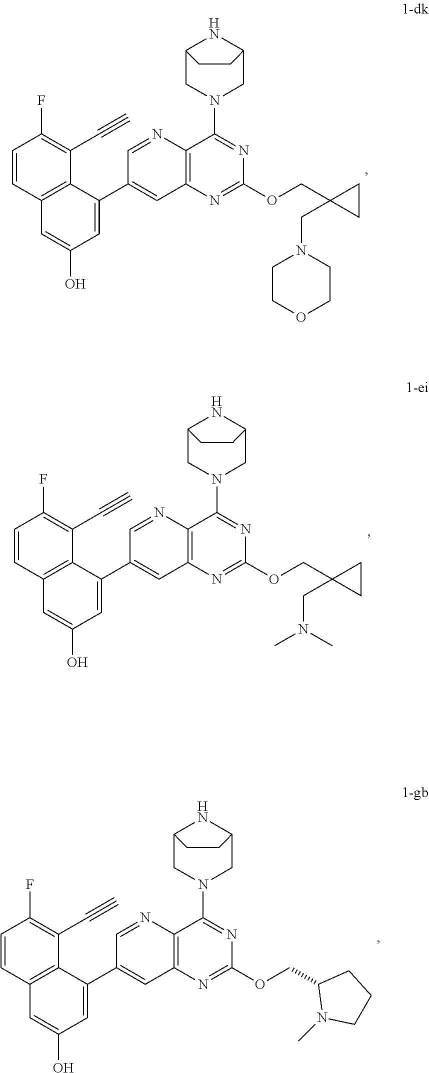

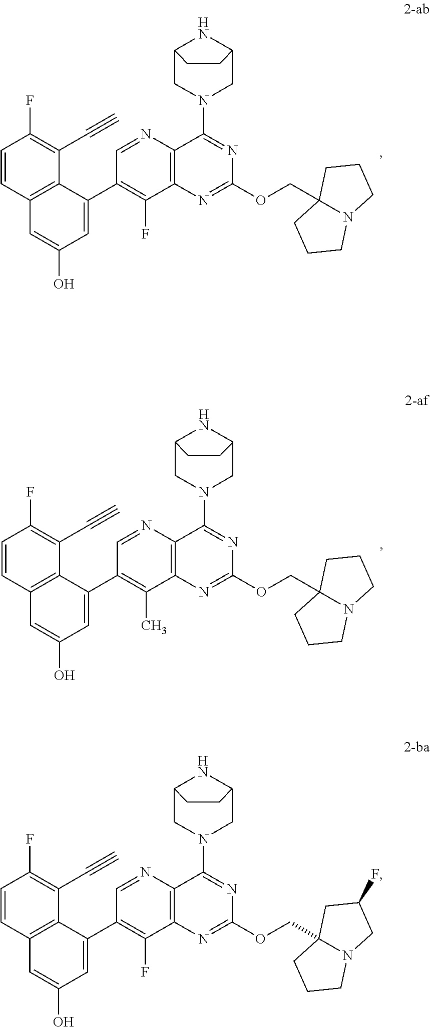 [SMILES: C#Cc1c(F)ccc2cc(O)cc(-c3cnc4c(N5CC6CCC(C5)N6)nc(OCC56CCCN5CCC6)nc4c3F)c12 ; C#Cc1c(F)ccc2cc(O)cc(-c3cnc4c(N5CC6CCC(C5)N6)nc(OCC56CCCN5CCC6)nc4c3C)c12 ; C#Cc1c(F)ccc2cc(O)cc(-c3cnc4c(N5CC6CCC(C5)N6)nc(OC[C@@]56CCCN5C[C@H](F)C6)nc4c3F)c12]

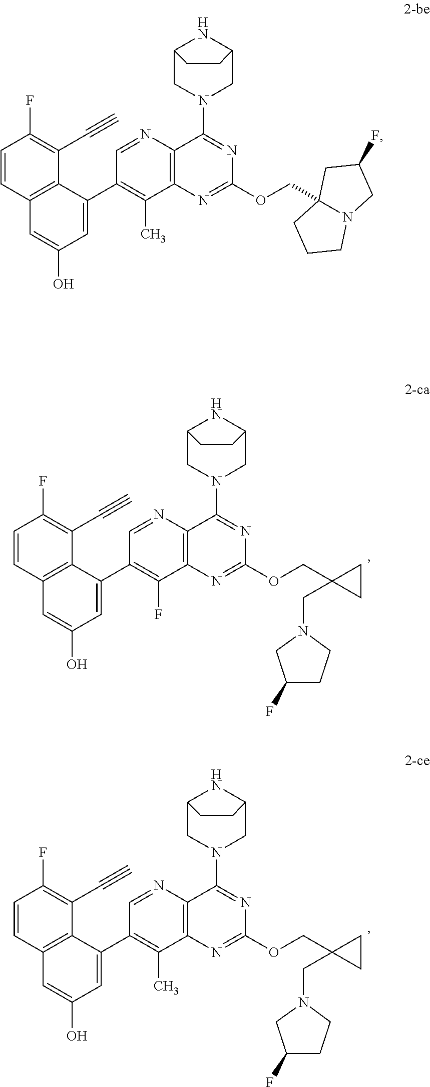 [SMILES: C#Cc1c(F)ccc2cc(O)cc(-c3cnc4c(N5CC6CCC(C5)N6)nc(OC[C@@]56CCCN5C[C@H](F)C6)nc4c3C)c12 ; C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cnc4c(N5CC6CCC(C5)N6)nc(OCC5(CN6CC[C@@H](F)C6)CC5)nc4c3C)c12]

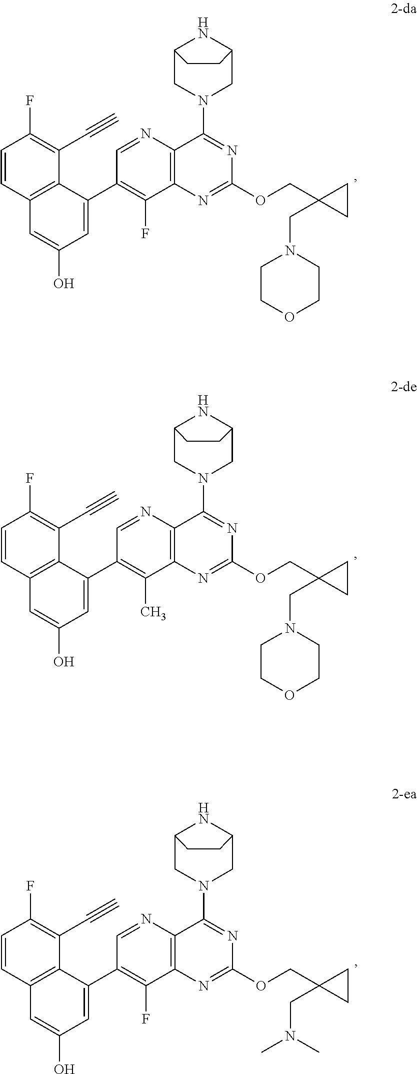 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cnc4c(N5CC6CCC(C5)N6)nc(OCC5(CN6CCOCC6)CC5)nc4c3C)c12 ; C#Cc1c(F)ccc2cc(O)cc(-c3cnc4c(N5CC6CCC(C5)N6)nc(OCC5(CN(C)C)CC5)nc4c3F)c12]

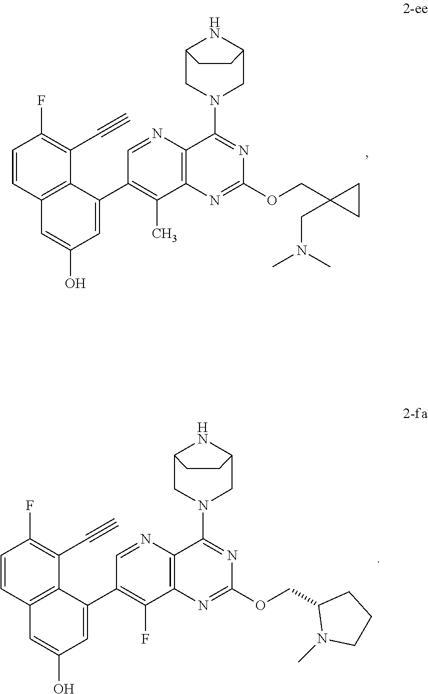

Compounds disclosed herein can be administered to a patient as the neat or freebase chemical, but are preferably administered as a pharmaceutical composition. Accordingly, the invention encompasses pharmaceutical compositions comprising a compound or a salt (including a pharmaceutically acceptable salt) of a compound, such as a compound of Formula I, together with at least one pharmaceutically acceptable carrier. The pharmaceutical composition may contain a compound or salt of Formula I as the only active agent, but preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formula I and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formula I, and an additional active agent. For example, the pharmaceutical composition may contain a molar ratio of about 0.5:1, about 1:1, about 2:1, about 3:1 or from about 1.5:1 to about 4:1 of an additional active agent to a compound of Formula I. Particularly preferred forms of Formula I for use in a pharmaceutical composition includes compounds 1-ap, 1-bo, 1-ca, 1-dk, 1-ei, 1-gb, 2-ab, 2-af, 2-ba, 2-be, 2-ca, 2-ce, 2-da, 2-de, 2-ea, 2-ee, and 2-fa or a salt, solvate or prodrug thereof, together with a pharmaceutically acceptable carrier.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt %) of a compound of Formula I and usually at least about 5 wt % of a compound of Formula I. Some embodiments contain from about 25 wt % to about 50 wt % or from about 5 wt % to about 75 wt % of the compound of Formula I.

### Treatment Methods

The compounds of Formula I, as well as pharmaceutical compositions comprising the compounds, are useful for diagnosis or treatment of a disease, disorder, or medical condition mediated through KRAS, especially the KRAS mutant G12D, and including various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer, pancreatic cancer, as well as combinations of these.

According to the present invention, a method of KRAS-mediated diseases or conditions comprises providing to a patient in need of such treatment a therapeutically effective amount of a compound of Formula I. In one embodiment, the patient is a mammal, and more specifically a human. As will be understood by one skilled in the art, the invention also encompasses methods of treating non-human patients such as companion animals, e.g., cats, dogs, and livestock animals.

A therapeutically effective amount of a pharmaceutical composition is preferably an amount sufficient to reduce or ameliorate the symptoms of a disease or condition. In the case of KRAS-mediated diseases for example, a therapeutically effective amount may be an amount sufficient to reduce or ameliorate cancer. A therapeutically effective amount of a compound or pharmaceutical composition described herein will also provide a sufficient concentration of a compound of Formula I when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound of Formula I to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I are provided daily to a patient. Frequency of dosage may also vary depending on the compound used and the particular disease treated. However, for treatment of most KRAS-mediated diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I may be administered singularly (i.e., sole therapeutic agent of a regime) to treat or prevent KRAS-mediated diseases and conditions such as various cancers, or may be administered in combination with another active agent. Forms of Formula I may be used in combination, with or without another active agent. For example, in one embodiment, one, two, three, four or more compounds of Formula I may be combined with or without an additional active agent to form a therapeutic combination. One or more compounds of Formula I may be administered in coordination with a regime of one or more other active agents such as anticancer cytotoxic agents. In an embodiment, a method of treating or diagnosing KRAS-mediated cancer in a mammal includes administering to said mammal a therapeutically effective amount of a compound of Formula I, optionally in combination with one or more additional active ingredients.

As will be appreciated by one skilled in the art, the methods of treatment provided herein are also useful for treatment of mammals other than humans, including for veterinary applications such as to treat horses and livestock, e.g. cattle, sheep, cows, goats, swine and the like, and pets (companion animals) such as dogs and cats.

For diagnostic or research applications, a wide variety of mammals will be suitable subjects including rodents (e.g., mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, saliva, feces, and urine) and cell and tissue samples of the above subjects will be suitable for use.

In one embodiment, the invention provides a method of treating a disease, disorder, or medical condition mediated through KRAS, especially the KRAS mutant G12D, including various cancers, in a patient identified as in need of such treatment, the method comprising providing to the patient an effective amount of a compound of Formula I. The compounds of Formula I provided herein may be administered alone, or in combination with one or more other active agents.

In another embodiment, the method of treating or diagnosing KRAS-mediated diseases or conditions may additionally comprise administering the compound of Formula I in combination with one or more additional compounds, wherein at least one of the additional compounds is an active agent, to a patient in need of such treatment. The one or more additional compounds may include additional therapeutic compounds, including anticancer cytotoxic agents and/or anticancer therapeutic compounds such as doxorubicin, paclitaxel, docetaxel, cisplatin, camptothecin, temozolomide, avastin, Herceptin, Erbitux, EGFR inhibitors, osimertinib, rezivertinib, CDK 4/6 inhibitors, abemaciclib, palbociclib, ribociclib, c-MET inhibitors, capmatinib, volitinib, ALK inhibitors, crizotinib, alectinib, ceritinib, brigatinib, entrectinib, lorlatinib, PD-1 antagonists, PD-L1 antagonists, ipilimumab, embrolizumab, nivolumab and the like, as well as combinations of these.

### EXAMPLES

### Chemical Synthesis

The compounds of Formula I described herein, and/or the pharmaceutically acceptable salts thereof, can be synthesized from commercially available starting materials by methods well known to those skilled in the art of synthetic organic chemistry. The following general synthetic Schemes 1-4 illustrate representative methods to prepare most of the example compounds. In the specific examples where the Suzuki cross-coupling reaction of arylboronic acids/esters with organohalides/pseudohalides (Beketskaya, I. P. et al., Coordin. Chem. Rev. 2019, 385, 137-173) is either impractical or unsuccessful, then the corresponding Stille cross-coupling reaction of organostannanes with organohalides/pseudohalides may be used as an alternative (Espinet, P. et al., ACS Catal. 2015, 5, 3040-3053). The listed starting materials, reactions, reagents, solvents, temperatures, catalysts and ligands are not limited to what is depicted for purely illustrative purposes. Certain abbreviations and acronyms well known to those trained in the art that may be used in Schemes 1-7 and in the Examples are listed hereinbelow for clarity.

Scheme 1 illustrates the synthesis of pyrido[2,3-d]pyrimidine examples 1a-c of the Formula I where W is nitrogen, Y and X are both CR⁵ and L is either O, S or NR³. Reaction of a 2,6-dichloronicotinamide derivative 1d with oxalyl chloride (1e) in toluene at reflux furnishes the corresponding 7-chloropyrido[2,3-d]pyrimidine-2,4(1H,3H)-dione (1f). Chlorination of if with phosphorous oxychloride with warming in the presence of Hünig's base of provides the corresponding 2,4,7-trichloropyrido[2,3-d]pyrimidine 1g. Subsequent reaction of 1g with 3,8-diazabicyclo[3.2.I]octane derivative 1h followed by treatment with (Boc)₂O affords intermediate 1i. Treatment of compounds 1j-1l with a base in an appropriate solvent followed by reaction with intermediate 1i yields the corresponding compounds 1m-1o. The base can be selected from K₂CO₃, Cs₂CO₃, DIEA or potassium t-butoxide, potassium bis(trimethylsilyl)amide or sodium hydride. Depending upon the base, the appropriate solvent can be chosen from THF, 1,4-dioxane, acetonitrile, DMA, NMP and alcohols. In the particular case where L is 0 (1j), an alternative palladium-mediated coupling reaction with 1i can be used to obtain compound 1m. Typical conditions for this coupling reaction are PdCl₂, DTBPF, and K₂CO₃ in a mixture of 1,4-dioxane and water at elevated temperature. Subsequent Suzuki coupling of compounds 1m-1o with either boronic acid 1p or a corresponding borate ester in the presence of Pd(dppf)Cl₂ and KOAc yields the corresponding coupled products 1q-1s. Removal of the Boc groups from compounds 1q-1s under acidic conditions such as a mixture of TFA in DCM affords the desired products 1a-1c.

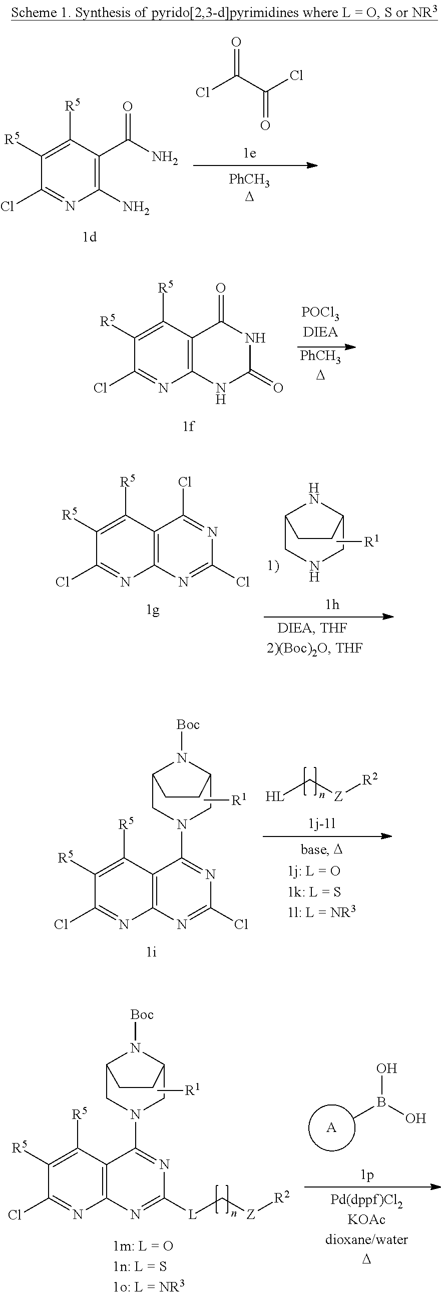

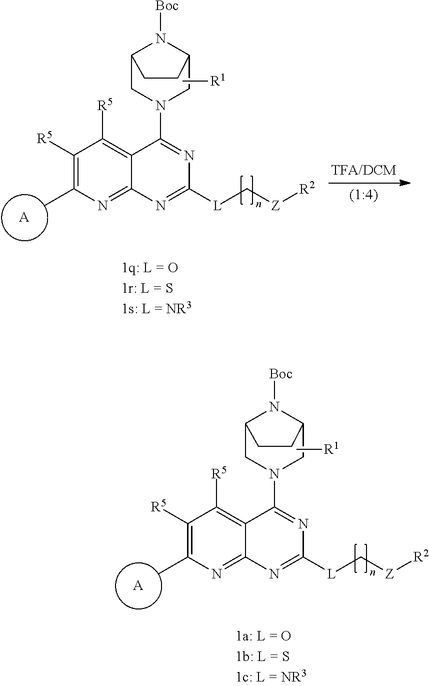

Scheme 2 illustrates the synthesis of pyrimido[4,5-d]pyrimidine examples 2a-c of the Formula I where W and X are both nitrogen, Y is CR⁵ and L is either 0, S or NR³. Suzuki coupling of compound 2d with either boronic acid 1p or a corresponding borate ester in the presence of Pd(OAc)₂, DTBPF and K₃PO₄ furnishes the corresponding coupled product 2e. Reaction of 2e with methyl chloroformate in the presence of sodium hydride yields carbamate 2f. Dropwise addition of 30% H₂O₂ to a solution of 2f in the presence of sodium hydroxide generates compound 2g. Chlorination of 2g with phosphorous oxychloride in the presence of Hunig's base of provides compound 2h. Subsequent reaction of 2 h with 3,8-diazabicyclo[3.2.1]octane derivative 1h followed by treatment with (Boc)₂O affords intermediate 2i. Treatment of compounds 1j-1l with a base in an appropriate solvent followed by reaction with intermediate 2i yields the corresponding compounds 2j-2l. The base can be selected from K₂CO₃, Cs₂CO₃, DIEA or potassium t-butoxide, potassium bis(trimethylsilyl)amide or sodium hydride. Depending upon the base, the appropriate solvent can be chosen from THF, 1,4-dioxane, acetonitrile, DMA, NMP and alcohols. In the particular case where L is O (1j), an alternative palladium-mediated coupling reaction with 2i can be used to obtain compound 2j. Typical conditions for this coupling reaction are PdCl₂, DTBPF, and K₂CO₃ in a mixture of 1,4-dioxane and water at elevated temperature. Removal of the Boc groups from compounds 2j-2l under acidic conditions such as a mixture of TFA in DCM affords the desired amine products 2a-2c.

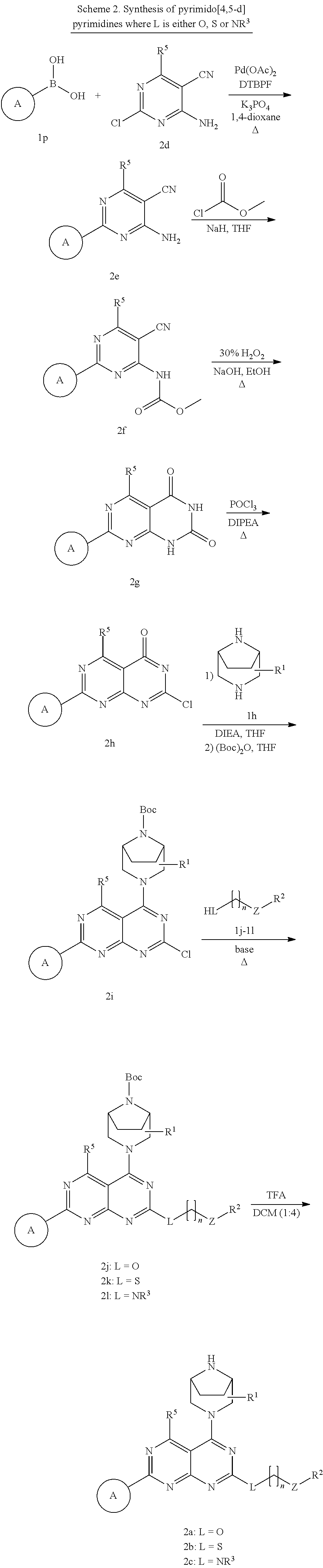

Scheme 3 illustrates the synthesis of pyrido[3,2-d]pyrimidine examples 3a-c of the Formula I where Y is nitrogen, W and X are both CR⁵ and L is either 0, S or NR³. Reaction of 3-aminopicolinic acid 3d with urea in the presence of aqueous 0.2 M sodium hydroxide provides pyrido[3,2-d]pyrimidine-2,4(1H,3H)-dione 3e. Chlorination of 3e with a mixture of POCl₃ and PCl₅ the corresponding trichlorinated product 3f. Subsequent reaction of 3f with 3,8-diazabicyclo[3.2.1]octane derivative 1h followed by treatment with (Boc)₂O affords intermediate 3g. Treatment of compounds 1i-k with a base in an appropriate solvent followed by reaction with intermediate 3g yields the corresponding compounds 3h-3j. The base can be selected from K₂CO₃, Cs₂CO₃, DIEA or potassium t-butoxide, potassium bis(trimethylsilyl)amide or sodium hydride. Depending upon the base, the appropriate solvent can be chosen from THF, 1,4-dioxane, acetonitrile, DMA, NMP and alcohols. Alternatively, 1i-1k and 3g can be coupled with Pd(OAc)₂ in the presence of BINAP and Cs₂CO₃ in toluene at elevated temperature to produce compounds 3h-3j. Subsequent Suzuki coupling of compounds 3h-3j with either boronic acid 1p or a corresponding borate ester in the presence of Pd(dppf)Cl₂ and KOAc yields the corresponding coupled products 3k-3m. Alternative conditions for this Suzuki coupling reaction are PdCl₂, DTBPF, and K₂CO₃ in a mixture of 1,4-dioxane and water at elevated temperature. Removal of the Boc groups from compounds 3k-3m under acidic conditions such as HCl in 1,4-dioxane or TFA in dichloromethane affords the desired amine products 3a-3c.

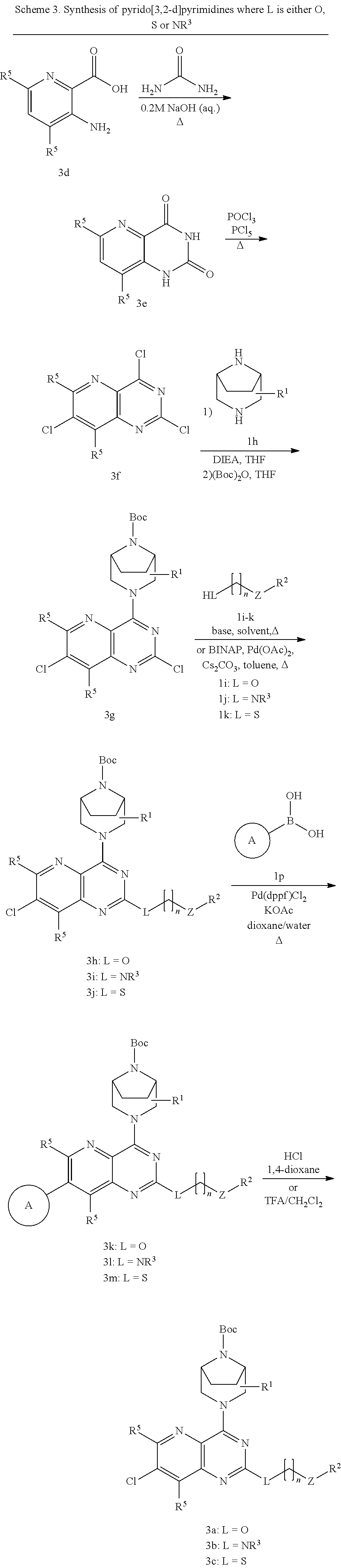

Scheme 4 illustrates the synthesis of examples of the Formula I represented by 4 where L is methylene. The reaction of terminal acetylene 4b with a strong base such as sodium hydride generates the corresponding acetylide anion, which can then be reacted with compound 4a to provide 4c. Alternatively, the Sonogashira coupling of 4b with 4b using a Pd catalyst such as Pd(dppf)₂Cl₂ can furnish compound 4c (Plenio, H. and Schulz, M. J. Org. Chem. 2012, 77 (6), 2798-2807). Suzuki coupling 4c with 1p under standard conditions followed by of catalytic hydrogenation furnished 4d. Removal of the Boc group under acidic conditions such as TFA in dichloromethane affords the desired amine product 4.

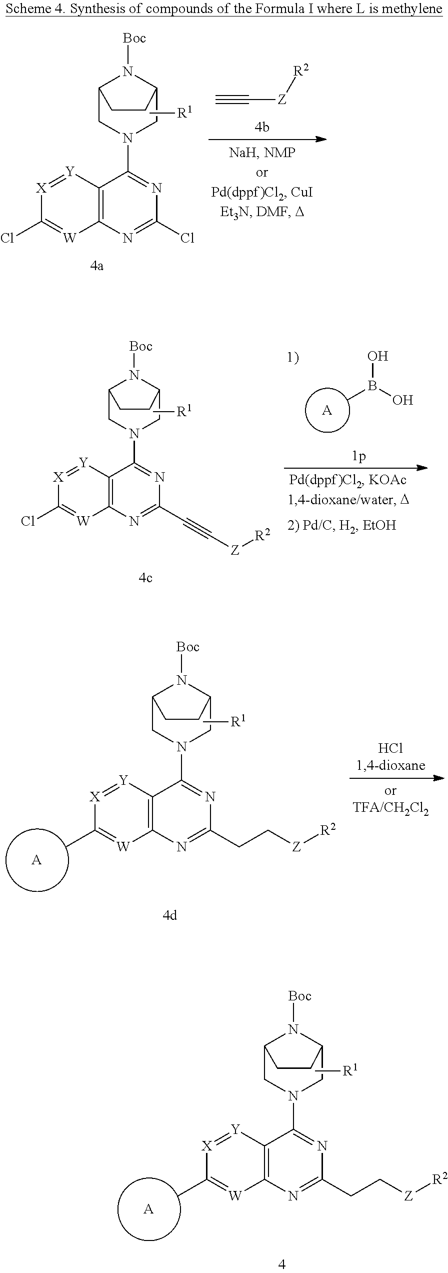

Scheme 5 illustrates the synthesis of compounds 5a and 5b of Formula I where R⁷ or R⁸ are fluorine and L is either O, S or NR³. Oxidation of commercially available 7-bromopyrido[3,2-d]pyrimidine-2,4-diol (5c) with urea-hydrogen peroxide complex in the presence of trifluoroacetic anhydride at 0° C. in an aprotic solvent such as DMF provides N-oxide 5d. Subsequent reaction of 5d with POCl₃ in the presence of Hunig's base generates a roughly 1:1 mixture of trichloro compounds 5e and 5f. Treatment of the 5e/5f mixture with 1h in the presence of Hunig's base in a solvent followed by reaction with (Boc)₂O furnishes the corresponding products 5g and 5h, which may be separated by chromatography. Treatment of compounds 1i-k with a base such as sodium hydride, Hunig's base, K₂CO₃ or a Cs₂CO₃/DABCO mixture followed by reaction with either 5g or 5h in a polar aprotic solvent such as N-methyl-2-pyrrolidone at RT or elevated temperature affords compounds 5i and 5j, respectively. Alternatively, 5g or 5 h and 1i-k can be coupled with Pd(OAc)₂ the presence of BINAP and Cs₂CO₃ in toluene at elevated temperature to produce 5i and 5j, respectively. Reaction of either 5i or 5j with a fluoride source such as potassium fluoride or cesium fluoride at elevated temperature in a polar aprotic solvent like DMSO furnishes the corresponding fluoro products 5k and 51. A standard Suzuki coupling procedure with either compounds 5k or 51 and 1p in a solvent mixture such as 1,4-dioxane and water can be employed to prepare compounds 5m and 5n, respectively. Removal of the Boc protecting group of 5m or 5n under acidic conditions such as anhydrous HCl in 1,4-dioxane or TFA in DCM will produce the corresponding compounds 5a-5b of Formula I where R⁷ or R⁸ are fluorine and L is either 0, S or NR³.

Similarly, the compounds 5a and 5b of Formula I where R⁷ or R⁸ are chlorine and L is either 0, S or NR³ can be prepared in an analogous fashion by elimination of the KF or CsF fluorination step.

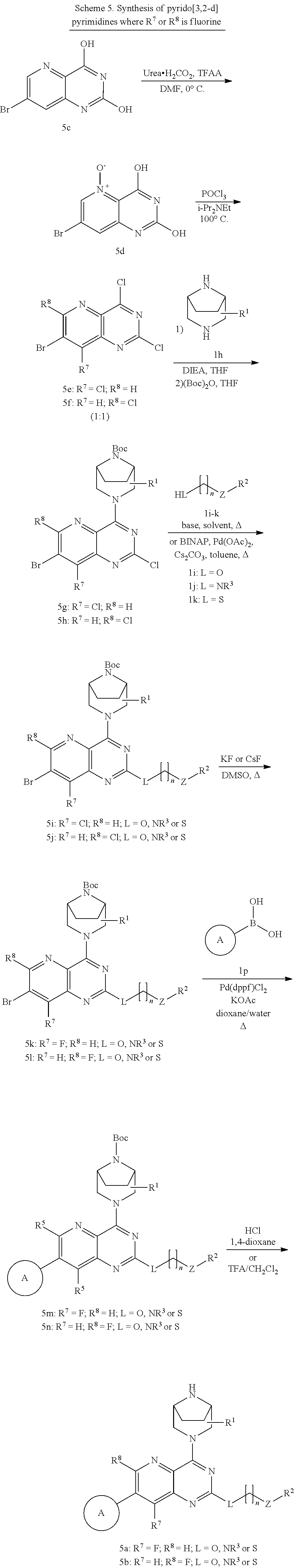

Scheme 6 illustrates an alternate synthesis of pyrido[3,2-d]pyrimidines 6a-6c of Formula I where L is either 0, S or NR³. Reaction of either dibromo or dichloro pyridine derivative 6d with (1Z)—N-[(methylsulfonyl)oxy]-ethanimidoyl chloride (6e; CAS #1228558-17-5) according to the general procedure described by P. S. Fier (J. Am. Chem. Soc. 2017, 139(28), 9499-9736) provides 3,5-dihalo-4-fluoropicolinonitrile (6f). Alternatively, compound 6f can be prepared by the oxidation of 6d with H₂02-urea complex in the presence of trifluoroacetic anhydride followed by treatment of the corresponding N-oxides with trimethylsilyl cyanide in the presence of dimethylcarbamoyl chloride in a solvent such as dichloromethane. Regioselective Suzuki coupling of 6f with boronic acid 1p as generally described in WO2021117767A1 affords product 6g. Subsequent reaction of 6g with 2,4-dimethoxybenzylamine (6h) according to the procedure described in WO2021041671A1 in the presence of Hünig's base while heating in a suitable solvent such as 1,4-dioxane furnishes compound 6i. Alternatively, 6i can be prepared by a Buchwald-Hartwig amination procedure between 6g and 6h under standard conditions. The Pinner reaction of 6i in methanol in the presence of HCl conducted at −78° C. to 0° C. followed by hydrolysis of the intermediate imino ester in the presence of saturated aqueous NaHCO₃ affords compound 6j. Reaction of 6j with trichloroacetyl isocyanate at 0° C. followed by treatment with anhydrous ammonia in methanol and warming to room temperature provides compound 6k. Reaction of 6k with POCl₃ in the presence of Hünig's base at elevated temperature yields the corresponding 2,4-dichloro-8-fluoropyrido[3,2-d]pyrimidine derivative 61. Reaction of compound 61 with 1h in the presence of Hunig's base in a solvent such as acetonitrile provides 6m. Treatment of 1i-1k with a suitable base such as potassium fluoride, Hünig's base, K₂CO₃ or a Cs₂CO₃/DABCO mixture in either neat 1i-1k or in a suitable aprotic solvent followed by reaction with 6m at elevated temperature affords compounds 6n-6p, respectively. Alternatively, li-1k and 6m can be coupled with Pd(OAc)₂ in the presence of BINAP and Cs₂CO₃ in toluene at elevated temperature to produce 6n-6p, respectively. Removal of the Boc protecting group of 6n-6p under acidic conditions such as anhydrous HCl in 1,4-dioxane or TFA in DCM provides compounds 6a-6c of the of Formula I where L is either 0, S or NR³.

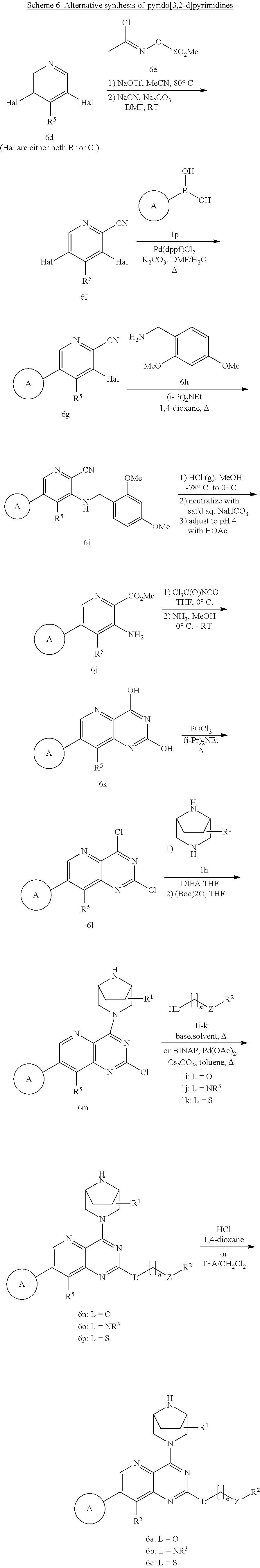

Scheme 7 illustrates yet another alternate synthesis of pyrido[3,2-d]pyrimidines 7a-7c of the Formula I where L is either 0, S or NR³. Nitration of a picolinic acid derivative 7d with nitric acid in concentrated sulfuric acid provides the corresponding nitro compound 7e. Esterification of 7e with catalytic sulfuric acid in methanol at reflux generates methyl ester 7f. Reduction of the nitro group of 7f with stannous chloride dihydrate in the presence of hydrochloric acid in an alcohol such as ethanol furnishes the corresponding amino derivative 7g. Hydrolysis of the ester moiety of 7g in a wet solvent such as acetonitrile or THF containing 6% water (v/v) in the presence of LiBr and triethylamine provides carboxylic acid 7h (S. Karlsson et al, Tet. Lett. 2007 48, 2497-2499). Treatment of 7 h with HATU and ammonium chloride in the presence of sodium bicarbonate in a polar aprotic solvent such as DMF generates the corresponding carboxamide derivative 7i. Reaction of 7i with triphosgene (bis(trichloromethyl) carbonate in an aprotic solvent such as 1,4-dioxane at 5° C. followed by heating at 110° C. yields 7-bromopyrido[3,2-d]pyrimidine-2,4(1H,3H)-dione derivative 7j. Reaction of 7j with phosphorous oxychloride in the presence of Hunig's base at 120° C. provides the corresponding dichloro derivative 7k. Reaction of 7k with 1h in the presence of Hünig's base in a solvent such as acetonitrile provides 71. Treatment of 1i-1k with a suitable base such as sodium hydride, Hünig's base, K₂CO₃ or a Cs₂CO₃/DABCO mixture followed by reaction with 71 at elevated temperature generates affords compounds 7m-7o, respectively. Alternatively, 1i-1k and 7l can be coupled with Pd(OAc)₂ the presence of BINAP and Cs₂CO₃ in toluene at elevated temperature to produce 7m-7o. A standard Suzuki coupling procedure between 7m-7o and 1p can be employed to prepare compounds 7p-7r. Removal of the Boc protecting group of 7p-7r under acidic conditions such as anhydrous HCl in 1,4-dioxane furnishes pyrido[3,2-d]pyrimidines 7a-7c of the Formula I where L is either O, S or NR³.

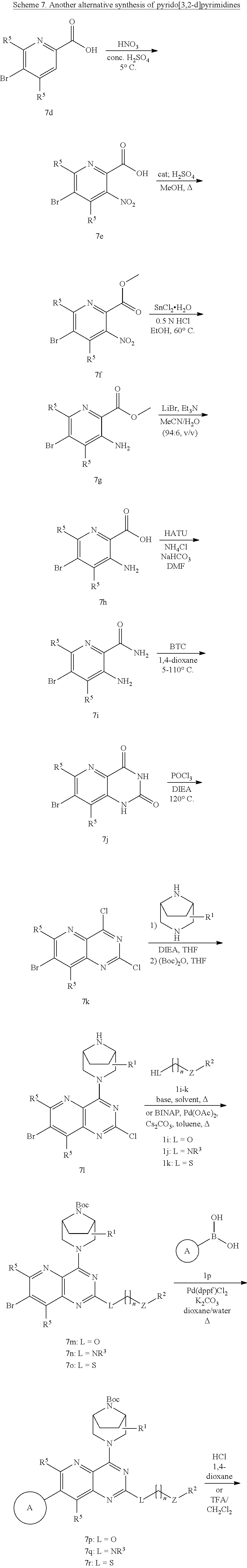

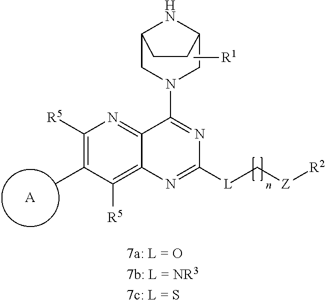

### Abbreviations and Acronyms

The following abbreviations and acronyms may be used in this application:
anhyd.=anhydrous;
aq.=aqueous;
B₂pin₂=bis(pinacolato)diboron;
Boc=tert-butoxycarbonyl;
n-Bu₃P=tri-n-butylphosphine;
Compd=compound;
d=day(s);
DCM=dichloromethane;
DIEA=DIPEA=N,N-diisopropylethylamine=Hunig's base
DMF=N,N-dimethylformamide;
DMSO=dimethylsulfoxide;
DMA=N,N-dimethylacetamide;
dppf=1,1′-bis(diphenylphosphino)ferrocene);
DTBPF=1,1′-bis(di-tert-butylphosphino)ferrocene;
EtOAc=ethyl acetate;
equiv=equivalents;
Ex=Example;
h=hour(s);
KOAc=potassium acetate;
LiHMDS=lithium bis(trimethylsilyl)amide [LiN(SiMe₃)₂];
MeOH=methanol;
NMP=N-methyl-2-pyrrolidone;
min=minutes;
Pd(dppf)Cl₂=[1,1′-bis(diphenylphosphino)ferrocene]dichloropalladium(II);
RT=room temperature;
satd.=saturated solution;
TEA=triethylamine;
TFA=trifluoroacetic acid;
THF=tetrahydrofuran;

### EXAMPLES

The present inventive concept has been described in terms of exemplary principles and embodiments, but those skilled in the art will recognize that variations may be made and equivalents substituted for what is described without departing from the scope and spirit of the disclosure as defined by the following claims.

### Example 1

4-((1R,5S)-3,8-Diazabicyclo[3.2.1]octan-3-yl)-7-(8-chloronaphthalen-1-yl)-2-((tetrahydro-1H-pyrrolizin-7a(5H)-yl)methoxy)pyrido[3,2-d]pyrimidine hydrochloride (1:3) (1-aa).

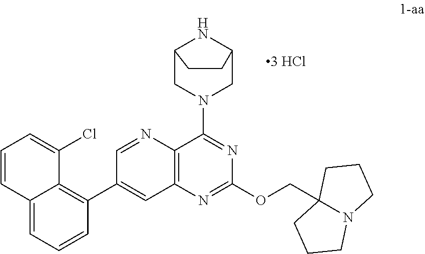 [SMILES: Clc1cccc2cccc(-c3cnc4c(N5CC6CCC(C5)N6)nc(OCC56CCCN5CCC6)nc4c3)c12]

Example 1 (1-aa) was prepared as shown below in Scheme 8.

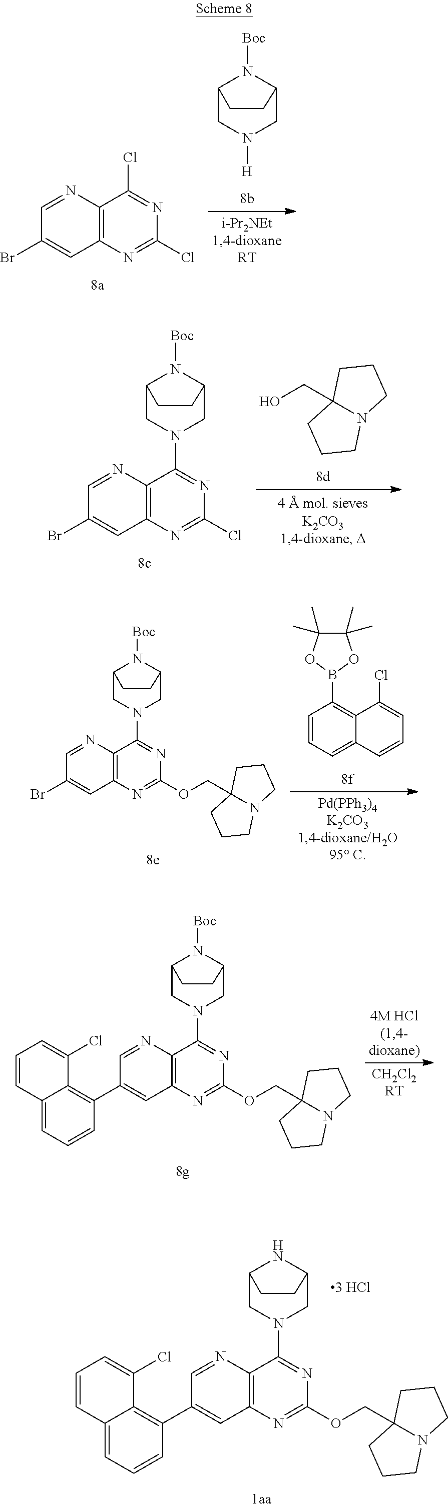

tert-Butyl (1R,5S)-3-(7-bromo-2-chloropyrido[3,2-d]pyrimidin-4-yl)-3,8-diazabicyclo[3.2.1]octane-8-carboxylate (8c). A mixture of 7-bromo-2,4-dichloropyrido[3,2-d]pyrimidine (8a; CAS #1215074-41-1; 500 mg, 1.80 mmol) and tert-butyl 3,8-diazabicyclo [3.2.1]octane-8-carboxylate (8b; CAS #149771-44-8; 420 mg, 1.98 mmol) in anhydrous 1,4-dioxane (5 mL) was treated slowly dropwise with diisopropylethylamine (1 mL, 5.40 mmol) and the resulting yellow suspension stirred at RT for 16 h. The reaction mixture was diluted with EtOAc, the insoluble solid filtered, washed with satd. aq. NaCl (3×), dried (MgSO₄), filtered, and concentrated in vacuo. The crude product was purified by silica gel column chromatography (eluting with a gradient of 5% to 25% EtOAc in hexane) to afford 700 mg (85%) of tert-butyl (1R,5S)-3-(7-bromo-2-chloropyrido[3,2-d]pyrimidin-4-yl)-3,8-diazabicyclo[3.2.1]octane-8-carboxylate (8c) as a white solid: HPLC-MS (ES⁺) m/z MH⁺=454.

tert-Butyl (1R,5S)-3-(7-bromo-2-((tetrahydro-1H-pyrrolizin-7a(5H)-yl)methoxy)pyrido[3,2-d]pyrimidin-4-yl)-3,8-diazabicyclo[3.2.1]octane-8-carboxylate (8e). A mixture of tert-butyl (1R,5S)-3-(7-bromo-2-chloropyrido[3,2-d]pyrimidin-4-yl)-3,8-diazabicyclo[3.2.1]octane-8-carboxylate (8c; 400 mg, 0.883 mmol) and (tetrahydro-1H-pyrrolizin-7a(5H)-yl)methanol (8d; CAS #78449-72-6; 1 mL, 7.06 mmol) was treated with K₂CO₃ (365 mg, 2.65 mmol) and oven-dried 4 Å molecular sieves (440 mg) in 1,4-dioxane (3 mL) and then heated at reflux with stirring for 16 h. The reaction mixture was cooled to RT, diluted with EtOAc, washed with satd. aq. NaCl (3×), dried (MgSO₄), filtered and concentrated in vacuo. The crude product was purified by silica gel column chromatography eluting with a gradient of 1% to 10% MeOH in DCM to afford 232 mg (47%) of tert-butyl (1R,5S)-3-(7-bromo-2-((tetrahydro-1H-pyrrolizin-7a(5H)-yl)methoxy)pyrido[3,2-d]pyrimidin-4-yl)-3,8-diazabicyclo[3.2.1]octane-8-carboxylate (8e) as a pale-yellow solid: HPLC-MS (ES⁺) m/z MH⁺=559.

tert-Butyl (1R,5S)-3-(7-(8-chloronaphthalen-1-yl)-2-((tetrahydro-1H-pyrrolizin-7a(5H)-yl)methoxy)pyrido[3,2-d]pyrimidin-4-yl)-3,8-diazabicyclo[3.2.1]octane-8-carboxylate (8g). A mixture of tert-butyl (1R,5S)-3-(7-bromo-2-((tetrahydro-1H-pyrrolizin-7a(5H)-yl)methoxy)pyrido[3,2-d]pyrimidin-4-yl)-3, 8-diazabicyclo[3.2.1]octane-8-carboxylate (8e; 410 mg, 0.734 mmol), 2-(8-chloronaphthalen-1-yl)-4,4,5,5-tetramethyl-1,3,2-dioxaborolane (8f; 423 mg, 1.47 mmol), K₂CO₃ (405 mg, 2.94 mmol) in 1,4-dioxane (4 mL) and water (1.40 mL) was degassed by sparging with N₂ for 45 minutes. Tetrakis(triphenylphosphine)palladium (0) (127 mg, 0.110 mmol) was added and the reaction mixture degassed by sparging with N₂ for an additional 15 minutes. The reaction mixture was then heated at 85° C. with stirring under a N₂ atmosphere for 16 h, cooled to RT and degassed by sparging with N₂ for 15 minutes. Additional tetrakis(triphenylphosphine)palladium (0) (65 mg, 0.056 mmol) was added and the reaction mixture degassed by sparging with N₂ for an additional 15 minutes. The reaction mixture was heated at 95° C. with stirring under a N₂ atmosphere for 96 h, cooled to RT, diluted with EtOAc and washed with satd. aq. NaCl (3×), dried (MgSO₄), filtered, and concentrated in vacuo. The crude product was purified by silica gel column chromatography eluting with a gradient of 0% to 15% MeOH in DCM to afford 130 mg (27%) of tert-butyl (1R,5S)-3-(7-(8-chloronaphthalen-1-yl)-2-((tetrahydro-1H-pyrrolizin-7a(5H)-yl)methoxy)pyrido[3,2-d]pyrimidin-4-yl)-3,8-diazabicyclo[3.2.1]octane-8-carboxylate (8g) as a light brown solid: HPLC-MS (ES⁺) m/z MH⁺=641; ¹H NMR (300 MHz, DMSO-d₆) δ 8.54 (d, J=2.2 Hz, 1H), 8.10 (dd, J=8.1, 12.5 Hz, 2H), 7.82 (d, J=2.2 Hz, 1H), 7.72-7.65 (m, 2H), 7.60-7.54 (m, 2H), 4.29 (br s, 2H), 4.01 (s, 2H), 2.97-2.90 (m, 2H), 2.51-2.49 (m, 2H), 2.59-2.53 (m, 2H), 2.01-1.65 (m, 12H), 1.61-1.50 (m, 2H), 1.45 (s, 9H).

4-((1R,5S)-3,8-Diazabicyclo[3.2.1]octan-3-yl)-7-(8-chloronaphthalen-1-yl)-2-((tetrahydro-1H-pyrrolizin-7a(5H)-yl)methoxy)pyrido[3,2-d]pyrimidine trihydrochloride (1aa). A solution of tert-butyl (1R,5S)-3-(7-(8-chloronaphthalen-1-yl)-2-((tetrahydro-1H-pyrrolizin-7a(5H)-yl)methoxy)pyrido[3,2-d]pyrimidin-4-yl)-3, 8-diazabicyclo[3.2.1]octane-8-carboxylate (8g; 124 mg, 0.193 mmol) in DCM (1 mL) under a N₂ atmosphere was treated with 4 M HCl in 1,4-dioxane (2 mL) and stirred at RT for 1.5 h. The reaction mixture was concentrated in vacuo, diluted with dichloromethane (1 mL) and concentrated in vacuo (this was repeated three times) to afford 104 mg (4-((1R,5S)-3,8-diazabicyclo[3.2.1]octan-3-yl)-7-(8-chloronaphthalen-1-yl)-2-((tetrahydro-1H-pyrrolizin-7a(5H)-yl)methoxy)pyrido[3,2-d]pyrimidine trihydrochloride (1aa) as a light brown solid: HPLC-MS (ES⁺) m/z MH⁺=541; ¹H NMR (300 MHz, DMSO-d₆) δ 10.9 (br s, 1H), 10.0 (br s, 1H), 9.71 (br s, 1H), 8.70 (d, J=2.0 Hz, 1H), 8.21 (d, J=7.5 Hz, 1H), 8.13 (d, J=7.1 Hz, 1H), 8.03 (d, J=2.0 Hz, 1H), 7.75-7.67 (m, 2H), 7.63-7.56 (m, 2H), 4.63 (s, 2H), 4.26 (s, 2H), 3.73-3.65 (m, 2H), 3.59-3.44 (m, 2H), 3.27-3.15 (m, 2H), 2.70 (m, 3H), 2.23-1.86 (m, 12H).

### Example 2

4-(4-((1R,5S)-3,8-Diazabicyclo[3.2.1]octan-3-yl)-2-((tetrahydro-1H-pyrrolizin-7a(5H)-yl)methoxy)pyrido[3,2-d]pyrimidin-7-yl)naphthalen-2-ol (1-ad).

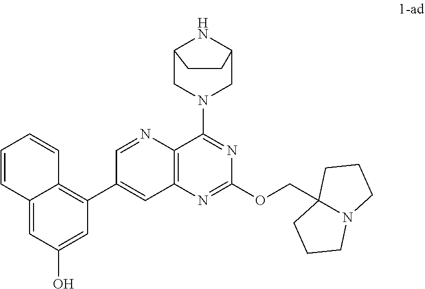 [SMILES: Oc1cc(-c2cnc3c(N4CC5CCC(C4)N5)nc(OCC45CCCN4CCC5)nc3c2)c2ccccc2c1]

Example 2 (1-ad) was prepared as shown below in Scheme 9.

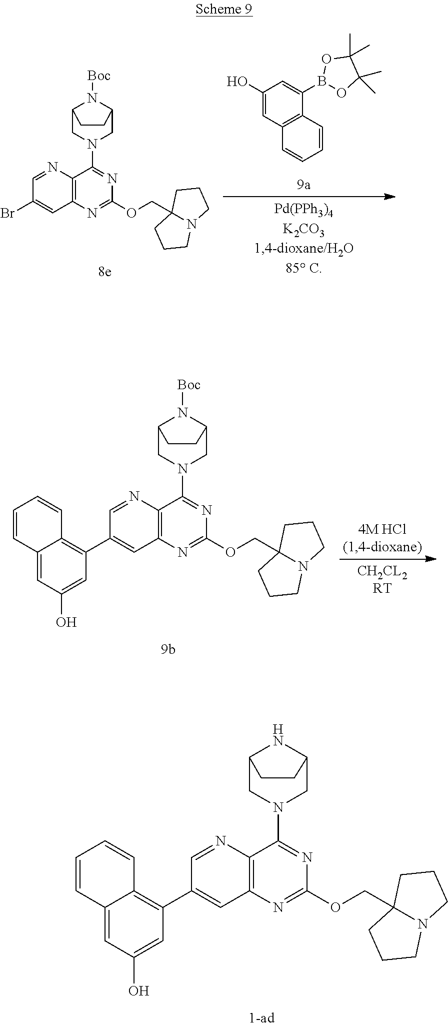 [SMILES: CC(C)(C)OC(=O)N1C2CCC1CN(c1nc(OCC34CCCN3CCC4)nc3cc(Br)cnc13)C2 ; CC1(C)OB(c2cc(O)cc3ccccc23)OC1(C)C ; CC(C)(C)OC(=O)N1C2CCC1CN(c1nc(OCC34CCCN3CCC4)nc3cc(-c4cc(O)cc5ccccc45)cnc13)C2 ; Oc1cc(-c2cnc3c(N4CC5CCC(C4)N5)nc(OCC45CCCN4CCC5)nc3c2)c2ccccc2c1]

tert-Butyl (1R,5S)-3-(7-(3-hydroxynaphthalen-1-yl)-2-((tetrahydro-1H-pyrrolizin-7a(5H)-yl)methoxy)pyrido[3,2-d]pyrimidin-4-yl)-3,8-diazabicyclo[3.2.1]octane-8-carboxylate (9b). A mixture of tert-butyl (1R,5S)-3-(7-bromo-2-((tetrahydro-1H-pyrrolizin-7a(5H)-yl)methoxy)pyridol[3,2-d]pyrimidin-4-yl)-3, 8-diazabicyclo[3.2.1]octane-8-carboxylate (8e; 457 mg, 0.819 mmol), 4-(4,4,5,5-tetramethyl-1,3,2-dioxaborolan-2-yl)naphthalen-2-ol (42; CAS #2043962-01-0; 442 mg, 1.64 mmol), K₂CO₃ (452 mg, 3.28 mmol) in 1,4-dioxane (8 mL) and water (1.60 mL) was degassed by sparging with N₂ for 30 minutes. Tetrakis(triphenyl-phosphine)palladium (0) (142 mg, 0.123 mmol) was added and the reaction mixture degassed by sparging with N₂ for an additional 20 minutes. After sparging was complete, the reaction mixture was heated at 85° C. with stirring under a N₂ atmosphere for 16 h. The reaction mixture was cooled to RT, diluted with EtOAc, washed with satd. aq. NaCl (3×), dried (MgSO₄), filtered, and concentrated in vacuo. The crude product was purified by silica gel column chromatography (eluting with a gradient of 0% to 20% i-PrOH containing 1% Et₃N (v/v) in DCM) to afford 260 mg (51%) of tert-butyl (1R,5S)-3-(7-(3-hydroxynaphthalen-1-yl)-2-((tetrahydro-1H-pyrrolizin-7a(5H)-yl)methoxy)pyrido [3,2-d]pyrimidin-4-yl)-3,8-diazabicyclo [3.2.1]octane-8-carboxylate (9b) as a tan solid: HPLC-MS (ES⁺) m/z MH⁺=623.

4-(4-((1R,5S)-3,8-Diazabicyclo[3.2.1]octan-3-yl)-2-((tetrahydro-1H-pyrrolizin-7a(5H)-yl)methoxy)pyrido[3,2-d]pyrimidin-7-yl)naphthalen-2-ol (1-ad). The title compound was prepared in an analogous manner as described for Example 1 (1-aa) and the crude product was treated with NH₄OH and purified by chromatography on silica gel eluting with gradient of 0-20% MeOH containing 5% NH₄OH (v/v) to afford 4-(4-((1R,5S)-3,8-diazabicyclo[3.2.1]octan-3-yl)-2-((tetrahydro-1H-pyrrolizin-7a(5H)-yl)methoxy)pyrido[3,2-d]pyrimidin-7-yl)naphthalen-2-ol (1-ad) as a tan solid: HPLC-MS (ES⁺) m/z MH⁺=523; ¹H NMR (300 MHz, DMSO-d₆) δ 10.0 (br s, 1H), 8.63 (d, J=2.1 Hz, 1H), 7.87 (d, J=2.1 Hz, 1H), 7.81 (d, J=8.1 Hz, 1H), 7.62 (d, J=8.4 Hz, 1H), 7.45 (t, J=7.1 Hz, 1H), 7.30-7.25 (m, 2H), 7.13 (d, J=2.4 Hz, 1H), 3.99 (s, 2H), 3.54 (br s, 2H), 3.35 (s, 4H), 2.95-2.88 (m, 2H), 2.56-2.51 (m, 2H), 1.92-1.50 (m, 13H).

### Example 3

4-((1R,5S)-3,8-Diazabicyclo[3.2.1]octan-3-yl)-7-(8-chloronaphthalen-1-yl)-2-(((S)-1-methylpyrrolidin-2-yl)methoxy)pyrido[2,3-d]pyrimidine (1-es).

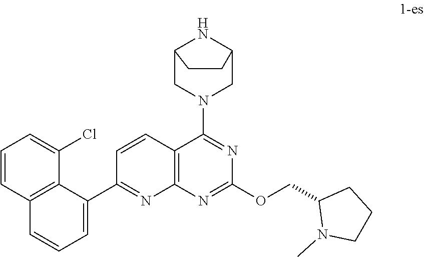

Example 3 (1-es) was prepared as shown below in Scheme 10.

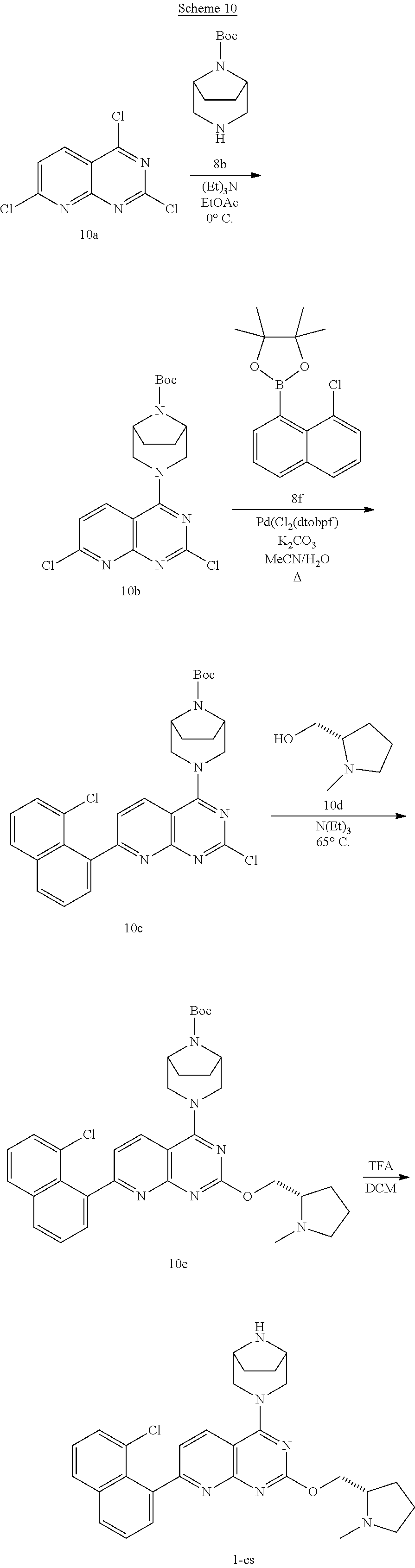

tert-Butyl (1R,5S)-3-(2,7-dichloropyrido[2,3-d]pyrimidin-4-yl)-3,8-diazabicyclo[3.2.1]octane-8-carboxylate (10b). A solution of tert-butyl (1R,5S)-3,8-diazabicyclo[3.2.1]octane-8-carboxylate (8b; 542 mg, 2.55 mmol) in ethyl acetate (10 mL) was added dropwise at 0 C over 15 minutes to a stirred solution of 2,4,7-trichloropyrido[2,3-d]pyrimidine (10a; CAS #938443-20-0; 589 mg, 2.51 mmol) and triethylamine (0.42 mL, 3.01 mmol) in ethyl acetate (50 mL) under N₂. After 15 minutes, the resulting suspension was then partitioned between water and additional ethyl acetate. The organic extract was washed with satd. NaCl (aq.), dried (CaSO₄), filtered and concentrated in vacuo. The residue was recrystallized from ethyl acetate/heptane to provide 1.02 g of tert-butyl (1R,5S)-3-(2,7-dichloropyrido[2,3-d]pyrimidin-4-yl)-3,8-diazabicyclo[3.2.1]octane-8-carboxylate (10b) as a light-yellow powder: HPLC-MS (ES⁺) m/z MH⁺=410; ¹H NMR (300 MHz, DMSO-d₆) δ 8.53 (d, J=8.7 Hz, 1H), 7.52 (d, J=8.7 Hz, 1H), 4.35 (d, J=12.0 Hz, 2H), 4.23 (br s, 2H), 3.62 (d, J=12.6 Hz, 2H), 1.81-1.78 (m, 2H), 1.66-1.62 (m, 2H), 1.46 (s, 9H).

tert-Butyl (1R,5S)-3-(2-chloro-7-(8-chloronaphthalen-1-yl)pyrido[2,3-d]pyrimidin-4-yl)-3,8-diazabicyclo[3.2.1]octane-8-carboxylate (10c). [1,1′-Bis(di-tert-butylphosphino)-ferrocene] dichloropalladium(II) (70 mg, 0.11 mmol) was added to a stirred, degassed, yellow suspension of tert-butyl (1R,5S)-3-(2,7-dichloropyrido[2,3-d]pyrimidin-4-yl)-3,8-diazabicyclo[3.2.1]octane-8-carboxylate, (10b; 412 mg, 1.0 mmol), 2-(8-chloronaphthalen-1-yl)-4,4,5,5-tetramethyl-1,3,2-dioxaborolane (8f; 578 mg, 2.0 mmol), and potassium carbonate (210 mg, 1.5 mmol) in a 50% (v/v) mixture of water in acetonitrile (10 mL). The suspension was sparged at RT with N₂ while stirring for 15 minutes. The sparge tube was replaced with a condenser and the mixture heated at reflux under N₂ for 1 h. The cooled reaction mixture was partitioned between satd. NaCl (aq.) and ethyl acetate. The organic phase was dried (CaSO₄), filtered and concentrated in vacuo. The residue was purified by chromatography on silica gel eluting with a gradient of 0 to 40% EtOAc in hexanes. The combined product containing fractions were evaporated and recrystallized from ethyl acetate/heptane to furnish 172 mg of tert-butyl (1R,5S)-3-(2-chloro-7-(8-chloronaphthalen-1-yl)pyrido[2,3-d]pyrimidin-4-yl)-3,8-diazabicyclo[3.2.1]octane-8-carboxylate (10c) as a white powder: HPLC-MS (ES⁺) m/z MH⁺=536; ¹H NMR (300 MHz, DMSO-d₆) δ 8.56 (d, J=8.6 Hz, 1H), 8.19 (d, J=7.9 Hz, 1H), 8.11 (d, J=8.0 Hz, 1H), 7.74-7.65 (m, 2H), 7.61-7.56 (m, 3H), 4.43-4.40 (m, 2H), 3.68 (br s, 1H), 3.64 (br s, 1H), 1.85-1.83 (m, 2H), 1.72-1.69 (m, 2H), 1.47 (s, 9H).

tert-Butyl (1R,5S)-3-(7-(8-chloronaphthalen-1-yl)-2-(((S)-1-methylpyrrolidin-2-yl)methoxy)pyrido[2,3-d]pyrimidin-4-yl)-3,8-diazabicyclo[3.2.1]octane-8-carboxylate (7e). (S)-(1-methylpyrrolidin-2-yl)methanol (10d; CAS #34381-71-0; 0.50 mL, 4.2 mmol) and triethylamine (0.1 mL, 0.72 mmol) were added to tert-butyl (1R,5S)-3-(2-chloro-7-(8-chloronaphthalen-1-yl)pyrido[2,3-d]pyrimidin-4-yl)-3,8-diazabicyclo[3.2.1]octane-8-carboxylate (7c; 72 mg, 0.13 mmol) and the mixture was heated at 650 C while stirring for 2 days. The cooled reaction mixture was then partitioned between satd. NaHCO₃(aq.) and ethyl acetate. NaCl (aq.), dried (CaSO₄), filtered and concentrated in vacuo to afford 125 mg of tert-butyl (1R,5S)-3-(7-(8-chloronaphthalen-1-yl)-2-(((S)-1-methylpyrrolidin-2-yl)methoxy)pyrido[2,3-d]pyrimidin-4-yl)-3,8-diazabicyclo[3.2.1]octane-8-carboxylate (10e) as an off white solid: HPLC-MS (ES⁺) m/z MH⁺=615.

4-((1R,5S)-3,8-Diazabicyclo[3.2.1]octan-3-yl)-7-(8-chloronaphthalen-1-yl)-2-(((S)-1-methylpyrrolidin-2-yl)methoxy)pyrido[2,3-d]pyrimidine (1-es). Trifluoroacetic acid (5.0 mL, 65 mmol) was added at RT to a stirred solution of tert-butyl (1R,5S)-3-(7-(8-chloronaphthalen-1-yl)-2-(((S)-1-methylpyrrolidin-2-yl)methoxy)pyrido [2,3-d]pyrimidin-4-yl)-3,8-diazabicyclo[3.2.1]octane-8-carboxylate (10e; 125 mg, 0.20 mmol) in anhydrous DCM (20 mL) and stirred for 1h. The reaction mixture was washed with 10% (w/v) NaOH(aq.) and concentrated in vacuo. The residue was purified by chromatography on silica gel eluting with a gradient of 0 to 5% methanol (containing 2% NH₄OH) in DCM to provide 52 mg of 4-((1R,5S)-3,8-diazabicyclo[3.2.1]octan-3-yl)-7-(8-chloronaphthalen-1-yl)-2-(((S)-1-methylpyrrolidin-2-yl)methoxy)pyrido[2,3-d]pyrimidine (1-es) as a light-yellow solid: HPLC-MS (ES⁺) m/z MH⁺=515; ¹H NMR (300 MHz, DMSO-d₆) δ 8.38 (d, J=8.5 Hz, 1H), 8.16 (dd, J=8.3, 1.2 Hz, 1H), 8.09 (dd, J=8.1, 1.3 Hz, 1H), 7.72-7.63 (m, 2H), 7.60-7.54 (m, 2H), 7.37 (d, J=8.4 Hz, 1H), 4.35-4.22 (m, 2H), 4.20-4.00 (m, 2H), 3.62-3.51 (m, 5H), 2.99-2.93 (m, 1H), 2.64-2.55 (m, 1H), 2.36 (s, 3H), 2.24-2.15 (m, 1H), 2.01-1.90 (m, 1H), 1.70-1.64 (m, 5H), 1.23 (br s, 2H).

### Example 4

4-((1R,5S)-3,8-diazabicyclo[3.2.1]octan-3-yl)-7-(8-chloronaphthalen-1-yl)-2-((tetrahydro-1H-pyrrolizin-7a(5H)-yl)methoxy)pyrido[2,3-d]pyrimidine (1-ab).

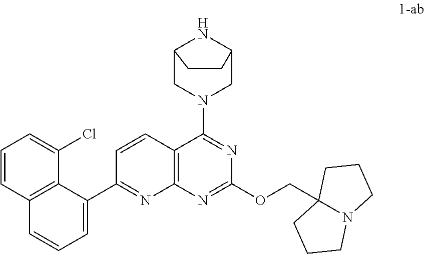 [SMILES: Clc1cccc2cccc(-c3ccc4c(N5CC6CCC(C5)N6)nc(OCC56CCCN5CCC6)nc4n3)c12]

Example 4 (1-ab) was prepared as shown below in Scheme 11.

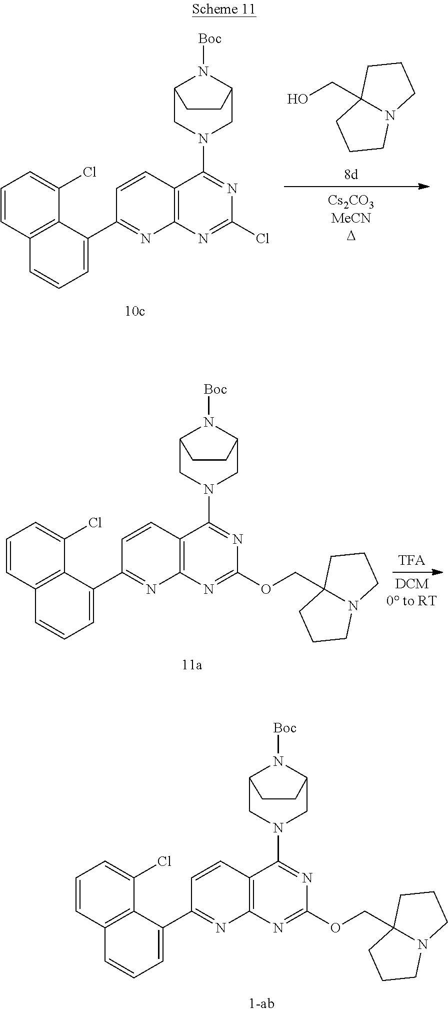 [SMILES: CC(C)(C)OC(=O)N1C2CCC1CN(c1nc(Cl)nc3nc(-c4cccc5cccc(Cl)c45)ccc13)C2 ; OCC12CCCN1CCC2 ; CC(C)(C)OC(=O)N1C2CCC1CN(c1nc(OCC34CCCN3CCC4)nc3nc(-c4cccc5cccc(Cl)c45)ccc13)C2 ; CC(C)(C)OC(=O)N1C2CCC1CN(c1nc(OCC34CCCN3CCC4)nc3nc(-c4cccc5cccc(Cl)c45)ccc13)C2]

tert-Butyl (1R,5S)-3-(7-(8-chloronaphthalen-1-yl)-2-((tetrahydro-1H-pyrrolizin-7a(5H)-yl)methoxy)pyrido[2,3-d]pyrimidin-4-yl)-3,8-diazabicyclo[3.2.1]octane-8-carboxylate (11a). Cesium carbonate (167 mg, 0.52 mmol) was added to a stirred solution of tert-butyl (1R,5S)-3-(2-chloro-7-(8-chloronaphthalen-1-yl)pyrido[2,3-d]pyrimidin-4-yl)-3,8-diazabicyclo[3.2.1]octane-8-carboxylate (10c; 138 mg, 0.26 mmol) and (tetrahydro-1H-pyrrolizin-7a(5H)-yl)methanol (8d; 110 mg, 0.78 mmol) in anhydrous acetonitrile (5.2 mL) and heated at reflux under N₂ for 3 days. The cooled reaction mixture was partitioned between satd. NaCl (aq.) and ethyl acetate and the organic extract was dried (CaSO₄), filtered and concentrated in vacuo. The residue was purified by chromatography on silica gel eluting with a gradient of 1 to 5% methanol (containing 2% NH₄OH) in DCM to yield 48 mg of tert-butyl (1R,5S)-3-(7-(8-chloronaphthalen-1-yl)-2-((tetrahydro-1H-pyrrolizin-7a(5H)-yl)methoxy)pyrido [2,3-d]pyrimidin-4-yl)-3, 8-diazabicyclo[3.2.1]octane-8-carboxylate (11a) as a clear yellow oil: HPLC-MS (ES⁺) m/z MH⁺=641.

4-((1R,5S)-3,8-Diazabicyclo[3.2.1]octan-3-yl)-7-(8-chloronaphthalen-1-yl)-2-((tetrahydro-1H-pyrrolizin-7a(5H)-yl)methoxy)pyrido[2,3-d]pyrimidine (1-ab). Trifluoroacetic acid (2.0 mL, 26.1 mmol) was added under N₂ to a stirred solution at 0° C. of tert-butyl (1R,5S)-3-(7-(8-chloronaphthalen-1-yl)-2-((tetrahydro-1H-pyrrolizin-7a(5H)-yl)methoxy)pyrido [2,3-d]pyrimidin-4-yl)-3,8-diazabicyclo [3.2.1]octane-8-carboxylate (11a; 48 mg, 0.07 mmol) in anhydrous DCM (10 mL). The solution was stirred at 0° C. for 1 h and then slowly warmed to RT over 2 h. The reaction mixture was diluted with 10 mL of DCM, extracted with 5% (w/v) NaOH (aq.), dried (CaSO₄), filtered and concentrated in vacuo to afford 38.6 mg of 4-((1R,5S)-3,8-diazabicyclo[3.2.1]octan-3-yl)-7-(8-chloronaphthalen-1-yl)-2-((tetrahydro-1H-pyrrolizin-7a(5H)-yl)methoxy)pyrido[2,3-d]pyrimidine (1-ab) as a waxy light-yellow solid: HPLC-MS (ES⁺) m/z MH⁺=541; ¹H NMR (300 MHz, DMSO-d₆) δ 8.37 (d, 1H, J=8.5 Hz), 8.17 (dd, 1H, J=8.3, 1.2 Hz), 8.09 (dd, 1H, J=8.1, 1.3 Hz), 7.72-7.63 (m, 2H), 7.60-7.54 (m, 2H), 7.35 (d, 1H, J=8.4 Hz), 4.33-4.31 (m, 1H), 4.22-4.19 (m, 1H), 4.02 (s, 2H), 3.57-3.45 (m, 4H), 2.97-2.90 (m, 2H), 2.59-2.54 (m, 2H), 1.93-1.71 (m, 6H), 1.65-1.53 (m, 7H).

### Example 5

4-(4-((1R,5S)-3,8-Diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-5-ethynyl-6-fluoronaphthalen-2-ol (1-bo).

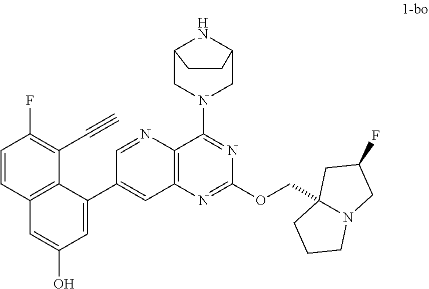

Example 5 (1-bo) was prepared as shown below in Scheme 12.

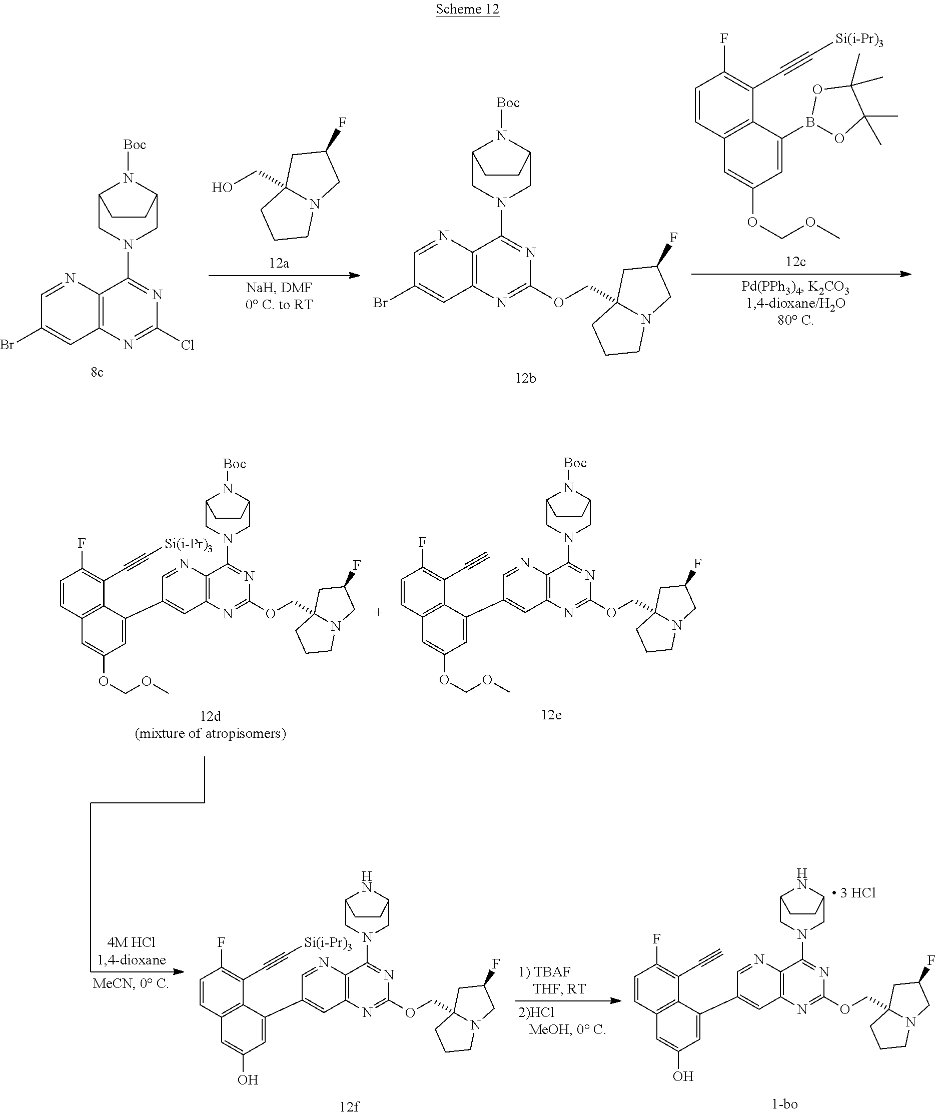

tert-Butyl (1R,5S)-3-(7-bromo-2-(((2R,7aS)-2-fluorotetrahydro-1H-pyrrolizin-7a(5H)-yl)methoxy)pyrido[3,2-d]pyrimidin-4-yl)-3,8-diazabicyclo[3.2.1]octane-8-carboxylate (12b). Sodium hydride (66 mg, 1.65 mmol, 60% w/w oil dispersion) was added to a solution of (2R,7aS)-2-fluorotetrahydro-1H-pyrrolizin-7a(5H)-yl)methanol (12a; CAS #2097518-76-6; 259 mg, 1.65 mmol) in anhydrous THF (11 mL) at 0° C. while stirring under a N₂ atmosphere. After 1 h, tert-butyl (1R,5S)-3-(7-bromo-2-chloropyrido[3,2-d]pyrimidin-4-yl)-3,8-diazabicyclo [3.2.1]octane-8-carboxylate (8c; 500 mg, 1.10 mmol) was added in one portion and the mixture warmed to RT. After 16 h, the mixture was diluted with EtOAc, washed with satd. aq. NaCl (2×), dried (MgSO₄), filtered and concentrated in vacuo. The crude product was purified by silica gel column chromatography eluting with a gradient of 0-10% MeOH in DCM to afford 401 mg (63%) of tert-butyl (1R,5S)-3-(7-bromo-2-(((2R,7aS)-2-fluorotetrahydro-1H-pyrrolizin-7a(5H)-yl)methoxy)pyrido[3,2-d]pyrimidin-4-yl)-3,8-diazabicyclo [3.2.1]octane-8-carboxylate (12b) as an off-white solid: HPLC-MS (ES⁺) m/z [M+H⁺]=577; ¹H NMR (300 MHz, CDCl₃) δ 8.49 (d, J=2.2 Hz, 1H), 8.05 (d, J=2.2 Hz, 1H), 5.27 (d, J=53.3 Hz, 1H), 4.35 (br s, 2H), 4.17 (d, J=10.2 Hz, 1H), 4.04 (d, J=10.1 Hz, 1H), 3.41 (br s, 1H), 3.25 (s, 2H), 3.18-2.90 (m, 2H), 2.37-2.06 (m, 4H), 2.00-1.64 (m, 9H), 1.50 (s, 9H).

tert-Butyl (1R,5S)-3-(7-(7-fluoro-3-(methoxymethoxy)-8-((triisopropylsilyl)ethynyl)-naphthalen-1-yl)-2-(((2R,7aS)-2-fluorotetrahydro-1H-pyrrolizin-7a(5H)-yl)methoxy)-pyrido[3,2-d]pyrimidin-4-yl)-3,8-diazabicyclo[3.2.1]octane-8-carboxylate (12d). tert-Butyl (1R,5S)-3-(7-bromo-2-(((2R,7aS)-2-fluorotetrahydro-1H-pyrrolizin-7a(5H)-yl)methoxy)-pyrido[3,2-d]pyrimidin-4-yl)-3,8-diazabicyclo[3.2.1]octane-8-carboxylate (12b; 300 mg, 0.521 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (12c; CAS #2621932-37-2; 293 mg, 0.573 mmol) and K₂CO₃(294 mg, 2.14 mmol) were combined in dioxane (6.5 mL) and water (1 mL) and the mixture was degassed by sparging with N₂ while stirring for 20 minutes. Tetrakis(triphenyl-phosphine)palladium (0) (60 mg, 0.052 mmol) was added and the reaction mixture degassed by sparging with N₂ while stirring for an additional 20 minutes. The reaction mixture was heated at 80° C. while stirring under a N₂ atmosphere for 16 h, cooled to RT, diluted with EtOAc and filtered through Celite. The filtrate was washed with satd. aq. NaCl (3×), dried (MgSO₄), filtered and concentrated in vacuo. The crude product was a mixture of 12e and 12f which was purified by silica gel column chromatography eluting with a gradient of 10-50% acetone in DCM) to afford 143 mg (31%) tert-butyl (1R,5S)-3-(7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido [3,2-d]pyrimidin-4-yl)-3, 8-diazabicyclo[3.2.1]octane-8-carboxylate (12d) as a mixture of atropisomers that appears as a yellow solid (HPLC-MS (ES⁺) m/z [M+H⁺]=883) and 136 mg (36%) of 12e as an orange-brown solid (HPLC-MS (ES⁺) m/z [M+H⁺]=727).

4-(4-((1R,5S)-3,8-Diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-6-fluoro-5-((triisopropylsilyl)-ethynyl)naphthalen-2-ol (12f). A solution of tert-Butyl (1R,5S)-3-(7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido [3,2-d]pyrimidin-4-yl)-3,8-diazabicyclo[3.2.1]octane-8-carboxylate (12d; 133 mg, 0.15 mmol) in anhydrous CH₃CN (3 mL) at 0° C. was treated with 4 M HCl in dioxane (1 mL) while stirring under a N₂ atmosphere. After 1 h, the reaction mixture was absorbed onto Celite and purified by silica gel column chromatography eluting with a gradient of 2-12% MeOH in DCM containing 10% NH₄OH (v/v) to afford 75 mg (68%) of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-6-fluoro-5-((triisopropylsilyl)ethynyl)naphthalen-2-ol (12f) as a light yellow solid: HPLC-MS (ES⁺) m/z [M+H⁺]=739; ¹H NMR (300 MHz, DMSO-d₆) δ 10.0 (br s, 1H), 8.54 (br d, J=2.1 Hz, 1H), 7.97 (dd, J=6.0, 9.2 Hz, 1H), 7.76 (br t, J=1.9 Hz, 1H), 7.47 (t, J=8.9 Hz, 1H), 7.35 (d, J=2.4 Hz, 1H), 7.05 (br d, J=2.3 Hz, 1H), 5.28 (d, J=53.9 Hz, 1H), 4.06 (dd, J=4.7, 10.6 Hz, 1H), 3.92 (d, J=10.4 Hz, 1H), 3.54 (br s, 2H), 3.20-3.05 (m, 4H), 3.00 (br s, 2H), 2.90-2.75 (m, 2H), 2.19-1.56 (m, 1OH), 0.82 (d, J=7.4 Hz, 9H), 0.78 (d, J=7.4 Hz, 9H), 0.63-0.44 (m, 3H).

4-(4-((1R,5S)-3,8-Diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-5-ethynyl-6-fluoronaphthalen-2-ol trihydrochloride (1-bo). Tetrabutylammonium fluoride (0.1 mL, 0.11 mmol, 1 M in THF) was added while stirring at RT under a N₂ atmosphere to a solution of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-6-fluoro-5-((triisopropylsilyl)ethynyl)naphthalen-2-ol (12f; 65 mg, 0.088 mmol) in anhydrous THF (2.0 mL). After 1 h, the reaction mixture was diluted with EtOAc and washed sequentially with H₂O (2×) and satd. aq. NaCl (2×). The EtOAc layer was dried (MgSO₄), filtered and concentrated in vacuo. The aqueous layers were concentrated in vacuo to dryness and then triturated with DCM and the resulting DCM extracts were combined, dried (MgSO₄), filtered and concentrated in vacuo. A solution of the crude residue in MeOH was treated at 0° C. under a N₂ atmosphere with 3 M HCl in MeOH (0.2 mL, 0.598 mmol). Diethyl ether was slowly added until a solid precipitated and the mixture was stirred at RT for 3 days. The resulting solid precipitate was isolated by filtration under a N₂ atmosphere, washed with Et₂O and dried under vacuum to afford 13 mg (25%) of 4-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[3,2-d]pyrimidin-7-yl)-5-ethynyl-6-fluoronaphthalen-2-ol trihydrochloride (1-bo) as a brown solid: HPLC-MS (ES⁺) m/z [M+H⁺]=583; ¹H NMR (300 MHz, DMSO-d₆) S 11.2 (br s, 1H), 10.3 (br s, 1H), 9.80 (br s, 1H), 9.43 (br s, 1H), 8.64 (br d, J=1.9 Hz, 1H), 7.99 (dd, J=6.2, 9.3 Hz, 1H), 7.91 (br t, J=1.6 Hz, 1H), 7.48 (t, J=9.0 Hz, 1H), 7.40 (br d, J=2.4 Hz, 1H), 7.14 (br d, J=2.2 Hz, 1H), 5.58 (d, J=52.8 Hz, 1H), 4.62 (br s, 2H), 4.25 (br s, 2H), 4.10 (br s, 1H), 3.87 (br s, 1H), 3.82-3.66 (m, 4H), 3.22-3.09 (m, 2H), 2.77-2.50 (m, 2H), 2.38-1.79 (m, 10H).

### Nucleotide Exchange Assay

The biological activity of the Examples was determined in a KRAS G12D/SOS1 Nucleotide Exchange Assay that was performed by Reaction Biology Corporation (RBC), 1 Great Valley Parkway, Suite 2 Malvern, PA 19355, USA. The assay evaluates the SOS1-mediated Bodipy-GDP to GTP exchange observed with KRAS G12D.

The compounds and reference standard (MRTX1133) were tested in 10 concentration IC₅₀ mode with 3-fold serial dilution at a starting concentration of 10 μM. The compound pre-incubation time was 30 min at RT and the curve fits were performed when the activities at the highest concentration of compounds were less than 65%.

Reaction Buffer: 40 mM HEPES 7.4, 10 mM MgCl₂, 1 mM DTT 0.002% Triton ×100, 0.5% DMSO.

Enzyme: SOS1 (RBC cat #MSC-11-502). Recombinant human SOS1 (Genbank accession #NM_033360.3; aa 564-1049, expressed in E. Coli with C-terminal StrepII).

KRAS G12D: Recombinant human KRAS G12D (aa 2-169, expressed in E. coli with N-terminal TEV cleavable his-tag). KRAS is pre-loaded with a 5-fold excess of Bodipy-GDP. The excess Bodipy-GDP is separated from loaded protein using a spin desalting column.

Final concentrations: KRAS-bodipy-GDP was 0.125 μM; SOS1 was 750 nM; and GTP was 25 μM.

### Reaction Procedure:

1. Deliver 10 μL of 1.5× KRAS solution in freshly prepared reaction buffer to reaction wells.
2. Deliver compounds in 100% DMSO into buffer using acoustic technology (Echo550; nanoliter range).
3. Incubate compounds with KRAS for 30 minutes at room temperature.
4. Prepare 3× (SOS1+GTP) solution in reaction buffer.
5. Deliver 5 μL of SOS1+GTP solution into reactions wells (deliver GTP only to column 1 for no SOS1 control).
6. Monitor reaction progress via decrease in fluorescence signal for 30 minutes at RT using a PHERAstar (BMG Labtech plate reader (Ex/Em=485/520).

Data Analysis: The fluorescence data was normalized using the equation below and fitted to “one phase exponential decay” equation using GraphPad prism software. The plateau was fixed to zero (use for non-covalent inhibitors) and rate ×1000 was used to calculate the IC₅₀ values.

$$Ynorm = 1 - \frac{( Yraw - Ao )}{( M - A o )}$$

where Y_{raw} is defined as fluorescence at time t, Ao is the average initial fluorescence with no SOS1, and M is the minimum fluorescence at the end of the reaction at the maximum SOS1.

The background subtracted signals (no SOS1 protein wells were used as background) were converted to % activity relative to DMSO controls. Data was analyzed using GraphPad Prism 4 with “sigmoidal dose-response (variable slope)”; 4 parameters with Hill Slope. The constraints were bottom (constant equal to 0) and top (must be less than 120).

KRAS G12D Activity*

| Compound | IC₅₀ (nM) | |
|---|---|---|
| MRTX1133 | 11 ± 2 | (N = 3) |
| Example 1 | 610 | |
| (Compound 1-aa) | | |
| Example 2 | 35 ± 5.7 | (N = 2) |

| | |
|---|---|
| (Compound 1-ad) | |
| Example 3 | >10,000 |
| (Compound 1-es) | |
| Example 4 | 8,600 |
| (Compound 1-ab) | |
| Example 5 | 28 |
| (Compound 1-bo) | |

*IC₅₀ values as analyzed by the rate constant method (plateau = 0) for reversible inhibitors with Bodipy-GDP/KRAS G12D as the substrate and 0.5% DMSO in the reaction mixture. MRTX1133 is a KRAS G12D reference standard, see Wang et al in J. Med. Chem. 2022, 65 (4), 3123-3133.

### KRAS G12D Cellular Assay

The KRAS G12D cellular activity of Example 5 (compound 1-bo) and the reference standard MRTX1133 were determined in a target engagement cellular assay (NanoBRET™) in transiently transfected HEK293 cells by Reaction Biology Corporation (RBC), 1 Great Valley Parkway, Suite 2 Malvern, PA 19355, USA. HEK293 were cultivated to 70-80% confluence prior to the assay followed by trypsinizing and collection of the cells. MRTX1133 was used as the KRAS G12D reference compound. Each test compound solution was delivered from a compound source plate to the wells of 384-well white non-binding surface plate by an Echo 550 prior to the assay.

A 10 μg/mL solution of DNA in Opti-MEM was prepared without serum that consisted of 1 μg KRAS 2B (G12D) large-bit vector and 1 μg KRAS 2B (G12D) small-bit vector, and 8 μg transfection carrier DNA. This mixture was subsequently treated with 30 μL of FuGENE HD Transfection Reagent into each milliliter of DNA mixture to form a lipid:DNA complex. The resulting mixture was then gently mixed by inversion and incubated at ambient temperature for 20 minutes to allow complexes to form. A mixture of 1 part of lipid:DNA complex with 20 parts of suspended HEK293 cells was added to a sterile conical tube and mixed gently by inversion. The cells+lipid:DNA complex mixture was then added to a sterile tissue culture dish and incubated for 24 hours. The medium was removed from the dish via aspiration followed by trypsinizing and allowing the cells to dissociate from the tissue culture dish. The trypsin was subsequently neutralized by using medium containing serum and centrifugation at 200×g for 5 minutes to pellet the cells in the conical tube. The cell density was adjusted to 2×105 cells/mL in Opti-MEM without phenol red. One part of Complete 20×NanoBRET™ RAS Tracer Reagent was dispensed to 20 parts of cells in the conical tube and mixed gently by inversion. The resulting cell suspension was dispensed into a white, 384-well NBS plate containing the test compounds (starting at 10 μM, 10-dose with 3-fold dilution) at 37° C., 5% CO₂ for 2 hours. The final concentration for RAS tracer K2 was 1 μM. The NBS plate was removed from the incubator and allowed to equilibrate to room temperature for 15 minutes.

Freshly prepared substrate solution (3×) in the assay medium was added to each well of the 384-well NBS plate and incubated for 3 minutes at room temperature. The donor emission wavelength (460 nm) and acceptor emission wavelength (600 nm) were measured using an Envision 2104 plate reader. The raw BRET ratio values were generated by dividing the acceptor emission value (600 nm) by the donor emission value (460 nm) for each sample. In order to correct for the background, the BRET ratio in the absence of tracer (average of no-tracer control samples) was subtracted from the BRET ratio of each sample. The BRET ratio was calculated using the following equation: BRET Ratio=[(Acceptor sample÷Donor sample)−(Acceptor no-tracer control÷Donor no-tracer control)]. The normalized BRET response (%) was calculated by the following equation: (BRET ratio of test compound/BRET ratio of DMSO control)*100%. The IC₅₀ curves were plotted and IC₅₀ values were calculated with GraphPad Prism 4 based on a sigmoidal dose-response equation.

KRAS G12D Activity in Transiently Transfected HEK293 Cells*

| Compound | IC₅₀ (nM) |
|---|---|
| MRTX1133 | <0.5 nM |
| Example 1 | <0.5 nM |
| (Compound 1-bo) | |

*NanoBRET ™ target engagement cellular assay (KRAS G12D). MRTX1133 is a KRAS G12D reference standard, see Wang et al in J. Med. Chem. 2022, 65 (4), 3123-3133.

1. A compound of Formula I: 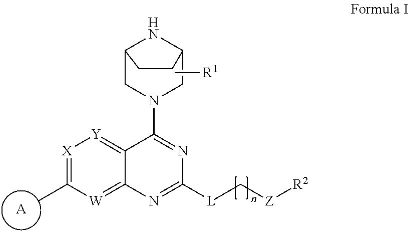
or a pharmaceutically acceptable salt, solvate, or prodrug thereof, wherein
A is chosen from aryl or heteroaryl optionally substituted with one or more of hydrogen, halogen, hydroxy, —C₁₋₆ alkyl, —C₂₋₆ alkenyl, —C₂₋₆ alkynyl, —C₀₋₃ alkyl(C₃₋₆ cycloalkyl), —C₁₋₆ alkyl(halo), —C₁₋₆ alkyl(OH), —O(C₁₋₄ alkyl), —C₁₋₃ alkyl(C₁₋₄ alkoxy), —CN, —CO₂R³,
—CO₂N(R³)₂, —NO₂, —N(R³)₂, —P(O)(R⁴)₂, —SR³, —S(O)R³, —SO₂R³ or a 5-6 membered heterocyclic ring;
W is chosen from CR⁵ or N with the proviso that if W is N, then Y is CR⁵;
X is chosen from CR⁵ or N with the proviso that if X is N, then Y is CR⁵ and W is N;
Y is chosen from CR⁵ or N with the proviso that if Y is N, then X and W are both CR⁵;
L is a bond, O, S, or NR³;
n is 0-2;
Z is C(R³)₂ or a cyclic compound chosen from C₃₋₇ cycloalkyl, a saturated or partially unsaturated 4- to 7-membered nitrogen-containing ring, a saturated or partially unsaturated 7- to 10-membered nitrogen-containing bridged bicyclic ring;
R¹ is chosen from hydrogen, hydroxy, halogen, —C₁₋₃ alkyl, —C₁₋₃ alkyl(OH), —C₁₋₃ alkyl(halo), —C₁₋₃ alkyl(C1-3 alkoxy), —C₁₋₃ alkyl(CN) or —C₁₋₃ alkyl(P(O)R³₂);
R² is chosen from hydrogen, halogen, hydroxy, —C₁₋₄ alkyl, —C₂₋₄ alkenyl, —C₂₋₄ alkynyl, —C₀₋₃ alkyl(C₃₋₆ cycloalkyl), —C₁₋₄ alkyl(halo), —C₁₋₄ alkyl(OH), —O(C₁₋₄ alkyl), —C₁₋₃ alkyl(C₁₋₃ alkoxy), —CN, —CO₂R³, —CO₂N(R³)₂, —NO₂, —N(R³)₂, —PO(R⁴)₂, —SR³, —S(O)R³, —SO₂R³,
or —(C₀₋₃ alkyl)R⁶;
Each R³ is the same or different and is chosen from hydrogen, C₁₋₄ alkyl, aryl or heteroaryl;
Each R⁴ is the same or different and is chosen from hydrogen, hydroxy, C₁₋₄ alkyl, aryl, heteroaryl, C₁₋₄ alkoxy, aryloxy or heteroaryloxy;
Each R⁵ may be the same or different and chosen from hydrogen, halogen, C₁₋₄ alkyl, C₁₋₄ perdeuteroalkyl, —(C₀₋₂ alkyl)alkenyl, —(C₀₋₂ alkyl)alkynyl, —(C₀₋₂ alkyl)cycloalkyl, —C₁₋₄ haloalkyl, —O(C₁₋₄ alkyl), —S(C₁₋₄ alkyl), —(C₀₋₂ alkyl)cyano, —O(C₁₋₄haloalkyl) or —S(C₁₋₄ haloalkyl);
R⁶ is chosen from N(R³)₂ or a 4- to 7-membered saturated or unsaturated heterocyclic ring containing one or more heteroatoms selected from the group N, O and S.

2. The compound of claim 1 wherein the compound of Formula I is selected from compounds 1-aa through 1-gd and 2-aa through 2-gc, and pharmaceutically acceptable salts, solvates, or prodrugs thereof:

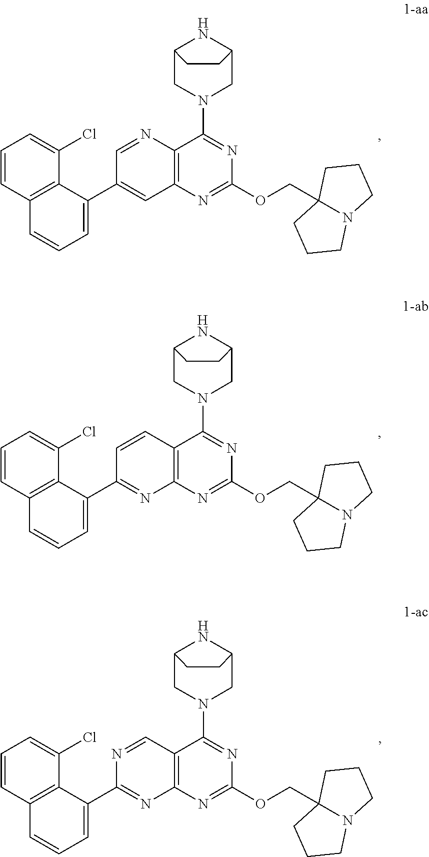 [SMILES: Clc1cccc2cccc(-c3cnc4c(N5CC6CCC(C5)N6)nc(OCC56CCCN5CCC6)nc4c3)c12 ; Clc1cccc2cccc(-c3ccc4c(N5CC6CCC(C5)N6)nc(OCC56CCCN5CCC6)nc4n3)c12 ; Clc1cccc2cccc(-c3ncc4c(N5CC6CCC(C5)N6)nc(OCC56CCCN5CCC6)nc4n3)c12]

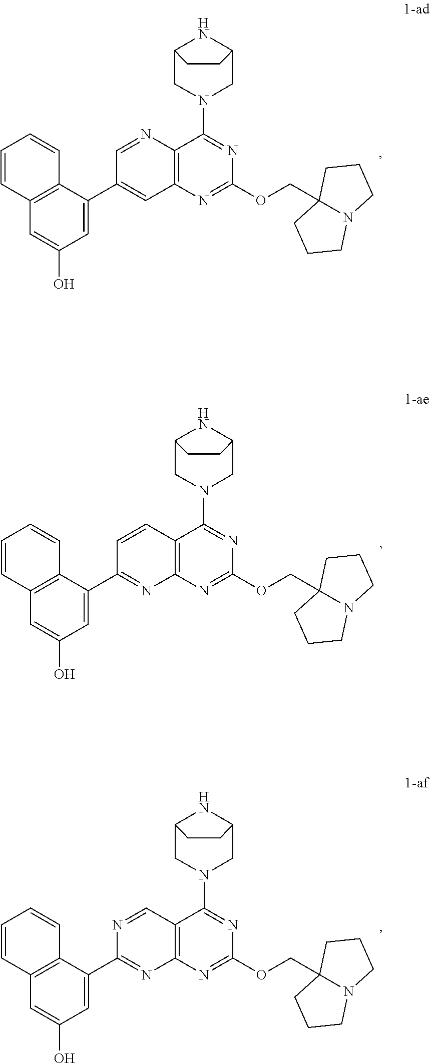 [SMILES: Oc1cc(-c2cnc3c(N4CC5CCC(C4)N5)nc(OCC45CCCN4CCC5)nc3c2)c2ccccc2c1 ; Oc1cc(-c2ccc3c(N4CC5CCC(C4)N5)nc(OCC45CCCN4CCC5)nc3n2)c2ccccc2c1 ; Oc1cc(-c2ncc3c(N4CC5CCC(C4)N5)nc(OCC45CCCN4CCC5)nc3n2)c2ccccc2c1]

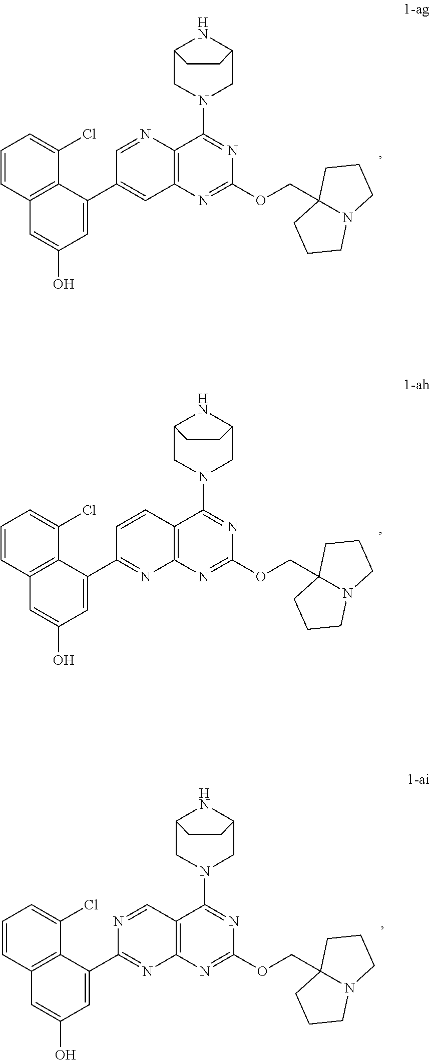 [SMILES: Oc1cc(-c2cnc3c(N4CC5CCC(C4)N5)nc(OCC45CCCN4CCC5)nc3c2)c2c(Cl)cccc2c1 ; Oc1cc(-c2ccc3c(N4CC5CCC(C4)N5)nc(OCC45CCCN4CCC5)nc3n2)c2c(Cl)cccc2c1 ; Oc1cc(-c2ncc3c(N4CC5CCC(C4)N5)nc(OCC45CCCN4CCC5)nc3n2)c2c(Cl)cccc2c1]

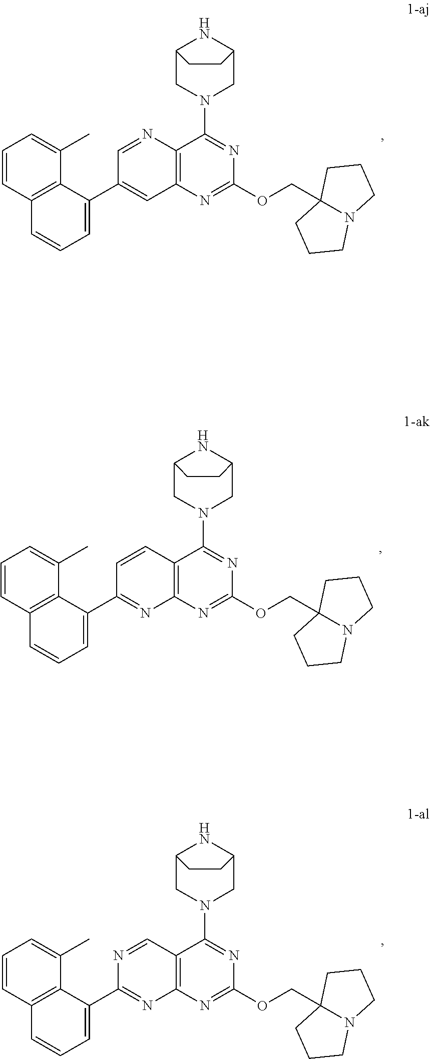 [SMILES: Cc1cccc2cccc(-c3cnc4c(N5CC6CCC(C5)N6)nc(OCC56CCCN5CCC6)nc4c3)c12 ; Cc1cccc2cccc(-c3ccc4c(N5CC6CCC(C5)N6)nc(OCC56CCCN5CCC6)nc4n3)c12 ; Cc1cccc2cccc(-c3ncc4c(N5CC6CCC(C5)N6)nc(OCC56CCCN5CCC6)nc4n3)c12]

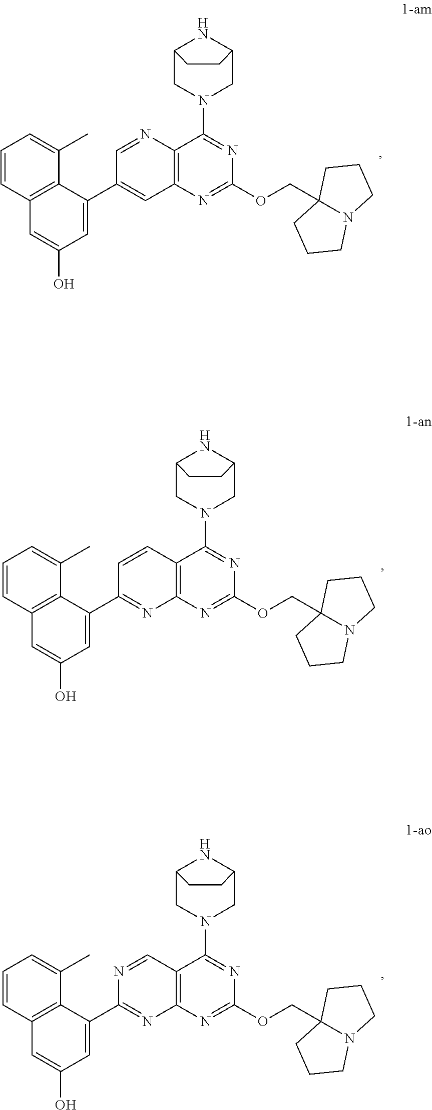 [SMILES: Cc1cccc2cc(O)cc(-c3cnc4c(N5CC6CCC(C5)N6)nc(OCC56CCCN5CCC6)nc4c3)c12 ; Cc1cccc2cc(O)cc(-c3ccc4c(N5CC6CCC(C5)N6)nc(OCC56CCCN5CCC6)nc4n3)c12 ; Cc1cccc2cc(O)cc(-c3ncc4c(N5CC6CCC(C5)N6)nc(OCC56CCCN5CCC6)nc4n3)c12]

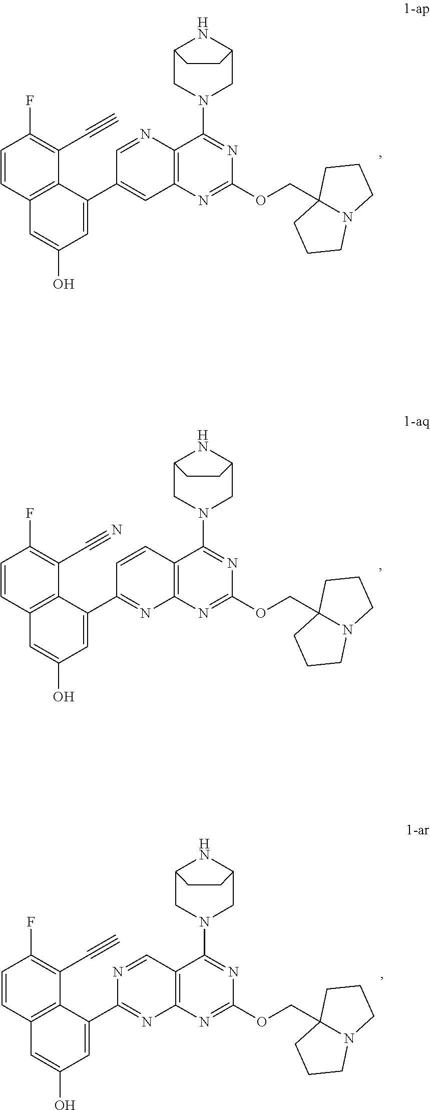 [SMILES: C#Cc1c(F)ccc2cc(O)cc(-c3cnc4c(N5CC6CCC(C5)N6)nc(OCC56CCCN5CCC6)nc4c3)c12 ; N#Cc1c(F)ccc2cc(O)cc(-c3ccc4c(N5CC6CCC(C5)N6)nc(OCC56CCCN5CCC6)nc4n3)c12 ; C#Cc1c(F)ccc2cc(O)cc(-c3ncc4c(N5CC6CCC(C5)N6)nc(OCC56CCCN5CCC6)nc4n3)c12]

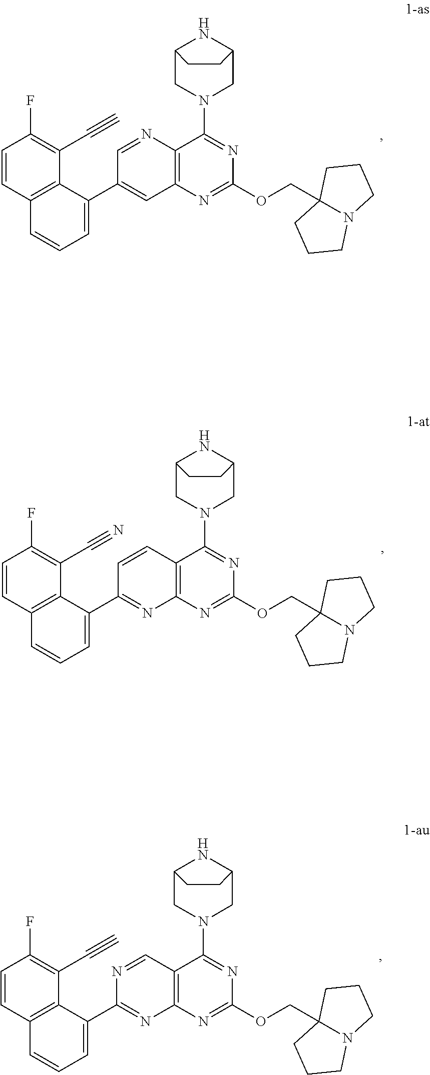 [SMILES: C#Cc1c(F)ccc2cccc(-c3cnc4c(N5CC6CCC(C5)N6)nc(OCC56CCCN5CCC6)nc4c3)c12 ; N#Cc1c(F)ccc2cccc(-c3ccc4c(N5CC6CCC(C5)N6)nc(OCC56CCCN5CCC6)nc4n3)c12 ; C#Cc1c(F)ccc2cccc(-c3ncc4c(N5CC6CCC(C5)N6)nc(OCC56CCCN5CCC6)nc4n3)c12]

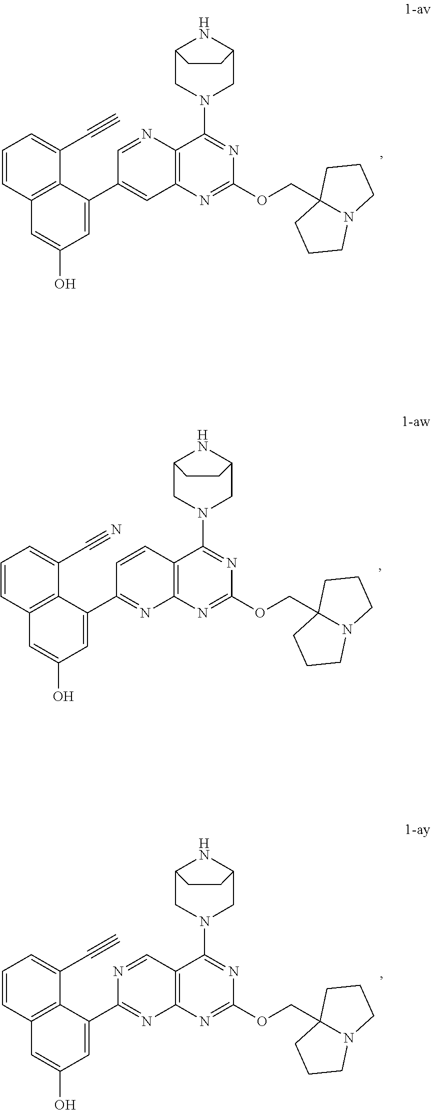 [SMILES: C#Cc1cccc2cc(O)cc(-c3cnc4c(N5CC6CCC(C5)N6)nc(OCC56CCCN5CCC6)nc4c3)c12 ; N#Cc1cccc2cc(O)cc(-c3ccc4c(N5CC6CCC(C5)N6)nc(OCC56CCCN5CCC6)nc4n3)c12 ; C#Cc1cccc2cc(O)cc(-c3ncc4c(N5CC6CCC(C5)N6)nc(OCC56CCCN5CCC6)nc4n3)c12]

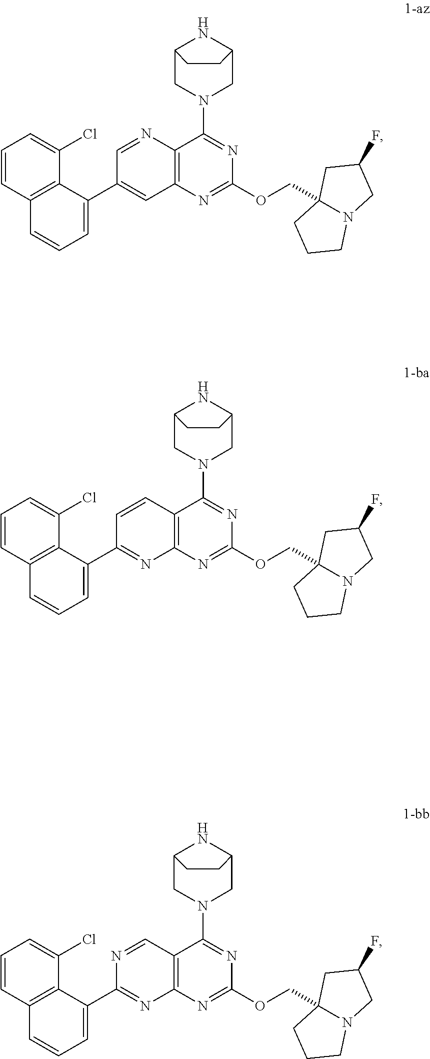 [SMILES: F[C@H]1CN2CCC[C@@]2(COc2nc(N3CC4CCC(C3)N4)c3ncc(-c4cccc5cccc(Cl)c45)cc3n2)C1 ; F[C@H]1CN2CCC[C@@]2(COc2nc(N3CC4CCC(C3)N4)c3ccc(-c4cccc5cccc(Cl)c45)nc3n2)C1 ; F[C@H]1CN2CCC[C@@]2(COc2nc(N3CC4CCC(C3)N4)c3cnc(-c4cccc5cccc(Cl)c45)nc3n2)C1]

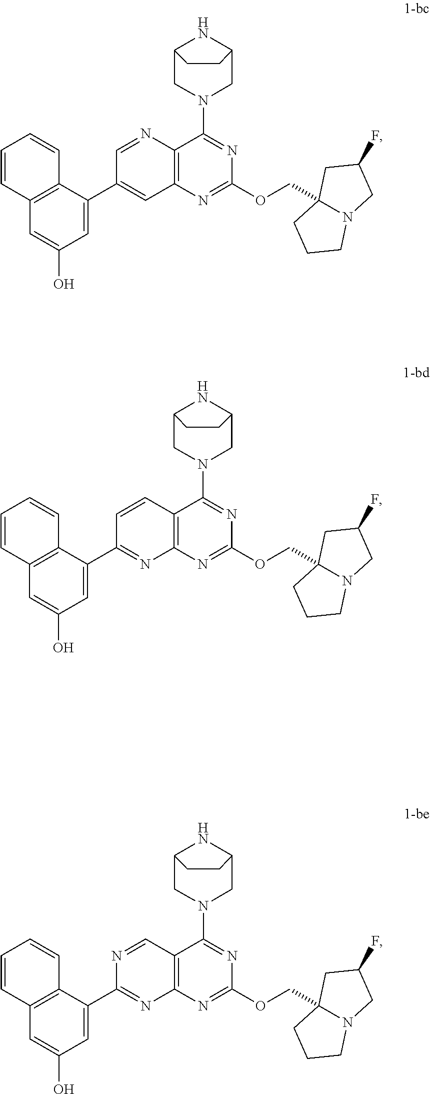 [SMILES: Oc1cc(-c2cnc3c(N4CC5CCC(C4)N5)nc(OC[C@@]45CCCN4C[C@H](F)C5)nc3c2)c2ccccc2c1 ; Oc1cc(-c2ccc3c(N4CC5CCC(C4)N5)nc(OC[C@@]45CCCN4C[C@H](F)C5)nc3n2)c2ccccc2c1 ; Oc1cc(-c2ncc3c(N4CC5CCC(C4)N5)nc(OC[C@@]45CCCN4C[C@H](F)C5)nc3n2)c2ccccc2c1]

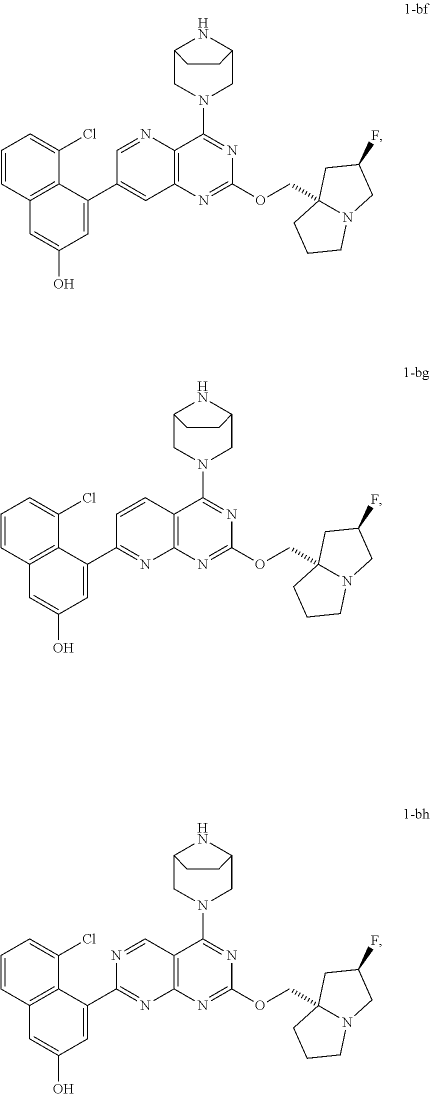 [SMILES: Oc1cc(-c2cnc3c(N4CC5CCC(C4)N5)nc(OC[C@@]45CCCN4C[C@H](F)C5)nc3c2)c2c(Cl)cccc2c1 ; Oc1cc(-c2ccc3c(N4CC5CCC(C4)N5)nc(OC[C@@]45CCCN4C[C@H](F)C5)nc3n2)c2c(Cl)cccc2c1 ; Oc1cc(-c2ncc3c(N4CC5CCC(C4)N5)nc(OC[C@@]45CCCN4C[C@H](F)C5)nc3n2)c2c(Cl)cccc2c1]

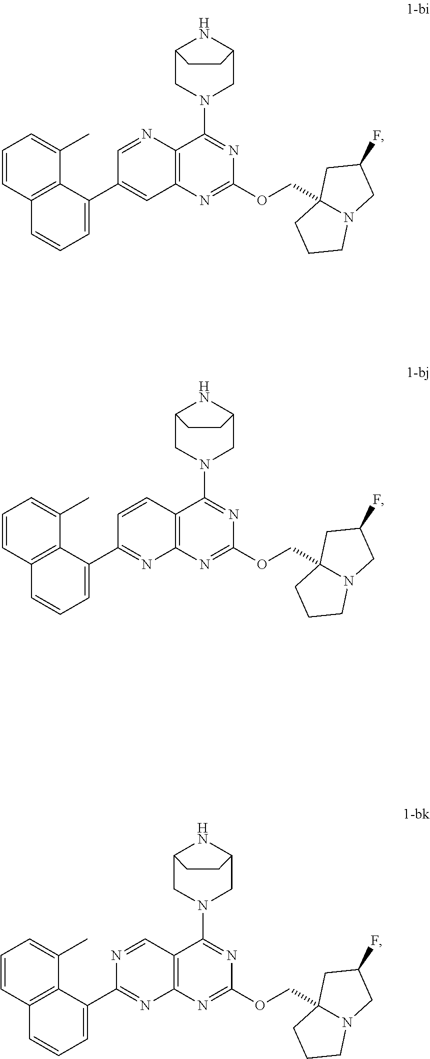 [SMILES: Cc1cccc2cccc(-c3cnc4c(N5CC6CCC(C5)N6)nc(OC[C@@]56CCCN5C[C@H](F)C6)nc4c3)c12 ; Cc1cccc2cccc(-c3ccc4c(N5CC6CCC(C5)N6)nc(OC[C@@]56CCCN5C[C@H](F)C6)nc4n3)c12 ; Cc1cccc2cccc(-c3ncc4c(N5CC6CCC(C5)N6)nc(OC[C@@]56CCCN5C[C@H](F)C6)nc4n3)c12]

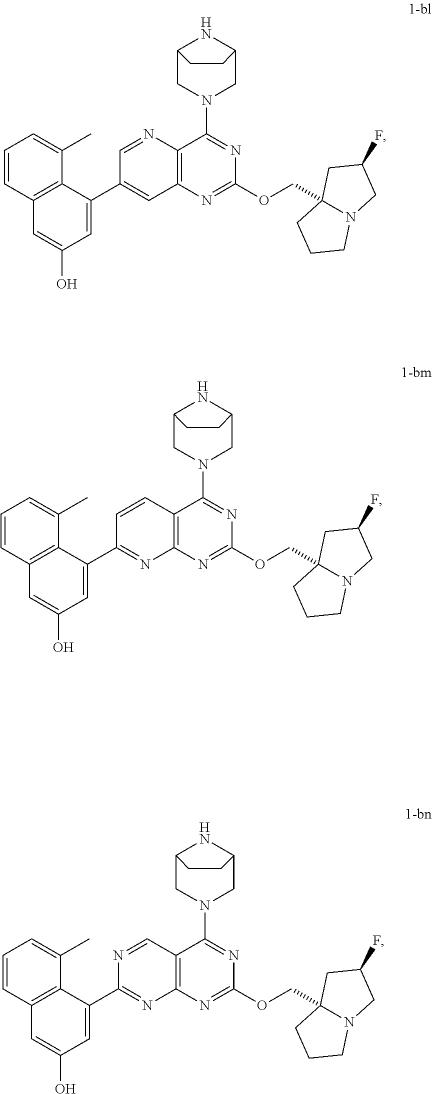 [SMILES: Cc1cccc2cc(O)cc(-c3cnc4c(N5CC6CCC(C5)N6)nc(OC[C@@]56CCCN5C[C@H](F)C6)nc4c3)c12 ; Cc1cccc2cc(O)cc(-c3ccc4c(N5CC6CCC(C5)N6)nc(OC[C@@]56CCCN5C[C@H](F)C6)nc4n3)c12 ; Cc1cccc2cc(O)cc(-c3ncc4c(N5CC6CCC(C5)N6)nc(OC[C@@]56CCCN5C[C@H](F)C6)nc4n3)c12]

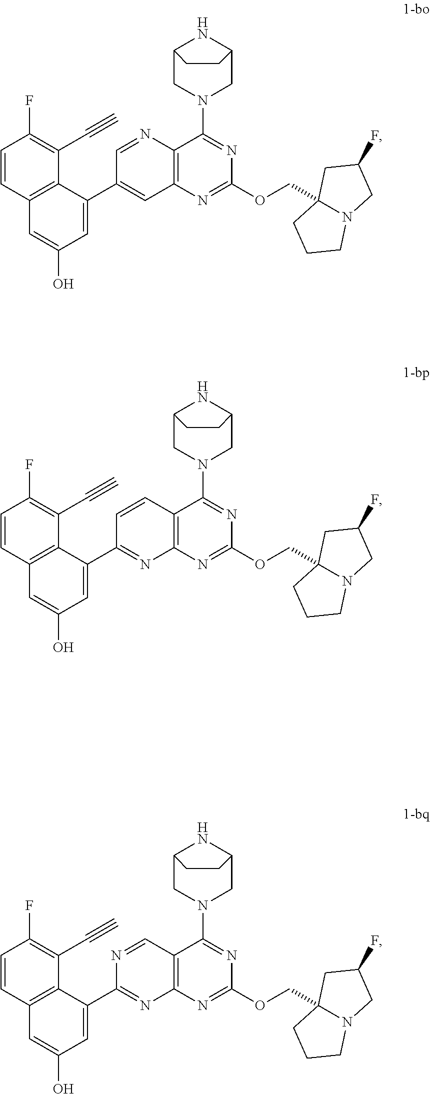 [SMILES: C#Cc1c(F)ccc2cc(O)cc(-c3cnc4c(N5CC6CCC(C5)N6)nc(OC[C@@]56CCCN5C[C@H](F)C6)nc4c3)c12 ; C#Cc1c(F)ccc2cc(O)cc(-c3ccc4c(N5CC6CCC(C5)N6)nc(OC[C@@]56CCCN5C[C@H](F)C6)nc4n3)c12 ; C#Cc1c(F)ccc2cc(O)cc(-c3ncc4c(N5CC6CCC(C5)N6)nc(OC[C@@]56CCCN5C[C@H](F)C6)nc4n3)c12]

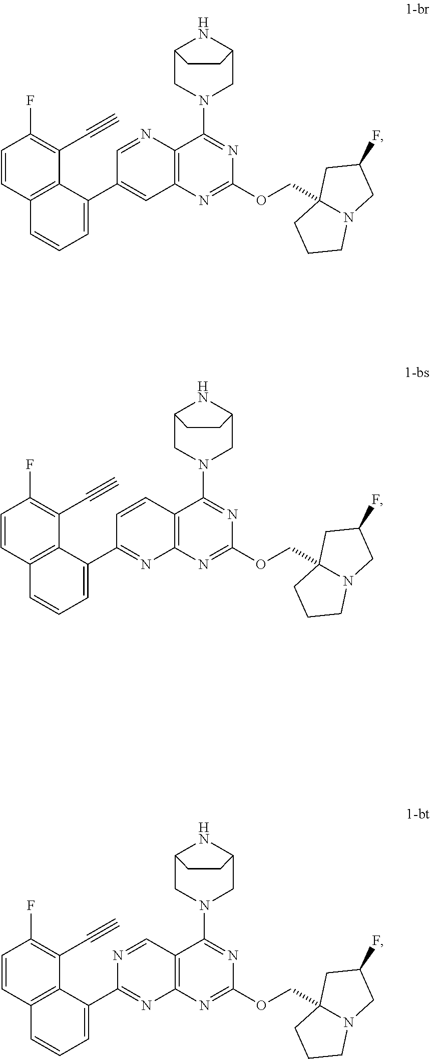 [SMILES: C#Cc1c(F)ccc2cccc(-c3cnc4c(N5CC6CCC(C5)N6)nc(OC[C@@]56CCCN5C[C@H](F)C6)nc4c3)c12 ; C#Cc1c(F)ccc2cccc(-c3ccc4c(N5CC6CCC(C5)N6)nc(OC[C@@]56CCCN5C[C@H](F)C6)nc4n3)c12 ; C#Cc1c(F)ccc2cccc(-c3ncc4c(N5CC6CCC(C5)N6)nc(OC[C@@]56CCCN5C[C@H](F)C6)nc4n3)c12]

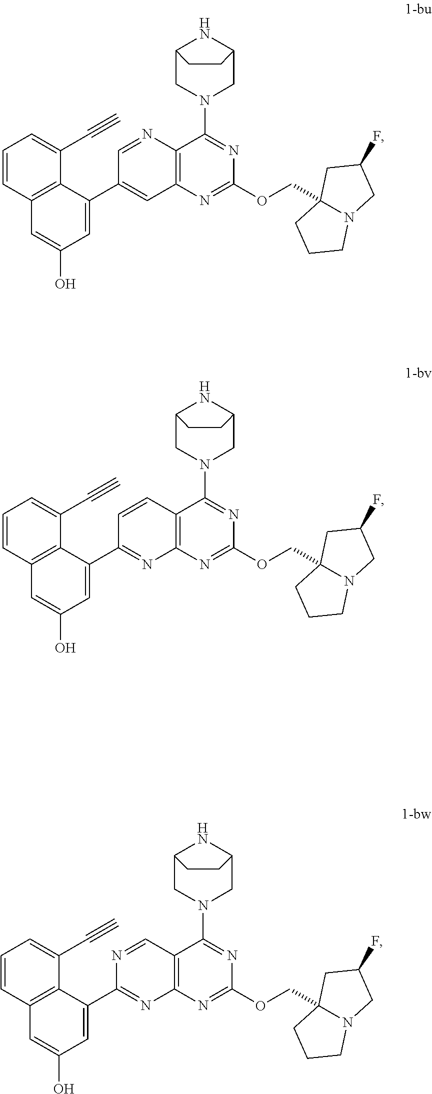 [SMILES: C#Cc1cccc2cc(O)cc(-c3cnc4c(N5CC6CCC(C5)N6)nc(OC[C@@]56CCCN5C[C@H](F)C6)nc4c3)c12 ; C#Cc1cccc2cc(O)cc(-c3ccc4c(N5CC6CCC(C5)N6)nc(OC[C@@]56CCCN5C[C@H](F)C6)nc4n3)c12 ; C#Cc1cccc2cc(O)cc(-c3ncc4c(N5CC6CCC(C5)N6)nc(OC[C@@]56CCCN5C[C@H](F)C6)nc4n3)c12]

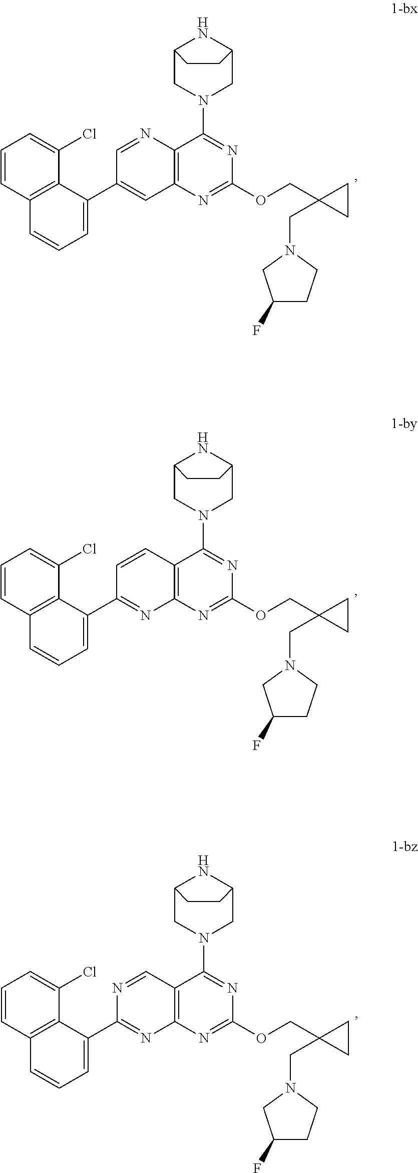 [SMILES: F[C@@H]1CCN(CC2(COc3nc(N4CC5CCC(C4)N5)c4ncc(-c5cccc6cccc(Cl)c56)cc4n3)CC2)C1 ; F[C@@H]1CCN(CC2(COc3nc(N4CC5CCC(C4)N5)c4ccc(-c5cccc6cccc(Cl)c56)nc4n3)CC2)C1 ; F[C@@H]1CCN(CC2(COc3nc(N4CC5CCC(C4)N5)c4cnc(-c5cccc6cccc(Cl)c56)nc4n3)CC2)C1]

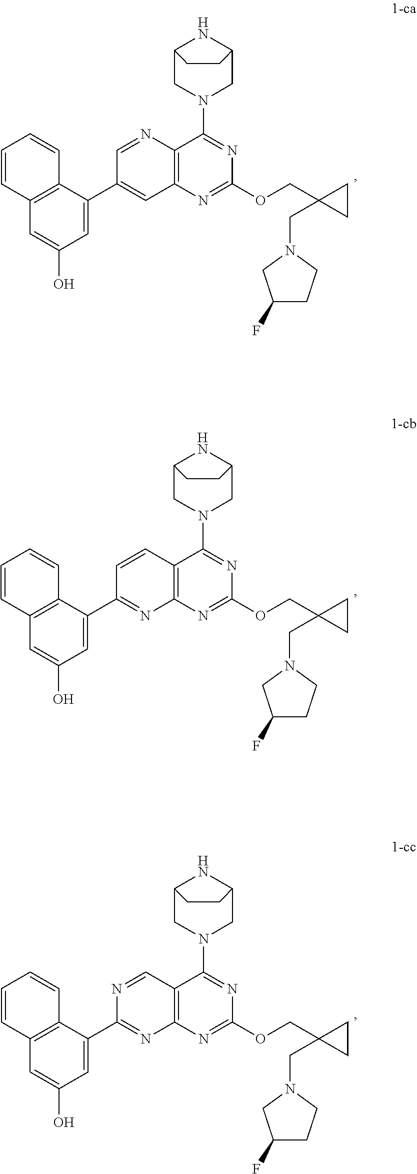 [SMILES: Oc1cc(-c2cnc3c(N4CC5CCC(C4)N5)nc(OCC4(CN5CC[C@@H](F)C5)CC4)nc3c2)c2ccccc2c1 ; Oc1cc(-c2ccc3c(N4CC5CCC(C4)N5)nc(OCC4(CN5CC[C@@H](F)C5)CC4)nc3n2)c2ccccc2c1 ; Oc1cc(-c2ncc3c(N4CC5CCC(C4)N5)nc(OCC4(CN5CC[C@@H](F)C5)CC4)nc3n2)c2ccccc2c1]

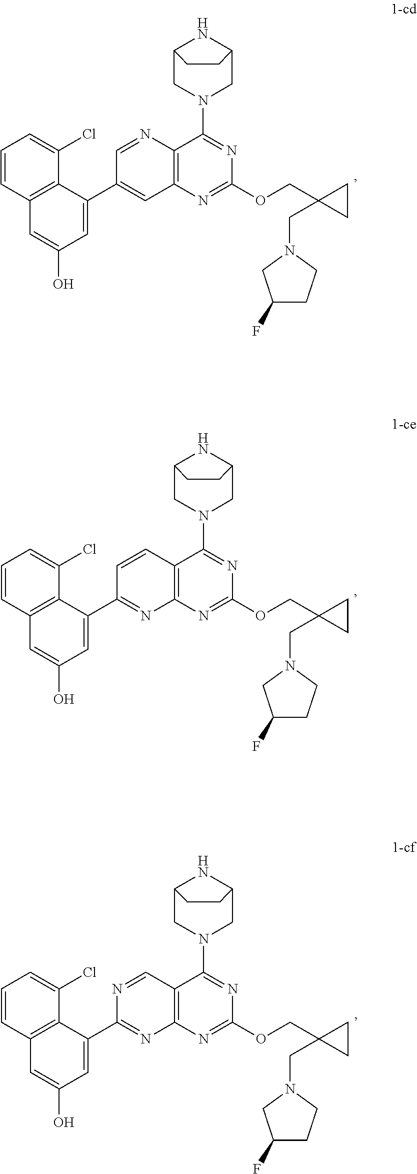 [SMILES: Oc1cc(-c2cnc3c(N4CC5CCC(C4)N5)nc(OCC4(CN5CC[C@@H](F)C5)CC4)nc3c2)c2c(Cl)cccc2c1 ; Oc1cc(-c2ccc3c(N4CC5CCC(C4)N5)nc(OCC4(CN5CC[C@@H](F)C5)CC4)nc3n2)c2c(Cl)cccc2c1 ; Oc1cc(-c2ncc3c(N4CC5CCC(C4)N5)nc(OCC4(CN5CC[C@@H](F)C5)CC4)nc3n2)c2c(Cl)cccc2c1]

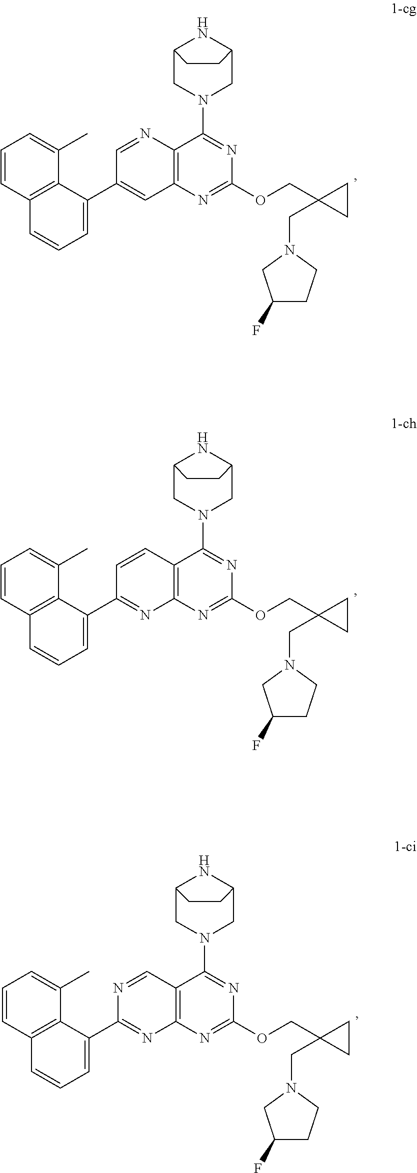 [SMILES: Cc1cccc2cccc(-c3cnc4c(N5CC6CCC(C5)N6)nc(OCC5(CN6CC[C@@H](F)C6)CC5)nc4c3)c12 ; Cc1cccc2cccc(-c3ccc4c(N5CC6CCC(C5)N6)nc(OCC5(CN6CC[C@@H](F)C6)CC5)nc4n3)c12 ; Cc1cccc2cccc(-c3ncc4c(N5CC6CCC(C5)N6)nc(OCC5(CN6CC[C@@H](F)C6)CC5)nc4n3)c12]

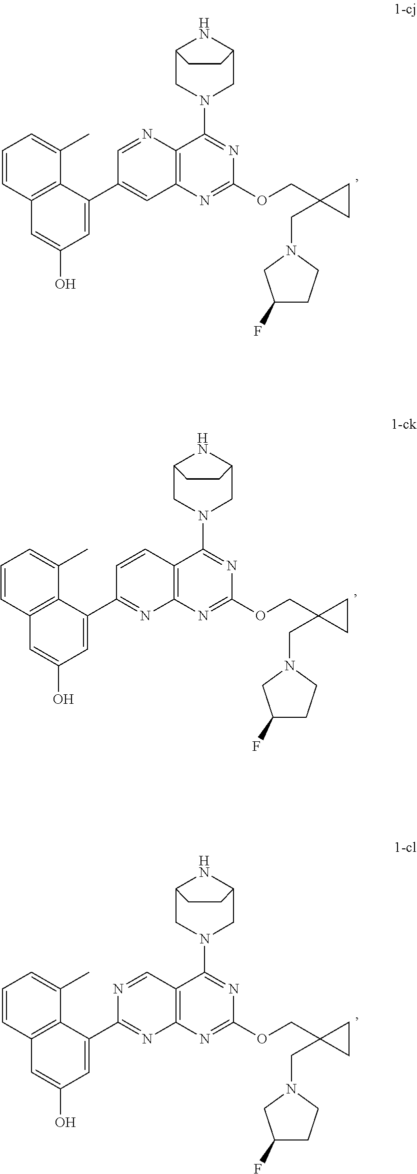 [SMILES: Cc1cccc2cc(O)cc(-c3cnc4c(N5CC6CCC(C5)N6)nc(OCC5(CN6CC[C@@H](F)C6)CC5)nc4c3)c12 ; Cc1cccc2cc(O)cc(-c3ccc4c(N5CC6CCC(C5)N6)nc(OCC5(CN6CC[C@@H](F)C6)CC5)nc4n3)c12 ; Cc1cccc2cc(O)cc(-c3ncc4c(N5CC6CCC(C5)N6)nc(OCC5(CN6CC[C@@H](F)C6)CC5)nc4n3)c12]

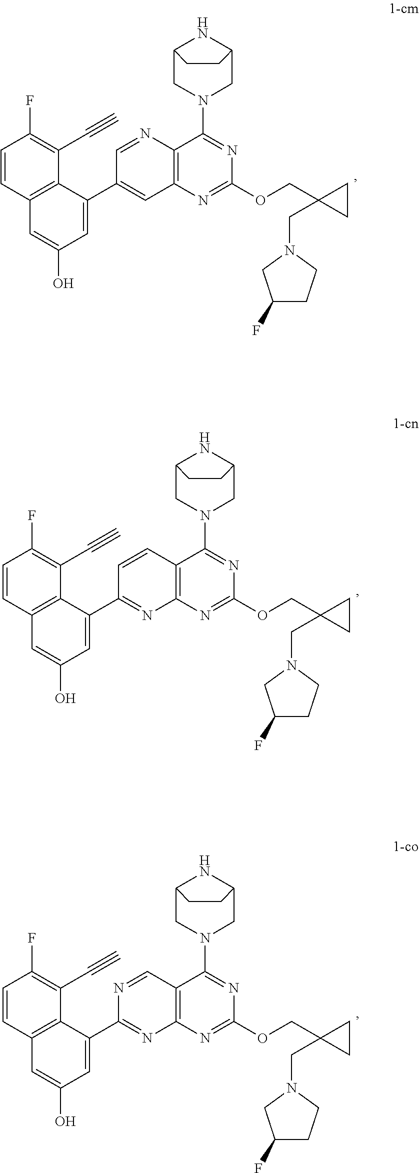 [SMILES: C#Cc1c(F)ccc2cc(O)cc(-c3cnc4c(N5CC6CCC(C5)N6)nc(OCC5(CN6CC[C@@H](F)C6)CC5)nc4c3)c12 ; C#Cc1c(F)ccc2cc(O)cc(-c3ccc4c(N5CC6CCC(C5)N6)nc(OCC5(CN6CC[C@@H](F)C6)CC5)nc4n3)c12 ; C#Cc1c(F)ccc2cc(O)cc(-c3ncc4c(N5CC6CCC(C5)N6)nc(OCC5(CN6CC[C@@H](F)C6)CC5)nc4n3)c12]

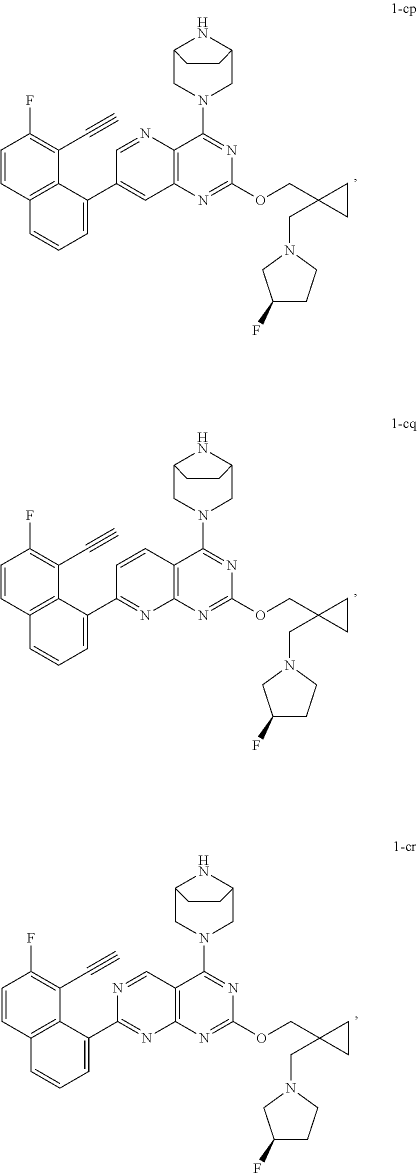 [SMILES: C#Cc1c(F)ccc2cccc(-c3cnc4c(N5CC6CCC(C5)N6)nc(OCC5(CN6CC[C@@H](F)C6)CC5)nc4c3)c12 ; C#Cc1c(F)ccc2cccc(-c3ccc4c(N5CC6CCC(C5)N6)nc(OCC5(CN6CC[C@@H](F)C6)CC5)nc4n3)c12 ; C#Cc1c(F)ccc2cccc(-c3ncc4c(N5CC6CCC(C5)N6)nc(OCC5(CN6CC[C@@H](F)C6)CC5)nc4n3)c12]

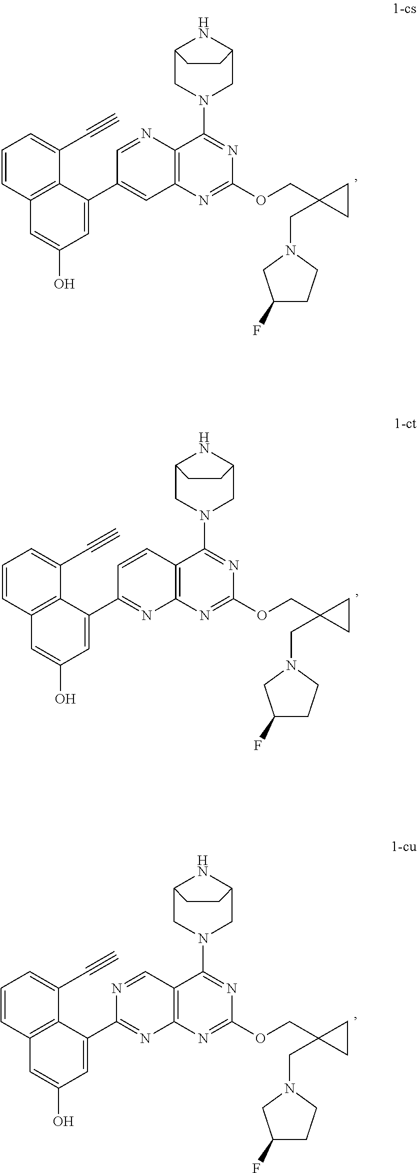 [SMILES: C#Cc1cccc2cc(O)cc(-c3cnc4c(N5CC6CCC(C5)N6)nc(OCC5(CN6CC[C@@H](F)C6)CC5)nc4c3)c12 ; C#Cc1cccc2cc(O)cc(-c3ccc4c(N5CC6CCC(C5)N6)nc(OCC5(CN6CC[C@@H](F)C6)CC5)nc4n3)c12 ; C#Cc1cccc2cc(O)cc(-c3ncc4c(N5CC6CCC(C5)N6)nc(OCC5(CN6CC[C@@H](F)C6)CC5)nc4n3)c12]

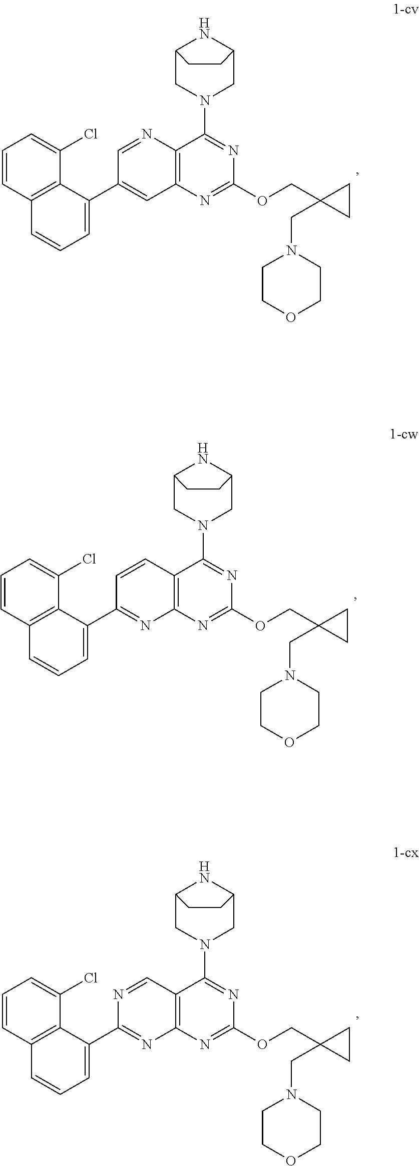 [SMILES: Clc1cccc2cccc(-c3cnc4c(N5CC6CCC(C5)N6)nc(OCC5(CN6CCOCC6)CC5)nc4c3)c12 ; Clc1cccc2cccc(-c3ccc4c(N5CC6CCC(C5)N6)nc(OCC5(CN6CCOCC6)CC5)nc4n3)c12 ; Clc1cccc2cccc(-c3ncc4c(N5CC6CCC(C5)N6)nc(OCC5(CN6CCOCC6)CC5)nc4n3)c12]

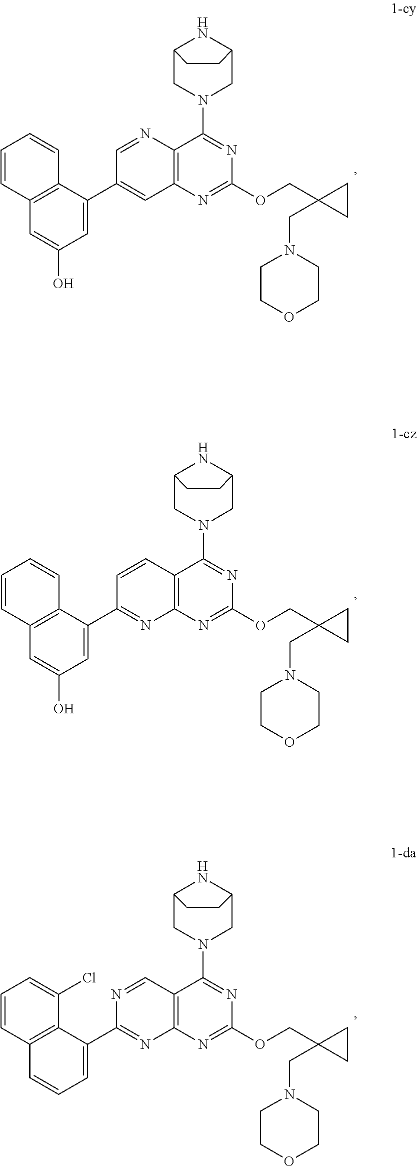 [SMILES: Oc1cc(-c2cnc3c(N4CC5CCC(C4)N5)nc(OCC4(CN5CCOCC5)CC4)nc3c2)c2ccccc2c1 ; Oc1cc(-c2ccc3c(N4CC5CCC(C4)N5)nc(OCC4(CN5CCOCC5)CC4)nc3n2)c2ccccc2c1 ; Clc1cccc2cccc(-c3ncc4c(N5CC6CCC(C5)N6)nc(OCC5(CN6CCOCC6)CC5)nc4n3)c12]

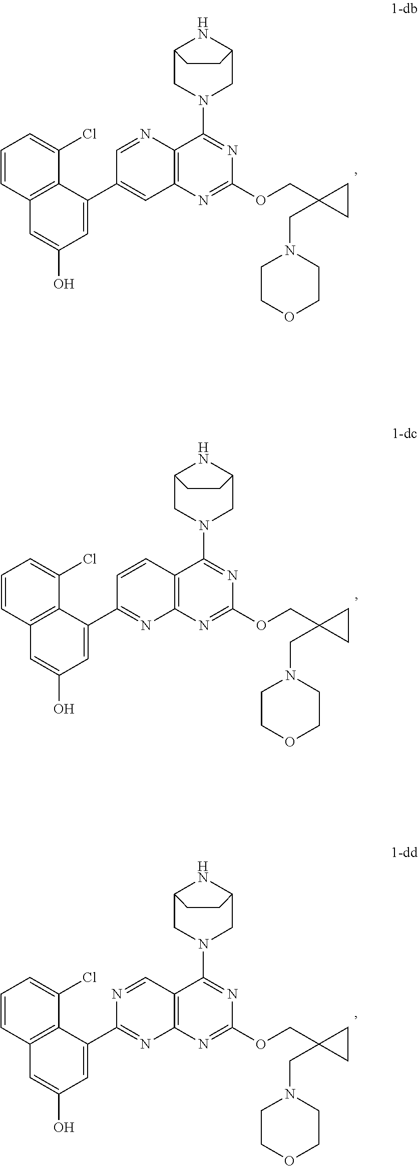 [SMILES: Oc1cc(-c2cnc3c(N4CC5CCC(C4)N5)nc(OCC4(CN5CCOCC5)CC4)nc3c2)c2c(Cl)cccc2c1 ; Oc1cc(-c2ccc3c(N4CC5CCC(C4)N5)nc(OCC4(CN5CCOCC5)CC4)nc3n2)c2c(Cl)cccc2c1 ; Oc1cc(-c2ncc3c(N4CC5CCC(C4)N5)nc(OCC4(CN5CCOCC5)CC4)nc3n2)c2c(Cl)cccc2c1]

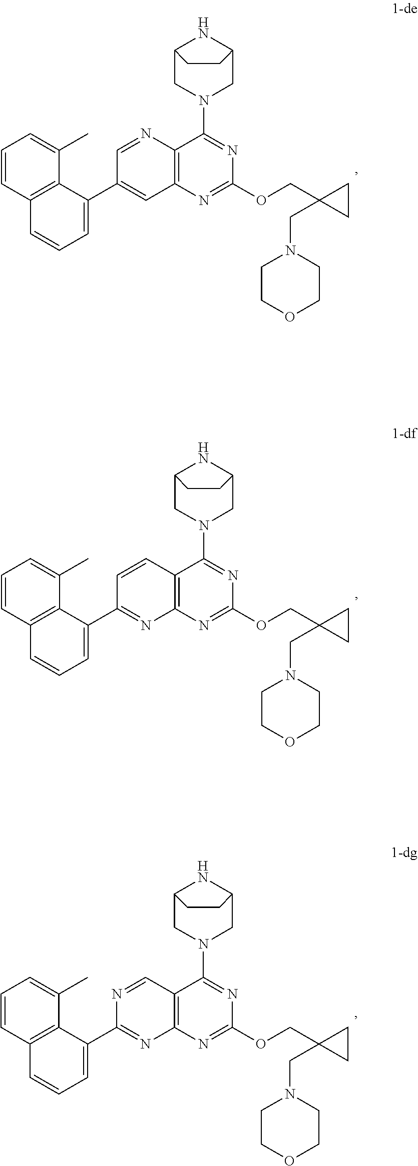 [SMILES: Cc1cccc2cccc(-c3cnc4c(N5CC6CCC(C5)N6)nc(OCC5(CN6CCOCC6)CC5)nc4c3)c12 ; Cc1cccc2cccc(-c3ccc4c(N5CC6CCC(C5)N6)nc(OCC5(CN6CCOCC6)CC5)nc4n3)c12 ; Cc1cccc2cccc(-c3ncc4c(N5CC6CCC(C5)N6)nc(OCC5(CN6CCOCC6)CC5)nc4n3)c12]

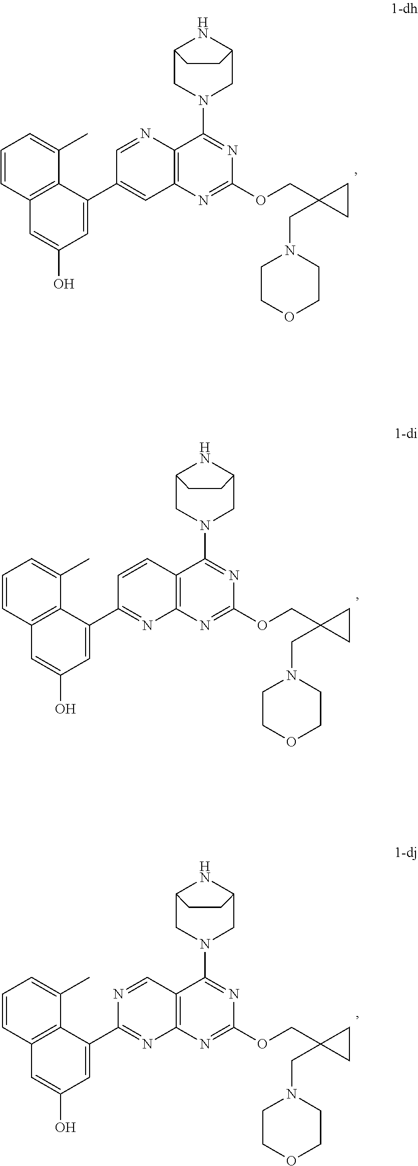 [SMILES: Cc1cccc2cc(O)cc(-c3cnc4c(N5CC6CCC(C5)N6)nc(OCC5(CN6CCOCC6)CC5)nc4c3)c12 ; Cc1cccc2cc(O)cc(-c3ccc4c(N5CC6CCC(C5)N6)nc(OCC5(CN6CCOCC6)CC5)nc4n3)c12 ; Cc1cccc2cc(O)cc(-c3ncc4c(N5CC6CCC(C5)N6)nc(OCC5(CN6CCOCC6)CC5)nc4n3)c12]

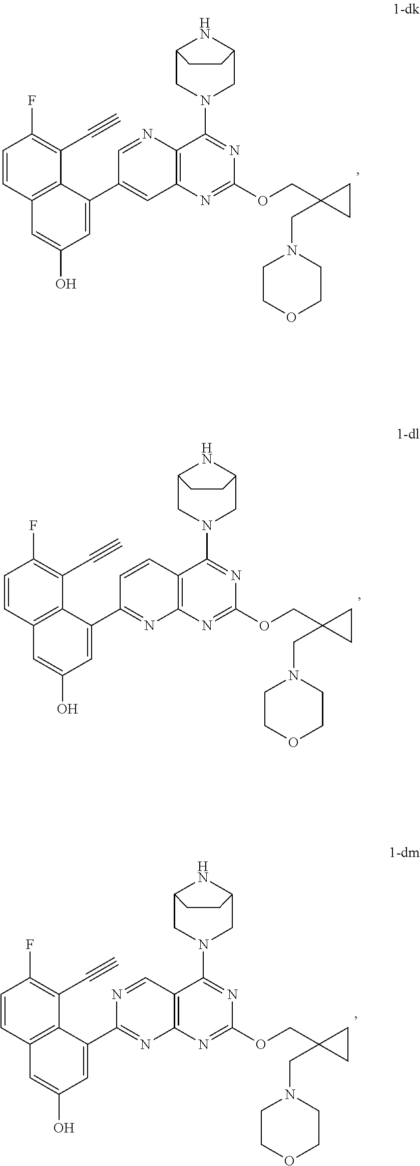 [SMILES: C#Cc1c(F)ccc2cc(O)cc(-c3cnc4c(N5CC6CCC(C5)N6)nc(OCC5(CN6CCOCC6)CC5)nc4c3)c12 ; C#Cc1c(F)ccc2cc(O)cc(-c3ccc4c(N5CC6CCC(C5)N6)nc(OCC5(CN6CCOCC6)CC5)nc4n3)c12 ; C#Cc1c(F)ccc2cc(O)cc(-c3ncc4c(N5CC6CCC(C5)N6)nc(OCC5(CN6CCOCC6)CC5)nc4n3)c12]

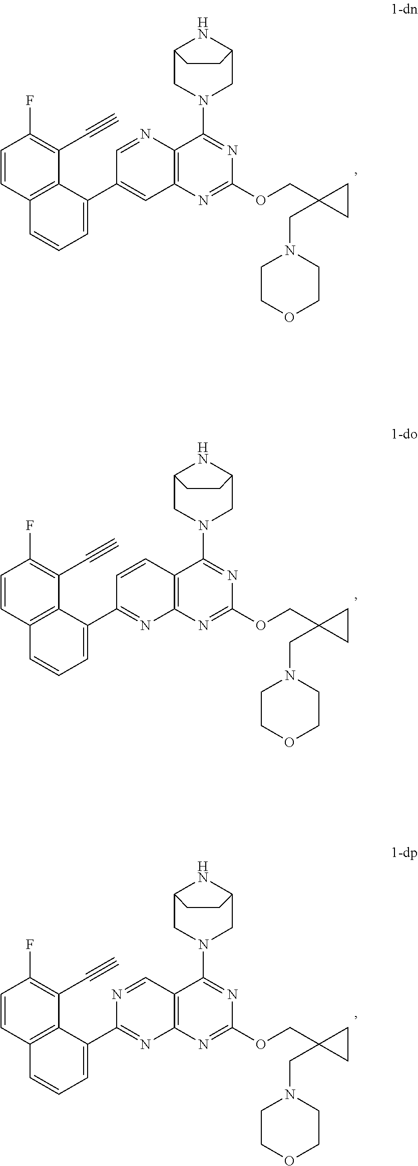 [SMILES: C#Cc1c(F)ccc2cccc(-c3cnc4c(N5CC6CCC(C5)N6)nc(OCC5(CN6CCOCC6)CC5)nc4c3)c12 ; C#Cc1c(F)ccc2cccc(-c3ccc4c(N5CC6CCC(C5)N6)nc(OCC5(CN6CCOCC6)CC5)nc4n3)c12 ; C#Cc1c(F)ccc2cccc(-c3ncc4c(N5CC6CCC(C5)N6)nc(OCC5(CN6CCOCC6)CC5)nc4n3)c12]

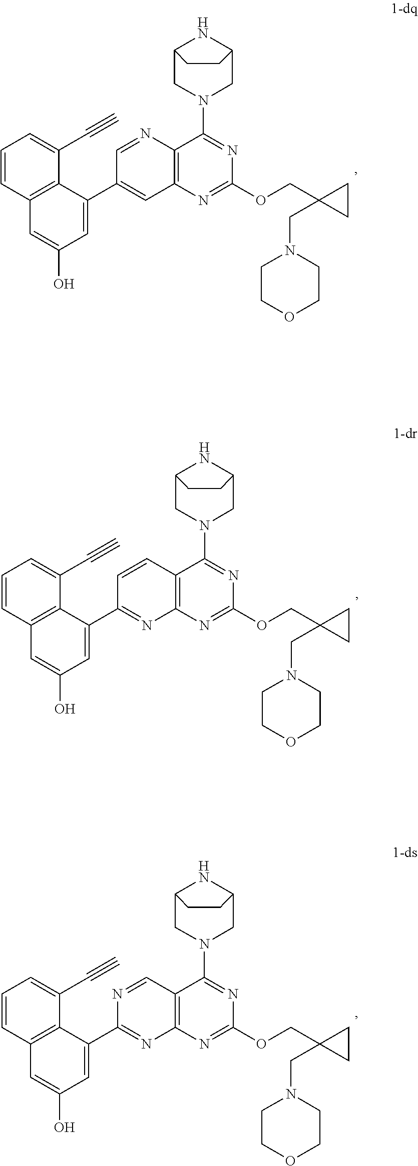 [SMILES: C#Cc1cccc2cc(O)cc(-c3cnc4c(N5CC6CCC(C5)N6)nc(OCC5(CN6CCOCC6)CC5)nc4c3)c12 ; C#Cc1cccc2cc(O)cc(-c3ccc4c(N5CC6CCC(C5)N6)nc(OCC5(CN6CCOCC6)CC5)nc4n3)c12 ; C#Cc1cccc2cc(O)cc(-c3ncc4c(N5CC6CCC(C5)N6)nc(OCC5(CN6CCOCC6)CC5)nc4n3)c12]

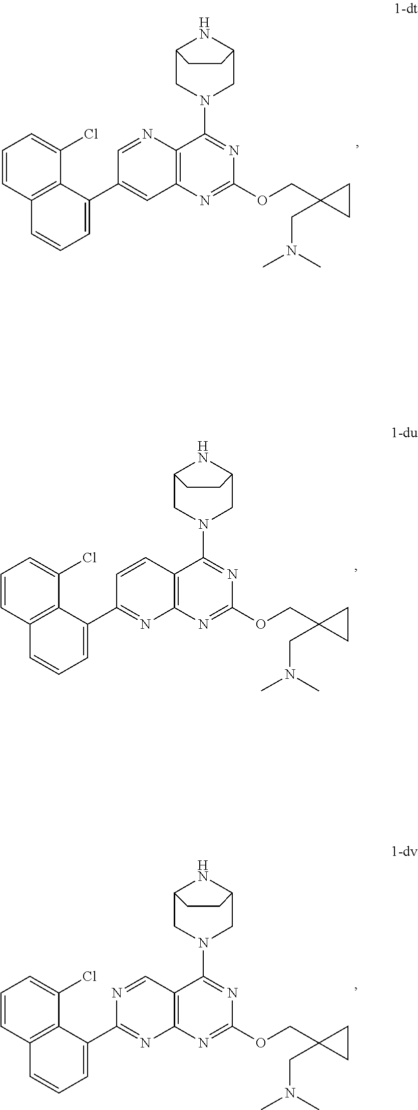 [SMILES: CN(C)CC1(COc2nc(N3CC4CCC(C3)N4)c3ncc(-c4cccc5cccc(Cl)c45)cc3n2)CC1 ; CN(C)CC1(COc2nc(N3CC4CCC(C3)N4)c3ccc(-c4cccc5cccc(Cl)c45)nc3n2)CC1 ; CN(C)CC1(COc2nc(N3CC4CCC(C3)N4)c3cnc(-c4cccc5cccc(Cl)c45)nc3n2)CC1]

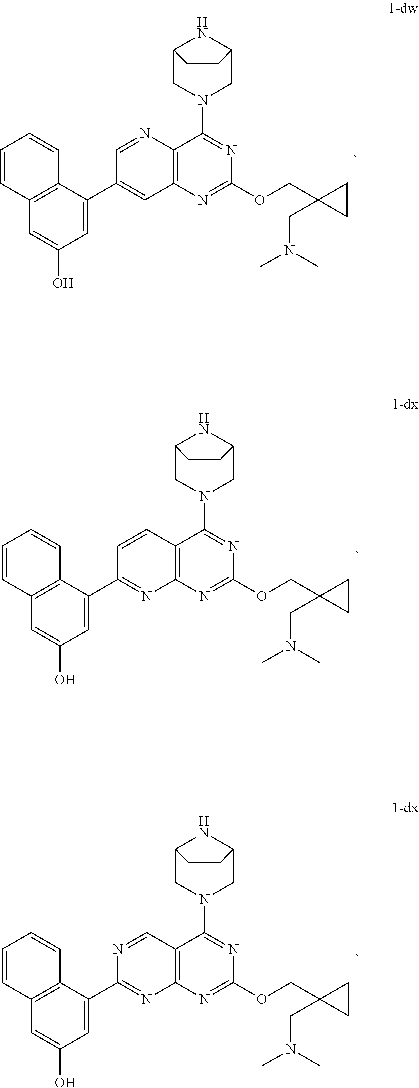 [SMILES: CN(C)CC1(COc2nc(N3CC4CCC(C3)N4)c3ncc(-c4cc(O)cc5ccccc45)cc3n2)CC1 ; CN(C)CC1(COc2nc(N3CC4CCC(C3)N4)c3ccc(-c4cc(O)cc5ccccc45)nc3n2)CC1 ; CN(C)CC1(COc2nc(N3CC4CCC(C3)N4)c3cnc(-c4cc(O)cc5ccccc45)nc3n2)CC1]

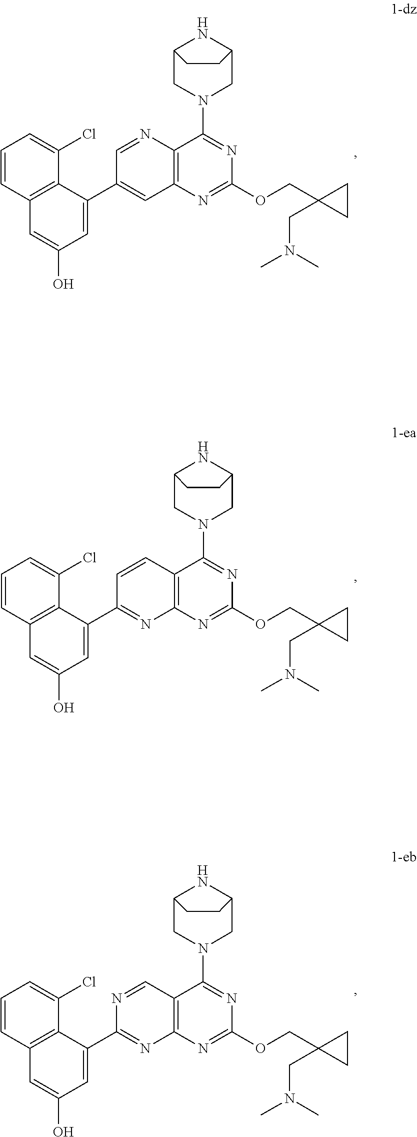 [SMILES: CN(C)CC1(COc2nc(N3CC4CCC(C3)N4)c3ncc(-c4cc(O)cc5cccc(Cl)c45)cc3n2)CC1 ; CN(C)CC1(COc2nc(N3CC4CCC(C3)N4)c3ccc(-c4cc(O)cc5cccc(Cl)c45)nc3n2)CC1 ; CN(C)CC1(COc2nc(N3CC4CCC(C3)N4)c3cnc(-c4cc(O)cc5cccc(Cl)c45)nc3n2)CC1]

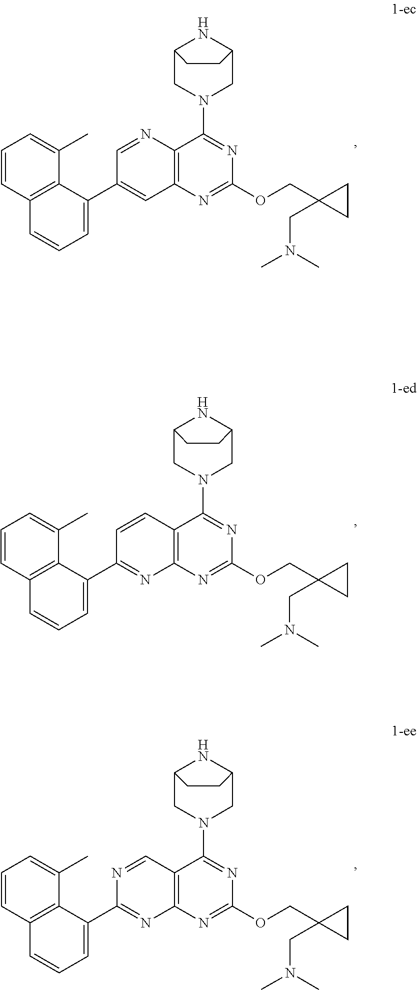 [SMILES: Cc1cccc2cccc(-c3cnc4c(N5CC6CCC(C5)N6)nc(OCC5(CN(C)C)CC5)nc4c3)c12 ; Cc1cccc2cccc(-c3ccc4c(N5CC6CCC(C5)N6)nc(OCC5(CN(C)C)CC5)nc4n3)c12 ; Cc1cccc2cccc(-c3ncc4c(N5CC6CCC(C5)N6)nc(OCC5(CN(C)C)CC5)nc4n3)c12]

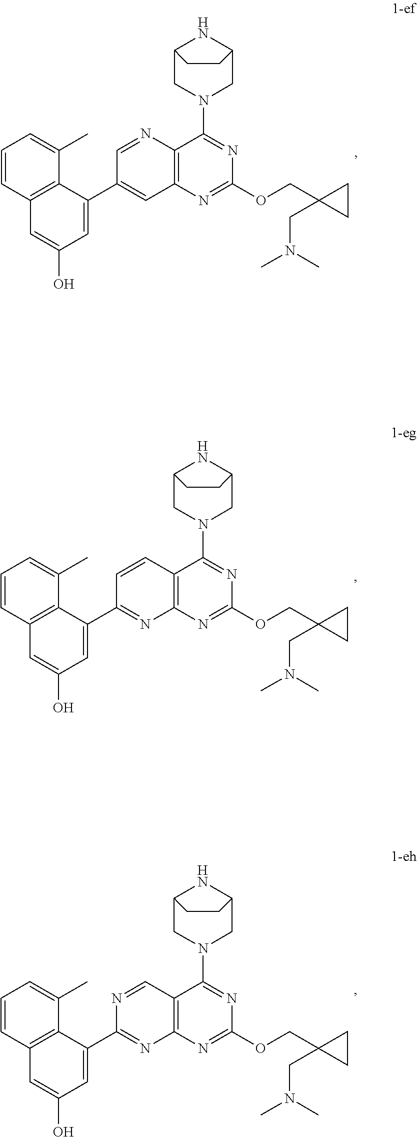 [SMILES: Cc1cccc2cc(O)cc(-c3cnc4c(N5CC6CCC(C5)N6)nc(OCC5(CN(C)C)CC5)nc4c3)c12 ; Cc1cccc2cc(O)cc(-c3ccc4c(N5CC6CCC(C5)N6)nc(OCC5(CN(C)C)CC5)nc4n3)c12 ; Cc1cccc2cc(O)cc(-c3ncc4c(N5CC6CCC(C5)N6)nc(OCC5(CN(C)C)CC5)nc4n3)c12]

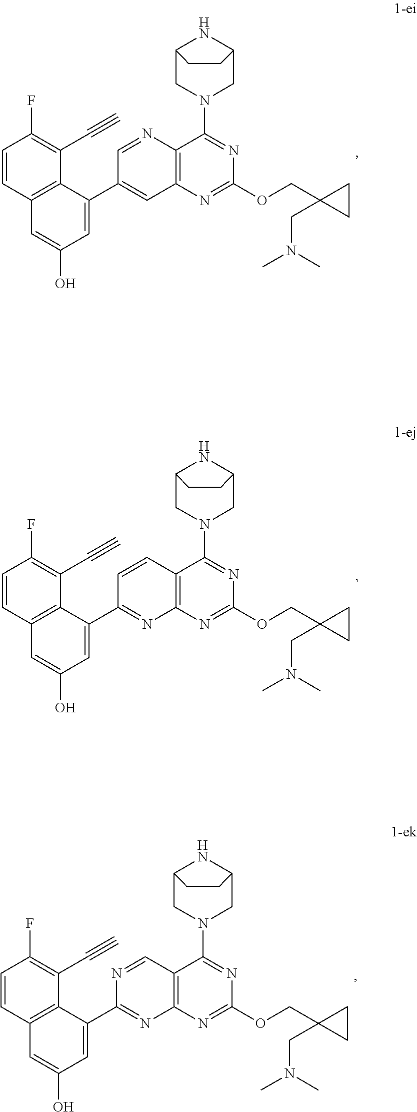 [SMILES: C#Cc1c(F)ccc2cc(O)cc(-c3cnc4c(N5CC6CCC(C5)N6)nc(OCC5(CN(C)C)CC5)nc4c3)c12 ; C#Cc1c(F)ccc2cc(O)cc(-c3ccc4c(N5CC6CCC(C5)N6)nc(OCC5(CN(C)C)CC5)nc4n3)c12 ; C#Cc1c(F)ccc2cc(O)cc(-c3ncc4c(N5CC6CCC(C5)N6)nc(OCC5(CN(C)C)CC5)nc4n3)c12]

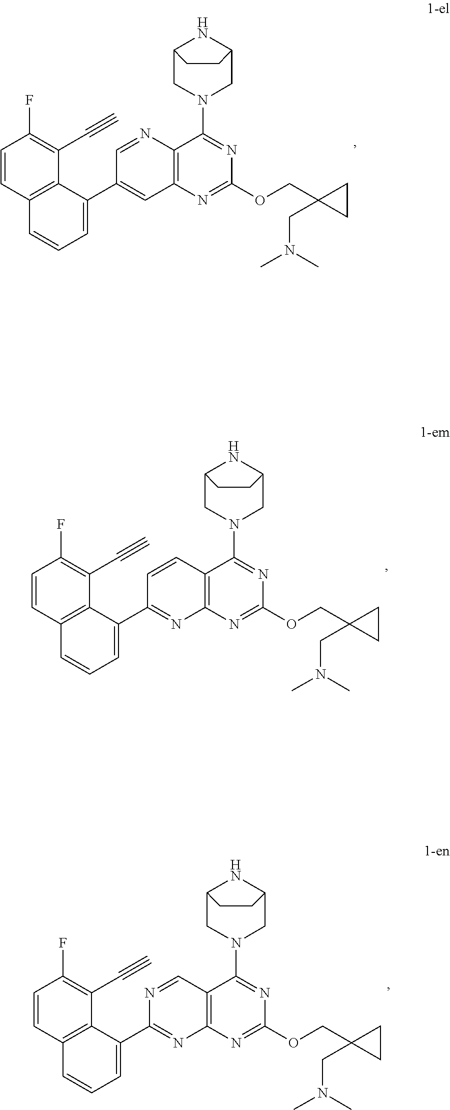 [SMILES: C#Cc1c(F)ccc2cccc(-c3cnc4c(N5CC6CCC(C5)N6)nc(OCC5(CN(C)C)CC5)nc4c3)c12 ; C#Cc1c(F)ccc2cccc(-c3ccc4c(N5CC6CCC(C5)N6)nc(OCC5(CN(C)C)CC5)nc4n3)c12 ; C#Cc1c(F)ccc2cccc(-c3ncc4c(N5CC6CCC(C5)N6)nc(OCC5(CN(C)C)CC5)nc4n3)c12]

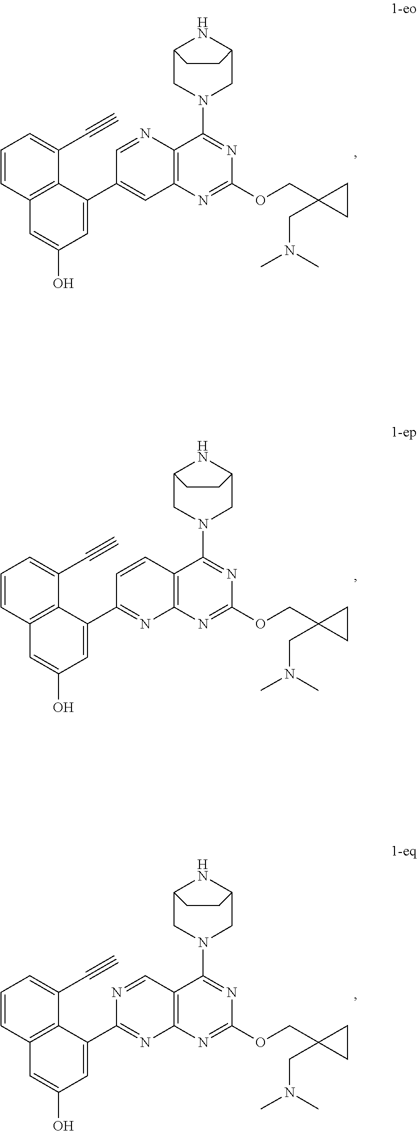 [SMILES: C#Cc1cccc2cc(O)cc(-c3cnc4c(N5CC6CCC(C5)N6)nc(OCC5(CN(C)C)CC5)nc4c3)c12 ; C#Cc1cccc2cc(O)cc(-c3ccc4c(N5CC6CCC(C5)N6)nc(OCC5(CN(C)C)CC5)nc4n3)c12 ; C#Cc1cccc2cc(O)cc(-c3ncc4c(N5CC6CCC(C5)N6)nc(OCC5(CN(C)C)CC5)nc4n3)c12]

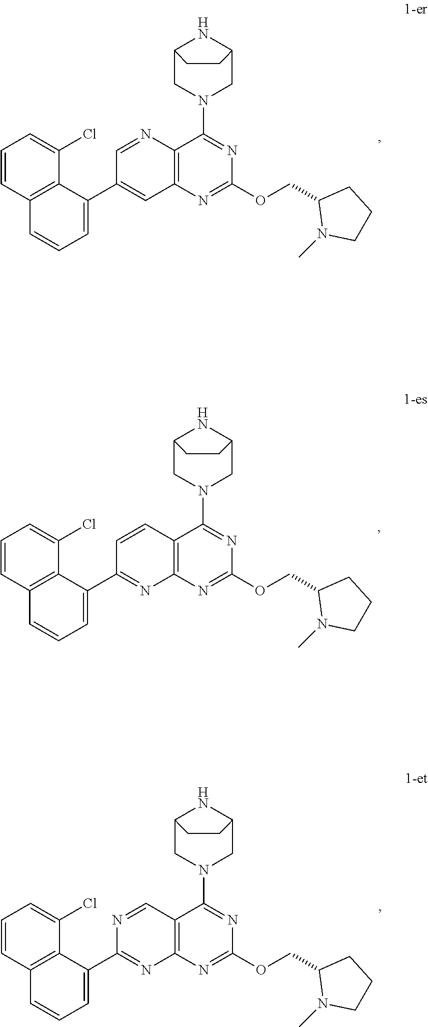

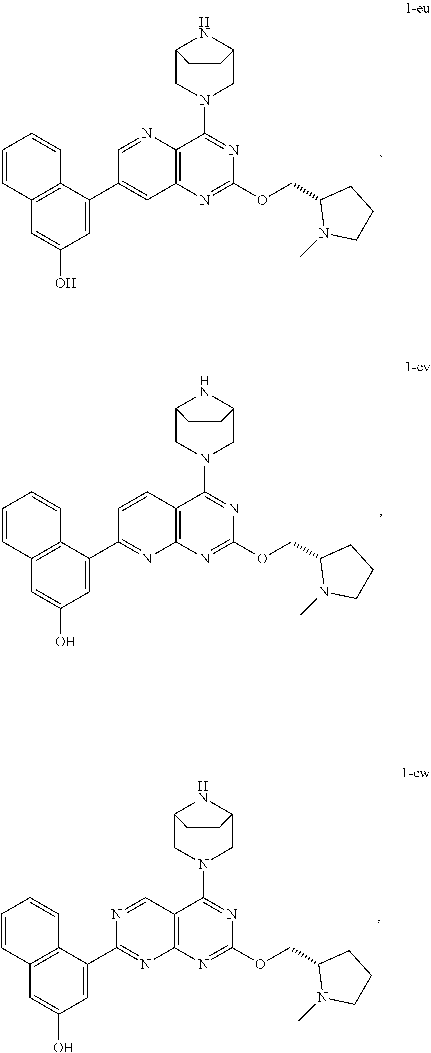

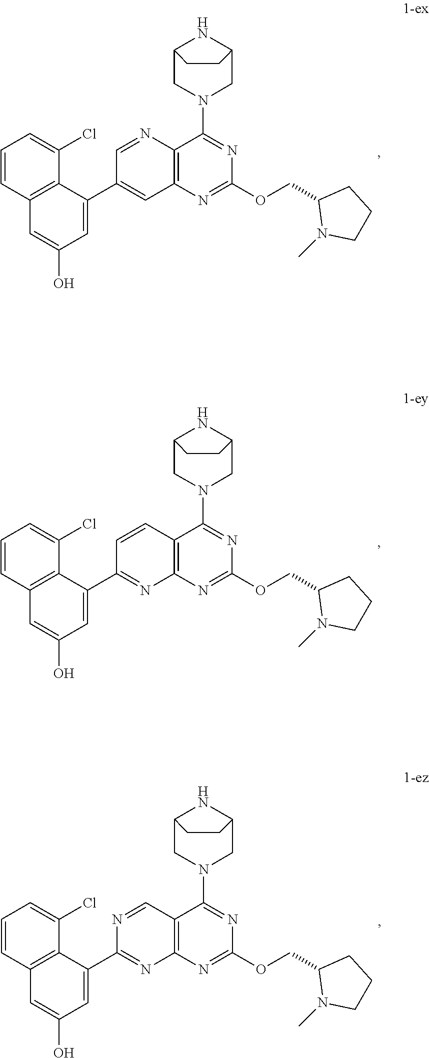

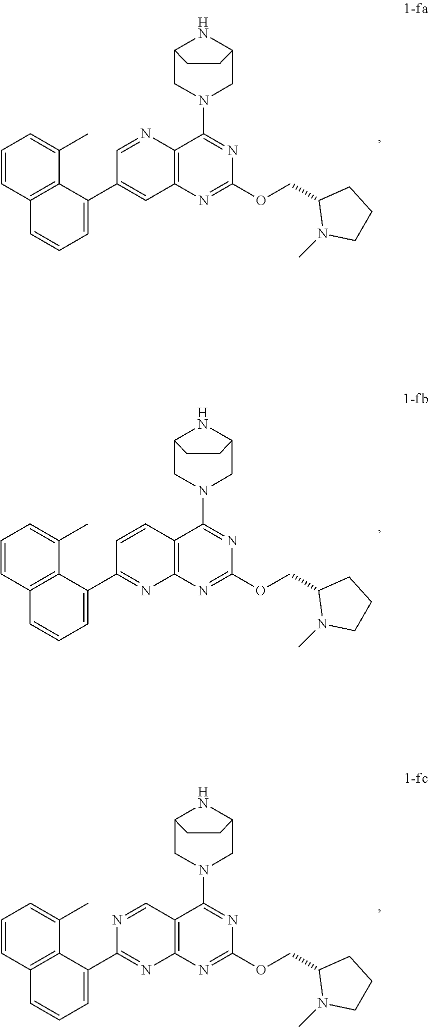

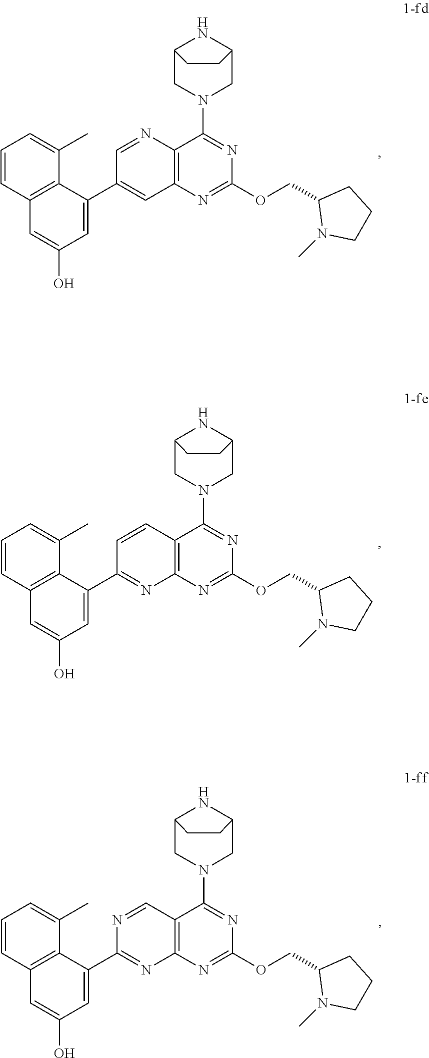

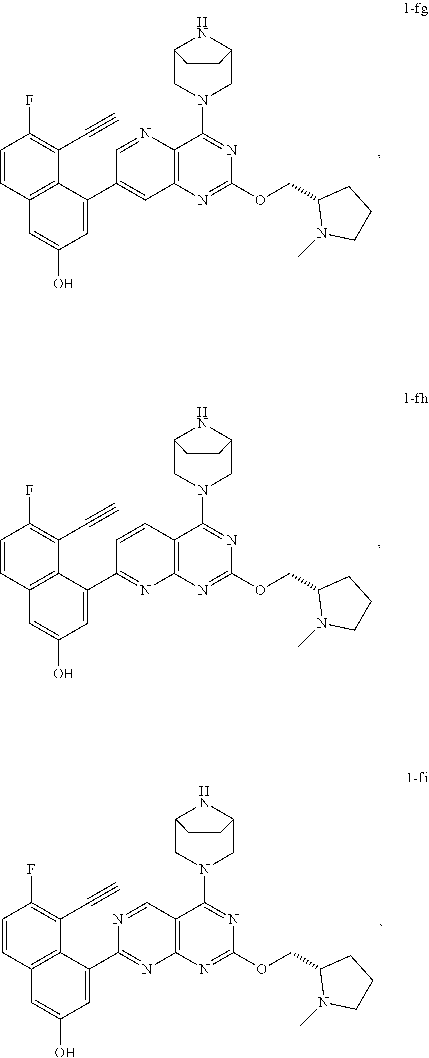

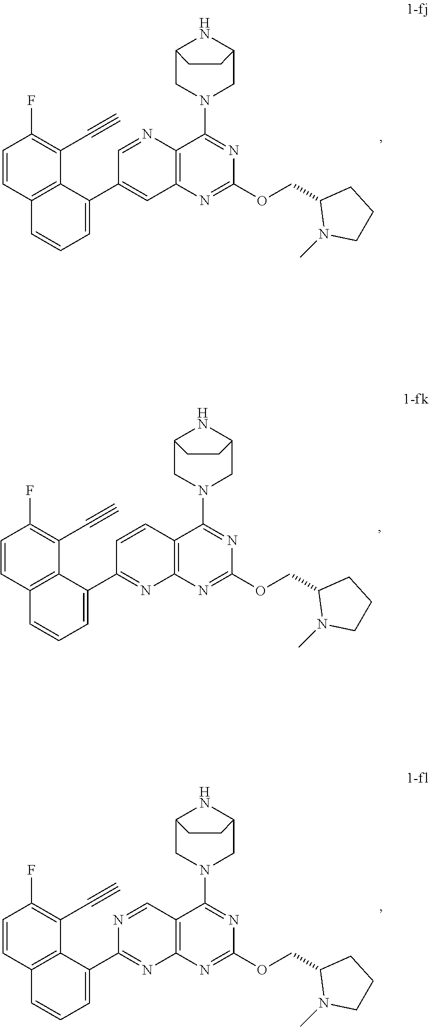

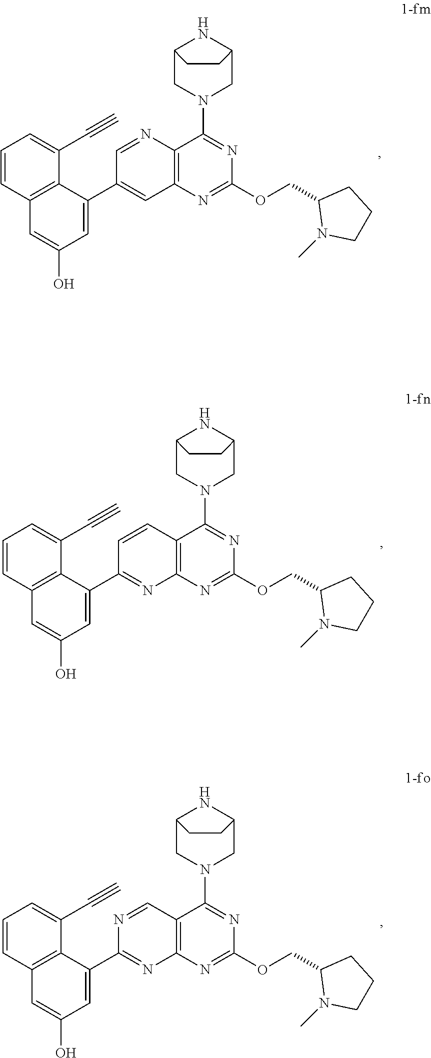

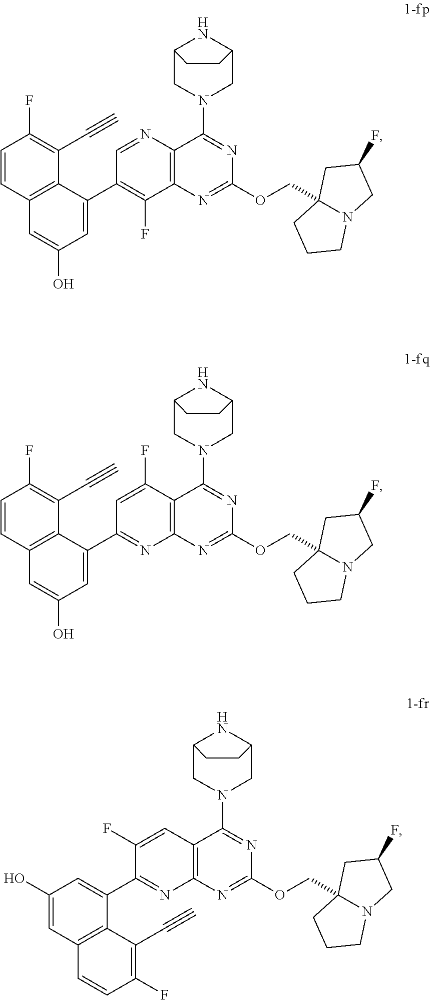 [SMILES: C#Cc1c(F)ccc2cc(O)cc(-c3cnc4c(N5CC6CCC(C5)N6)nc(OC[C@@]56CCCN5C[C@H](F)C6)nc4c3F)c12 ; C#Cc1c(F)ccc2cc(O)cc(-c3cc(F)c4c(N5CC6CCC(C5)N6)nc(OC[C@@]56CCCN5C[C@H](F)C6)nc4n3)c12 ; C#Cc1c(F)ccc2cc(O)cc(-c3nc4nc(OC[C@@]56CCCN5C[C@H](F)C6)nc(N5CC6CCC(C5)N6)c4cc3F)c12]

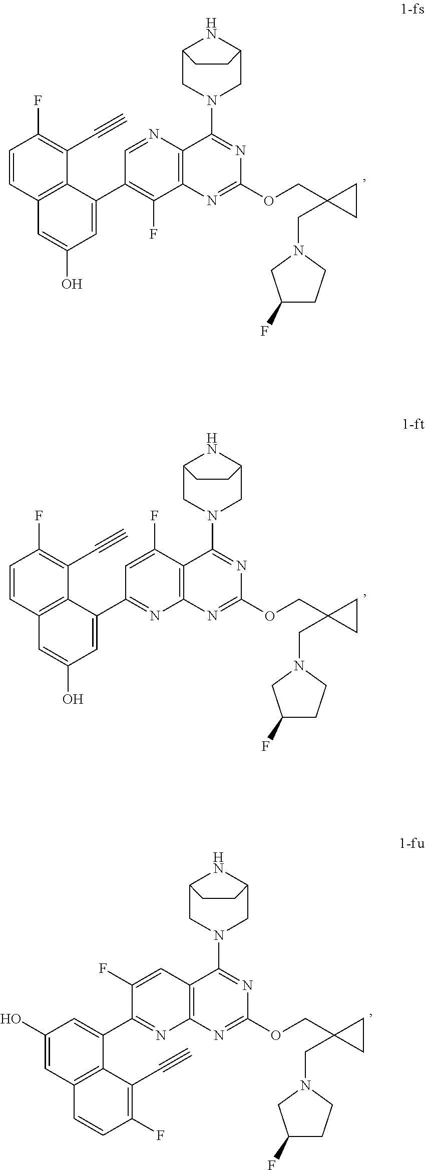 [SMILES: C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cc(F)c4c(N5CC6CCC(C5)N6)nc(OCC5(CN6CC[C@@H](F)C6)CC5)nc4n3)c12 ; C#Cc1c(F)ccc2cc(O)cc(-c3nc4nc(OCC5(CN6CC[C@@H](F)C6)CC5)nc(N5CC6CCC(C5)N6)c4cc3F)c12]

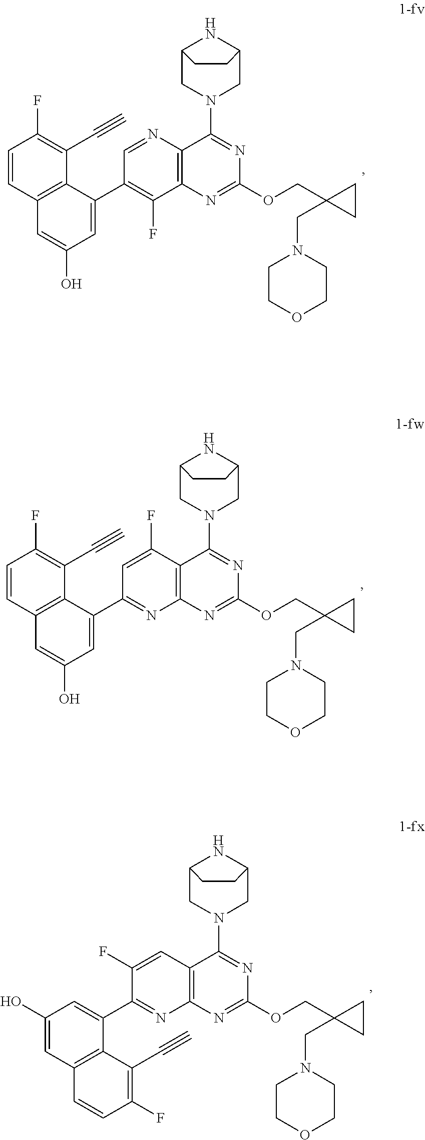 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cc(F)c4c(N5CC6CCC(C5)N6)nc(OCC5(CN6CCOCC6)CC5)nc4n3)c12 ; C#Cc1c(F)ccc2cc(O)cc(-c3nc4nc(OCC5(CN6CCOCC6)CC5)nc(N5CC6CCC(C5)N6)c4cc3F)c12]

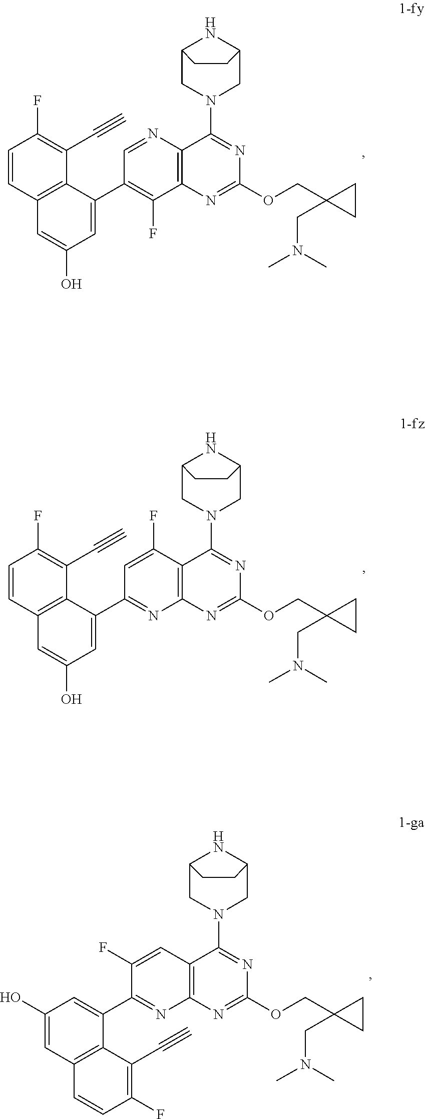 [SMILES: C#Cc1c(F)ccc2cc(O)cc(-c3cnc4c(N5CC6CCC(C5)N6)nc(OCC5(CN(C)C)CC5)nc4c3F)c12 ; C#Cc1c(F)ccc2cc(O)cc(-c3cc(F)c4c(N5CC6CCC(C5)N6)nc(OCC5(CN(C)C)CC5)nc4n3)c12 ; C#Cc1c(F)ccc2cc(O)cc(-c3nc4nc(OCC5(CN(C)C)CC5)nc(N5CC6CCC(C5)N6)c4cc3F)c12]

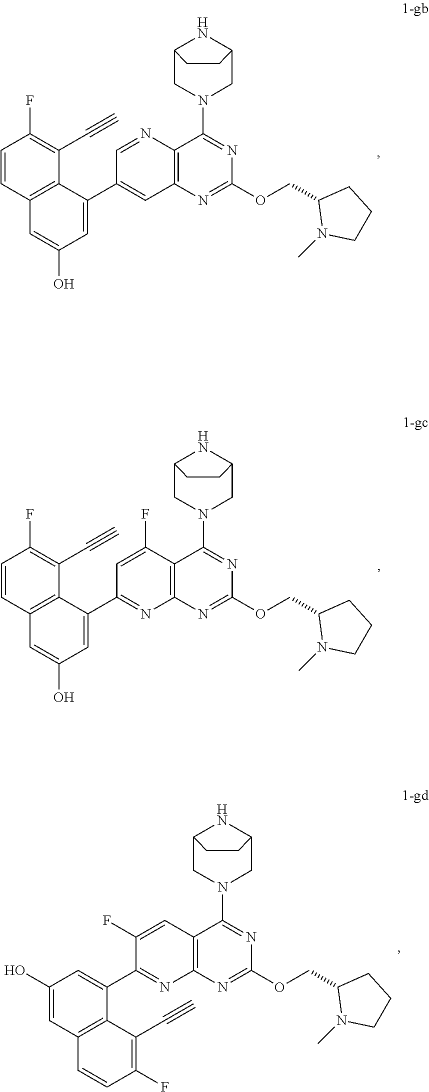

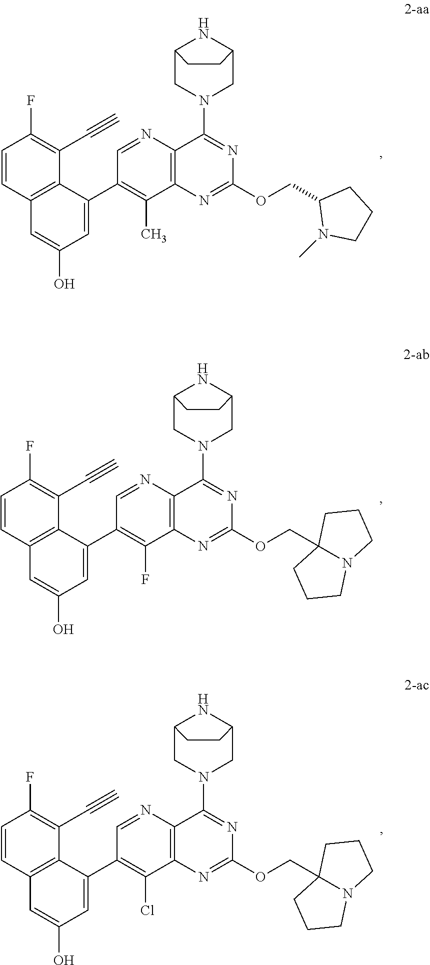 [SMILES: C#Cc1c(F)ccc2cc(O)cc(-c3cnc4c(N5CC6CCC(C5)N6)nc(OC[C@@H]5CCCN5C)nc4c3C)c12 ; C#Cc1c(F)ccc2cc(O)cc(-c3cnc4c(N5CC6CCC(C5)N6)nc(OCC56CCCN5CCC6)nc4c3F)c12 ; C#Cc1c(F)ccc2cc(O)cc(-c3cnc4c(N5CC6CCC(C5)N6)nc(OCC56CCCN5CCC6)nc4c3Cl)c12]

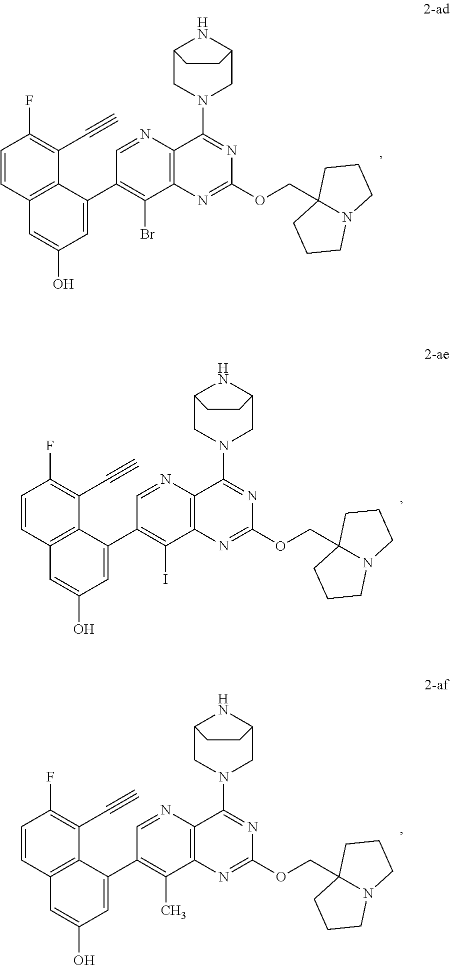 [SMILES: C#Cc1c(F)ccc2cc(O)cc(-c3cnc4c(N5CC6CCC(C5)N6)nc(OCC56CCCN5CCC6)nc4c3Br)c12 ; C#Cc1c(F)ccc2cc(O)cc(-c3cnc4c(N5CC6CCC(C5)N6)nc(OCC56CCCN5CCC6)nc4c3I)c12 ; C#Cc1c(F)ccc2cc(O)cc(-c3cnc4c(N5CC6CCC(C5)N6)nc(OCC56CCCN5CCC6)nc4c3C)c12]

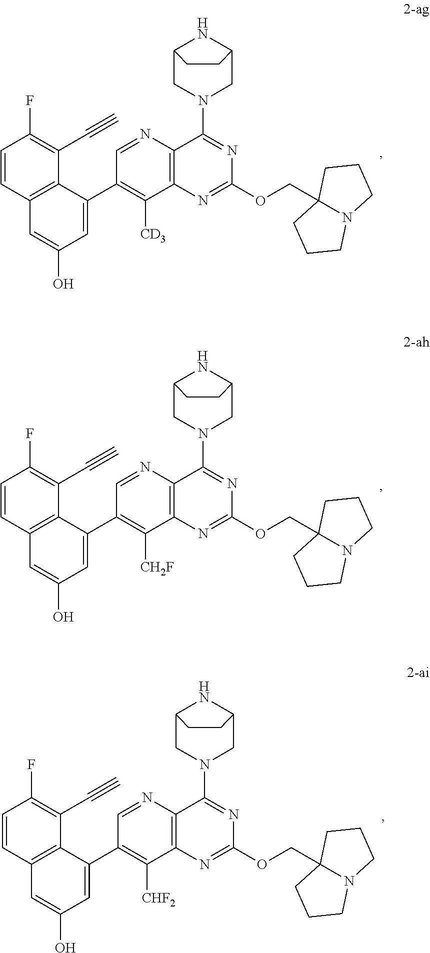

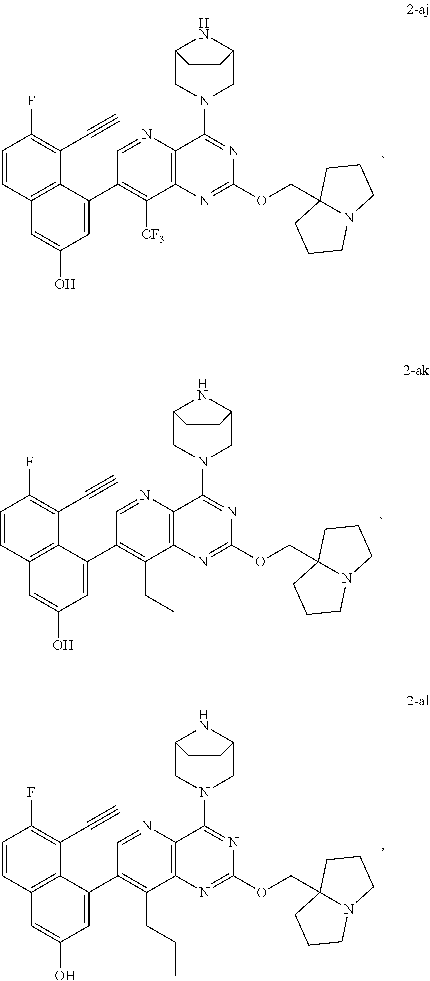 [SMILES: C#Cc1c(F)ccc2cc(O)cc(-c3cnc4c(N5CC6CCC(C5)N6)nc(OCC56CCCN5CCC6)nc4c3C(F)(F)F)c12 ; C#Cc1c(F)ccc2cc(O)cc(-c3cnc4c(N5CC6CCC(C5)N6)nc(OCC56CCCN5CCC6)nc4c3CC)c12 ; C#Cc1c(F)ccc2cc(O)cc(-c3cnc4c(N5CC6CCC(C5)N6)nc(OCC56CCCN5CCC6)nc4c3CCC)c12]

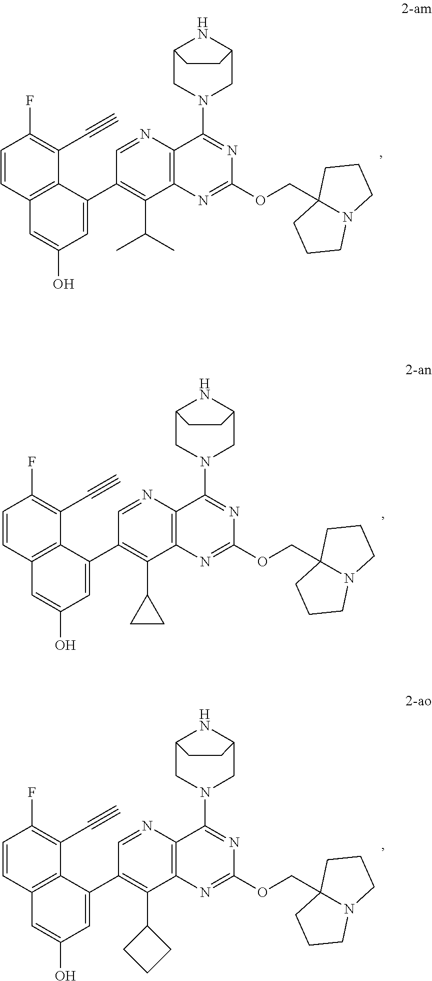 [SMILES: C#Cc1c(F)ccc2cc(O)cc(-c3cnc4c(N5CC6CCC(C5)N6)nc(OCC56CCCN5CCC6)nc4c3C(C)C)c12 ; C#Cc1c(F)ccc2cc(O)cc(-c3cnc4c(N5CC6CCC(C5)N6)nc(OCC56CCCN5CCC6)nc4c3C3CC3)c12 ; C#Cc1c(F)ccc2cc(O)cc(-c3cnc4c(N5CC6CCC(C5)N6)nc(OCC56CCCN5CCC6)nc4c3C3CCC3)c12]

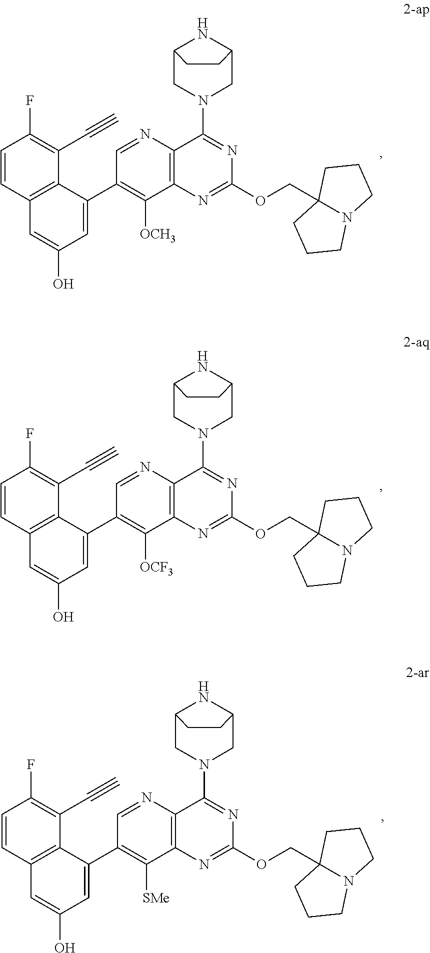 [SMILES: C#Cc1c(F)ccc2cc(O)cc(-c3cnc4c(N5CC6CCC(C5)N6)nc(OCC56CCCN5CCC6)nc4c3OC)c12 ; C#Cc1c(F)ccc2cc(O)cc(-c3cnc4c(N5CC6CCC(C5)N6)nc(OCC56CCCN5CCC6)nc4c3OC(F)(F)F)c12 ; C#Cc1c(F)ccc2cc(O)cc(-c3cnc4c(N5CC6CCC(C5)N6)nc(OCC56CCCN5CCC6)nc4c3SC)c12]

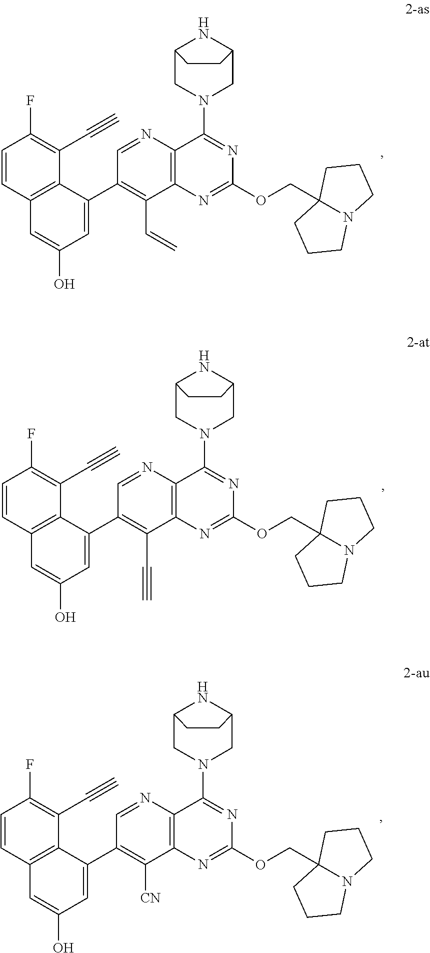 [SMILES: C#Cc1c(F)ccc2cc(O)cc(-c3cnc4c(N5CC6CCC(C5)N6)nc(OCC56CCCN5CCC6)nc4c3C=C)c12 ; C#Cc1c(-c2cc(O)cc3ccc(F)c(C#C)c23)cnc2c(N3CC4CCC(C3)N4)nc(OCC34CCCN3CCC4)nc12 ; C#Cc1c(F)ccc2cc(O)cc(-c3cnc4c(N5CC6CCC(C5)N6)nc(OCC56CCCN5CCC6)nc4c3C#N)c12]

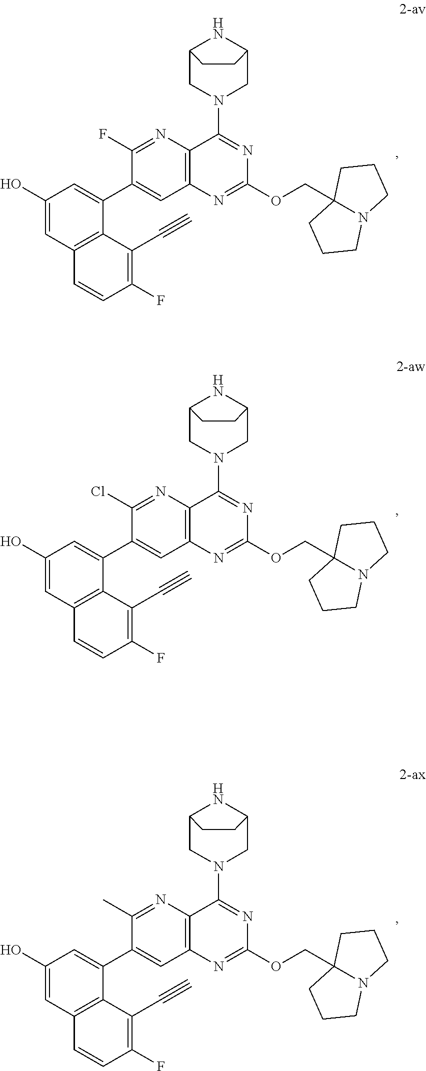 [SMILES: C#Cc1c(F)ccc2cc(O)cc(-c3cc4nc(OCC56CCCN5CCC6)nc(N5CC6CCC(C5)N6)c4nc3F)c12 ; C#Cc1c(F)ccc2cc(O)cc(-c3cc4nc(OCC56CCCN5CCC6)nc(N5CC6CCC(C5)N6)c4nc3Cl)c12 ; C#Cc1c(F)ccc2cc(O)cc(-c3cc4nc(OCC56CCCN5CCC6)nc(N5CC6CCC(C5)N6)c4nc3C)c12]

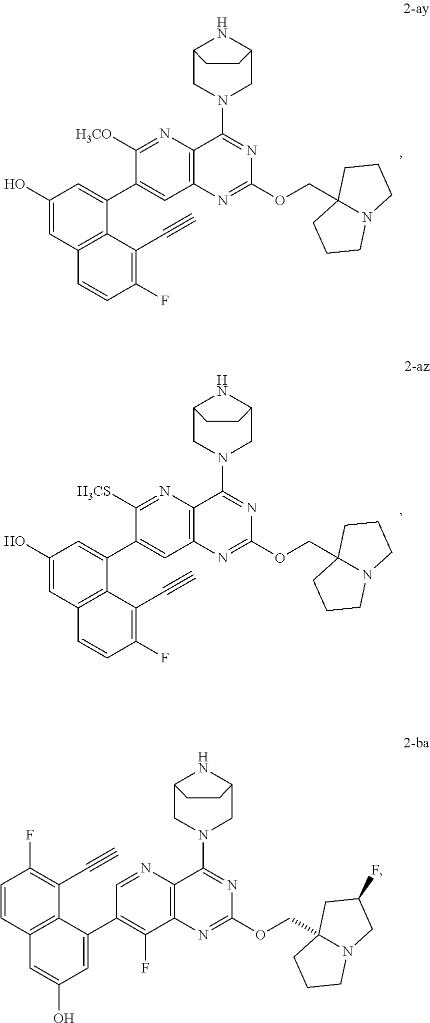

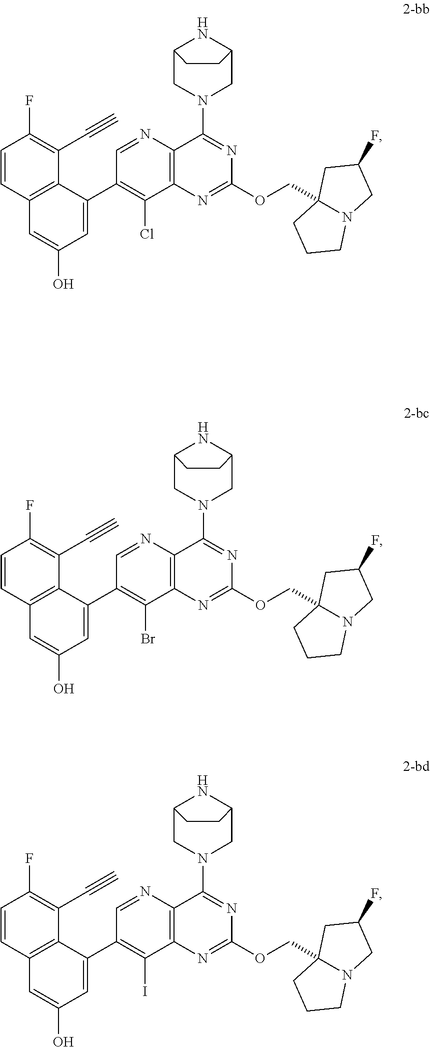 [SMILES: C#Cc1c(F)ccc2cc(O)cc(-c3cnc4c(N5CC6CCC(C5)N6)nc(OC[C@@]56CCCN5C[C@H](F)C6)nc4c3Cl)c12 ; C#Cc1c(F)ccc2cc(O)cc(-c3cnc4c(N5CC6CCC(C5)N6)nc(OC[C@@]56CCCN5C[C@H](F)C6)nc4c3Br)c12 ; C#Cc1c(F)ccc2cc(O)cc(-c3cnc4c(N5CC6CCC(C5)N6)nc(OC[C@@]56CCCN5C[C@H](F)C6)nc4c3I)c12]

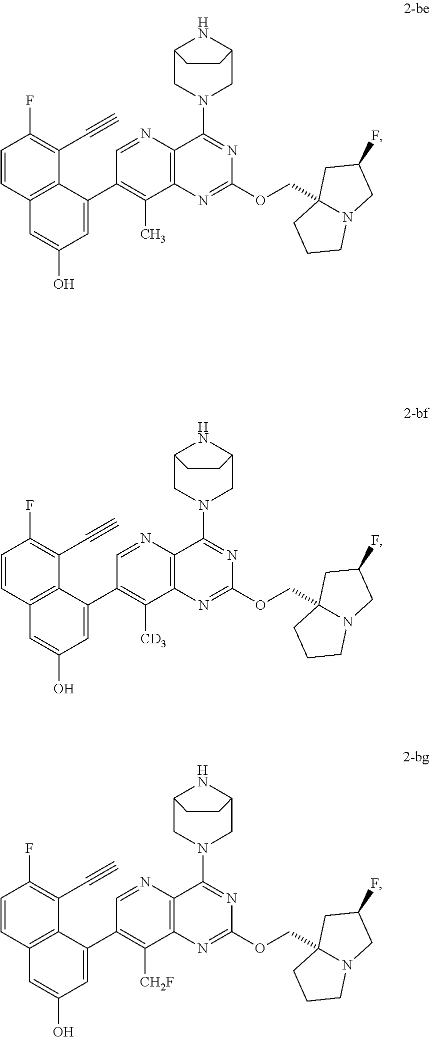

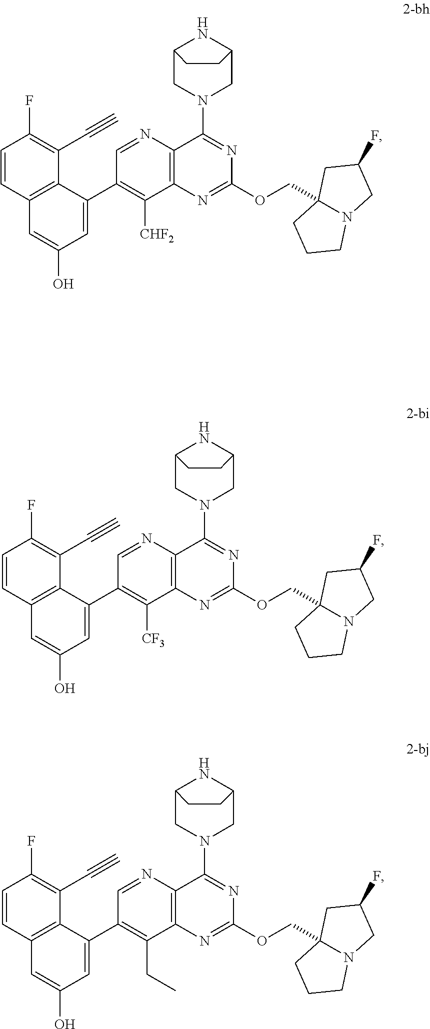 [SMILES: C#Cc1c(F)ccc2cc(O)cc(-c3cnc4c(N5CC6CCC(C5)N6)nc(OC[C@@]56CCCN5C[C@H](F)C6)nc4c3C(F)F)c12 ; C#Cc1c(F)ccc2cc(O)cc(-c3cnc4c(N5CC6CCC(C5)N6)nc(OC[C@@]56CCCN5C[C@H](F)C6)nc4c3C(F)(F)F)c12 ; C#Cc1c(F)ccc2cc(O)cc(-c3cnc4c(N5CC6CCC(C5)N6)nc(OC[C@@]56CCCN5C[C@H](F)C6)nc4c3CC)c12]

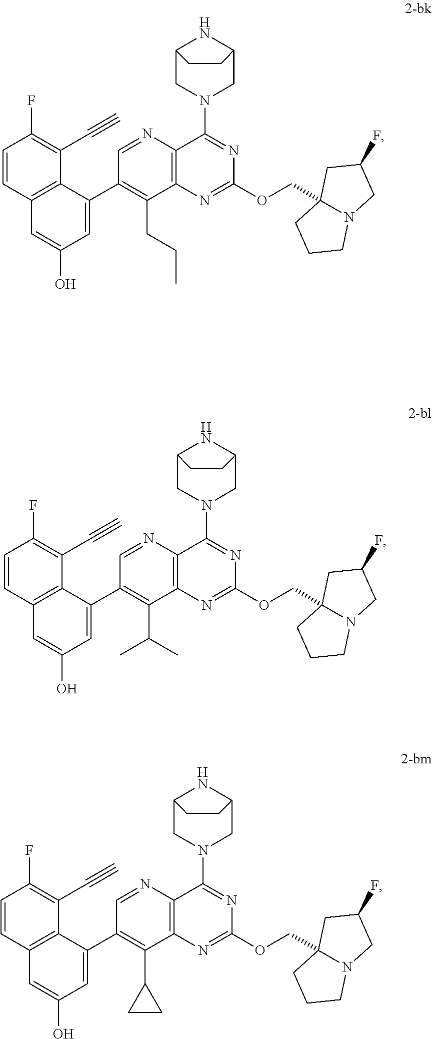 [SMILES: C#Cc1c(F)ccc2cc(O)cc(-c3cnc4c(N5CC6CCC(C5)N6)nc(OC[C@@]56CCCN5C[C@H](F)C6)nc4c3CCC)c12 ; C#Cc1c(F)ccc2cc(O)cc(-c3cnc4c(N5CC6CCC(C5)N6)nc(OC[C@@]56CCCN5C[C@H](F)C6)nc4c3C(C)C)c12 ; C#Cc1c(F)ccc2cc(O)cc(-c3cnc4c(N5CC6CCC(C5)N6)nc(OC[C@@]56CCCN5C[C@H](F)C6)nc4c3C3CC3)c12]

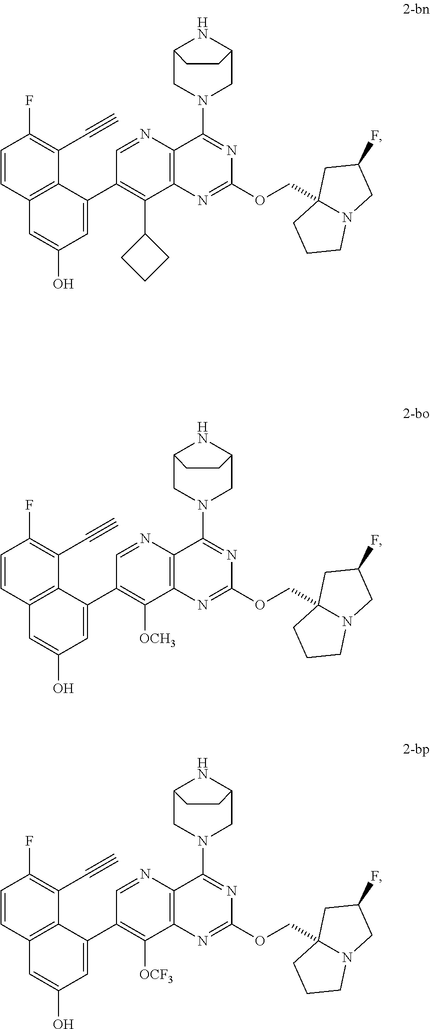 [SMILES: C#Cc1c(F)ccc2cc(O)cc(-c3cnc4c(N5CC6CCC(C5)N6)nc(OC[C@@]56CCCN5C[C@H](F)C6)nc4c3C3CCC3)c12 ; C#Cc1c(F)ccc2cc(O)cc(-c3cnc4c(N5CC6CCC(C5)N6)nc(OC[C@@]56CCCN5C[C@H](F)C6)nc4c3OC)c12 ; C#Cc1c(F)ccc2cc(O)cc(-c3cnc4c(N5CC6CCC(C5)N6)nc(OC[C@@]56CCCN5C[C@H](F)C6)nc4c3OC(F)(F)F)c12]

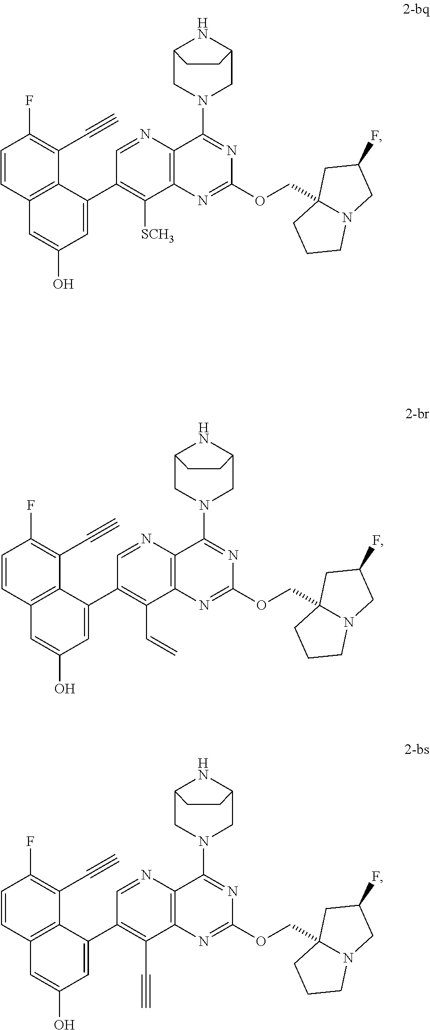 [SMILES: C#Cc1c(F)ccc2cc(O)cc(-c3cnc4c(N5CC6CCC(C5)N6)nc(OC[C@@]56CCCN5C[C@H](F)C6)nc4c3SC)c12 ; C#Cc1c(F)ccc2cc(O)cc(-c3cnc4c(N5CC6CCC(C5)N6)nc(OC[C@@]56CCCN5C[C@H](F)C6)nc4c3C=C)c12 ; C#Cc1c(-c2cc(O)cc3ccc(F)c(C#C)c23)cnc2c(N3CC4CCC(C3)N4)nc(OC[C@@]34CCCN3C[C@H](F)C4)nc12]

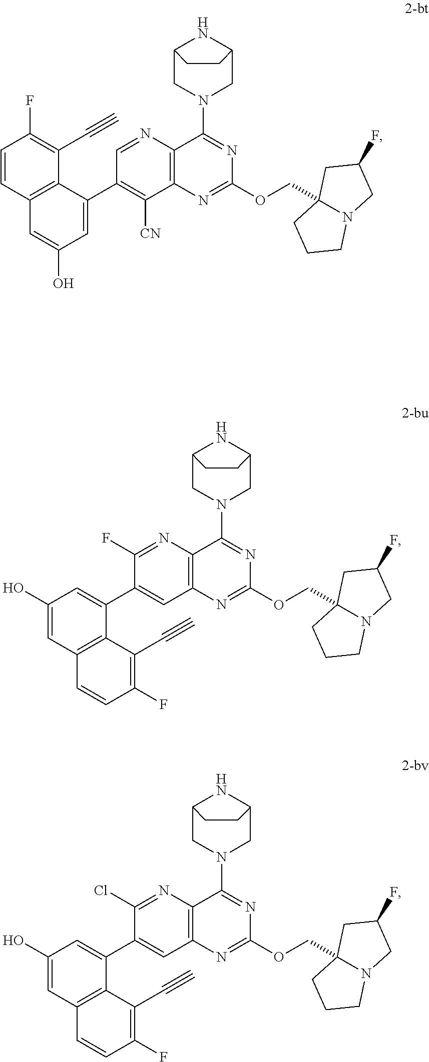 [SMILES: C#Cc1c(F)ccc2cc(O)cc(-c3cnc4c(N5CC6CCC(C5)N6)nc(OC[C@@]56CCCN5C[C@H](F)C6)nc4c3C#N)c12 ; C#Cc1c(F)ccc2cc(O)cc(-c3cc4nc(OC[C@@]56CCCN5C[C@H](F)C6)nc(N5CC6CCC(C5)N6)c4nc3F)c12 ; C#Cc1c(F)ccc2cc(O)cc(-c3cc4nc(OC[C@@]56CCCN5C[C@H](F)C6)nc(N5CC6CCC(C5)N6)c4nc3Cl)c12]

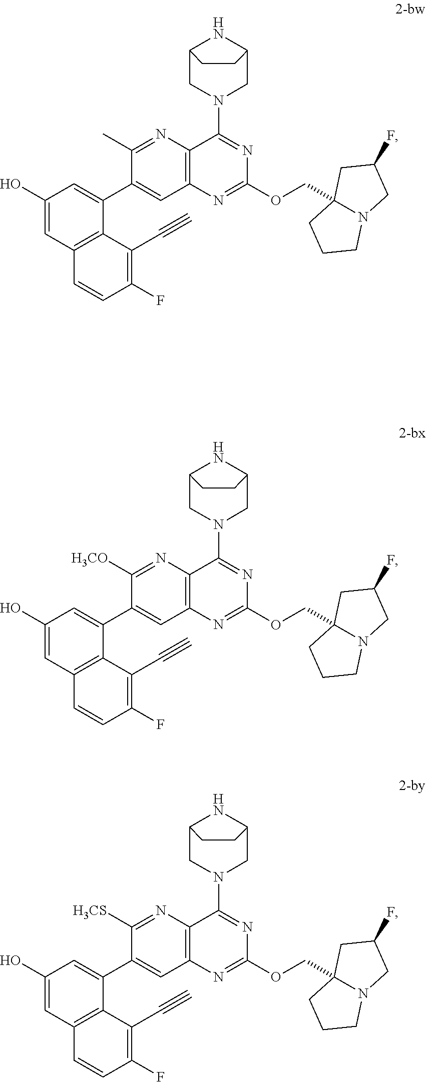

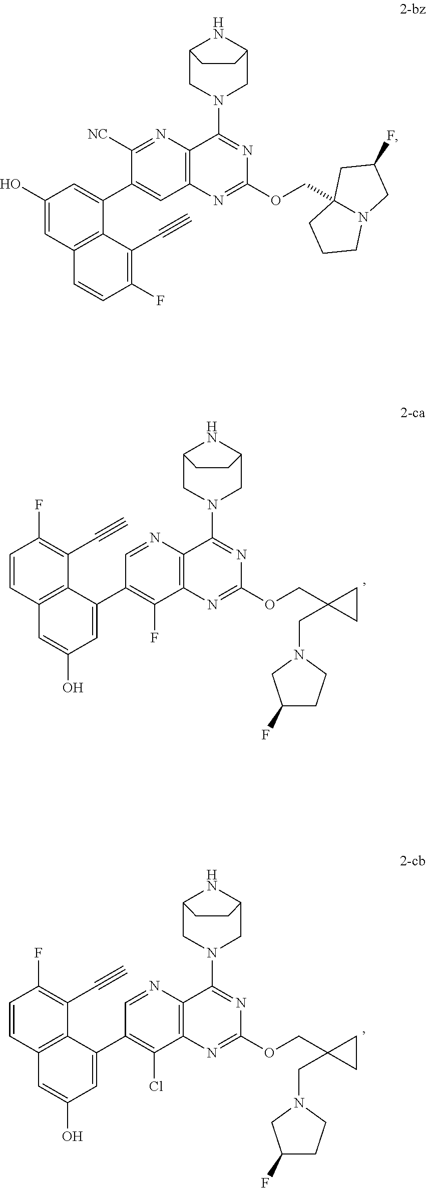 [SMILES: C#Cc1c(F)ccc2cc(O)cc(-c3cc4nc(OC[C@@]56CCCN5C[C@H](F)C6)nc(N5CC6CCC(C5)N6)c4nc3C#N)c12 ; C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cnc4c(N5CC6CCC(C5)N6)nc(OCC5(CN6CC[C@@H](F)C6)CC5)nc4c3Cl)c12]

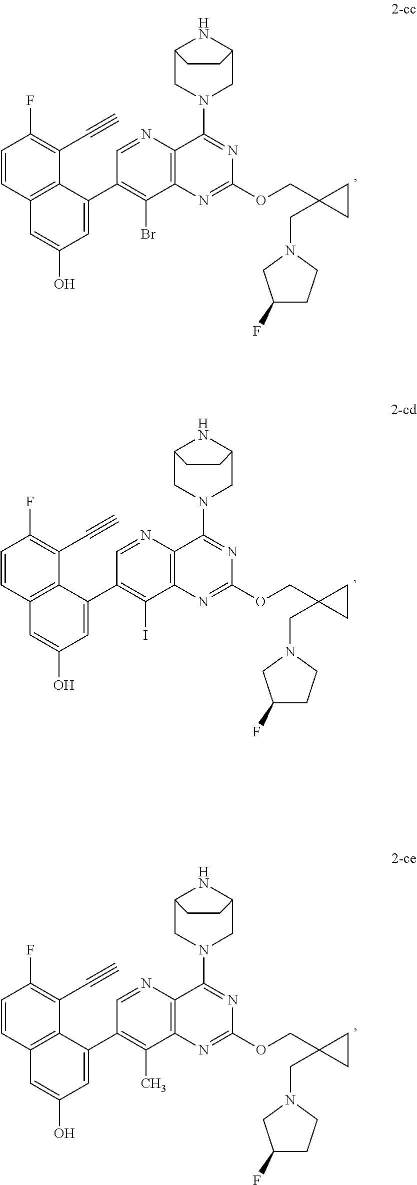 [SMILES: C#Cc1c(F)ccc2cc(O)cc(-c3cnc4c(N5CC6CCC(C5)N6)nc(OCC5(CN6CC[C@@H](F)C6)CC5)nc4c3Br)c12 ; C#Cc1c(F)ccc2cc(O)cc(-c3cnc4c(N5CC6CCC(C5)N6)nc(OCC5(CN6CC[C@@H](F)C6)CC5)nc4c3I)c12 ; C#Cc1c(F)ccc2cc(O)cc(-c3cnc4c(N5CC6CCC(C5)N6)nc(OCC5(CN6CC[C@@H](F)C6)CC5)nc4c3C)c12]

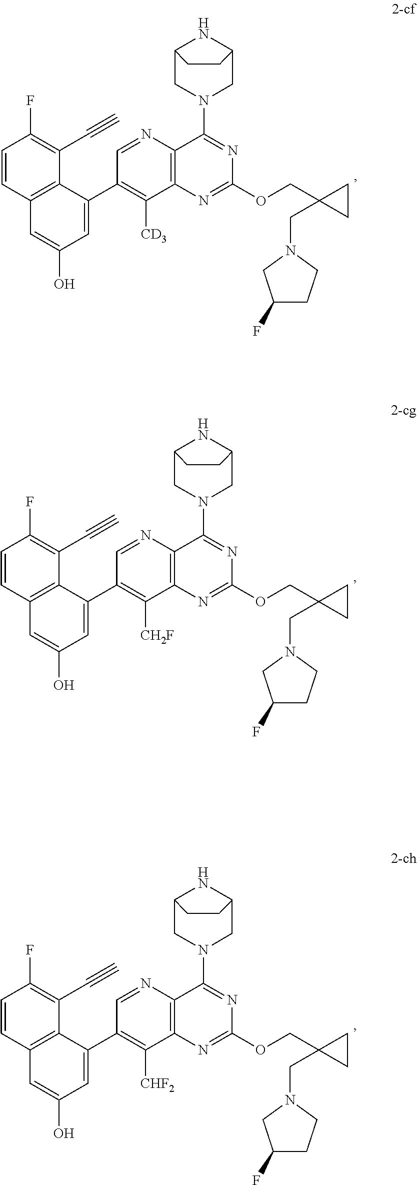

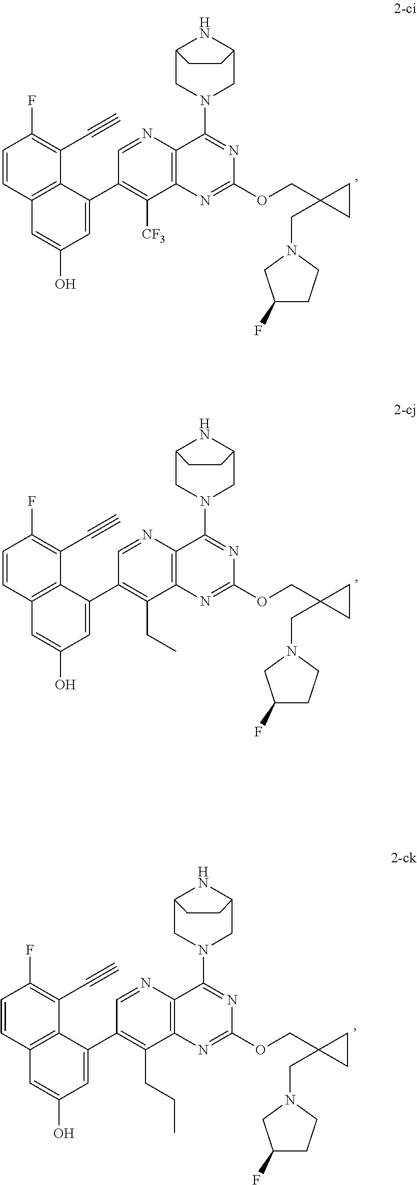 [SMILES: C#Cc1c(F)ccc2cc(O)cc(-c3cnc4c(N5CC6CCC(C5)N6)nc(OCC5(CN6CC[C@@H](F)C6)CC5)nc4c3C(F)(F)F)c12 ; C#Cc1c(F)ccc2cc(O)cc(-c3cnc4c(N5CC6CCC(C5)N6)nc(OCC5(CN6CC[C@@H](F)C6)CC5)nc4c3CC)c12 ; C#Cc1c(F)ccc2cc(O)cc(-c3cnc4c(N5CC6CCC(C5)N6)nc(OCC5(CN6CC[C@@H](F)C6)CC5)nc4c3CCC)c12]

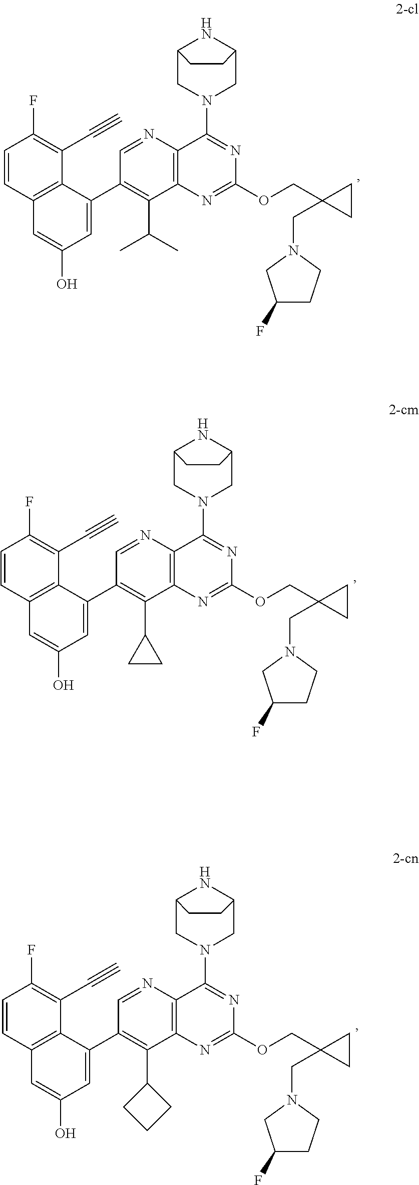 [SMILES: C#Cc1c(F)ccc2cc(O)cc(-c3cnc4c(N5CC6CCC(C5)N6)nc(OCC5(CN6CC[C@@H](F)C6)CC5)nc4c3C(C)C)c12 ; C#Cc1c(F)ccc2cc(O)cc(-c3cnc4c(N5CC6CCC(C5)N6)nc(OCC5(CN6CC[C@@H](F)C6)CC5)nc4c3C3CC3)c12 ; C#Cc1c(F)ccc2cc(O)cc(-c3cnc4c(N5CC6CCC(C5)N6)nc(OCC5(CN6CC[C@@H](F)C6)CC5)nc4c3C3CCC3)c12]

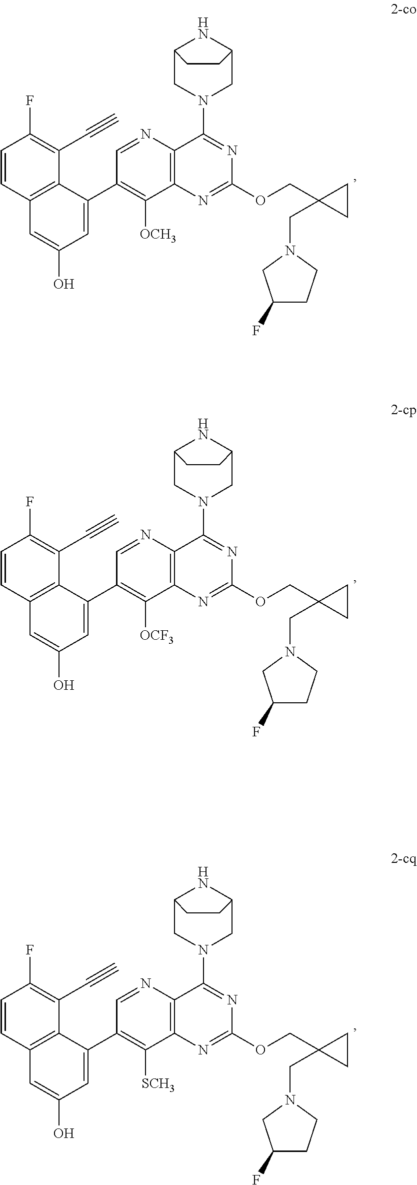 [SMILES: C#Cc1c(F)ccc2cc(O)cc(-c3cnc4c(N5CC6CCC(C5)N6)nc(OCC5(CN6CC[C@@H](F)C6)CC5)nc4c3OC)c12 ; C#Cc1c(F)ccc2cc(O)cc(-c3cnc4c(N5CC6CCC(C5)N6)nc(OCC5(CN6CC[C@@H](F)C6)CC5)nc4c3OC(F)(F)F)c12 ; C#Cc1c(F)ccc2cc(O)cc(-c3cnc4c(N5CC6CCC(C5)N6)nc(OCC5(CN6CC[C@@H](F)C6)CC5)nc4c3SC)c12]

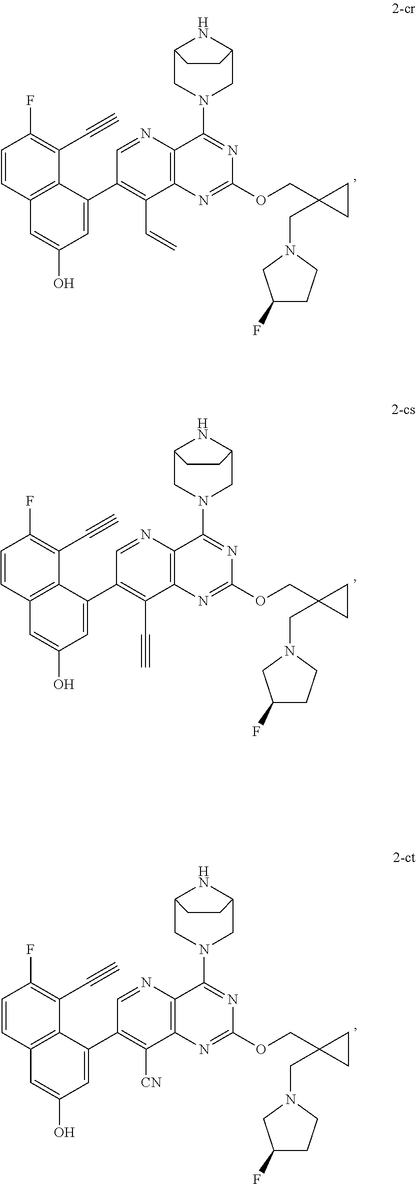 [SMILES: C#Cc1c(F)ccc2cc(O)cc(-c3cnc4c(N5CC6CCC(C5)N6)nc(OCC5(CN6CC[C@@H](F)C6)CC5)nc4c3C=C)c12 ; C#Cc1c(-c2cc(O)cc3ccc(F)c(C#C)c23)cnc2c(N3CC4CCC(C3)N4)nc(OCC3(CN4CC[C@@H](F)C4)CC3)nc12 ; C#Cc1c(F)ccc2cc(O)cc(-c3cnc4c(N5CC6CCC(C5)N6)nc(OCC5(CN6CC[C@@H](F)C6)CC5)nc4c3C#N)c12]

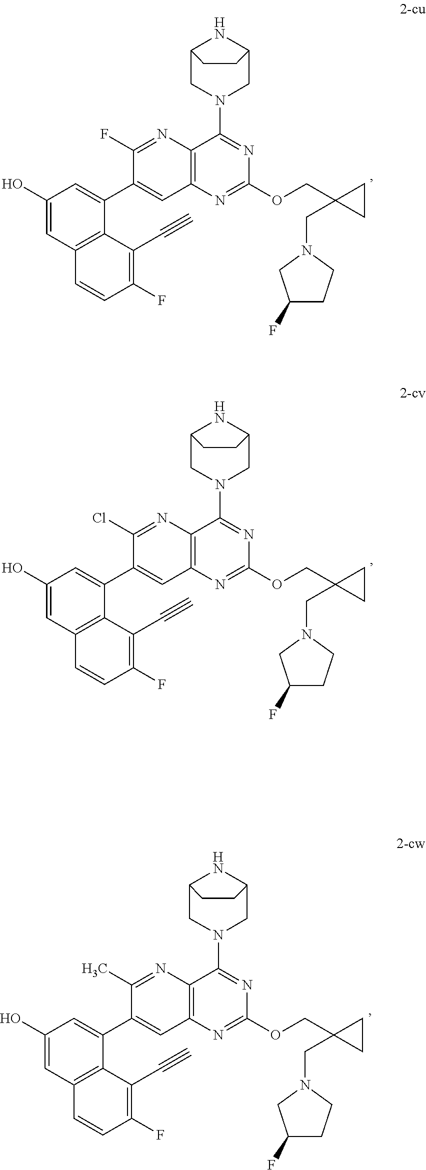 [SMILES: C#Cc1c(F)ccc2cc(O)cc(-c3cc4nc(OCC5(CN6CC[C@@H](F)C6)CC5)nc(N5CC6CCC(C5)N6)c4nc3F)c12 ; C#Cc1c(F)ccc2cc(O)cc(-c3cc4nc(OCC5(CN6CC[C@@H](F)C6)CC5)nc(N5CC6CCC(C5)N6)c4nc3Cl)c12 ; C#Cc1c(F)ccc2cc(O)cc(-c3cc4nc(OCC5(CN6CC[C@@H](F)C6)CC5)nc(N5CC6CCC(C5)N6)c4nc3C)c12]

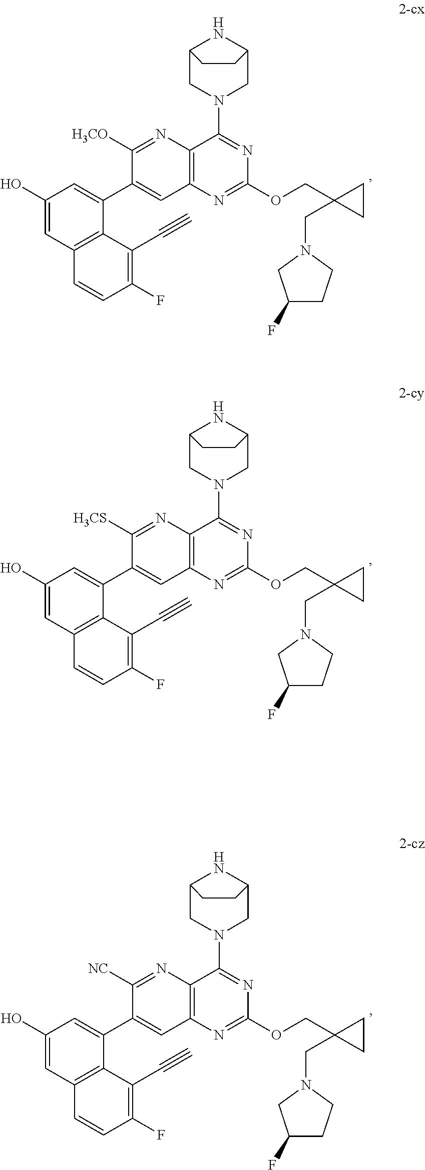

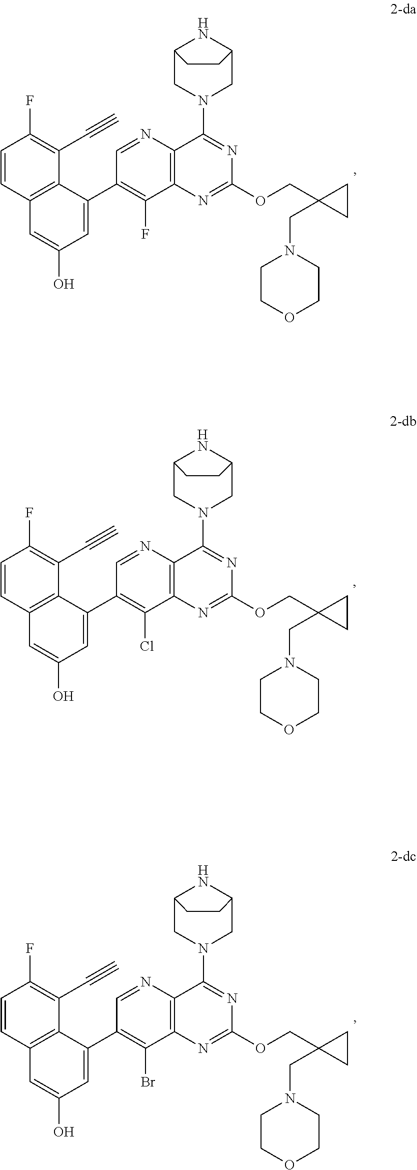 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cnc4c(N5CC6CCC(C5)N6)nc(OCC5(CN6CCOCC6)CC5)nc4c3Cl)c12 ; C#Cc1c(F)ccc2cc(O)cc(-c3cnc4c(N5CC6CCC(C5)N6)nc(OCC5(CN6CCOCC6)CC5)nc4c3Br)c12]

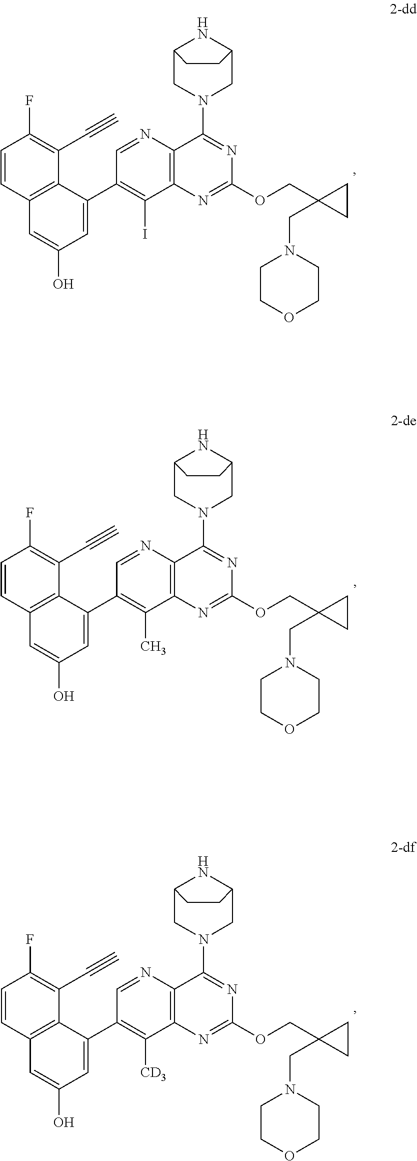 [SMILES: C#Cc1c(F)ccc2cc(O)cc(-c3cnc4c(N5CC6CCC(C5)N6)nc(OCC5(CN6CCOCC6)CC5)nc4c3I)c12 ; C#Cc1c(F)ccc2cc(O)cc(-c3cnc4c(N5CC6CCC(C5)N6)nc(OCC5(CN6CCOCC6)CC5)nc4c3C)c12 ; [2H]C([2H])([2H])c1c(-c2cc(O)cc3ccc(F)c(C#C)c23)cnc2c(N3CC4CCC(C3)N4)nc(OCC3(CN4CCOCC4)CC3)nc12]

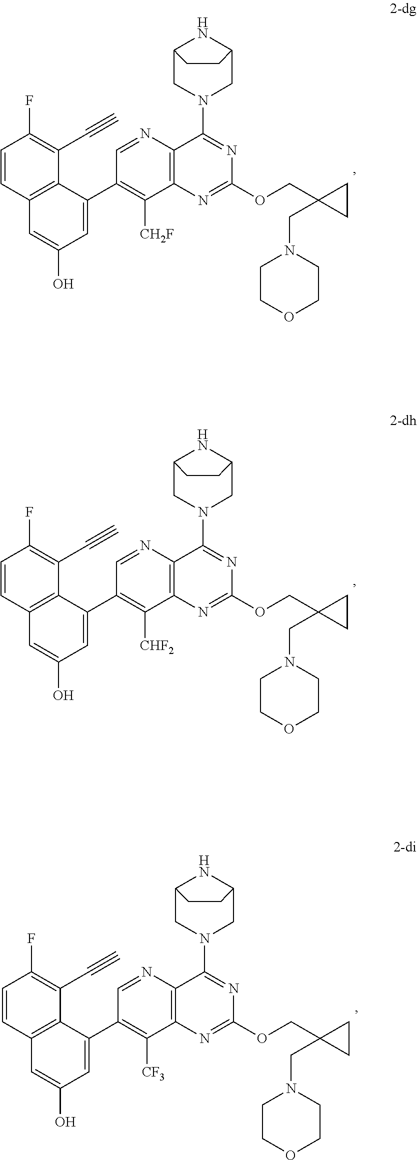

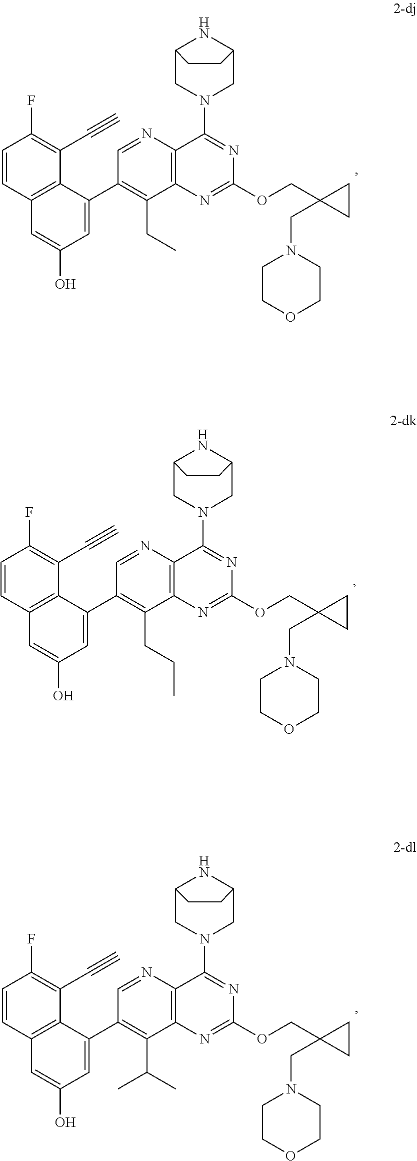 [SMILES: C#Cc1c(F)ccc2cc(O)cc(-c3cnc4c(N5CC6CCC(C5)N6)nc(OCC5(CN6CCOCC6)CC5)nc4c3CC)c12 ; C#Cc1c(F)ccc2cc(O)cc(-c3cnc4c(N5CC6CCC(C5)N6)nc(OCC5(CN6CCOCC6)CC5)nc4c3CCC)c12 ; C#Cc1c(F)ccc2cc(O)cc(-c3cnc4c(N5CC6CCC(C5)N6)nc(OCC5(CN6CCOCC6)CC5)nc4c3C(C)C)c12]

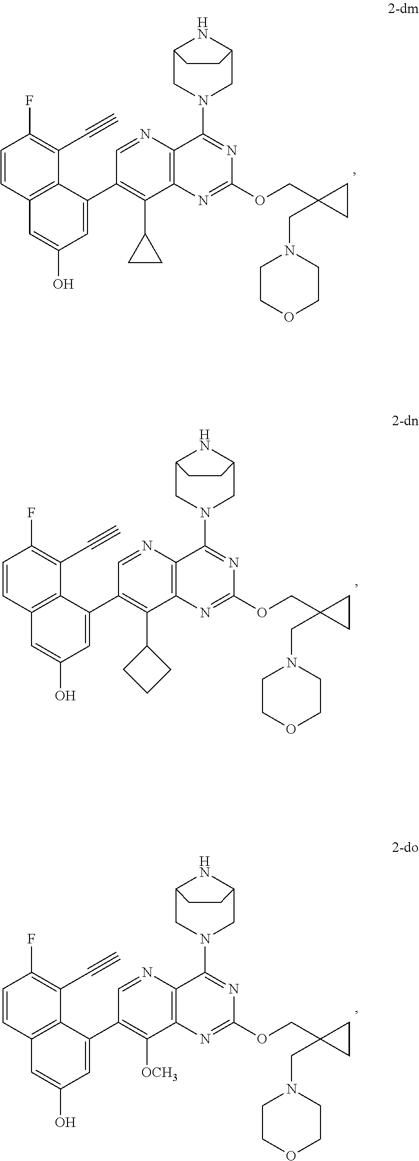 [SMILES: C#Cc1c(F)ccc2cc(O)cc(-c3cnc4c(N5CC6CCC(C5)N6)nc(OCC5(CN6CCOCC6)CC5)nc4c3C3CC3)c12 ; C#Cc1c(F)ccc2cc(O)cc(-c3cnc4c(N5CC6CCC(C5)N6)nc(OCC5(CN6CCOCC6)CC5)nc4c3C3CCC3)c12 ; C#Cc1c(F)ccc2cc(O)cc(-c3cnc4c(N5CC6CCC(C5)N6)nc(OCC5(CN6CCOCC6)CC5)nc4c3OC)c12]

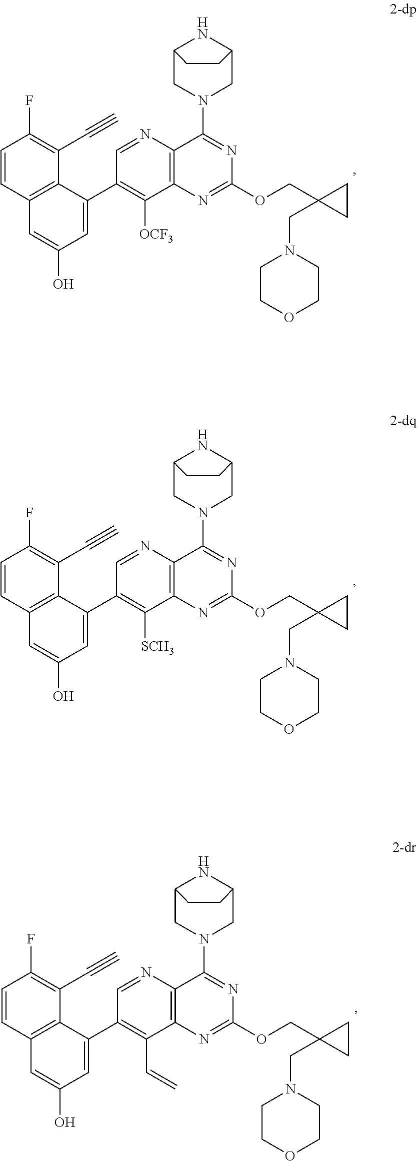 [SMILES: C#Cc1c(F)ccc2cc(O)cc(-c3cnc4c(N5CC6CCC(C5)N6)nc(OCC5(CN6CCOCC6)CC5)nc4c3OC(F)(F)F)c12 ; C#Cc1c(F)ccc2cc(O)cc(-c3cnc4c(N5CC6CCC(C5)N6)nc(OCC5(CN6CCOCC6)CC5)nc4c3SC)c12 ; C#Cc1c(F)ccc2cc(O)cc(-c3cnc4c(N5CC6CCC(C5)N6)nc(OCC5(CN6CCOCC6)CC5)nc4c3C=C)c12]

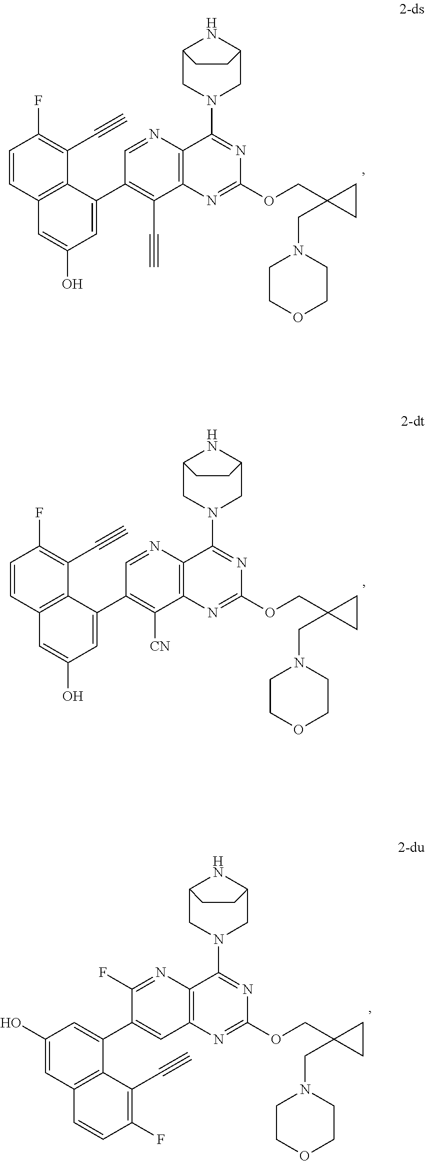 [SMILES: C#Cc1c(-c2cc(O)cc3ccc(F)c(C#C)c23)cnc2c(N3CC4CCC(C3)N4)nc(OCC3(CN4CCOCC4)CC3)nc12 ; C#Cc1c(F)ccc2cc(O)cc(-c3cnc4c(N5CC6CCC(C5)N6)nc(OCC5(CN6CCOCC6)CC5)nc4c3C#N)c12 ; C#Cc1c(F)ccc2cc(O)cc(-c3cc4nc(OCC5(CN6CCOCC6)CC5)nc(N5CC6CCC(C5)N6)c4nc3F)c12]

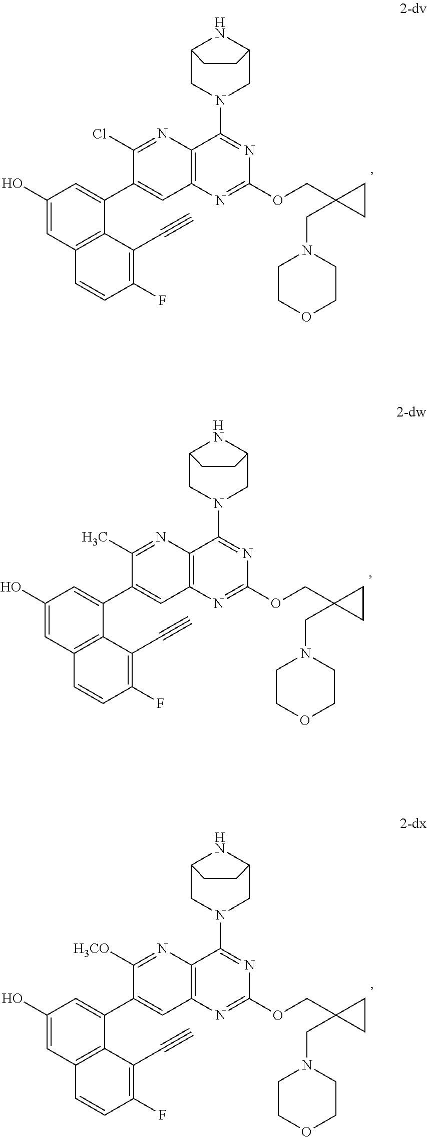 [SMILES: C#Cc1c(F)ccc2cc(O)cc(-c3cc4nc(OCC5(CN6CCOCC6)CC5)nc(N5CC6CCC(C5)N6)c4nc3Cl)c12 ; C#Cc1c(F)ccc2cc(O)cc(-c3cc4nc(OCC5(CN6CCOCC6)CC5)nc(N5CC6CCC(C5)N6)c4nc3C)c12 ; C#Cc1c(F)ccc2cc(O)cc(-c3cc4nc(OCC5(CN6CCOCC6)CC5)nc(N5CC6CCC(C5)N6)c4nc3OC)c12]

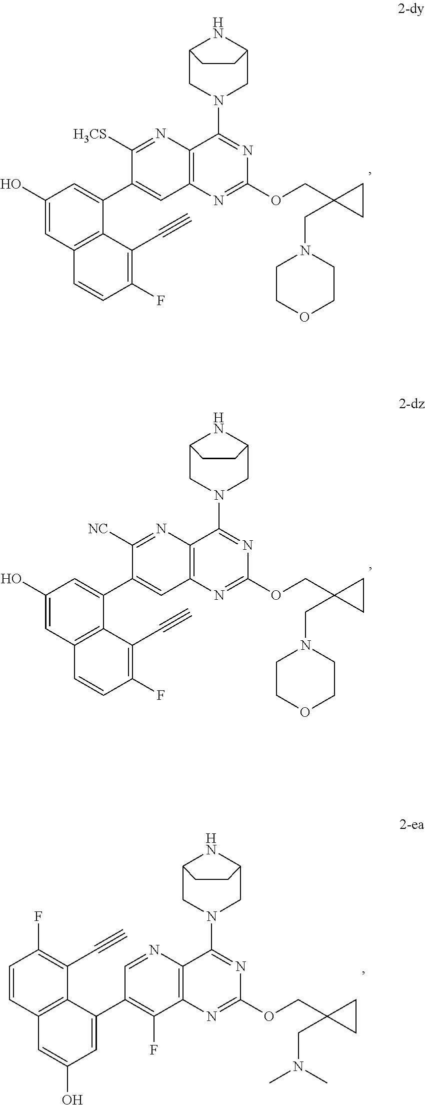

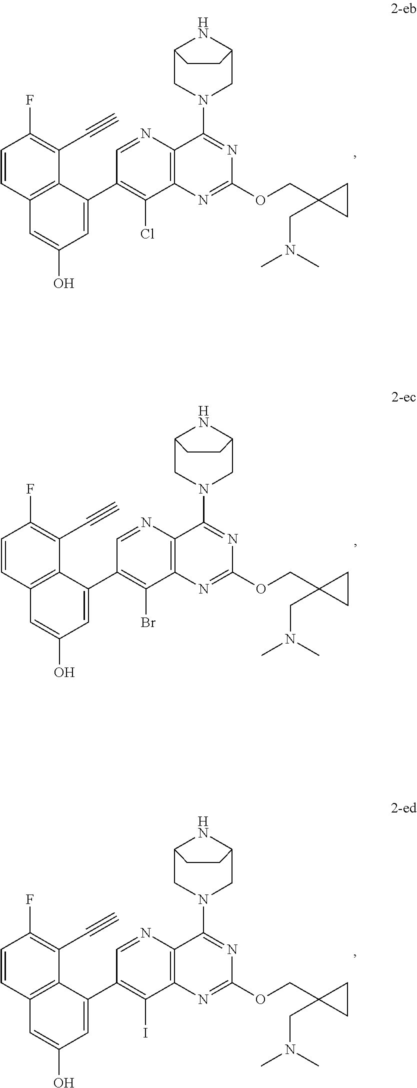 [SMILES: C#Cc1c(F)ccc2cc(O)cc(-c3cnc4c(N5CC6CCC(C5)N6)nc(OCC5(CN(C)C)CC5)nc4c3Cl)c12 ; C#Cc1c(F)ccc2cc(O)cc(-c3cnc4c(N5CC6CCC(C5)N6)nc(OCC5(CN(C)C)CC5)nc4c3Br)c12 ; C#Cc1c(F)ccc2cc(O)cc(-c3cnc4c(N5CC6CCC(C5)N6)nc(OCC5(CN(C)C)CC5)nc4c3I)c12]

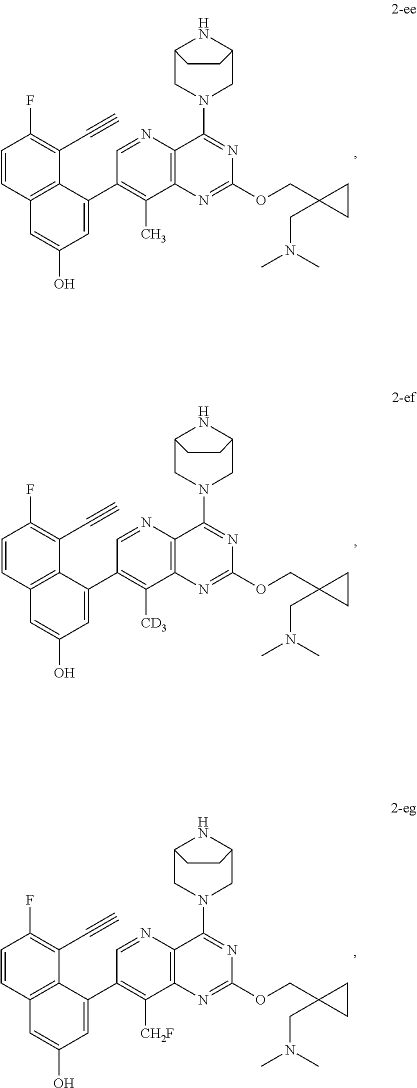

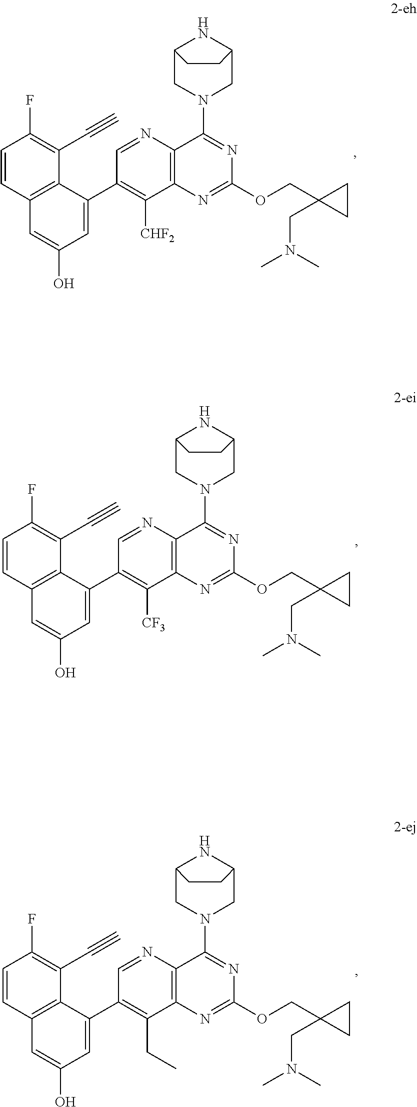 [SMILES: C#Cc1c(F)ccc2cc(O)cc(-c3cnc4c(N5CC6CCC(C5)N6)nc(OCC5(CN(C)C)CC5)nc4c3C(F)F)c12 ; C#Cc1c(F)ccc2cc(O)cc(-c3cnc4c(N5CC6CCC(C5)N6)nc(OCC5(CN(C)C)CC5)nc4c3C(F)(F)F)c12 ; C#Cc1c(F)ccc2cc(O)cc(-c3cnc4c(N5CC6CCC(C5)N6)nc(OCC5(CN(C)C)CC5)nc4c3CC)c12]

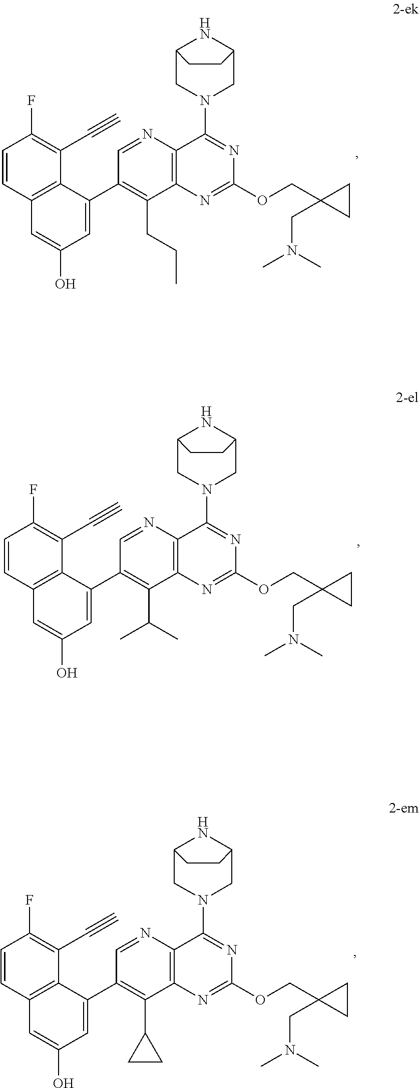 [SMILES: C#Cc1c(F)ccc2cc(O)cc(-c3cnc4c(N5CC6CCC(C5)N6)nc(OCC5(CN(C)C)CC5)nc4c3CCC)c12 ; C#Cc1c(F)ccc2cc(O)cc(-c3cnc4c(N5CC6CCC(C5)N6)nc(OCC5(CN(C)C)CC5)nc4c3C(C)C)c12 ; C#Cc1c(F)ccc2cc(O)cc(-c3cnc4c(N5CC6CCC(C5)N6)nc(OCC5(CN(C)C)CC5)nc4c3C3CC3)c12]

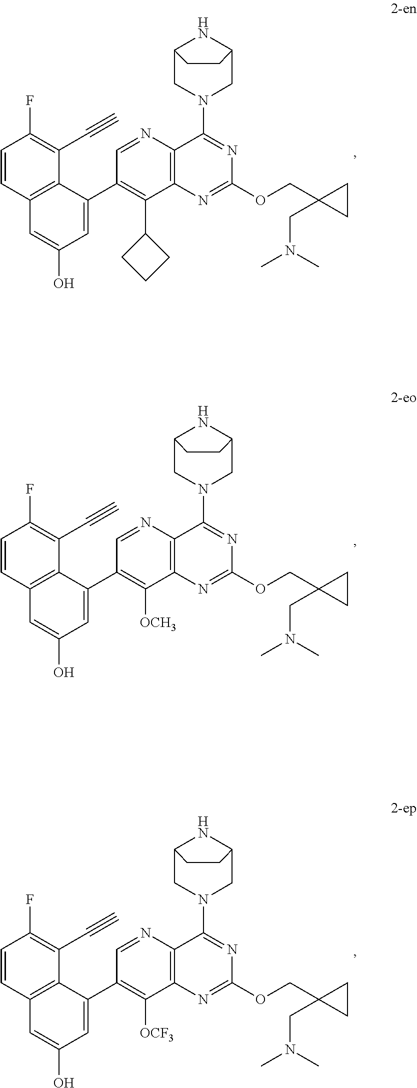 [SMILES: C#Cc1c(F)ccc2cc(O)cc(-c3cnc4c(N5CC6CCC(C5)N6)nc(OCC5(CN(C)C)CC5)nc4c3C3CCC3)c12 ; C#Cc1c(F)ccc2cc(O)cc(-c3cnc4c(N5CC6CCC(C5)N6)nc(OCC5(CN(C)C)CC5)nc4c3OC)c12 ; C#Cc1c(F)ccc2cc(O)cc(-c3cnc4c(N5CC6CCC(C5)N6)nc(OCC5(CN(C)C)CC5)nc4c3OC(F)(F)F)c12]

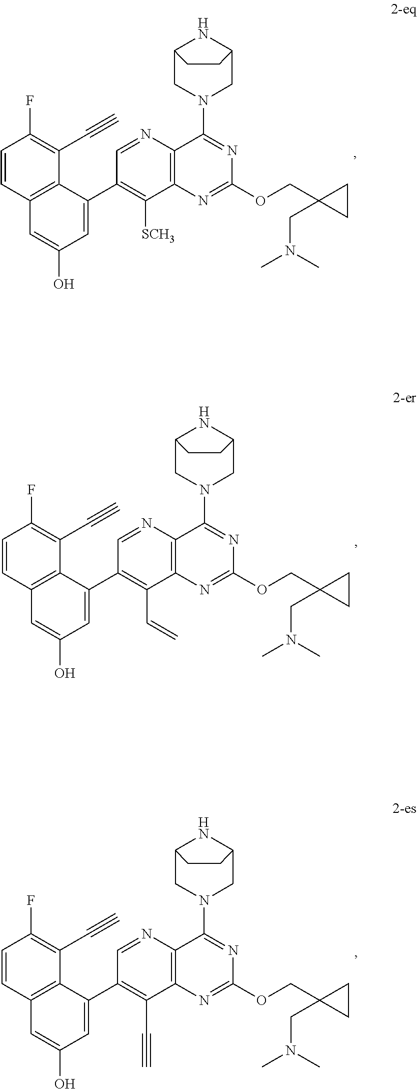 [SMILES: C#Cc1c(F)ccc2cc(O)cc(-c3cnc4c(N5CC6CCC(C5)N6)nc(OCC5(CN(C)C)CC5)nc4c3SC)c12 ; C#Cc1c(F)ccc2cc(O)cc(-c3cnc4c(N5CC6CCC(C5)N6)nc(OCC5(CN(C)C)CC5)nc4c3C=C)c12 ; C#Cc1c(-c2cc(O)cc3ccc(F)c(C#C)c23)cnc2c(N3CC4CCC(C3)N4)nc(OCC3(CN(C)C)CC3)nc12]

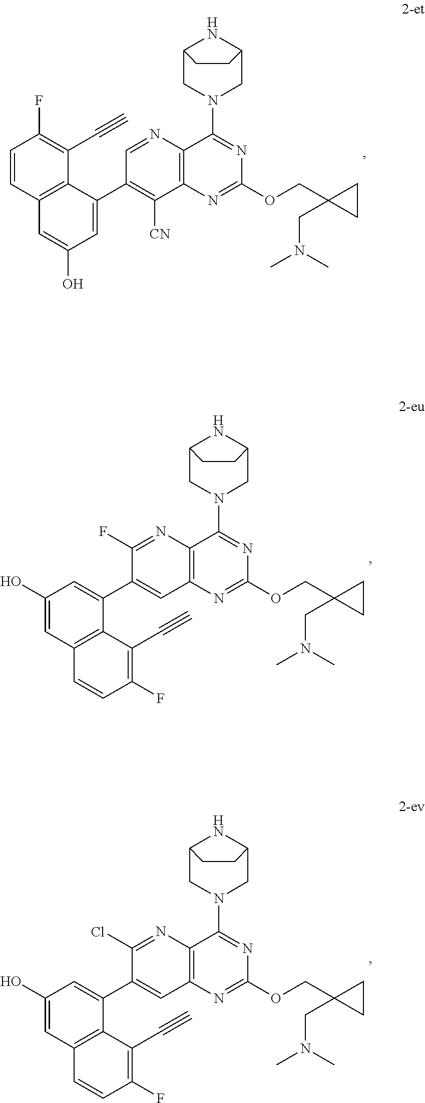 [SMILES: C#Cc1c(F)ccc2cc(O)cc(-c3cnc4c(N5CC6CCC(C5)N6)nc(OCC5(CN(C)C)CC5)nc4c3C#N)c12 ; C#Cc1c(F)ccc2cc(O)cc(-c3cc4nc(OCC5(CN(C)C)CC5)nc(N5CC6CCC(C5)N6)c4nc3F)c12 ; C#Cc1c(F)ccc2cc(O)cc(-c3cc4nc(OCC5(CN(C)C)CC5)nc(N5CC6CCC(C5)N6)c4nc3Cl)c12]

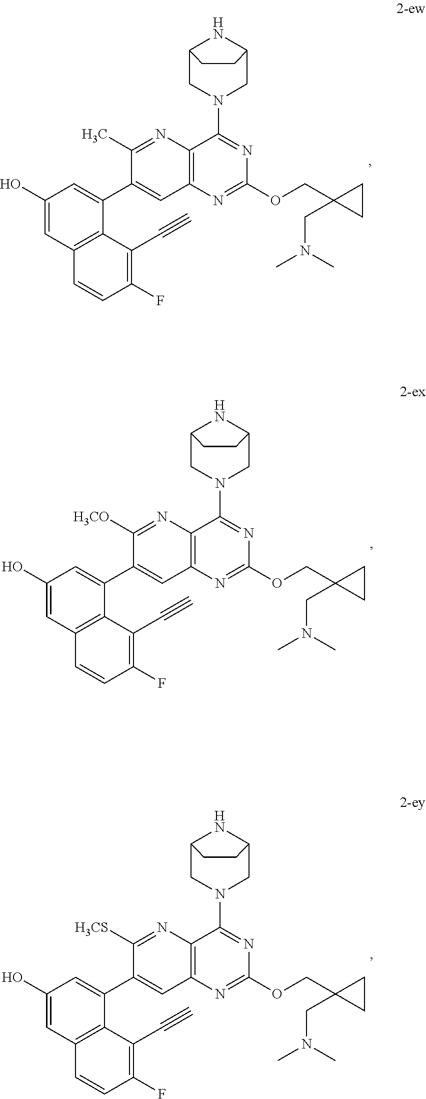

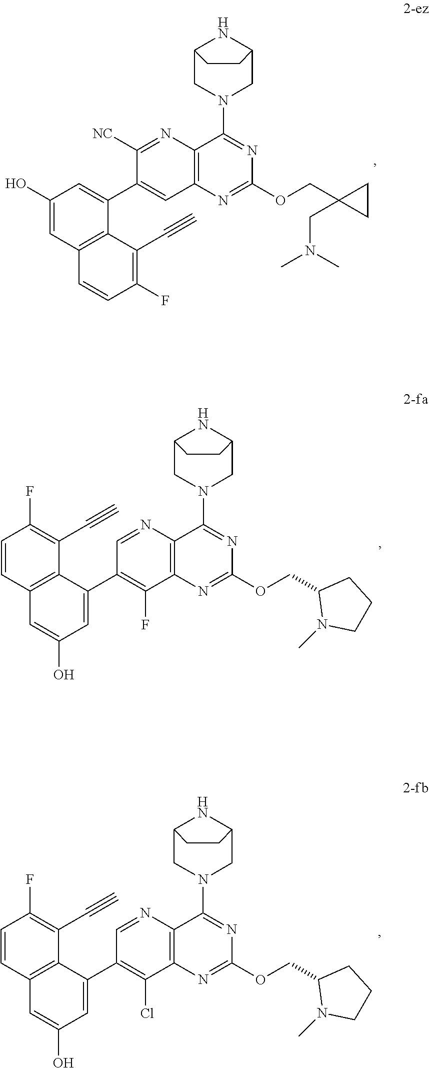

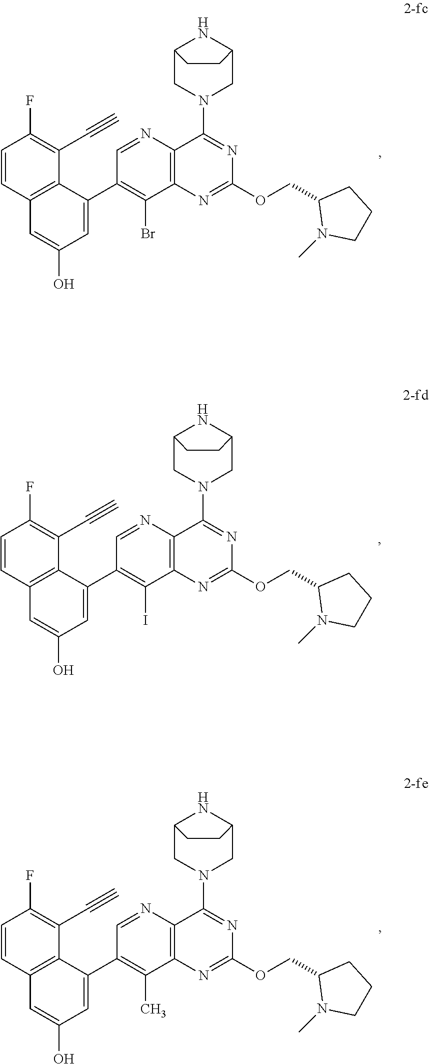

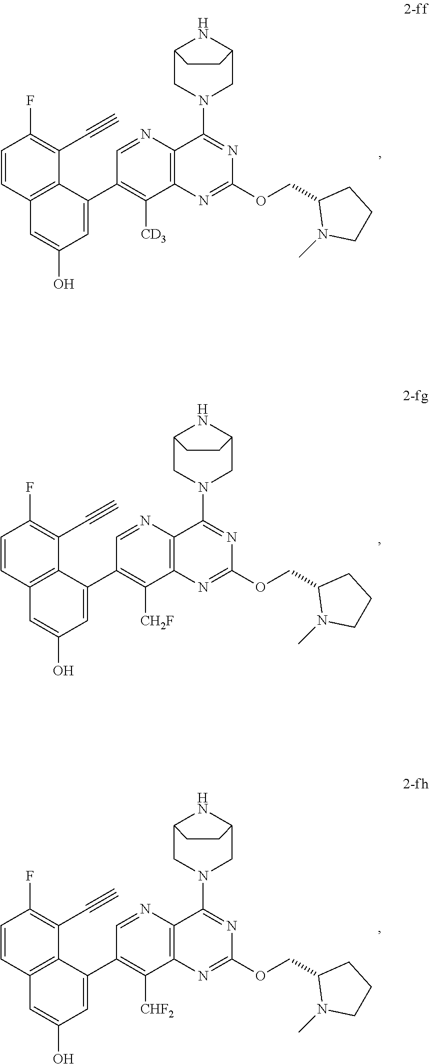

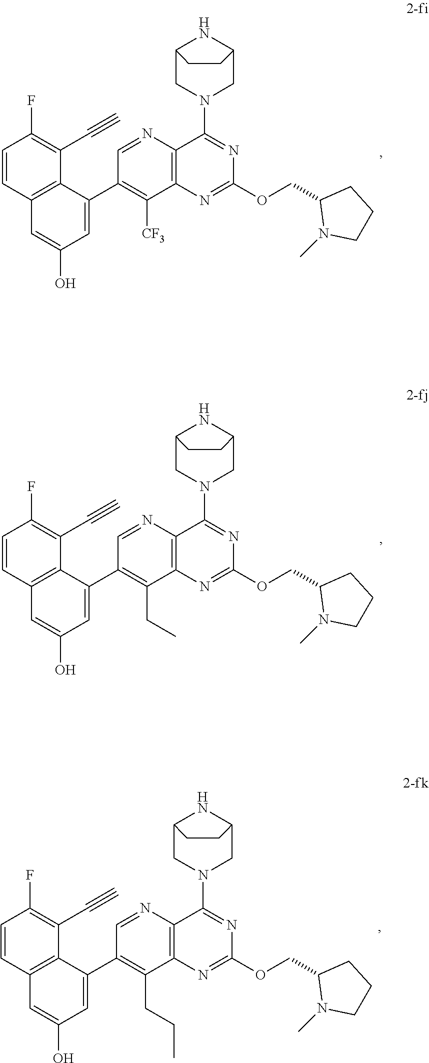

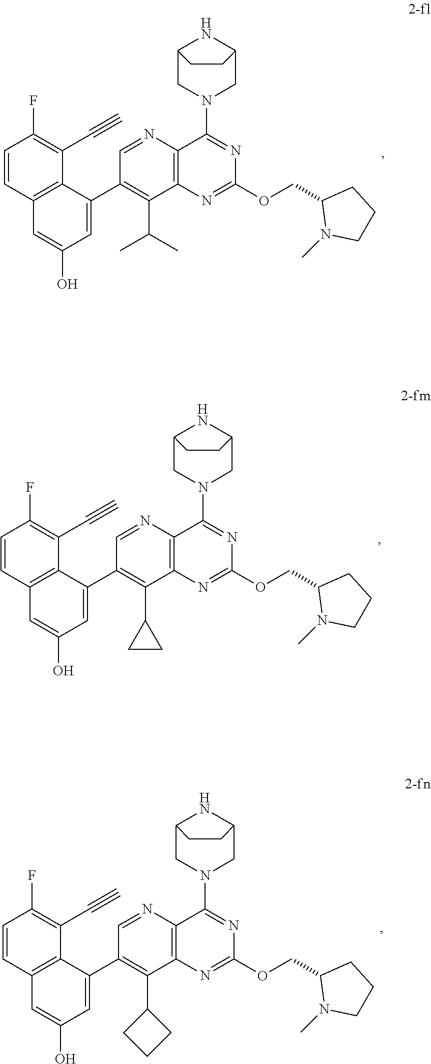

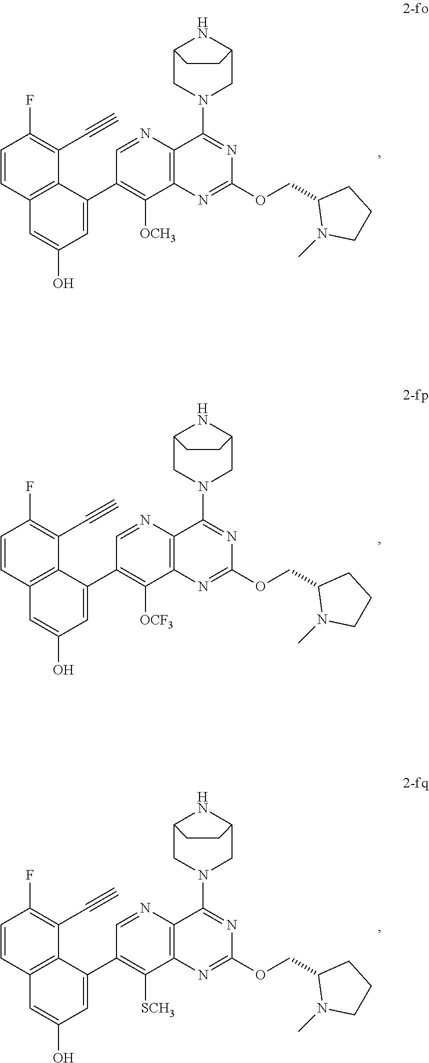

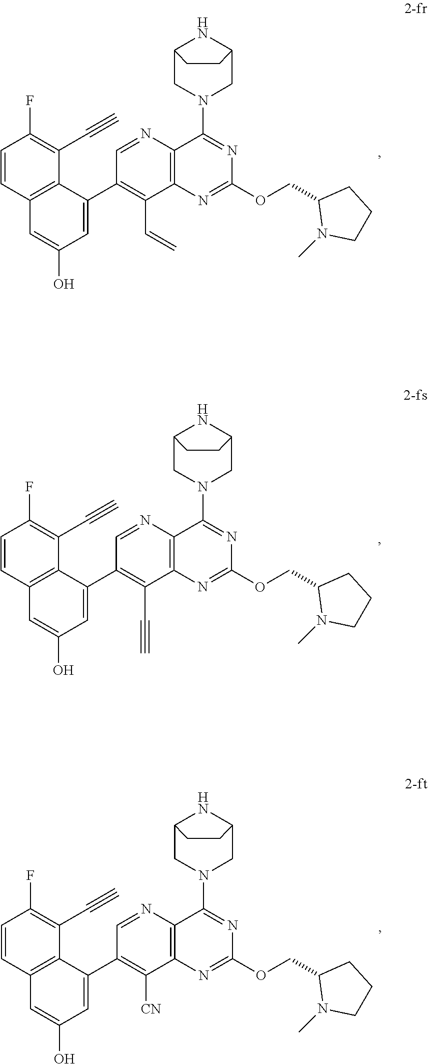

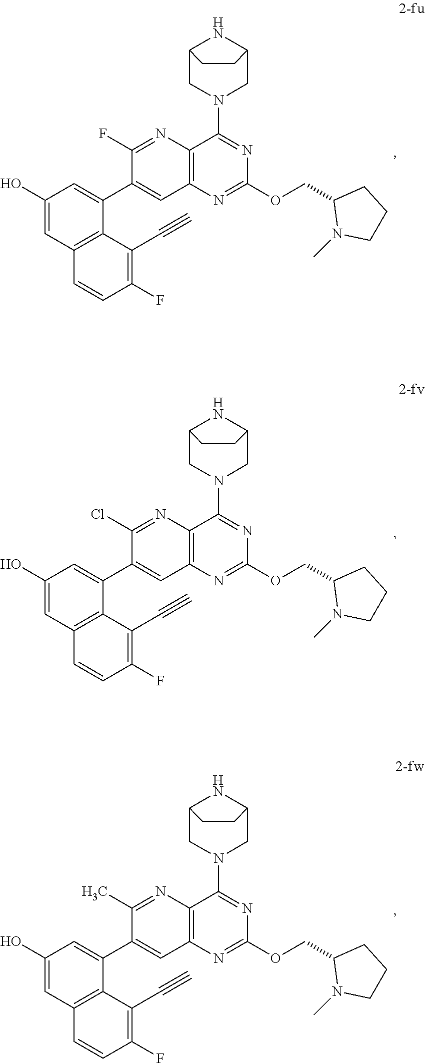

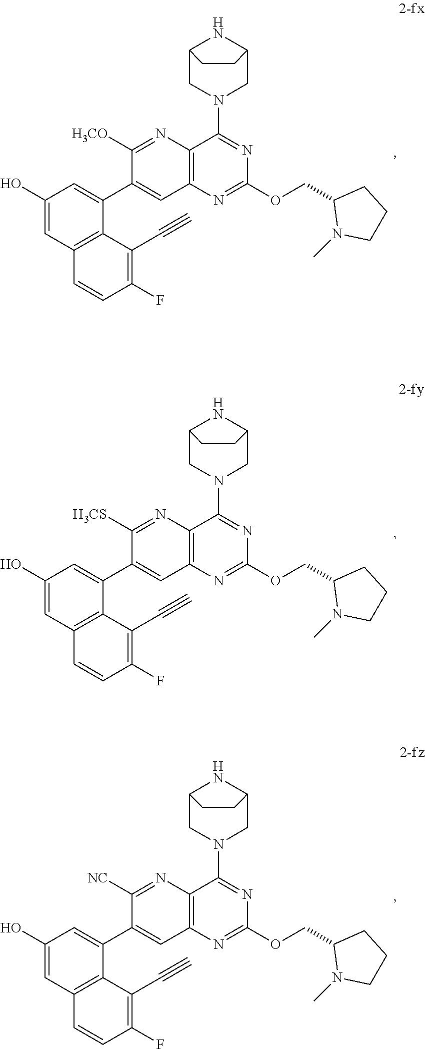

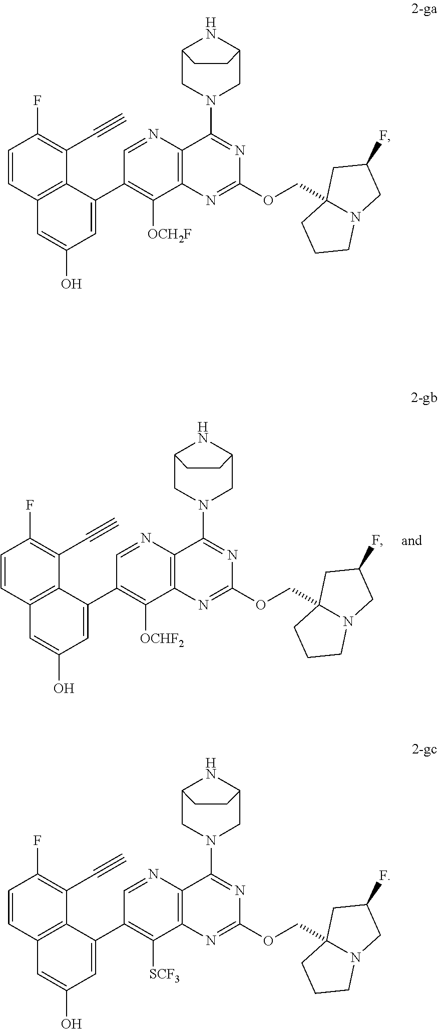

3. A pharmaceutical composition comprising at least one compound of claim 1, or a salt, solvate, or prodrug thereof, together with a pharmaceutically acceptable carrier.

4. A method of treating a disease, disorder, or medical condition in a patient, comprising the step of providing to a patient in need thereof a therapeutic agent, wherein the therapeutic agent comprises at least one compound claim 1, or a salt, solvate, or prodrug thereof.

5. The method of treating a disease, disorder, or medical condition of claim 4, wherein said disease, disorder, or medical condition comprises various cancers.

6. The method of treating a disease, disorder, or medical condition of claim 5, wherein said disease, disorder, or medical condition is mediated through KRAS.

7. The method of treating a disease, disorder, or medical condition of claim 6, wherein said disease, disorder, or medical condition is mediated through the KRAS mutant G12D.

8. The method of treating a disease, disorder, or medical condition of claim 5, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer, pancreatic cancer, and combinations thereof.

9. The method of claim 4, further comprising administering to the patient in need thereof at least one additional therapeutic agent.

10. The method of claim 9, wherein the additional therapeutic agent is selected from doxorubicin, paclitaxel, docetaxel, cisplatin, camptothecin, temozolomide, avastin, Herceptin, Erbitux, EGFR inhibitors, osimertinib, rezivertinib, CDK 4/6 inhibitors, abemaciclib, palbociclib, ribociclib, c-MET inhibitors, capmatinib, volitinib, ALK inhibitors, crizotinib, alectinib, ceritinib, brigatinib, entrectinib, lorlatinib, PD-1 antagonists, PD-L1 antagonists, ipilimumab, embrolizumab, nivolumab, and combinations thereof.

11. The compound of claim 2 wherein the compound of Formula I is selected from at least one compound of 1-ap, 1-bo, 1-ca, 1-dk, 1-ei, 1-gb, 2-ab, 2-af, 2-ba, 2-be, 2-ca, 2-ce, 2-da, 2-de, 2-ea, 2-ee, and 2-fa or a salt, solvate, or prodrug thereof.

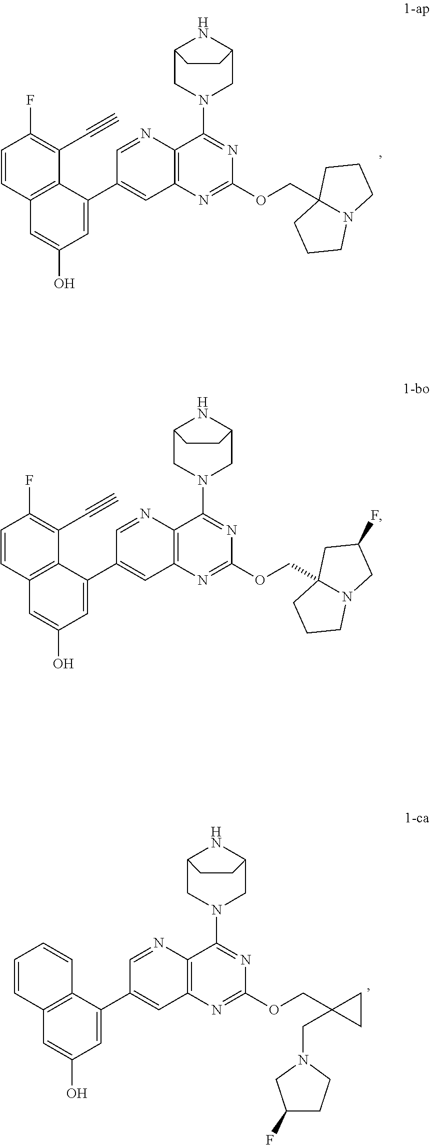 [SMILES: C#Cc1c(F)ccc2cc(O)cc(-c3cnc4c(N5CC6CCC(C5)N6)nc(OCC56CCCN5CCC6)nc4c3)c12 ; C#Cc1c(F)ccc2cc(O)cc(-c3cnc4c(N5CC6CCC(C5)N6)nc(OC[C@@]56CCCN5C[C@H](F)C6)nc4c3)c12 ; Oc1cc(-c2cnc3c(N4CC5CCC(C4)N5)nc(OCC4(CN5CC[C@@H](F)C5)CC4)nc3c2)c2ccccc2c1]

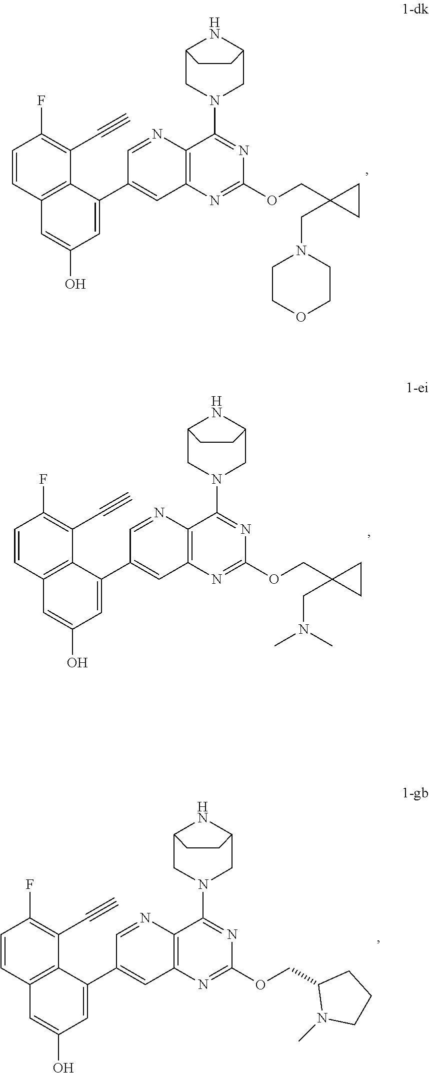

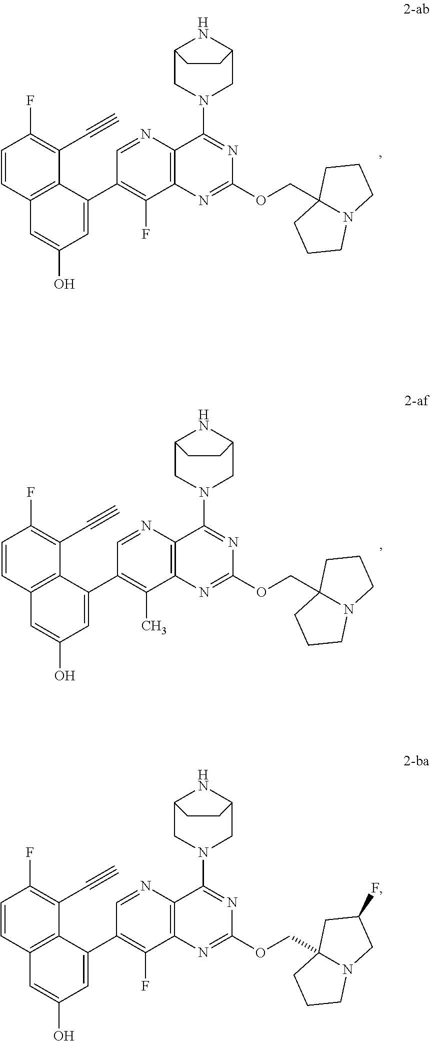 [SMILES: C#Cc1c(F)ccc2cc(O)cc(-c3cnc4c(N5CC6CCC(C5)N6)nc(OCC56CCCN5CCC6)nc4c3F)c12 ; C#Cc1c(F)ccc2cc(O)cc(-c3cnc4c(N5CC6CCC(C5)N6)nc(OCC56CCCN5CCC6)nc4c3C)c12 ; C#Cc1c(F)ccc2cc(O)cc(-c3cnc4c(N5CC6CCC(C5)N6)nc(OC[C@@]56CCCN5C[C@H](F)C6)nc4c3F)c12]

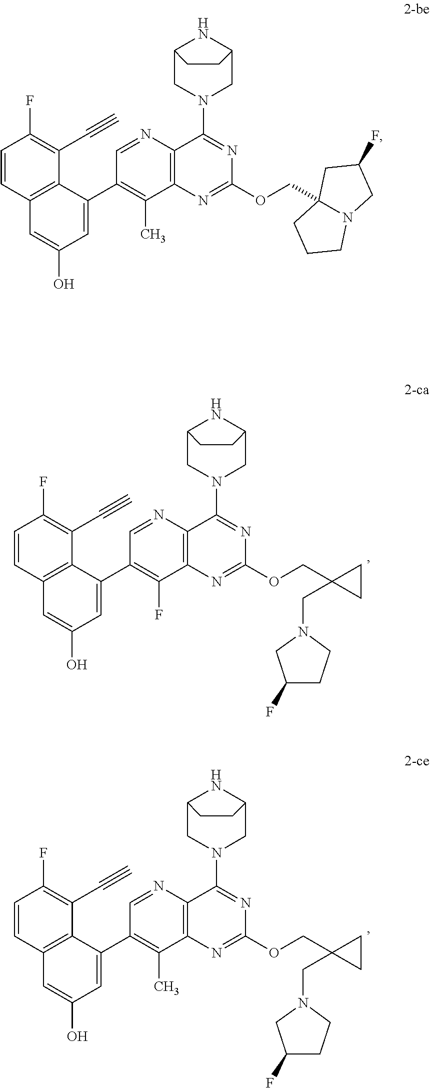 [SMILES: C#Cc1c(F)ccc2cc(O)cc(-c3cnc4c(N5CC6CCC(C5)N6)nc(OC[C@@]56CCCN5C[C@H](F)C6)nc4c3C)c12 ; C#Cc1c(F)ccc2cc(O)cc(-c3cnc4c(N5CC6CCC(C5)N6)nc(OCC5(CN6CC[C@@H](F)C6)CC5)nc4c3F)c12 ; C#Cc1c(F)ccc2cc(O)cc(-c3cnc4c(N5CC6CCC(C5)N6)nc(OCC5(CN6CC[C@@H](F)C6)CC5)nc4c3C)c12]

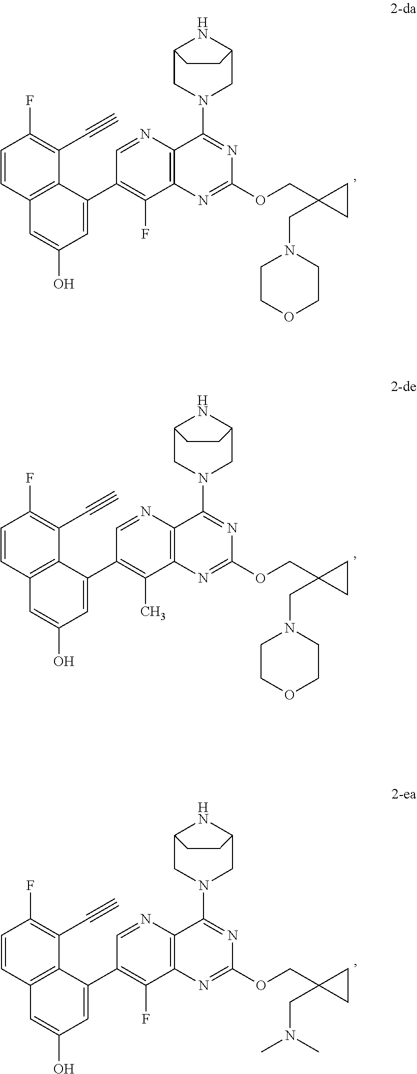 [SMILES: C#Cc1c(F)ccc2cc(O)cc(-c3cnc4c(N5CC6CCC(C5)N6)nc(OCC5(CN6CCOCC6)CC5)nc4c3F)c12 ; C#Cc1c(F)ccc2cc(O)cc(-c3cnc4c(N5CC6CCC(C5)N6)nc(OCC5(CN6CCOCC6)CC5)nc4c3C)c12 ; C#Cc1c(F)ccc2cc(O)cc(-c3cnc4c(N5CC6CCC(C5)N6)nc(OCC5(CN(C)C)CC5)nc4c3F)c12]

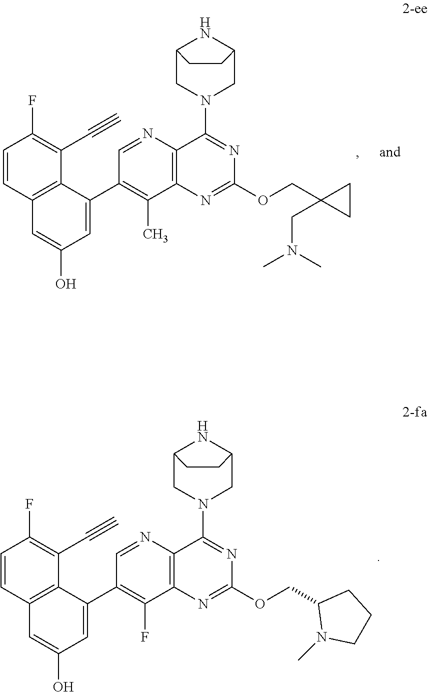

12. The pharmaceutical composition of claim 3, comprising at least one compound of 1-ap, 1-bo, 1-ca, 1-dk, 1-ei or 1-gb or a salt, solvate, or prodrug thereof, together with a pharmaceutically acceptable carrier.

13. The method of treating a disease, disorder or medical condition of claim 4, comprising the step of providing to a patient in need thereof the pharmaceutical composition of claim 12.

14. The method of claim 13, wherein said disease, disorder or medical condition comprises various cancers.

15. The method of treating a disease, disorder or medical condition of claim 13, wherein said disease, disorder, or medical condition is mediated through KRAS.

16. The method of treating a disease, disorder or medical condition of claim 15, wherein said disease, disorder, or medical condition is mediated through KRAS mutant G12D.

17. The method of treating a disease, disorder or medical condition of claim 14, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer, pancreatic cancer, and combinations thereof.

18. A method for treating a KRAS G12D-associated cancer, comprising administering to a patient in need thereof a therapeutic agent, wherein the therapeutic agent comprises at least one compound of claim 1, or a salt, solvate, or prodrug thereof.

19. The method of treating a KRAS G12D-associated cancer of claim 18, wherein said cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, small cell lung cancer, cholangiocarcinomas, chondrosarcoma, colon cancer, colorectal cancer, rectal cancer, pancreatic cancer and combinations thereof.

20. The method of claim 18, further comprising administering to the patient in need thereof at least one additional therapeutic agent.

21. The method of claim 20, wherein the additional therapeutic agent is selected from doxorubicin, paclitaxel, docetaxel, cisplatin, camptothecin, temozolomide, avastin, Herceptin, Erbitux, EGFR inhibitors, osimertinib, rezivertinib, CDK 4/6 inhibitors, abemaciclib, palbociclib, ribociclib, c-MET inhibitors, capmatinib, volitinib, ALK inhibitors, crizotinib, alectinib, ceritinib, brigatinib, entrectinib, lorlatinib, PD-1 antagonists, PD-L1 antagonists, ipilimumab, embrolizumab, nivolumab and combinations thereof.
